(19) United States
(12) Patent Application Publication
(10) Pub. No.: US 2023/0031818 A1
(43) Pub. Date: Feb. 2, 2023

(54) METHODS AND COMPOSITIONS FOR TREATING COLORECTAL CANCER

(71) Applicant: Second Genome, Inc., Brisbane, CA (US)

(72) Inventors: Jayamary Divya Ravichandar, Brisbane, CA (US); Todd Zachary DeSantis, Brisbane, CA (US)

(21) Appl. No.: 17/758,777

(22) PCT Filed: Jan. 15, 2021
(86) PCT No.: PCT/US2021/013751

§ 371 Date: Jul. 13, 2022

Related U.S. Application Data
(60) Provisional application No. 62/962,827, filed on Jan. 17, 2020.

(51) Int. Cl. C12Q 1/689; A61K 35/74; A61P 35/00; C12Q 1/6886; A61K 9/20; A61K 9/00

(52) U.S. Cl. CPC C12Q 1/689; A61K 35/74; A61P 35/00; C12Q 1/6886; A61K 9/20; A61K 9/0031; C12Q 2600/112

(57) ABSTRACT

Methods and compositions are provided herein for treating colorectal cancer in a subject, using one or more bacterial strains such as Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, or Streptococcus hongkongensis HKU30.

### CROSS-REFERENCE TO RELATED APPLICATION

This application claims priority to U.S. Provisional Patent Application Ser. No. 62/962,827, filed Jan. 17, 2020; the entire contents of which are herein incorporated by reference.

### DESCRIPTION OF THE TEXT FILE SUBMITTED ELECTRONICALLY

The contents of the text file submitted electronically herewith are incorporated herein by reference in their entirety: A computer readable format copy of the Sequence Listing filename: 47192_0049P01.txt, date created, Jan. 17, 2020, file size≈410 kilobytes.

### TECHNICAL FIELD

The present disclosure is related to bacterial strains and compositions thereof, and using such bacterial strains and compositions thereof for treating colorectal cancer in a subject.

### BACKGROUND

The microbiome of the gastrointestinal tract comprises a diverse array of microorganisms, primarily prokaryotes, which play a significant role in the health of the host organism. The complexity of the microbiome, in terms of both its population makeup and composite function, has recently become an intense area of study as research increasingly shows that manipulation of the microbiome can provide health benefits and may be effective in treating a number of diseases and disorders. Currently, a number of probiotics are marketed which contain live bacteria and yeast and are believed to augment the benefits of these microbes which naturally occur in the human body. Increasingly, live biotherapeutic products (LBPs) are being developed for controlled clinical studies and regulatory approval in the treatment of disease.

Colorectal cancer is the third most prevalent cancer worldwide and is increasing in individuals less than 50 years old. Within the gastrointestinal tract, the colon is the section that is more prone to developing cancer (see, e.g., Saus et al. Mol. Aspects Med. 2019 October; 69:93-106), and it is estimated that the colon contains approximately 70% of all microbes in the human body (Sekirov et al. Physiol. Rev. 2010 July; 90(3):859-904). Colorectal cancer has also been associated with specific dietary factors and eating patterns that affect the gut microbiota (Saus et al. Mol. Aspects Med. 2019 October; 69:93-106). Moreover, some studies have reported alterations in the gut microbiome that are associated with colorectal cancer. Early diagnosis of colorectal cancer (e.g., a tumor in stages 0, I or II) is associated with an 80% survival rate over five years. However, the survival rate decreases to only 10% if the colorectal cancer is diagnosed later (stage IV). Microbe-based therapies could aid in the prevention and treatment of colorectal cancer.

### SUMMARY

Provided herein are methods and compositions for treating a subject in need thereof.

Provided herein are methods of identifying a subject as having colorectal cancer that include (a) identifying a subject having a sample that has (i) an increased level of one or more (e.g. two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or twenty-four) bacterial species selected from the group consisting of: Alistipes indistinctus, Azotobacter sp., Bacteroides asaccharolyticus, Bacteroides sp., Bilophila wadsworthia, Clostridium aldenense, Clostridium bolteae, Clostridium lactatifermentans, Clostridium sp., Desulfovibrio piger, Dialister pneumosintes, Eubacterium biforme, Fusobacterium mortiferum, Hungatella hathewayi, Megasphaera micronuciformis, Micromonas micros, Paraprevotella clara, Peptostreptococcus stomatis, Porphyromonas somerae, Roseburia inulinivorans, Ruminococcus gnavus, Shigella dysenteriae, Subdoligranulum variabile, Veillonella alcalescens; and/or (ii) a decreased level of one or more (e.g. two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, or twenty-one) bacterial species selected from the group consisting of: Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, and Streptococcus hongkongensis, as having colorectal cancer; or (b) identifying a subject having a sample that does not have (i) an increased level of one or more (e.g. two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or twenty-four) bacterial species selected from the group consisting of: Alistipes indistinctus, Azotobacter sp., Bacteroides asaccharolyticus, Bacteroides sp., Bilophila wadsworthia, Clostridium aldenense, Clostridium bolteae, Clostridium lactatifermentans, Clostridium sp., Desulfovibrio piger, Dialister pneumosintes, Eubacterium biforme, Fusobacterium mortiferum, Hungatella hathewayi, Megasphaera micronuciformis, Micromonas micros, Paraprevotella clara, Peptostreptococcus stomatis, Porphyromonas somerae, Roseburia inulinivorans, Ruminococcus gnavus, Shigella dysenteriae, Subdoligranulum variabile, Veillonella alcalescens; and/or (ii) a decreased level of one or more (e.g. two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, or twenty-one) bacterial species selected from the group consisting of: Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, and Streptococcus hongkongensis, as not having colorectal cancer.

Also provided herein are methods of diagnosing a subject as having colorectal cancer that include (a) identifying a subject having a sample that has (i) an increased level of one or more (e.g. two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or twenty-four) bacterial species selected from the group consisting of: Alistipes indistinctus, Azotobacter sp., Bacteroides asaccharolyticus, Bacteroides sp., Bilophila wadsworthia, Clostridium aldenense, Clostridium bolteae, Clostridium lactatifermentans, Clostridium sp., Desulfovibrio piger, Dialister pneumosintes, Eubacterium biforme, Fusobacterium mortiferum, Hungatella hathewayi, Megasphaera micronuciformis, Micromonas micros, Paraprevotella clara, Peptostreptococcus stomatis, Porphyromonas somerae, Roseburia inulinivorans, Ruminococcus gnavus, Shigella dysenteriae, Subdoligranulum variabile, Veillonella alcalescens; and/or (ii) a decreased level of one or more (e.g. two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, or twenty-one) bacterial species selected from the group consisting of: Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, and Streptococcus hongkongensis, as having colorectal cancer; or (b) identifying a subject having a sample that does not have (i) an increased level of one or more (e.g. two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or twenty-four) bacterial species selected from the group consisting of: Alistipes indistinctus, Azotobacter sp., Bacteroides asaccharolyticus, Bacteroides sp., Bilophila wadsworthia, Clostridium aldenense, Clostridium bolteae, Clostridium lactatifermentans, Clostridium sp., Desulfovibrio piger, Dialister pneumosintes, Eubacterium biforme, Fusobacterium mortiferum, Hungatella hathewayi, Megasphaera micronuciformis, Micromonas micros, Paraprevotella clara, Peptostreptococcus stomatis, Porphyromonas somerae, Roseburia inulinivorans, Ruminococcus gnavus, Shigella dysenteriae, Subdoligranulum variabile, Veillonella alcalescens; and/or (ii) a decreased level of one or more (e.g. two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, or twenty-one) bacterial species selected from the group consisting of: Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, and Streptococcus hongkongensis, as not having colorectal cancer.

Also provided herein are methods for treating a subject in need thereof that include administering to the subject a composition comprising an effective amount of a bacterial strain selected from the group consisting of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, and a combination thereof. In some embodiments of any of the methods herein, the subject has colorectal cancer.

Also provided herein are methods for treating colorectal cancer in a subject that include administering to the subject a composition comprising an effective amount of a bacterial species selected from the group consisting of: Bacteroides eggerthii, Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, Streptococcus hongkongensis, and a combination thereof.

Also provided herein are methods for treating colorectal cancer in a subject that include (a) detecting a dysbiosis associated with colorectal cancer in a sample from the subject; and (b) administering to the subject a composition comprising an effective amount of a bacterial species selected from the group consisting of: Bacteroides eggerthii, Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, Streptococcus hongkongensis, and a combination thereof.

In some embodiments of any of the methods herein, the sample is a fecal sample. In some embodiments of any of the methods herein, the sample is a biopsy sample. In some embodiments of any of the methods herein, the biopsy sample is a colorectal biopsy sample.

In some embodiments of any of the methods herein, detecting the dysbiosis associated with colorectal cancer comprises determining bacterial gene expression in the sample from the subject. In some embodiments of any of the methods herein, detecting the dysbiosis associated with colorectal cancer comprises determining bacterial composition in the sample from the subject. In some embodiments of any of the methods herein, detecting the dysbiosis associated with colorectal cancer comprises determining that Alistipes indistinctus, Azotobacter sp., Bacteroides asaccharolyticus, Bacteroides sp., Bilophila wadsworthia, Clostridium aldenense, Clostridium bolteae, Clostridium lactatifermentans, Clostridium sp., Desulfovibrio piger, Dialister pneumosintes, Eubacterium biforme, Fusobacterium mortiferum, Hungatella hathewayi, Megasphaera micronuciformis, Micromonas micros, Paraprevotella clara, Peptostreptococcus stomatis, Porphyromonas somerae, Roseburia inulinivorans, Ruminococcus gnavus, Shigella dysenteriae, Subdoligranulum variabile, Veillonella alcalescens, or a combination thereof, is increased in the sample from subject.

In some embodiments of any of the methods herein, detecting the dysbiosis associated with colorectal cancer comprises determining that Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, Streptococcus hongkongensis, or a combination thereof, is decreased in the sample from subject. In some embodiments of any of the methods herein, Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, Streptococcus hongkongensis, or a combination thereof, is decreased in the gastrointestinal tract of the subject.

Also provided herein are methods for treating a subject in need thereof that include decreasing a population of an increased bacterial strain in the subject, wherein the increased bacterial species is selected from the group consisting of: Alistipes indistinctus, Azotobacter sp., Bacteroides asaccharolyticus, Bacteroides sp., Bilophila wadsworthia, Clostridium aldenense, Clostridium bolteae, Clostridium lactatifermentans, Clostridium sp., Desulfovibrio piger, Dialister pneumosintes, Eubacterium biforme, Fusobacterium mortiferum, Hungatella hathewayi, Megasphaera micronuciformis, Micromonas micros, Paraprevotella clara, Peptostreptococcus stomatis, Porphyromonas somerae, Roseburia inulinivorans, Ruminococcus gnavus, Shigella dysenteriae, Subdoligranulum variabile, Veillonella alcalescens, and a combination thereof. In some embodiments of any of the methods herein, the subject has colorectal cancer.

In some embodiments of any of the methods herein, decreasing the population of an increased bacterial strain comprises administering to the subject a bacteriophage. In some embodiments of any of the methods herein, decreasing the population of an increased bacterial species comprises administering to the subject a composition comprising an effective amount of a bacterial species selected from the group consisting of: Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, Streptococcus hongkongensis, and a combination thereof.

In some embodiments of any of the methods herein, the colorectal cancer is adenocarcinoma, a carcinoid tumor, a gastrointestinal stromal tumor, or lymphoma.

In some embodiments of any of the methods herein, the bacterial species Bacteroides eggerthii comprises the strain Bacteroides eggerthii 1 2 48FAA. In some embodiments of any of the methods herein, the bacterial species Bacteroides eggerthii comprises the strain Bacteroides eggerthii CCUG 9559. In some embodiments of any of the methods herein, the bacterial species Bacteroides goldsteinii comprises the strain Bacteroides goldsteinii ATCC BAA 1180. In some embodiments of any of the methods herein, the bacterial species Bacteroides pectinophilus comprises the strain Bacteroides pectinophilus N3. In some embodiments of any of the methods herein, the bacterial species Bacteroides plebeius comprises the strain Bacteroides plebeius M12. In some embodiments of any of the methods herein, the bacterial species Bacteroides vulgatus comprises the strain Bacteroides vulgatus 8482. In some embodiments of any of the methods herein, the bacterial species Barnesiella intestinihominis comprises the strain Barnesiella intestinihominis DSM 21032. In some embodiments of any of the methods herein, the bacterial species Bifidobacterium stercoris comprises the strain Bifidobacterium stercoris EG1. In some embodiments of any of the methods herein, the bacterial species Clostridium sp. comprises the strain Clostridium sp. 40. In some embodiments of any of the methods herein, the bacterial species Clostridium spiroforme comprises the strain Clostridium spiroforme CCM 6168. In some embodiments of any of the methods herein, the bacterial species Eubacterium eligens comprises the strain Eubacterium eligens DSM 3376. In some embodiments of any of the methods herein, the bacterial species Eubacterium hallii comprises the strain Eubacterium hallii DSM 3353. In some embodiments of any of the methods herein, the bacterial species Megamonas funiformis comprises the species Megamonas funiformis DSM 19343. In some embodiments of any of the methods herein, the bacterial species Megasphaera elsdenii comprises the strain Megasphaera elsdenii LC1. In some embodiments of any of the methods herein, the bacterial species Parabacteroides johnsonii comprises the strain Parabacteroides johnsonii M-165. In some embodiments of any of the methods herein, the bacterial species Butyricimonas virosa comprises the strain Butyricimonas virosa MT12. In some embodiments of any of the methods herein, the bacterial species Clostridium citroniae comprises the strain Clostridium citroniae DSM 19261. In some embodiments of any of the methods herein, the bacterial species Lactobacillus ruminis comprises the strain Lactobacillus ruminis RF3. In some embodiments of any of the methods herein, the bacterial species Methanosphaera stadtmanae comprises the strain Methanosphaera stadtmanae MCB-3. In some embodiments of any of the methods herein, the bacterial species Ruminococcus sp. comprises the strain Ruminococcus sp. 18P13. In some embodiments of any of the methods herein, the bacterial species Slackia piriformis comprises the strain Slackia piriformis DSM 22477. In some embodiments of any of the methods herein, the bacterial species Streptococcus hongkongensis comprises the strain Streptococcus hongkongensis HKU30.

In some embodiments of any of the methods herein, the bacterial species improves intestinal barrier function of the subject.

In some embodiments of any of the methods herein, the Bacteroides eggerthii 1 2 48FAA has a 16S RNA gene that is at least 95% identical to SEQ ID NO:1. In some embodiments of any of the methods herein, the Bacteroides eggerthii 1 2 48FAA has a 16S RNA gene that is at least 95% identical to SEQ ID NO:2. In some embodiments of any of the methods herein, the Bacteroides eggerthii 1 2 48FAA has a 16S RNA gene that is at least 95% identical to SEQ ID NO:3. In some embodiments of any of the methods herein, the Bacteroides eggerthii 1 2 48FAA has a 16S RNA gene that is at least 95% identical to SEQ ID NO:4. In some embodiments of any of the methods herein, the Bacteroides eggerthii 1 2 48FAA has a 16S RNA gene that is at least 95% identical to SEQ ID NO:5. In some embodiments of any of the methods herein, the Bacteroides eggerthii 1 2 48FAA has a 16S RNA gene that is at least 95% identical to SEQ ID NO:6.

In some embodiments of any of the methods herein, the Bacteroides eggerthii CCUG 9559 has a 16S RNA gene that is at least 95% identical to SEQ ID NO:7. In some embodiments of any of the methods herein, the Bacteroides eggerthii CCUG 9559 has a 16S RNA gene that is at least 95% identical to SEQ ID NO:8. In some embodiments of any of the methods herein, the Bacteroides eggerthii CCUG 9559 has a 16S RNA gene that is at least 95% identical to SEQ ID NO:9. In some embodiments of any of the methods herein, the Bacteroides eggerthii CCUG 9559 has a 16S RNA gene that is at least 95% identical to SEQ ID NO:10. In some embodiments of any of the methods herein, the Bacteroides eggerthii CCUG 9559 has a 16S RNA gene that is at least 95% identical to SEQ ID NO:11. In some embodiments of any of the methods herein, the Bacteroides eggerthii CCUG 9559 has a 16S RNA gene that is at least 95% identical to SEQ ID NO:12.

In some embodiments of any of the methods herein, the Bacteroides goldsteinii ATCC BAA 1180 has a 16S RNA gene that is at least 95% identical to SEQ ID NO:13. In some embodiments of any of the methods herein, the Bacteroides goldsteinii ATCC BAA 1180 has a 16S RNA gene that is at least 95% identical to SEQ ID NO:14. In some embodiments of any of the methods herein, the Bacteroides goldsteinii ATCC BAA 1180 has a 16S RNA gene that is at least 95% identical to SEQ ID NO:15. In some embodiments of any of the methods herein, the Bacteroides goldsteinii ATCC BAA 1180 has a 16S RNA gene that is at least 95% identical to SEQ ID NO:16. In some embodiments of any of the methods herein, the Bacteroides goldsteinii ATCC BAA 1180 has a 16S RNA gene that is at least 95% identical to SEQ ID NO:17. In some embodiments of any of the methods herein, the Bacteroides goldsteinii ATCC BAA 1180 has a 16S RNA gene that is at least 95% identical to SEQ ID NO:18. In some embodiments of any of the methods herein, the Bacteroides goldsteinii ATCC BAA 1180 has a 16S RNA gene that is at least 95% identical to SEQ ID NO:19.

In some embodiments of any of the methods herein, the Bacteroides pectinophilus N3 has a 16S RNA gene that is at least 95% identical to SEQ ID NO:20. In some embodiments of any of the methods herein, the Bacteroides pectinophilus N3 has a 16S RNA gene that is at least 95% identical to SEQ ID NO:21. In some embodiments of any of the methods herein, the Bacteroides pectinophilus N3 has a 16S RNA gene that is at least 95% identical to SEQ ID NO:22.

In some embodiments of any of the methods herein, the Bacteroides pectinophilus N3 has a 16S RNA gene that is at least 95% identical to SEQ ID NO:23. In some embodiments of any of the methods herein, the Bacteroides pectinophilus N3 has a 16S RNA gene that is at least 95% identical to SEQ ID NO:24. In some embodiments of any of the methods herein, the Bacteroides pectinophilus N3 has a 16S RNA gene that is at least 95% identical to SEQ ID NO:25.

In some embodiments of any of the methods herein, the Bacteroides plebeius M12 has a 16S RNA gene that is at least 95% identical to SEQ ID NO:26. In some embodiments of any of the methods herein, the Bacteroides plebeius M12 has a 16S RNA gene that is at least 95% identical to SEQ ID NO:27. In some embodiments of any of the methods herein, the Bacteroides plebeius M12 has a 16S RNA gene that is at least 95% identical to SEQ ID NO:28.

In some embodiments of any of the methods herein, the Bacteroides vulgatus 8482 has a 16S RNA gene that is at least 95% identical to SEQ ID NO:29. In some embodiments of any of the methods herein, the Bacteroides vulgatus 8482 has a 16S RNA gene that is at least 95% identical to SEQ ID NO:30. In some embodiments of any of the methods herein, any one of claims 25-71, the Bacteroides vulgatus 8482 has a 16S RNA gene that is at least 95% identical to SEQ ID NO:31. In some embodiments of any of the methods herein, any one of claims 25-71, the Bacteroides vulgatus 8482 has a 16S RNA gene that is at least 95% identical to SEQ ID NO:32. In some embodiments of any of the methods herein, any one of claims 25-71, the Bacteroides vulgatus 8482 has a 16S RNA gene that is at least 95% identical to SEQ ID NO:33. In some embodiments of any of the methods herein, any one of claims 25-71, the Bacteroides vulgatus 8482 has a 16S RNA gene that is at least 95% identical to SEQ ID NO:34. In some embodiments of any of the methods herein, the Bacteroides vulgatus 8482 has a 16S RNA gene that is at least 95% identical to SEQ ID NO:35. In some embodiments of any of the methods herein, the Bacteroides vulgatus 8482 has a 16S RNA gene that is at least 95% identical to SEQ ID NO:36.

In some embodiments of any of the methods herein, the Bacteroides vulgatus 8482 has a 16S RNA gene that is at least 95% identical to SEQ ID NO:37. In some embodiments of any of the methods herein, the Bacteroides vulgatus 8482 has a 16S RNA gene that is at least 95% identical to SEQ ID NO:38. In some embodiments of any of the methods herein, the Bacteroides vulgatus 8482 has a 16S RNA gene that is at least 95% identical to SEQ ID NO:39. In some embodiments of any of the methods herein, the Bacteroides vulgatus 8482 has a 16S RNA gene that is at least 95% identical to SEQ ID NO:40.

In some embodiments of any of the methods herein, the Barnesiella intestinihominis DSM 21032 has a 16S RNA gene that is at least 95% identical to SEQ ID NO:41. In some embodiments of any of the methods herein, the Barnesiella intestinihominis DSM 21032 has a 16S RNA gene that is at least 95% identical to SEQ ID NO:42. In some embodiments of any of the methods herein, the Barnesiella intestinihominis DSM 21032 has a 16S RNA gene that is at least 95% identical to SEQ ID NO:43. In some embodiments of any of the methods herein, the Barnesiella intestinihominis DSM 21032 has a 16S RNA gene that is at least 95% identical to SEQ ID NO:44. In some embodiments of any of the methods herein, the Barnesiella intestinihominis DSM 21032 has a 16S RNA gene that is at least 95% identical to SEQ ID NO:45. In some embodiments of any of the methods herein, the Barnesiella intestinihominis DSM 21032 has a 16S RNA gene that is at least 95% identical to SEQ ID NO: 46. In some embodiments of any of the methods herein, the Barnesiella intestinihominis DSM 21032 has a 16S RNA gene that is at least 95% identical to SEQ ID NO:47. In some embodiments of any of the methods herein, the Barnesiella intestinihominis DSM 21032 has a 16S RNA gene that is at least 95% identical to SEQ ID NO:48. In some embodiments of any of the methods herein, the Barnesiella intestinihominis DSM 21032 has a 16S RNA gene that is at least 95% identical to SEQ ID NO:49. In some embodiments of any of the methods herein, the Barnesiella intestinihominis DSM 21032 has a 16S RNA gene that is at least 95% identical to SEQ ID NO:50.

In some embodiments of any of the methods herein, the Bifidobacterium stercoris EG1 has a 16S RNA gene that is at least 95% identical to SEQ ID NO:51. In some embodiments of any of the methods herein, the Bifidobacterium stercoris EG1 has a 16S RNA gene that is at least 95% identical to SEQ ID NO:52. In some embodiments of any of the methods herein, the Bifidobacterium stercoris EG1 has a 16S RNA gene that is at least 95% identical to SEQ ID NO:53. In some embodiments of any of the methods herein, the Bifidobacterium stercoris EG1 has a 16S RNA gene that is at least 95% identical to SEQ ID NO:54.

In some embodiments of any of the methods herein, the Clostridium sp. 40 has a 16S RNA gene that is at least 95% identical to SEQ ID NO:57.

In some embodiments of any of the methods herein, the Clostridium spiroforme CCM 6168 has a 16S RNA gene that is at least 95% identical to SEQ ID NO:58. In some embodiments of any of the methods herein, the Clostridium spiroforme CCM 6168 has a 16S RNA gene that is at least 95% identical to SEQ ID NO:59. In some embodiments of any of the methods herein, the Clostridium spiroforme CCM 6168 has a 16S RNA gene that is at least 95% identical to SEQ ID NO:60. In some embodiments of any of the methods herein, the Clostridium spiroforme CCM 6168 has a 16S RNA gene that is at least 95% identical to SEQ ID NO:61. In some embodiments of any of the methods herein, the Clostridium spiroforme CCM 6168 has a 16S RNA gene that is at least 95% identical to SEQ ID NO:62. In some embodiments of any of the methods herein, the Clostridium spiroforme CCM 6168 has a 16S RNA gene that is at least 95% identical to SEQ ID NO:63.

In some embodiments of any of the methods herein, the Eubacterium eligens DSM 3376 has a 16S RNA gene that is at least 95% identical to SEQ ID NO:64. In some embodiments of any of the methods herein, the Eubacterium eligens DSM 3376 has a 16S RNA gene that is at least 95% identical to SEQ ID NO:65. In some embodiments of any of the methods herein, the Eubacterium eligens DSM 3376 has a 16S RNA gene that is at least 95% identical to SEQ ID NO:66. In some embodiments of any of the methods herein, the Eubacterium eligens DSM 3376 has a 16S RNA gene that is at least 95% identical to SEQ ID NO:67. In some embodiments of any of the methods herein, the Eubacterium eligens DSM 3376 has a 16S RNA gene that is at least 95% identical to SEQ ID NO:68. In some embodiments of any of the methods herein, the Eubacterium eligens DSM 3376 has a 16S RNA gene that is at least 95% identical to SEQ ID NO:69. In some embodiments of any of the methods herein, the Eubacterium eligens DSM 3376 has a 16S RNA gene that is at least 95% identical to SEQ ID NO:70.

In some embodiments of any of the methods herein, the Eubacterium hallii DSM 3353 has a 16S RNA gene that is at least 95% identical to SEQ ID NO:71. In some embodiments of any of the methods herein, the Eubacterium hallii DSM 3353 has a 16S RNA gene that is at least 95% identical to SEQ ID NO:72.

In some embodiments of any of the methods herein, the Megamonas funiformis DSM 19343 has a 16S RNA gene that is at least 95% identical to SEQ ID NO:80. In some embodiments of any of the methods herein, the Megamonas funiformis DSM 19343 has a 16S RNA gene that is at least 95% identical to SEQ ID NO:81. In some embodiments of any of the methods herein, the Megamonas funiformis DSM 19343 has a 16S RNA gene that is at least 95% identical to SEQ ID NO:82. In some embodiments of any of the methods herein, the Megamonas funiformis DSM 19343 has a 16S RNA gene that is at least 95% identical to SEQ ID NO:83. In some embodiments of any of the methods herein, the Megamonas funiformis DSM 19343 has a 16S RNA gene that is at least 95% identical to SEQ ID NO:84. In some embodiments of any of the methods herein, the Megamonas funiformis DSM 19343 has a 16S RNA gene that is at least 95% identical to SEQ ID NO:85. In some embodiments of any of the methods herein, the Megamonas funiformis DSM 19343 has a 16S RNA gene that is at least 95% identical to SEQ ID NO:86.

In some embodiments of any of the methods herein, the Megasphaera elsdenii LC1 has a 16S RNA gene that is at least 95% identical to SEQ ID NO:87. In some embodiments of any of the methods herein, the Megasphaera elsdenii LC1 has a 16S RNA gene that is at least 95% identical to SEQ ID NO:88. In some embodiments of any of the methods herein, the Megasphaera elsdenii LC1 has a 16S RNA gene that is at least 95% identical to SEQ ID NO:89. In some embodiments of any of the methods herein, the Megasphaera elsdenii LC1 has a 16S RNA gene that is at least 95% identical to SEQ ID NO:90. In some embodiments of any of the methods herein, the Megasphaera elsdenii LC1 has a 16S RNA gene that is at least 95% identical to SEQ ID NO:91. In some embodiments of any of the methods herein, the Megasphaera elsdenii LC1 has a 16S RNA gene that is at least 95% identical to SEQ ID NO:92. In some embodiments of any of the methods herein, the Megasphaera elsdenii LC1 has a 16S RNA gene that is at least 95% identical to SEQ ID NO:93. In some embodiments of any of the methods herein, the Megasphaera elsdenii LC1 has a 16S RNA gene that is at least 95% identical to SEQ ID NO:94. In some embodiments of any of the methods herein, the Megasphaera elsdenii LC1 has a 16S RNA gene that is at least 95% identical to SEQ ID NO:95.

In some embodiments of any of the methods herein, the Parabacteroides johnsonii M 165 has a 16S RNA gene that is at least 95% identical to SEQ ID NO:104. In some embodiments of any of the methods herein, the Parabacteroides johnsonii M 165 has a 16S RNA gene that is at least 95% identical to SEQ ID NO:105. In some embodiments of any of the methods herein, the Parabacteroides johnsonii M 165 has a 16S RNA gene that is at least 95% identical to SEQ ID NO:106. In some embodiments of any of the methods herein, the Parabacteroides johnsonii M 165 has a 16S RNA gene that is at least 95% identical to SEQ ID NO:107.

In some embodiments of any of the methods herein, the Butyricimonas virosa MT12 has a 16S RNA gene that is at least 95% identical to SEQ ID NO:55.

In some embodiments of any of the methods herein, the Clostridium citroniae DSM 19261 has a 16S RNA gene that is at least 95% identical to SEQ ID NO:56.

In some embodiments of any of the methods herein, the Lactobacillus ruminis RF3 has a 16S RNA gene that is at least 95% identical to SEQ ID NO:73. In some embodiments of any of the methods herein, the Lactobacillus ruminis RF3 has a 16S RNA gene that is at least 95% identical to SEQ ID NO:74. In some embodiments of any of the methods herein, the Lactobacillus ruminis RF3 has a 16S RNA gene that is at least 95% identical to SEQ ID NO:75. In some embodiments of any of the methods herein, the Lactobacillus ruminis RF3 has a 16S RNA gene that is at least 95% identical to SEQ ID NO:76. In some embodiments of any of the methods herein, the Lactobacillus ruminis RF3 has a 16S RNA gene that is at least 95% identical to SEQ ID NO:77. In some embodiments of any of the methods herein, the Lactobacillus ruminis RF3 has a 16S RNA gene that is at least 95% identical to SEQ ID NO:78. In some embodiments of any of the methods herein, the Lactobacillus ruminis RF3 has a 16S RNA gene that is at least 95% identical to SEQ ID NO:79.

In some embodiments of any of the methods herein, the Methanosphaera stadtmanae MCB-3 has a 16S RNA gene that is at least 95% identical to SEQ ID NO:96. In some embodiments of any of the methods herein, the Methanosphaera stadtmanae MCB-3 has a 16S RNA gene that is at least 95% identical to SEQ ID NO:97. In some embodiments of any of the methods herein, the Methanosphaera stadtmanae MCB-3 has a 16S RNA gene that is at least 95% identical to SEQ ID NO:98. In some embodiments of any of the methods herein, the Methanosphaera stadtmanae MCB-3 has a 16S RNA gene that is at least 95% identical to SEQ ID NO:99. In some embodiments of any of the methods herein, the Methanosphaera stadtmanae MCB-3 has a 16S RNA gene that is at least 95% identical to SEQ ID NO:100. In some embodiments of any of the methods herein, the Methanosphaera stadtmanae MCB-3 has a 16S RNA gene that is at least 95% identical to SEQ ID NO:101. In some embodiments of any of the methods herein, the Methanosphaera stadtmanae MCB-3 has a 16S RNA gene that is at least 95% identical to SEQ ID NO:102. In some embodiments of any of the methods herein, the Methanosphaera stadtmanae MCB-3 has a 16S RNA gene that is at least 95% identical to SEQ ID NO:103.

In some embodiments of any of the methods herein, the Ruminococcus sp. 18P13 has a 16S RNA gene that is at least 95% identical to SEQ ID NO:108. In some embodiments of any of the methods herein, the Ruminococcus sp. 18P13 has a 16S RNA gene that is at least 95% identical to SEQ ID NO:109. In some embodiments of any of the methods herein, the Ruminococcus sp. 18P13 has a 16S RNA gene that is at least 95% identical to SEQ ID NO:110. In some embodiments of any of the methods herein, the Ruminococcus sp. 18P13 has a 16S RNA gene that is at least 95% identical to SEQ ID NO:111.

In some embodiments of any of the methods herein, the Slackia piriformis DSM 22477 has a 16S RNA gene that is at least 95% identical to SEQ ID NO:112. In some embodiments of any of the methods herein, the Slackia piriformis DSM 22477 has a 16S RNA gene that is at least 95% identical to SEQ ID NO:113. In some embodiments of any of the methods herein, the Slackia piriformis DSM 22477 has a 16S RNA gene that is at least 95% identical to SEQ ID NO:114. In some embodiments of any of the methods herein, the Slackia piriformis DSM 22477 has a 16S RNA gene that is at least 95% identical to SEQ ID NO:115. In some embodiments of any of the methods herein, the Slackia piriformis DSM 22477 has a 16S RNA gene that is at least 95% identical to SEQ ID NO:116. In some embodiments of any of the methods herein, the Slackia piriformis DSM 22477 has a 16S RNA gene that is at least 95% identical to SEQ ID NO:117.

In some embodiments of any of the methods herein, the Streptococcus hongkongensis HKU30 has a 16S RNA gene that is at least 95% identical to SEQ ID NO:118. In some embodiments of any of the methods herein, the Streptococcus hongkongensis HKU30 has a 16S RNA gene that is at least 95% identical to SEQ ID NO:119.

In some embodiments of any of the methods herein, the bacterial species in the composition is viable.

In some embodiments of any of the methods herein, the bacterial strain is lyophilized.

In some embodiments of any of the methods herein, the composition further comprises one or more cryopreservants.

In some embodiments of any of the methods herein, the effective amount of the bacterial strain comprises at least about 1×10³ colony forming units (CFU) of the bacterial strain. In some embodiments of any of the methods herein, the effective amount of the bacterial strain comprises about 1×10⁴ to about 1×10¹⁵ CFU of the bacterial strain. In some embodiments of any of the methods herein, the effective amount of the bacterial strain comprises about 1×10⁶ to about 1×10¹⁰ CFU of the bacterial strain.

In some embodiments of any of the methods herein, the bacterial strain in the composition is non-viable. In some embodiments of any of the methods herein, the non-viable bacterial strain is heat-killed, irradiated, or lysed.

In some embodiments of any of the methods herein, the method comprises administering the composition to the subject once, twice, or three times per day.

In some embodiments of any of the methods herein, the composition is formulated for oral administration. In some embodiments of any of the methods herein the composition is formulated for rectal administration. In some embodiments of any of the methods herein, the composition is formulated as a tablet, a capsule, a powder, or a liquid. In some embodiments of any of the methods herein, the composition is formulated as a tablet. In some embodiments of any of the methods herein, the tablet is coated. In some embodiments of any of the methods herein, the coating comprises an enteric coating.

In some embodiments of any of the methods herein, the method further comprises administering another treatment of colorectal cancer and/or other adjunct therapy to the subject. In some embodiments of any of the methods herein, the composition comprising the bacterial strain treatment and the treatment for colorectal cancer and/or adjunct therapy are administered simultaneously. In some embodiments of any of the methods herein, the composition comprising the bacterial strain treatment and the treatment for colorectal cancer and/or adjunct therapy are administered sequentially. In some embodiments of any of the methods herein, the treatment for colorectal cancer and/or adjunct therapy comprises a probiotic. In some embodiments of any of the methods herein, the treatment for colorectal cancer and/or adjunct therapy comprises surgery, radiation therapy, ablation, embolization, or a combination thereof.

In some embodiments of any of the methods herein, the treatment for colorectal cancer and/or adjunct therapy comprises a therapeutic agent. In some embodiments of any of the methods herein, the therapeutic agent comprises a chemotherapeutic agent, targeted therapy, immunotherapy, a cell therapy, or a combination thereof. In some embodiments of any of the methods herein, the chemotherapeutic agent comprises aflibercept, capecitabine, fluorouracil, irinotecan, leucovorin, oxaliplatin, trifluridine, tipiracil, or a combination thereof. In some embodiments of any of the methods herein, the targeted therapy comprises bevacizumab, cetuximab, ziv-aflibercept, panitumumab, regorafenib, or a combination thereof. In some embodiments of any of the methods herein, the combination of therapeutic agents is selected from the group consisting of: FOLFOX, FOLFIRI, CAPDX, FOLFIRI-bevacizumab, FOLFIRI-cetuximab, or XELOX, and a combination thereof.

In some embodiments of any of the methods herein, the immunotherapy comprises a checkpoint inhibitor. In some embodiments of any of the methods herein, the checkpoint inhibitor targets one or more (e.g., two or more, three or more, four or more, five or more, six or more, seven or more, eight or more, nine or more, or ten) of: CTLA-4, PD-1, PD-L1, BTLA, LAG-3, A2AR, TIM-3, B7-H3, VISTA, and IDO.

In some embodiments of any of the methods herein, the composition comprising the bacterial strain further comprises the therapeutic agent. In some embodiments of any of the methods herein, the cell therapy is a CAR T-cell therapy.

In some embodiments of any of the methods herein, the subject is a human.

Also provided herein are methods for treating a subject in need thereof that include administering to the subject a composition comprising an effective amount of a bacterial strain selected from the group consisting of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, and a combination thereof. In some embodiments of any of the methods herein, the subject has colorectal cancer.

Also provided herein are methods for treating colorectal cancer in a subject that include administering to the subject a composition comprising an effective amount of a bacterial strain selected from the group consisting of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, and a combination thereof.

Also provided herein are methods for treating colorectal cancer in a subject that include (a) detecting a dysbiosis associated with colorectal cancer in a sample from the subject; and (b) administering to the subject a composition comprising an effective amount of a bacterial strain selected from the group consisting of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, and a combination thereof.

In some embodiments of any of the methods herein, the sample is a fecal sample. In some embodiments of any of the methods herein, the sample is a biopsy sample. In some embodiments of any of the methods herein, the biopsy sample is a colorectal biopsy sample.

In some embodiments of any of the methods herein, detecting the dysbiosis associated with colorectal cancer comprises determining bacterial gene expression in the sample from the subject. In some embodiments of any of the methods herein, detecting the dysbiosis associated with colorectal cancer comprises determining bacterial composition in the sample from the subject.

In some embodiments of any of the methods herein, detecting the dysbiosis associated with colorectal cancer comprises determining that Alistipes indistinctus DSM 22520, Azotobacter sp. A1 2, Bacteroides asaccharolyticus B440, Bacteroides sp. 2_2_4, Bilophila wadsworthia 3 1 6, Clostridium aldenense RMA9741, Clostridium bolteae DSM 15670, Clostridium lactatifermentans G17, Clostridium sp. LIP1, Desulfovibrio piger DSM 749, Dialister pneumosintes DSM 11619, Eubacterium biforme DSM 3989, Fusobacterium mortiferum ATCC 9817, Hungatella hathewayi 1313, Megasphaera micronuciformis DSM 17226, Micromonas micros ACM 5086, Paraprevotella clara DSM 19731, Peptostreptococcus stomatis W2278, Porphyromonas somerae WAL 6690, Roseburia inulinivorans A2-194, Ruminococcus gnavus VPI C7-9, Shigella dysenteriae CECT 584, Subdoligranulum variabile BI-114, Veillonella alcalescens 259, or a combination thereof, is increased in the sample from subject.

In some embodiments of any of the methods herein, detecting the dysbiosis associated with colorectal cancer comprises determining that Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, or a combination thereof, is decreased in the sample from subject.

In some embodiments of any of the methods herein, Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, or a combination thereof, is decreased in the gastrointestinal tract of the subject.

Also provided herein are methods for treating a subject in need thereof that include decreasing a population of an increased bacterial strain in the subject, wherein the increased bacterial strain is selected from the group consisting of: Alistipes indistinctus DSM 22520, Azotobacter sp. A1 2, Bacteroides asaccharolyticus B440, Bacteroides sp. 2_2_4, Bilophila wadsworthia 3 1 6, Clostridium aldenense RMA9741, Clostridium bolteae DSM 15670, Clostridium lactatifermentans G17, Clostridium sp. LIP1, Desulfovibrio piger DSM 749, Dialister pneumosintes DSM 11619, Eubacterium biforme DSM 3989, Fusobacterium mortiferum ATCC 9817, Hungatella hathewayi 1313, Megasphaera micronuciformis DSM 17226, Micromonas micros ACM 5086, Paraprevotella clara DSM 19731, Peptostreptococcus stomatis W2278, Porphyromonas somerae WAL 6690, Roseburia inulinivorans A2-194, Ruminococcus gnavus VPI C7-9, Shigella dysenteriae CECT 584, Subdoligranulum variabile BI-114, Veillonella alcalescens 259, and a combination thereof.

In some embodiments of any of the methods herein, the subject has colorectal cancer.

In some embodiments of any of the methods herein, decreasing the population of an increased bacterial strain comprises administering to the subject a bacteriophage. In some embodiments of any of the methods herein, decreasing the population of an increased bacterial strain comprises administering to the subject a composition comprising an effective amount of a bacterial strain selected from the group consisting of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, and a combination thereof.

In some embodiments of any of the methods herein, the colorectal cancer is adenocarcinoma, a carcinoid tumor, a gastrointestinal stromal tumor, or lymphoma.

In some embodiments of any of the methods herein, the bacterial strain comprises Bacteroides eggerthii 1 2 48FAA. In some embodiments of any of the methods herein, the bacterial strain comprises Bacteroides eggerthii CCUG 9559. In some embodiments of any of the methods herein, the bacterial strain comprises Bacteroides goldsteinii ATCC BAA 1180. In some embodiments of any of the methods herein, the bacterial strain comprises Bacteroides pectinophilus N3. In some embodiments of any of the methods herein, the bacterial strain comprises Bacteroides plebeius M12. In some embodiments of any of the methods herein, the bacterial strain comprises Bacteroides vulgatus 8482. In some embodiments of any of the methods herein, the bacterial strain comprises Barnesiella intestinihominis DSM 21032. In some embodiments of any of the methods herein, the bacterial strain comprises Bifidobacterium stercoris EG1. In some embodiments of any of the methods herein, the bacterial strain comprises Clostridium sp. 40. In some embodiments of any of the methods herein, the bacterial strain comprises Clostridium spiroforme CCM 6168. In some embodiments of any of the methods herein, the bacterial strain comprises Eubacterium eligens DSM 3376. In some embodiments of any of the methods herein, the bacterial strain comprises Eubacterium hallii DSM 3353. In some embodiments of any of the methods herein, the bacterial strain comprises Megamonas funiformis DSM 19343. In some embodiments of any of the methods herein, the bacterial strain comprises Megasphaera elsdenii LC1. In some embodiments of any of the methods herein, the bacterial strain comprises Parabacteroides johnsonii M-165. In some embodiments of any of the methods herein, the bacterial strain comprises Butyricimonas virosa MT12. In some embodiments of any of the methods herein, the bacterial strain comprises Clostridium citroniae DSM 19261. In some embodiments of any of the methods herein, the bacterial strain comprises Lactobacillus ruminis RF3. In some embodiments of any of the methods herein, the bacterial strain comprises Methanosphaera stadtmanae MCB-3. In some embodiments of any of the methods herein, the bacterial strain comprises Ruminococcus sp. 18P13. In some embodiments of any of the methods herein, the bacterial strain comprises Slackia piriformis DSM 22477. In some embodiments of any of the methods herein, the bacterial strain comprises Streptococcus hongkongensis HKU30.

In some embodiments of any of the methods herein, the bacterial strain improves intestinal barrier function of the subject.

In some embodiments of any of the methods herein, the Bacteroides eggerthii 1 2 48FAA has a 16S RNA gene that is at least 95% identical to SEQ ID NO:1. In some embodiments of any of the methods herein, the Bacteroides eggerthii 1 2 48FAA has a 16S RNA gene that is at least 95% identical to SEQ ID NO:2. In some embodiments of any of the methods herein, the Bacteroides eggerthii 1 2 48FAA has a 16S RNA gene that is at least 95% identical to SEQ ID NO:3. In some embodiments of any of the methods herein, the Bacteroides eggerthii 1 2 48FAA has a 16S RNA gene that is at least 95% identical to SEQ ID NO:4. In some embodiments of any of the methods herein, the Bacteroides eggerthii 1 2 48FAA has a 16S RNA gene that is at least 95% identical to SEQ ID NO:5. In some embodiments of any of the methods herein, the Bacteroides eggerthii 1 2 48FAA has a 16S RNA gene that is at least 95% identical to SEQ ID NO:6.

In some embodiments of any of the methods herein, the Bacteroides eggerthii CCUG 9559 has a 16S RNA gene that is at least 95% identical to SEQ ID NO:7. In some embodiments of any of the methods herein, the Bacteroides eggerthii CCUG 9559 has a 16S RNA gene that is at least 95% identical to SEQ ID NO:8. In some embodiments of any of the methods herein, the Bacteroides eggerthii CCUG 9559 has a 16S RNA gene that is at least 95% identical to SEQ ID NO:9. In some embodiments of any of the methods herein, the Bacteroides eggerthii CCUG 9559 has a 16S RNA gene that is at least 95% identical to SEQ ID NO:10. In some embodiments of any of the methods herein, the Bacteroides eggerthii CCUG 9559 has a 16S RNA gene that is at least 95% identical to SEQ ID NO:11. In some embodiments of any of the methods herein, the Bacteroides eggerthii CCUG 9559 has a 16S RNA gene that is at least 95% identical to SEQ ID NO:12.

In some embodiments of any of the methods herein, the Bacteroides goldsteinii ATCC BAA 1180 has a 16S RNA gene that is at least 95% identical to SEQ ID NO:13. In some embodiments of any of the methods herein, the Bacteroides goldsteinii ATCC BAA 1180 has a 16S RNA gene that is at least 95% identical to SEQ ID NO:14. In some embodiments of any of the methods herein, the Bacteroides goldsteinii ATCC BAA 1180 has a 16S RNA gene that is at least 95% identical to SEQ ID NO:15. In some embodiments of any of the methods herein, the Bacteroides goldsteinii ATCC BAA 1180 has a 16S RNA gene that is at least 95% identical to SEQ ID NO:16. In some embodiments of any of the methods herein, the Bacteroides goldsteinii ATCC BAA 1180 has a 16S RNA gene that is at least 95% identical to SEQ ID NO:17. In some embodiments of any of the methods herein, the Bacteroides goldsteinii ATCC BAA 1180 has a 16S RNA gene that is at least 95% identical to SEQ ID NO:18. In some embodiments of any of the methods herein, the Bacteroides goldsteinii ATCC BAA 1180 has a 16S RNA gene that is at least 95% identical to SEQ ID NO:19.

In some embodiments of any of the methods herein, the Bacteroides pectinophilus N3 has a 16S RNA gene that is at least 95% identical to SEQ ID NO:20. In some embodiments of any of the methods herein, the Bacteroides pectinophilus N3 has a 16S RNA gene that is at least 95% identical to SEQ ID NO:21. In some embodiments of any of the methods herein, the Bacteroides pectinophilus N3 has a 16S RNA gene that is at least 95% identical to SEQ ID NO:22. In some embodiments of any of the methods herein, the Bacteroides pectinophilus N3 has a 16S RNA gene that is at least 95% identical to SEQ ID NO:23. In some embodiments of any of the methods herein, the Bacteroides pectinophilus N3 has a 16S RNA gene that is at least 95% identical to SEQ ID NO:24. In some embodiments of any of the methods herein, the Bacteroides pectinophilus N3 has a 16S RNA gene that is at least 95% identical to SEQ ID NO:25.

In some embodiments of any of the methods herein, the Bacteroides plebeius M12 has a 16S RNA gene that is at least 95% identical to SEQ ID NO:26. In some embodiments of any of the methods herein, the Bacteroides plebeius M12 has a 16S RNA gene that is at least 95% identical to SEQ ID NO:27. In some embodiments of any of the methods herein, the Bacteroides plebeius M12 has a 16S RNA gene that is at least 95% identical to SEQ ID NO:28.

In some embodiments of any of the methods herein, the Bacteroides vulgatus 8482 has a 16S RNA gene that is at least 95% identical to SEQ ID NO:29. In some embodiments of any of the methods herein, the Bacteroides vulgatus 8482 has a 16S RNA gene that is at least 95% identical to SEQ ID NO:30. In some embodiments of any of the methods herein, the Bacteroides vulgatus 8482 has a 16S RNA gene that is at least 95% identical to SEQ ID NO:31. In some embodiments of any of the methods herein, the Bacteroides vulgatus 8482 has a 16S RNA gene that is at least 95% identical to SEQ ID NO:32. In some embodiments of any of the methods herein, the Bacteroides vulgatus 8482 has a 16S RNA gene that is at least 95% identical to SEQ ID NO:33. In some embodiments of any of the methods herein, the Bacteroides vulgatus 8482 has a 16S RNA gene that is at least 95% identical to SEQ ID NO:34. In some embodiments of any of the methods herein, the Bacteroides vulgatus 8482 has a 16S RNA gene that is at least 95% identical to SEQ ID NO:35. In some embodiments of any of the methods herein, the Bacteroides vulgatus 8482 has a 16S RNA gene that is at least 95% identical to SEQ ID NO:36. In some embodiments of any of the methods herein, the Bacteroides vulgatus 8482 has a 16S RNA gene that is at least 95% identical to SEQ ID NO:37. In some embodiments of any of the methods herein, the Bacteroides vulgatus 8482 has a 16S RNA gene that is at least 95% identical to SEQ ID NO:38. In some embodiments of any of the methods herein, the Bacteroides vulgatus 8482 has a 16S RNA gene that is at least 95% identical to SEQ ID NO:39. In some embodiments of any of the methods herein, the Bacteroides vulgatus 8482 has a 16S RNA gene that is at least 95% identical to SEQ ID NO:40.

In some embodiments of any of the methods herein, the Barnesiella intestinihominis DSM 21032 has a 16S RNA gene that is at least 95% identical to SEQ ID NO:41. In some embodiments of any of the methods herein, the Barnesiella intestinihominis DSM 21032 has a 16S RNA gene that is at least 95% identical to SEQ ID NO:42. In some embodiments of any of the methods herein, the Barnesiella intestinihominis DSM 21032 has a 16S RNA gene that is at least 95% identical to SEQ ID NO:43. In some embodiments of any of the methods herein, the Barnesiella intestinihominis DSM 21032 has a 16S RNA gene that is at least 95% identical to SEQ ID NO:44. In some embodiments of any of the methods herein, the Barnesiella intestinihominis DSM 21032 has a 16S RNA gene that is at least 95% identical to SEQ ID NO:45. In some embodiments of any of the methods herein, the Barnesiella intestinihominis DSM 21032 has a 16S RNA gene that is at least 95% identical to SEQ ID NO:46. In some embodiments of any of the methods herein, the Barnesiella intestinihominis DSM 21032 has a 16S RNA gene that is at least 95% identical to SEQ ID NO:47. In some embodiments of any of the methods herein, the Barnesiella intestinihominis DSM 21032 has a 16S RNA gene that is at least 95% identical to SEQ ID NO:48. In some embodiments of any of the methods herein, the Barnesiella intestinihominis DSM 21032 has a 16S RNA gene that is at least 95% identical to SEQ ID NO:49. In some embodiments of any of the methods herein, in the Barnesiella intestinihominis DSM 21032 has a 16S RNA gene that is at least 95% identical to SEQ ID NO:50.

In some embodiments of any of the methods herein, the Bifidobacterium stercoris EG1 has a 16S RNA gene that is at least 95% identical to SEQ ID NO:51. In some embodiments of any of the methods herein, the Bifidobacterium stercoris EG1 has a 16S RNA gene that is at least 95% identical to SEQ ID NO:52. In some embodiments of any of the methods herein, the Bifidobacterium stercoris EG1 has a 16S RNA gene that is at least 95% identical to SEQ ID NO:53. In some embodiments of any of the methods herein, the Bifidobacterium stercoris EG1 has a 16S RNA gene that is at least 95% identical to SEQ ID NO:54.

In some embodiments of any of the methods herein, the Clostridium sp. 40 has a 16S RNA gene that is at least 95% identical to SEQ ID NO:57.

In some embodiments of any of the methods herein, the Clostridium spiroforme CCM 6168 has a 16S RNA gene that is at least 95% identical to SEQ ID NO:58. In some embodiments of any of the methods herein, the Clostridium spiroforme CCM 6168 has a 16S RNA gene that is at least 95% identical to SEQ ID NO:59. In some embodiments of any of the methods herein, the Clostridium spiroforme CCM 6168 has a 16S RNA gene that is at least 95% identical to SEQ ID NO:60. In some embodiments of any of the methods herein, the Clostridium spiroforme CCM 6168 has a 16S RNA gene that is at least 95% identical to SEQ ID NO:61. In some embodiments of any of the methods herein, the Clostridium spiroforme CCM 6168 has a 16S RNA gene that is at least 95% identical to SEQ ID NO:62. In some embodiments of any of the methods herein, the Clostridium spiroforme CCM 6168 has a 16S RNA gene that is at least 95% identical to SEQ ID NO:63.

In some embodiments of any of the methods herein, the Eubacterium eligens DSM 3376 has a 16S RNA gene that is at least 95% identical to SEQ ID NO:64. In some embodiments of any of the methods herein, the Eubacterium eligens DSM 3376 has a 16S RNA gene that is at least 95% identical to SEQ ID NO:65. In some embodiments of any of the methods herein, the Eubacterium eligens DSM 3376 has a 16S RNA gene that is at least 95% identical to SEQ ID NO:66. In some embodiments of any of the methods herein, the Eubacterium eligens DSM 3376 has a 16S RNA gene that is at least 95% identical to SEQ ID NO:67. In some embodiments of any of the methods herein, the Eubacterium eligens DSM 3376 has a 16S RNA gene that is at least 95% identical to SEQ ID NO:68. In some embodiments of any of the methods herein, the Eubacterium eligens DSM 3376 has a 16S RNA gene that is at least 95% identical to SEQ ID NO:69. In some embodiments of any of the methods herein, the Eubacterium eligens DSM 3376 has a 16S RNA gene that is at least 95% identical to SEQ ID NO:70.

In some embodiments of any of the methods herein, the Eubacterium hallii DSM 3353 has a 16S RNA gene that is at least 95% identical to SEQ ID NO:71. In some embodiments of any of the methods herein, the Eubacterium hallii DSM 3353 has a 16S RNA gene that is at least 95% identical to SEQ ID NO:72.

In some embodiments of any of the methods herein, the Megamonas funiformis DSM 19343 has a 16S RNA gene that is at least 95% identical to SEQ ID NO:80. In some embodiments of any of the methods herein, the Megamonas funiformis DSM 19343 has a 16S RNA gene that is at least 95% identical to SEQ ID NO:81. In some embodiments of any of the methods herein, the Megamonas funiformis DSM 19343 has a 16S RNA gene that is at least 95% identical to SEQ ID NO:82. In some embodiments of any of the methods herein, the Megamonas funiformis DSM 19343 has a 16S RNA gene that is at least 95% identical to SEQ ID NO:83. In some embodiments of any of the methods herein, the Megamonas funiformis DSM 19343 has a 16S RNA gene that is at least 95% identical to SEQ ID NO:84. In some embodiments of any of the methods herein, the Megamonas funiformis DSM 19343 has a 16S RNA gene that is at least 95% identical to SEQ ID NO:85. In some embodiments of any of the methods herein, the Megamonas funiformis DSM 19343 has a 16S RNA gene that is at least 95% identical to SEQ ID NO:86.

In some embodiments of any of the methods herein, the Megasphaera elsdenii LC1 has a 16S RNA gene that is at least 95% identical to SEQ ID NO:87. In some embodiments of any of the methods herein, the Megasphaera elsdenii LC1 has a 16S RNA gene that is at least 95% identical to SEQ ID NO:88. In some embodiments of any of the methods herein, the Megasphaera elsdenii LC1 has a 16S RNA gene that is at least 95% identical to SEQ ID NO:89. In some embodiments of any of the methods herein, the Megasphaera elsdenii LC1 has a 16S RNA gene that is at least 95% identical to SEQ ID NO:90. In some embodiments of any of the methods herein, the Megasphaera elsdenii LC1 has a 16S RNA gene that is at least 95% identical to SEQ ID NO:91. In some embodiments of any of the methods herein, the Megasphaera elsdenii LC1 has a 16S RNA gene that is at least 95% identical to SEQ ID NO:92. In some embodiments of any of the methods herein, the Megasphaera elsdenii LC1 has a 16S RNA gene that is at least 95% identical to SEQ ID NO:93. In some embodiments of any of the methods herein, the Megasphaera elsdenii LC1 has a 16S RNA gene that is at least 95% identical to SEQ ID NO:94. In some embodiments of any of the methods herein, the Megasphaera elsdenii LC1 has a 16S RNA gene that is at least 95% identical to SEQ ID NO:95.

In some embodiments of any of the methods herein, the Parabacteroides johnsonii M 165 has a 16S RNA gene that is at least 95% identical to SEQ ID NO:104. In some embodiments of any of the methods herein, the Parabacteroides johnsonii M 165 has a 16S RNA gene that is at least 95% identical to SEQ ID NO:105. In some embodiments of any of the methods herein, the Parabacteroides johnsonii M 165 has a 16S RNA gene that is at least 95% identical to SEQ ID NO:106. In some embodiments of any of the methods herein, the Parabacteroides johnsonii M 165 has a 16S RNA gene that is at least 95% identical to SEQ ID NO:107.

In some embodiments of any of the methods herein, the Butyricimonas virosa MT12 has a 16S RNA gene that is at least 95% identical to SEQ ID NO:55.

In some embodiments of any of the methods herein, the Clostridium citroniae DSM 19261 has a 16S RNA gene that is at least 95% identical to SEQ ID NO:56.

In some embodiments of any of the methods herein, the Lactobacillus ruminis RF3 has a 16S RNA gene that is at least 95% identical to SEQ ID NO:73. In some embodiments of any of the methods herein, the Lactobacillus ruminis RF3 has a 16S RNA gene that is at least 95% identical to SEQ ID NO:74. In some embodiments of any of the methods herein, the Lactobacillus ruminis RF3 has a 16S RNA gene that is at least 95% identical to SEQ ID NO:75. In some embodiments of any of the methods herein, the Lactobacillus ruminis RF3 has a 16S RNA gene that is at least 95% identical to SEQ ID NO:76. In some embodiments of any of the methods herein the Lactobacillus ruminis RF3 has a 16S RNA gene that is at least 95% identical to SEQ ID NO:77. In some embodiments of any of the methods herein, the Lactobacillus ruminis RF3 has a 16S RNA gene that is at least 95% identical to SEQ ID NO:78. In some embodiments of any of the methods herein, the Lactobacillus ruminis RF3 has a 16S RNA gene that is at least 95% identical to SEQ ID NO:79.

In some embodiments of any of the methods herein, the Methanosphaera stadtmanae MCB-3 has a 16S RNA gene that is at least 95% identical to SEQ ID NO:96. In some embodiments of any of the methods herein, the Methanosphaera stadtmanae MCB-3 has a 16S RNA gene that is at least 95% identical to SEQ ID NO:97. In some embodiments of any of the methods herein, the Methanosphaera stadtmanae MCB-3 has a 16S RNA gene that is at least 95% identical to SEQ ID NO:98. In some embodiments of any of the methods herein, the Methanosphaera stadtmanae MCB-3 has a 16S RNA gene that is at least 95% identical to SEQ ID NO:99. In some embodiments of any of the methods herein, the Methanosphaera stadtmanae MCB-3 has a 16S RNA gene that is at least 95% identical to SEQ ID NO:100. In some embodiments of any of the methods herein, the Methanosphaera stadtmanae MCB-3 has a 16S RNA gene that is at least 95% identical to SEQ ID NO:101. In some embodiments of any of the methods herein, the Methanosphaera stadtmanae MCB-3 has a 16S RNA gene that is at least 95% identical to SEQ ID NO:102. In some embodiments of any of the methods herein, the Methanosphaera stadtmanae MCB-3 has a 16S RNA gene that is at least 95% identical to SEQ ID NO:103.

In some embodiments of any of the methods herein, the Ruminococcus sp. 18P13 has a 16S RNA gene that is at least 95% identical to SEQ ID NO:108. In some embodiments of any of the methods herein, the Ruminococcus sp. 18P13 has a 16S RNA gene that is at least 95% identical to SEQ ID NO:109. In some embodiments of any of the methods herein, the Ruminococcus sp. 18P13 has a 16S RNA gene that is at least 95% identical to SEQ ID NO:110. In some embodiments of any of the methods herein, the Ruminococcus sp. 18P13 has a 16S RNA gene that is at least 95% identical to SEQ ID NO:111.

In some embodiments of any of the methods herein, the Slackia piriformis DSM 22477 has a 16S RNA gene that is at least 95% identical to SEQ ID NO:112. In some embodiments of any of the methods herein, the Slackia piriformis DSM 22477 has a 16S RNA gene that is at least 95% identical to SEQ ID NO:113. In some embodiments of any of the methods herein, the Slackia piriformis DSM 22477 has a 16S RNA gene that is at least 95% identical to SEQ ID NO:114. In some embodiments of any of the methods herein, the Slackia piriformis DSM 22477 has a 16S RNA gene that is at least 95% identical to SEQ ID NO:115. In some embodiments of any of the methods herein, the Slackia piriformis DSM 22477 has a 16S RNA gene that is at least 95% identical to SEQ ID NO:116. In some embodiments of any of the methods herein, the Slackia piriformis DSM 22477 has a 16S RNA gene that is at least 95% identical to SEQ ID NO:117.

In some embodiments of any of the methods herein, the Streptococcus hongkongensis HKU30 has a 16S RNA gene that is at least 95% identical to SEQ ID NO:118. In some embodiments of any of the methods herein, the Streptococcus hongkongensis HKU30 has a 16S RNA gene that is at least 95% identical to SEQ ID NO:119.

In some embodiments of any of the methods herein, the bacterial strain in the composition is viable. In some embodiments of any of the methods herein, the bacterial strain is lyophilized.

In some embodiments of any of the methods herein, the composition further comprises one or more cryopreservants.

In some embodiments of any of the methods herein, the effective amount of the bacterial strain comprises at least about 1×10³ colony forming units (CFU) of the bacterial strain. In some embodiments of any of the methods herein, the effective amount of the bacterial strain comprises about 1×10⁴ to about 1×10¹⁵ CFU of the bacterial strain. In some embodiments of any of the methods herein, the effective amount of the bacterial strain comprises about 1×10⁶ to about 1×10¹⁰ CFU of the bacterial strain.

In some embodiments of any of the methods herein, the bacterial strain in the composition is non-viable. In some embodiments of any of the methods herein, the non-viable bacterial strain is heat-killed, irradiated, or lysed.

In some embodiments of any of the methods herein, the method comprises administering the composition to the subject once, twice, or three times per day.

In some embodiments of any of the methods herein, the composition is formulated for oral administration. In some embodiments of any of the methods herein, the composition is formulated for rectal administration. In some embodiments of any of the methods herein, the composition is formulated as a tablet, a capsule, a powder, or a liquid. In some embodiments of any of the methods herein, the composition is formulated as a tablet. In some embodiments of any of the methods herein, the tablet is coated. In some embodiments of any of the methods herein, the coating comprises an enteric coating.

In some embodiments of any of the methods herein, the method further comprises administering another treatment of colorectal cancer and/or other adjunct therapy to the subject. In some embodiments of any of the methods herein, the composition comprising the bacterial strain treatment and the treatment for colorectal cancer and/or adjunct therapy are administered simultaneously. In some embodiments of any of the methods herein, the composition comprising the bacterial strain treatment and the treatment for colorectal cancer and/or adjunct therapy are administered sequentially. In some embodiments of any of the methods herein, the treatment for colorectal cancer and/or adjunct therapy comprises a probiotic. In some embodiments of any of the methods herein, the treatment for colorectal cancer and/or adjunct therapy comprises surgery, radiation therapy, ablation, embolization, or a combination thereof.

In some embodiments of any of the methods herein, the treatment for colorectal cancer and/or adjunct therapy comprises a therapeutic agent. In some embodiments of any of the methods herein, the therapeutic agent comprises a chemotherapeutic agent, targeted therapy, immunotherapy, a cell therapy, or a combination thereof. In some embodiments of any of the methods herein, the chemotherapeutic agent comprises aflibercept, capecitabine, fluorouracil, irinotecan, leucovorin, oxaliplatin, trifluridine, tipiracil, or a combination thereof. In some embodiments of any of the methods herein, the targeted therapy comprises bevacizumab, cetuximab, ziv-aflibercept, panitumumab, regorafenib, or a combination thereof. In some embodiments of any of the methods herein, the combination of therapeutic agents is selected from the group consisting of: FOLFOX, FOLFIRI, CAPDX, FOLFIRI-bevacizumab, FOLFIRI-cetuximab, or XELOX, and a combination thereof.

In some embodiments of any of the methods herein, the immunotherapy comprises a checkpoint inhibitor. In some embodiments of any of the methods herein, the checkpoint inhibitor targets one or more (e.g., two or more, three or more, four or more, five or more, six or more, seven or more, eight or more, nine or more, or ten) of: CTLA-4, PD-1, PD-L1, BTLA, LAG-3, A2AR, TIM-3, B7-H3, VISTA, and IDO.

In some embodiments of any of the methods herein, the composition comprising the bacterial strain further comprises the therapeutic agent.

In some embodiments of any of the methods herein, the cell therapy is a CAR T-cell therapy.

In some embodiments of any of the methods herein, the subject is a human.

As used herein, the phrase an “effective amount” of a bacterial strain can refer to an amount of the bacterial strain sufficient enough to reduce or eliminate one or more symptoms of the disorder or in some cases, to effect a cure upon administration. Effective amounts of a bacterial strain will vary with the bacterial strain chosen, the particular condition or conditions being treated, the severity of the condition, the duration of the treatment, the specific components of the composition being used, and like factors. An “effective amount” can also refer to an amount of a combination of two or more bacterial strains or a combination of a bacterial strain and a therapeutic agent sufficient to reduce or eliminate one or more symptoms of the disorder or in some cases, to effect a cure upon administration. For example, an “effective amount” can refer to an amount of a combination of bacterial strains or a combination of a bacterial strain and another treatment (e.g., a therapeutic agent) when an additive or synergistic effect is observed with the combination compared to administration of the bacterial strain(s) and/or therapeutic agent(s) alone.

As used herein, “subject” or “patient” refers to any subject, particularly a mammalian subject such as a human, for whom diagnosis, prognosis, or therapy is desired.

As used herein, “treatment” or “treating” of a disease, disorder, or condition encompasses alleviation of at least one symptom thereof, a reduction in the severity thereof, or the delay or inhibition of the progression thereof. Treatment need not mean that the disease, disorder, or condition is totally cured. A useful composition herein needs only to reduce the severity of a disease, disorder, or condition, reduce the severity of one or more symptoms associated therewith, or improve a patient or subject's quality of life.

The term “preventing” as used herein means the prevention of the onset, recurrence, or spread, in whole or in part, of the disease or condition as described herein, or a symptom thereof.

The term “administration” or “administering” refers to a method of giving an amount of a bacterial strain, or a composition thereof, or other treatment of colorectal cancer and/or adjunct therapy to a subject. The method of administration can vary depending on various factors, e.g., the components of the composition, the site of the disease, and the severity of the disease.

“Microbiome” refers to the collection of microorganisms and viruses and/or their genes from a given environment. For example, “microbiome” can refer to the collection of the microorganisms and viruses and/or their genes from the gastrointestinal tract of humans. “Microbiota” refers to the microorganisms in a specific environment.

“Dysbiosis” refers to a state of the microbiota or microbiome of the gut or other body area (e.g., mucosal or skin surfaces or any other microbiota niche) of a subject (i.e., the host) in which the diversity and/or function of the ecological network is disrupted, e.g., as compared to the state of the microbiota or microbiome of the gut or other body area in a control population. A control population can include individuals that meet one or more qualifications such as individuals that have not been diagnosed with a disease (e.g., the same disease as the subject); individuals that do not have a known genetic predisposition to a disease (e.g., the same disease as the subject); or individuals that do not have a known environmental predisposition to a disease (e.g., the same disease as the subject); or individuals that do not have a known predisposition that would prevent treatment of and/or recovery from a disease (e.g., the same disease as the subject). In some embodiments, the individuals in the control population meet one of the above control population qualifications. In some embodiments, the individuals in the control population meet two of the above control population qualifications. In some embodiments, the individuals in the control population meet three of the above control population qualifications. In some embodiments, the individuals in the control population meet four of the above control population qualifications. In some embodiments, the control population is homogenous with respect to at least one of the qualifications. Any disruption in the microbiota or microbiome of a subject (i.e., host) compared to the microbiota or microbiome of a control population can be considered a dysbiosis, even if such dysbiosis does not result in a detectable decrease in health of the subject. Dysbiosis in a subject may be unhealthy for the subject (e.g., result in a diseased state in the subject), it may be unhealthy for the subject under only certain conditions (e.g., result in diseased state under only certain conditions), or it may prevent the subject from becoming healthier (e.g., may prevent a subject from responding to treatment or recovering from a disease or disorder). Dysbiosis may be due to a decrease in diversity of the microbiota population composition (e.g., a depletion of one or more bacterial strains, an overgrowth of one or more bacterial strains, or a combination thereof), the overgrowth of one or more population of pathogens (e.g., a population of pathogenic bacteria) or pathobionts, the presence of and/or overgrowth of a symbiotic organism able to cause disease only when certain genetic and/or environmental conditions are present in a subject, or a shift to an ecological network that no longer provides a beneficial function to the host and therefore no longer promotes health.

As used herein the terms “microorganism” or “microbe” should be taken broadly. These terms are used interchangeably and include, but are not limited to, the two prokaryotic domains, Bacteria and Archaea, as well as eukaryotic fungi and protists. In some embodiments, the disclosure refers to a “bacterium” or a “microbe.” This characterization can refer to not only the identified taxonomic bacterial genera of the microbe, but also the identified taxonomic species, as well as the bacterial strains. A “strain” can include descendants of a single isolation in pure culture that is usually made up of a succession of cultures ultimately derived from an initial single colony. In some embodiments, a strain includes an isolate or a group of isolates that can be distinguished from other isolates of the same genus and species by phenotypic characteristics, genotypic characteristics, or both.

The term “relative abundance” as used herein, is the number or percentage of a microbe present in the gastrointestinal tract or any other microbiota niche of a subject, such as the ocular, placental, lung, cutaneous, urogenital, or oral microbiota niches, relative to the number or percentage of total microbes present in the gastrointestinal tract or the other microbiota niche of the subject. The relative abundance may also be determined for particular types of microbes such as bacteria, fungi, viruses, and/or protozoa, relative to the total number or percentage of bacteria, fungi, viruses, and/or protozoa present. Relative abundance can be determined by a number of methods readily known to the ordinarily skilled artisan, including, but not limited to, array or microarray hybridization, sequencing, quantitative PCR, and culturing and performance of colony forming unit (cfu, CFU) assays or plaque forming unit (pfu, PFU) assays performed on a sample from the gastrointestinal tract or other microbiota niche.

As used herein, terms such as “isolate” and “isolated” in reference to a microbe, are intended to mean that a microbe has been separated from at least one of the materials with which it is associated in a particular environment (for example gastrointestinal fluid, gastrointestinal tissue, human digestive fluid, human digestive tissue, etc.). Accordingly, an “isolated microbe” does not exist in its naturally occurring environment. In some embodiments, an isolated microbe, e.g., a bacterial strain, may exist as, for example, a biologically pure culture, or as spores (or other forms of the bacterial strain) in association with a pharmaceutically acceptable excipient suitable for human administration. In some embodiments, more than one microbe can be isolated. For example, “isolated microbes” can refer to a mixture of two or more microbes that have been separated from at least one of the materials with which they are associated in a particular environment.

In some embodiments, the isolated microbes exist as isolated and biologically pure cultures. As used herein, the term “biologically pure” refers to a composition comprising a species or strains of a microbe, wherein the composition is substantially free from the material from which the microbe was isolated or produced and from other microbes (e.g., other species or strains and other microbes of a different taxonomic classification). In some embodiments, “biologically pure” can refer to a composition that comprises a strain of a bacterial strain that is substantially free from the material from which the bacterial strain was isolated or produced and from other microbes, e.g., other strains of the same bacterial strain, other species of the same bacteria, and other bacteria and/or microbes of a different taxonomic classification). It will be appreciated by one of skill in the art, that an isolated and biologically pure culture of a particular microbe, denotes that said culture is substantially free (within scientific reason) of other living organisms and contains only the individual microbe in question. As used herein, “substantially free” means that a composition comprising a species or strain of a microbe is at least about 80%, about 85%, about 90%, about 91%, about 92%, about 93%, about 94%, about 95%, about 96%, about 97%, about 98%, about 99%, or more than about 99% free of the material from which the microbe was isolated or produced and from other microbes. In some embodiments, a biologically pure composition contains no other bacterial strain in quantities that can be detected by typical bacteriological techniques.

As used herein, the term “mutation” includes a natural or induced mutation comprising at least a single base or amino acid alteration in a DNA or protein sequence. For example, a mutation can include a base substitution, a deletion, an insertion, a transversion, or any other modification known to those skilled in the art, including a genetic modification introduced into a parent nucleotide or an amino acid sequence.

As used herein, “probiotic” refers to a substantially pure microbe (i.e., a single isolate) or a mixture of microbes, and may also include any additional components that can be administered to a subject (e.g., a human), for restoring or altering the microbiota or microbiome in the subject. In some embodiments, a probiotic or microbial inoculant composition can be administered with an agent to allow the microbe(s) to survive the environment of the gastrointestinal tract, i.e., to resist low pH and/or to grow in the gastrointestinal environment. In some embodiments, a composition as described herein includes a probiotic.

As used herein, “prebiotic” refers to an agent that increases the number and/or activity of one or more microbes. Such microbes can include microbes for restoring or altering the microbiota or microbiome of a subject. Non-limiting examples of a prebiotic include a fructooligosaccharide (e.g., oligofructose, inulin, or an inulin-type fructan), a galactooligosaccharide, an amino acid, an alcohol. See, for example, Ramirez-Farias et al. (2008. Br. J Nutr. 4:1-10) and Pool-Zobel and Sauer (2007. J Nutr. 137:2580-2584).

As used herein, a “live biotherapeutic product” or “LBP” refers to a biological product that: 1) contains live organisms, such as bacteria, and 2) is applicable to the prevention, treatment, and/or cure of a disease or condition of a subject.

A “combination” of two or more bacteria, e.g., bacterial strains, can refer to the physical co-existence of the bacteria, either in the same material or product. In some embodiments, a combination of two or more bacteria can include the temporal co-administration or co-localization of the two or more bacteria.

The terms “percent identity” or “identity” in the context of two or more nucleic acids or polypeptides, refers to the measurement of the similarity between the two or more sequences. The percent identity can be measured by any method known to one of skill in the art including using a sequence comparison software, an algorithm, and by visual inspection.

In general, the percent identity for two or more sequences (e.g., a nucleic acid or amino acid sequence), also referred to as the “percent sequence identity”, is calculated by determining the number of matched positions in the aligned nucleic acid or amino acid sequences, dividing the number of matched positions by the total number of aligned nucleotides or amino acids, respectively, and multiplying by 100. A matched position refers to a position in which identical nucleotides or amino acids occur at the same position in the aligned sequences. As an example, the total number of aligned nucleotides can refer to the minimum number of the 16S rRNA gene nucleotides that are necessary to align the second sequence, and does not include alignment (e.g., forced alignment) with non-16S rRNA gene sequences. The total number of aligned nucleotides may correspond to the entire 16S rRNA gene sequence or may correspond to fragments of the full-length 16S rRNA gene sequence.

Sequences can be aligned using an algorithm, for example, the algorithm as described by Altschul et al. (Nucleic Acids Res, 25:3389-3402, 1997) and incorporated into BLAST (basic local alignment search tool) programs, which are available at ncbi.nlm.nih.gov. BLAST searches or alignments can be performed to determine percent sequence identity between a 16S rRNA gene nucleic acid and any other sequence or portion thereof using the Altschul et al. algorithm. BLASTN can be used to align and compare the identity between nucleic acid sequences, while BLASTP can be used to align and compare the identity between amino acid sequences. When utilizing a BLAST program to calculate the percent identity between a 16S rRNA gene sequence and another sequence, the default parameters of the program are used.

Generally, a bacterial strain genomic sequence will contain multiple copies of 16S rRNA sequences. The 16S rRNA sequences can be used for making distinctions between species and strains. For example, if one or more of the 16S rRNA sequences shares less than 97% sequence identity from a reference sequence, then the two organisms from which the sequences were obtained can be of different species or strains.

The term “combination therapy” as used herein refers to a dosing regimen of one or more bacterial strains and one or more other treatments of colorectal cancer and/or adjunct therapies, wherein the bacterial strain and other treatment (e.g., a therapeutic agent) are administered together or separately in a manner prescribed by a medical care taker or according to a regulatory agency. As can be appreciated in the art, a combination therapy can be administered to a patient for a period of time. In some embodiments, the period of time occurs following the administration of one or more of: a different bacterial strain, a different treatment/therapeutic agent, and a different combination of treatments/therapeutic agents to the subject. In some embodiments, the period of time occurs before the administration of one or more of: a different bacterial strain, a different treatment/therapeutic agent, and a different combination of therapeutic treatments/agents to the subject.

The term “fixed combination” means that one or more bacterial strains as described herein, or a composition thereof, and at least one other treatment and/or adjunct therapy (e.g., a prebiotic, a probiotic, a chemotherapeutic agent, targeted therapy, immunotherapy, or a combination thereof), are both administered to a subject simultaneously in the form of a single composition or dosage.

The term “non-fixed combination” means that one or more bacterial strains as described herein, or a composition thereof, and at least one other treatment or adjunct therapy (e.g., a prebiotic, a probiotic, a chemotherapeutic agent, targeted therapy, immunotherapy, or a combination thereof) are formulated as separate compositions or dosages such that they may be administered to a subject simultaneously or sequentially with variable intervening time limits. These also apply to cocktail therapies, e.g., the administration of three or more therapeutic agents.

Reference to the term “about” has its usual meaning in the context of compositions to allow for reasonable variations in amounts that can achieve the same effect and also refers herein to a value of plus or minus 10% of the provided value. For example, “about 20” means or includes amounts from 18 to and including 22.

Unless otherwise required by context, singular terms shall include pluralities and plural terms shall include the singular. As used herein, the singular form “a,” “an,” and “the” include plural references unless indicated otherwise. For example, “an” excipient includes one or more excipients. It is understood that aspects and variations of the invention described herein include “consisting of” and/or “consisting essentially of” aspects and variations.

The details of one or more embodiments of the invention are set forth in the accompanying drawings and the description below. Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIG. 1 is an exemplary schematic of the analysis.

FIG. 2 has plots showing the tallies of strains detected (after prevalence filtering). The top panel tallies the strains detected in increasing number of datasets. The middle panel shows the number of strains that were significantly differentially abundant in 1 or more datasets. The bottom panel shows the number of strains that exhibit concordance in direction of the log 2 fold change, in increasing proportions of datasets in which they were detected. Only strains detected in at least two datasets are included.

FIG. 3 is a plot showing that significant findings from isolated dataset analysis rarely exhibits concordance in the direction of shifts across multiple datasets. Each row represents a strain identified as significantly differentially abundant by at least one isolated analysis. Asterisk denotes dataset(s) where significant changes of a strain was observed. Cell color indicates direction of the log 2 fold change: reduced (reduced) (green) or enriched (increased) (pink) in dysbiosis and not detected (white). Cells are shaded dark if the direction of log 2 fold change is concordant across all datasets a strain was detected in, i.e., the strain is associated with homeostasis or dysbiosis only. See FIG. 8 for details on contrasts analyzed within each cohort of subjects.

FIG. 4 is a plot showing the distribution of effect sizes per dataset. 16S-NGS refers to sequencing of the 16S rRNA gene via next generation sequencing (NGS).

FIG. 5 is a plot showing strains (dots) significantly differentially abundant in eubiotic or dysbiotic state by isolated-dataset analysis (grey squares) or MTMA (grey spheres) in colorectal cancer. Strains (dots) are sized by the number of datasets in which they were detected and colored as follows: significant by isolated analysis only (dark green); MTMA only (purple); or both (blue). Solid lines connect MTMA results to strains and dashed lines connect isolated analysis results to strains. Thick and thin lines indicate significant and non-significant findings, respectively. Red and green lines indicate enrichment (increase) and reduction (decrease) in dysbiotic state, respectively. Annotation for strains described here are provided in FIG. 9.

FIG. 6 is a forest plot demonstrating distribution of log 2 fold changes and 95% confidence intervals for strains that were identified as significantly differentially abundant by MTMA. Circles and triangles indicate log 2 fold change estimated by isolated analysis and MTMA, respectively. Error bars in the forest plots correspond to the 95% confidence interval. Green and blue indicate significant and nonsignificant findings, respectively, and grey indicates cases where an adjusted p-value could not be imputed by the statistical test. Annotation for strains described here are provided in FIG. 9.

FIG. 7 is a plot of MTMA-derived adjusted p-values and log 2 fold changes. Data points are shaded according to the proportion of datasets in which the strain was detected. Significantly dysbiosis-associated strains plot in the upper left quadrant, whereas homeostasis-associated strains plot in the upper right quadrant. FIG. 9 provides strain names for strain identifiers indicated in the plot.

FIG. 8 is a table showing details on contrasts analyzed within each cohort of subjects.

FIG. 9 is a table showing the association between the strain identifiers and strain names.

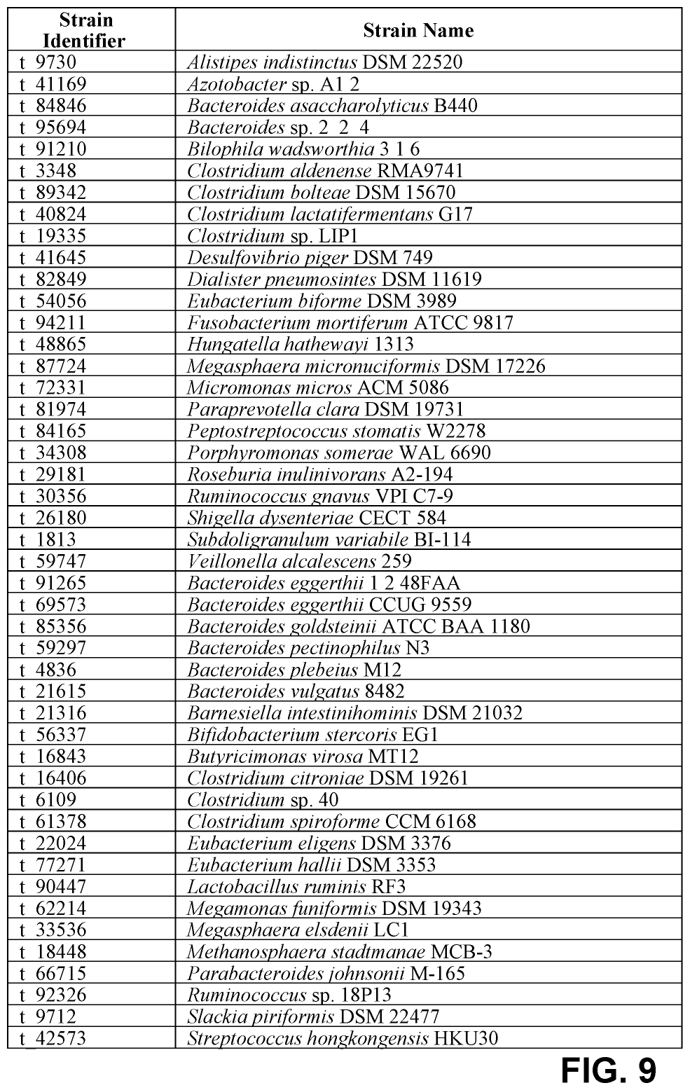

### DETAILED DESCRIPTION

This document provides compositions and methods for treating subjects in need thereof (e.g., subjects having colorectal cancer) using one or more bacterial strains. Colorectal cancer usually begins as polyps, e.g., small, noncancerous clumps of cells that form on the inside of the colon and/or rectum. There are two main types of polyps: adenomatous polyps (adenomas); and hyperplastic polyps and inflammatory polyps. Hyperplastic polyps and inflammatory polyps are generally not pre-cancerous whereas adenomatous polyps may be precursor lesions to colorectal cancer. See, e.g., Montalban-Arques and Scharl. EBioMedicine. 2019 October; 48:648-655.

In some embodiments, methods for treating a subject in need thereof are provided herein. In some embodiments, one or more of Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, and Streptococcus hongkongensis HKU30 is reduced (decreased) in a sample (e.g., fecal sample or a biopsy sample such as an intestinal biopsy sample or a colorectal biopsy sample) from the subject in need thereof. For example, one or more of Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, and Streptococcus hongkongensis HKU30 is reduced (decreased) in the sample (e.g., fecal sample or a biopsy sample such as an intestinal biopsy sample or a colorectal biopsy sample) from the subject in need thereof compared to a control sample (reference). Determining that one or more of Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, and Streptococcus hongkongensis HKU30 is reduced (decreased) in the sample from the subject in need thereof can comprise sequencing one or more nucleic acids from the bacteria. In some embodiments, the subject in need thereof has been diagnosed with colorectal cancer.

The methods provided herein can include administering to the subject a composition that includes an effective amount of a bacterial strain. In some embodiments, the bacterial strain can be selected from the group consisting of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, and a combination thereof (e.g., any two, any three, any four, any five, any six, any seven, any eight, any nine, any ten, any eleven, any twelve, any thirteen, any fourteen, any fifteen, any sixteen, any seventeen, any eighteen, any nineteen, any twenty, any twenty-one, or twenty two of the bacterial strains). In some embodiments, the bacterial strain can be selected from the group consisting of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, and a combination thereof (e.g., any two, any three, any four, any five, any six, any seven, any eight, any nine, any ten, any eleven, any twelve, any thirteen, any fourteen, any fifteen, any sixteen, or seventeen of the bacterial strains). In some embodiments, the bacterial strain can be selected from the group consisting of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, and a combination thereof (e.g., any two, any three, any four, any five, any six, any seven, any eight, any nine, any ten, any eleven, any twelve, any thirteen, any fourteen, or fifteen of the bacterial strains).

In some embodiments, the bacterial strain in the composition comprises Bacteroides eggerthii 1 2 48FAA. A complete genomic sequence for Bacteroides eggerthii 1 2 48FAA is available in the GenBank database as, e.g., Accession No. GCF_000273465 and/or GCF_000185605. In some embodiments, the Bacteroides eggerthii 1 2 48FAA included in a composition provided herein can have a genomic sequence with at least about 95% sequence identity to the genomic sequence published as Accession No. GCF_000273465 and/or GCF_000185605. For example, Bacteroides eggerthii 1 2 48FAA included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000273465 and/or GCF_000185605. In some embodiments, Bacteroides eggerthii 1 2 48FAA included in a composition provided herein has a 16S RNA gene that is at least 90% identical to one or more (e.g., one, two, three, four, five, or all) of SEQ ID NO:1, SEQ ID NO:2, SEQ ID NO:3, SEQ ID NO:4, SEQ ID NO:5, and SEQ ID NO:6. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, or all) of SEQ ID NO:1, SEQ ID NO:2, SEQ ID NO:3, SEQ ID NO:4, SEQ ID NO:5, and SEQ ID NO:6.

In some embodiments, the bacterial strain in the composition comprises Bacteroides eggerthii CCUG 9559. A complete genomic sequence for Bacteroides eggerthii CCUG 9559 is available in the GenBank database as, e.g., Accession No. GCF_000155815. In some embodiments, the Bacteroides eggerthii CCUG 9559 included in a composition provided herein can have a genomic sequence with at least about 95% sequence identity to the genomic sequence published as GCF_000155815. For example, Bacteroides eggerthii CCUG 9559 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000155815. In some embodiments, Bacteroides eggerthii CCUG 9559 included in a composition provided herein has a 16S RNA gene that is at least 90% identical to one or more (e.g., one, two, three, four, five, or all) of SEQ ID NO:7, SEQ ID NO:8, SEQ ID NO:9, SEQ ID NO:10, SEQ ID NO:11, and SEQ ID NO:12. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, or all) of SEQ ID NO:7, SEQ ID NO:8, SEQ ID NO:9, SEQ ID NO:10, SEQ ID NO:11, and SEQ ID NO:12.

In some embodiments, the bacterial strain in the composition comprises Bacteroides goldsteinii ATCC BAA 1180. A complete genomic sequence for Bacteroides goldsteinii ATCC BAA 1180 is available in the GenBank database as, e.g., Accession No. GCF_000969835. In some embodiments, the Bacteroides goldsteinii ATCC BAA 1180 included in a composition provided herein can have a genomic sequence with at least about 95% sequence identity to the genomic sequence published as GCF_000969835. For example, Bacteroides goldsteinii ATCC BAA 1180 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000969835. In some embodiments, Bacteroides goldsteinii ATCC BAA 1180 included in a composition provided herein has a 16S RNA gene that is at least 90% identical to one or more (e.g., one, two, three, four, five, six or all) of SEQ ID NO:13, SEQ ID NO:14, SEQ ID NO:15, SEQ ID NO:16, SEQ ID NO:17, SEQ ID NO:18, and SEQ ID NO:19 For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six or all) of SEQ ID NO:13, SEQ ID NO:14, SEQ ID NO:15, SEQ ID NO:16, SEQ ID NO:17, SEQ ID NO:18, and SEQ ID NO:19.

In some embodiments, the bacterial strain in the composition comprises Bacteroides pectinophilus N3. A complete genomic sequence for Bacteroides pectinophilus N3 is available in the GenBank database as, e.g., Accession No. GCF_000155855. In some embodiments, the Bacteroides pectinophilus N3 included in a composition provided herein can have a genomic sequence with at least about 95% sequence identity to the genomic sequence published as GCF_000155855. For example, Bacteroides pectinophilus N3 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000155855. In some embodiments, Bacteroides pectinophilus N3 included in a composition provided herein has a 16S RNA gene that is at least 90% identical to one or more (e.g., one, two, three, four, five, or all) of SEQ ID NO:20, SEQ ID NO:21, SEQ ID NO:22, SEQ ID NO:23, SEQ ID NO:24, and SEQ ID NO:25. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, or all) of SEQ ID NO:20, SEQ ID NO:21, SEQ ID NO:22, SEQ ID NO:23, SEQ ID NO:24, and SEQ ID NO:25.

In some embodiments, the bacterial strain in the composition comprises Bacteroides plebeius M12. A complete genomic sequence for Bacteroides plebeius M12 is available in the GenBank database as, e.g., Accession No. GCF_000187895. In some embodiments, the Bacteroides plebeius M12 included in a composition provided herein can have a genomic sequence with at least about 95% sequence identity to the genomic sequence published as GCF_000187895. For example, Bacteroides plebeius M12 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000187895. In some embodiments, Bacteroides plebeius M12 included in a composition provided herein has a 16S RNA gene that is at least 90% identical to one or more (e.g., one, two, or all) of SEQ ID NO:26, SEQ ID NO:27, and SEQ ID NO:28. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, or all) of SEQ ID NO:26, SEQ ID NO:27, and SEQ ID NO:28.

In some embodiments, the bacterial strain in the composition comprises Bacteroides vulgatus 8482. A complete genomic sequence for Bacteroides vulgatus 8482 is available in the GenBank database as, e.g., Accession No. GCF_000012825. In some embodiments, the Bacteroides vulgatus 8482 included in a composition provided herein can have a genomic sequence with at least about 95% sequence identity to the genomic sequence published as GCF_000012825. For example, Bacteroides vulgatus 8482 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000012825. In some embodiments, Bacteroides vulgatus 8482 included in a composition provided herein has a 16S RNA gene that is at least 90% identical to one or more (e.g., one, two, three, four, five, six, seven, eight, nine, ten, eleven or all) SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, and SEQ ID NO:40. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, seven, eight, nine, ten, eleven or all) SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, and SEQ ID NO:40.

In some embodiments, the bacterial strain in the composition comprises Barnesiella intestinihominis DSM 21032. A complete genomic sequence for Barnesiella intestinihominis DSM 21032 is available in the GenBank database as, e.g., Accession No. GCF_000296465. In some embodiments, the Barnesiella intestinihominis DSM 21032 included in a composition provided herein can have a genomic sequence with at least about 95% sequence identity to the genomic sequence published as GCF_000296465. For example, Barnesiella intestinihominis DSM 21032 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000296465. In some embodiments, Barnesiella intestinihominis DSM 21032 included in a composition provided herein has a 16S RNA gene that is at least 90% identical to one or more (e.g., one, two, three, four, five, six, seven, eight, nine, or all) of SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, and SEQ ID NO:50. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, seven, eight, nine, or all) of SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, and SEQ ID NO:50.

In some embodiments, the bacterial strain in the composition comprises Bifidobacterium stercoris EG1. A complete genomic sequence for Bifidobacterium stercoris EG1 is available in the GenBank database as, e.g., Accession No. GCF_000771705 or GCF_000741415. In some embodiments, the Bifidobacterium stercoris EG1 included in a composition provided herein can have a genomic sequence with at least about 95% sequence identity to the genomic sequence published as GCF_000771705 and/or GCF_000741415 For example, Bifidobacterium stercoris EG1 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000771705 and/or GCF_000741415. In some embodiments, Bifidobacterium stercoris EG1 included in a composition provided herein has a 16S RNA gene that is at least 90% identical to one or more (e.g., one, two, three, or all) of SEQ ID NO:51, SEQ ID NO:52, SEQ ID NO:53, and SEQ ID NO:54. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, or all) of SEQ ID NO:51, SEQ ID NO:52, SEQ ID NO:53, and SEQ ID NO:54.

In some embodiments, the bacterial strain in the composition comprises Clostridium sp. 40. In some embodiments, Clostridium sp. 40 included in a composition provided herein has a 16S RNA gene that is at least 90% identical to SEQ ID NO:57. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:57.

In some embodiments, the bacterial strain in the composition comprises Clostridium spiroforme CCM 6168. A complete genomic sequence for Clostridium spiroforme CCM 6168 is available in the GenBank database as, e.g., Accession No. GCF_000154805. In some embodiments, the Clostridium spiroforme CCM 6168 included in a composition provided herein can have a genomic sequence with at least about 95% sequence identity to the genomic sequence published as GCF_000154805. For example, Clostridium spiroforme CCM 6168 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000154805. In some embodiments, Clostridium spiroforme CCM 6168 included in a composition provided herein has a 16S RNA gene that is at least 90% identical to one or more (e.g., one, two, three, four, five, or all) of SEQ ID NO:58, SEQ ID NO:59, SEQ ID NO:60, SEQ ID NO:61, SEQ ID NO:62, and SEQ ID NO:63. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, or all) of SEQ ID NO:58, SEQ ID NO:59, SEQ ID NO:60, SEQ ID NO:61, SEQ ID NO:62, and SEQ ID NO:63.

In some embodiments, the bacterial strain in the composition comprises Eubacterium eligens DSM 3376. A complete genomic sequence for Eubacterium eligens DSM 3376 is available in the GenBank database as, e.g., Accession No. GCF_000146185. In some embodiments, the Eubacterium eligens DSM 3376 included in a composition provided herein can have a genomic sequence with at least about 95% sequence identity to the genomic sequence published as GCF_000146185. For example, Eubacterium eligens DSM 3376 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000146185. In some embodiments, Eubacterium eligens DSM 3376 included in a composition provided herein has a 16S RNA gene that is at least 90% identical to one or more (e.g., one, two, three, four, five, six, or all) of SEQ ID NO:64, SEQ ID NO:65, SEQ ID NO:66, SEQ ID NO:67, SEQ ID NO:68, SEQ ID NO:69, and SEQ ID NO:70. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, or all) of SEQ ID NO:64, SEQ ID NO:65, SEQ ID NO:66, SEQ ID NO:67, SEQ ID NO:68, SEQ ID NO:69, and SEQ ID NO:70.

In some embodiments, the bacterial strain in the composition comprises Eubacterium hallii DSM 3353. A complete genomic sequence for Eubacterium hallii DSM 3353 is available in the GenBank database as, e.g., Accession No. GCF_000173975. In some embodiments, the Eubacterium hallii DSM 3353 included in a composition provided herein can have a genomic sequence with at least about 95% sequence identity to the genomic sequence published as GCF_000173975. For example, Eubacterium hallii DSM 3353 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000173975. In some embodiments, Eubacterium hallii DSM 3353 included in a composition provided herein has a 16S RNA gene that is at least 90% identical to one or both of SEQ ID NO:71 and SEQ ID NO:72. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or both of SEQ ID NO:71 and SEQ ID NO:72.

In some embodiments, the bacterial strain in the composition comprises Megamonas funiformis DSM 19343. A complete genomic sequence for Megamonas funiformis DSM 19343 is available in the GenBank database as, e.g., Accession No. GCF_000245775. In some embodiments, the Megamonas funiformis DSM 19343 included in a composition provided herein can have a genomic sequence with at least about 95% sequence identity to GCF_000245775. In some embodiments, Megamonas funiformis DSM 19343 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000245775. In some embodiments, Megamonas funiformis DSM 19343 included in a composition provided herein has a 16S RNA gene that is at least 90% identical to one or more (e.g., one, two, three, four, five, six, or all) of SEQ ID NO:80, SEQ ID NO:81, SEQ ID NO:82, SEQ ID NO:83, SEQ ID NO:84, SEQ ID NO:85, and SEQ ID NO:86. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, or all) of SEQ ID NO:80, SEQ ID NO:81, SEQ ID NO:82, SEQ ID NO:83, SEQ ID NO:84, SEQ ID NO:85, and SEQ ID NO:86.

In some embodiments, the bacterial strain in the composition comprises Megasphaera elsdenii LC1. A complete genomic sequence for Megasphaera elsdenii LC1 is available in the GenBank database as, e.g., Accession No. GCF_000283495. In some embodiments, the Megasphaera elsdenii LC1 included in a composition provided herein can have a genomic sequence with at least about 95% sequence identity to the genomic sequence published as GCF_000283495. For example, Megasphaera elsdenii LC1 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000283495. In some embodiments, Megasphaera elsdenii LC1 included in a composition provided herein has a 16S RNA gene that is at least 90% identical to one or more (e.g., one, two, three, four, five, six, seven, eight, or all) of SEQ ID NO:87, SEQ ID NO:88, SEQ ID NO:89, SEQ ID NO:90, SEQ ID NO:91, SEQ ID NO:92, SEQ ID NO:93, SEQ ID NO:94, and SEQ ID NO:95. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, seven, eight, or all) of SEQ ID NO:87, SEQ ID NO:88, SEQ ID NO:89, SEQ ID NO:90, SEQ ID NO:91, SEQ ID NO:92, SEQ ID NO:93, SEQ ID NO:94, and SEQ ID NO:95.

In some embodiments, the bacterial strain in the composition comprises Parabacteroides johnsonii M-165. A complete genomic sequence for Parabacteroides johnsonii M-165 is available in the GenBank database as, e.g., Accession No. GCF_000156495. In some embodiments, the Parabacteroides johnsonii M-165 included in a composition provided herein can have a genomic sequence with at least about 95% sequence identity to the genomic sequence published as GCF_000156495. For example, Parabacteroides johnsonii M-165 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000156495. In some embodiments, Parabacteroides johnsonii M-165 included in a composition provided herein has a 16S RNA gene that is at least 90% identical to one or more (e.g., one, two, three, or all) of SEQ ID NO:104, SEQ ID NO:105, SEQ ID NO:106, and SEQ ID NO:107. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, or all) of NO:104, SEQ ID NO:105, SEQ ID NO:106, and SEQ ID NO:107.

In some embodiments, the bacterial strain in the composition comprises Butyricimonas virosa MT12. In some embodiments, Butyricimonas virosa MT12 included in a composition provided herein has a 16S RNA gene that is at least 90% identical to SEQ ID NO:55. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:55.

In some embodiments, the bacterial strain in the composition comprises Clostridium citroniae DSM 19261. In some embodiments, Clostridium citroniae DSM 19261 included in a composition provided herein has a 16S RNA gene that is at least 90% identical to SEQ ID NO:56. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:56.

In some embodiments, the bacterial strain in the composition comprises Lactobacillus ruminis RF3. A complete genomic sequence for Lactobacillus ruminis RF3 is available in the GenBank database as, e.g., Accession No. GCF_000224985. In some embodiments, the Lactobacillus ruminis RF3 included in a composition provided herein can have a genomic sequence with at least about 95% sequence identity to the genomic sequence published as GCF_000224985. For example, Lactobacillus ruminis RF3 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000224985. In some embodiments, Lactobacillus ruminis RF3 included in a composition provided herein has a 16S RNA gene that is at least 90% identical to one or more (e.g., one, two, three, four, five, six, or all) of SEQ ID NO:73, SEQ ID NO:74, SEQ ID NO:75, SEQ ID NO:76, SEQ ID NO:77, SEQ ID NO:78, and SEQ ID NO:79. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, or all) of SEQ ID NO:73, SEQ ID NO:74, SEQ ID NO:75, SEQ ID NO:76, SEQ ID NO:77, SEQ ID NO:78, and SEQ ID NO:79.

In some embodiments, the bacterial strain in the composition comprises Methanosphaera stadtmanae MCB-3. A complete genomic sequence for Methanosphaera stadtmanae MCB-3 is available in the GenBank database as, e.g., Accession No. GCF_000012545. In some embodiments, the Methanosphaera stadtmanae MCB-3 included in a composition provided herein can have a genomic sequence with at least about 95% sequence identity to the genomic sequence published as GCF_000012545. For example, Methanosphaera stadtmanae MCB-3 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000012545. In some embodiments, Methanosphaera stadtmanae MCB-3 included in a composition provided herein has a 16S RNA gene that is at least 90% identical to one or more (e.g., one, two, three, four, five, six, seven, or all) of SEQ ID NO:96, SEQ ID NO:97, SEQ ID NO:98, SEQ ID NO:99, SEQ ID NO:100, SEQ ID NO:101, SEQ ID NO:102, and SEQ ID NO:103. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, seven, or all) of SEQ ID NO:96, SEQ ID NO:97, SEQ ID NO:98, SEQ ID NO:99, SEQ ID NO:100, SEQ ID NO:101, SEQ ID NO:102, and SEQ ID NO:103.

In some embodiments, the bacterial strain in the composition comprises Ruminococcus sp. 18P13. In some embodiments, the bacterial strain comprises Ruminococcus sp. 18P13. A complete genomic sequence for Ruminococcus sp. 18P13 is available in the GenBank database as, e.g., Accession No. GCF_000210095 and GCF_001312825. In some embodiments, the Ruminococcus sp. 18P13 included in a composition provided herein can have a genomic sequence with at least about 95% sequence identity to the genomic sequence published as GCF_000210095 and/or GCF_001312825. For example, Ruminococcus sp. 18P13 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000210095 and/or GCF_001312825. In some embodiments, Ruminococcus sp. 18P13 included in a composition provided herein has a 16S RNA gene that is at least 90% identical to one or more (e.g., one, two, three, or all) of SEQ ID NO:108, SEQ ID NO:109, SEQ ID NO:110, and SEQ ID NO:111. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, or all) of SEQ ID NO:108, SEQ ID NO:109, SEQ ID NO:110, and SEQ ID NO:111.

In some embodiments, the bacterial strain in the composition comprises Slackia piriformis DSM 22477. A complete genomic sequence for Slackia piriformis DSM 22477 is available in the GenBank database as, e.g., Accession No. GCF_000296445. In some embodiments, the Slackia piriformis DSM 22477 included in a composition provided herein can have a genomic sequence with at least about 95% sequence identity to the genomic sequence published as GCF_000296445. For example, Slackia piriformis DSM 22477 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000296445. In some embodiments, Slackia piriformis DSM 22477 included in a composition provided herein has a 16S RNA gene that is at least 90% identical to one or more (e.g., one, two, three, four, five, or all) of SEQ ID NO:112, SEQ ID NO:113, SEQ ID NO:114, SEQ ID NO:115, SEQ ID NO:116, and SEQ ID NO:117. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, or all) of SEQ ID NO:112, SEQ ID NO:113, SEQ ID NO:114, SEQ ID NO:115, SEQ ID NO:116, and SEQ ID NO:117.

In some embodiments, the bacterial strain in the composition comprises Streptococcus hongkongensis HKU30. In some embodiments, Streptococcus hongkongensis HKU30 included in a composition provided herein has a 16S RNA gene that is at least 90% identical to one or both of SEQ ID NO:118 and SEQ ID NO:119. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or both of SEQ ID NO:118 and SEQ ID NO:119.

In some embodiments, the composition can include two or more bacterial strains selected from the group consisting of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, and Streptococcus hongkongensis HKU30. For example, the composition can include three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, or all twenty-two bacterial strains selected from the group consisting of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, and Streptococcus hongkongensis HKU30. Identifying characteristics of each strain are described above.

In some embodiments, the composition can include two or more bacterial strains selected from the group consisting of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, and Parabacteroides johnsonii M-165. For example, the composition can include three or more, four or more, five or more, six or more, seven or more, eight or more, nine or more, ten or more, eleven or more, twelve or more, thirteen or more, fourteen or more, or all fifteen bacterial strains selected from the group consisting of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, and Parabacteroides johnsonii M-165. Identifying characteristics of each strain are described above.

In some embodiments, a method can include detecting, in a sample from the subject, a dysbiosis associated with colorectal cancer, e.g., before administering to the subject an effective amount of a bacterial strain or a composition containing the bacterial strain. The sample can be a biopsy sample such as an intestinal biopsy sample or a colorectal biopsy sample. In some embodiments, the sample is a fecal sample.

In some embodiments, detecting the dysbiosis associated with colorectal cancer can include determining bacterial gene expression in the sample from the subject (e.g., fecal sample or a biopsy sample such as an intestinal biopsy sample or a colorectal biopsy sample). For example, the bacterial gene expression can be determined in the sample from the subject e.g., before administering to the subject an effective amount of a bacterial strain or a composition containing the bacterial strain and/or after administering to the subject an effective amount of a bacterial strain or a composition containing the bacterial strain. Determining the bacterial gene expression can include performing, for example, RNAseq and/or RT-qPCR. In some embodiments, detecting the dysbiosis associated with colorectal cancer comprises determining bacterial composition in the sample from the subject (e.g., fecal sample or a biopsy sample such as an intestinal biopsy sample or a colorectal biopsy sample). For example, the bacterial composition can be determined in a sample from the subject, e.g., before administering to the subject an effective amount of a bacterial strain or a composition containing the bacterial strain and/or after administering to the subject an effective amount of a bacterial strain or a composition containing the bacterial strain. Determining the bacterial composition can include, for example, sequencing one or more nucleic acids from the bacteria. In some embodiments, bacteria can be identified by their 16S rRNA gene sequence. In some embodiments, detecting the dysbiosis comprises determining that Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, or a combination thereof, is reduced (decreased) in the sample from subject (e.g., reduced (decreased) in the gastrointestinal tract of the subject). In some embodiments, detecting the dysbiosis comprises determining that Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, or a combination thereof, is reduced (decreased) in the sample from subject (e.g., reduced (decreased) in the gastrointestinal tract of the subject).

In some embodiments, detecting the dysbiosis associated with colorectal cancer comprises determining that Alistipes indistinctus DSM 22520, Azotobacter sp. A1 2, Bacteroides asaccharolyticus B440, Bacteroides sp. 2_2_4, Bilophila wadsworthia 3 1 6, Clostridium aldenense RMA9741, Clostridium bolteae DSM 15670, Clostridium lactatifermentans G17, Clostridium sp. LIP1, Desulfovibrio piger DSM 749, Dialister pneumosintes DSM 11619, Eubacterium biforme DSM 3989, Fusobacterium mortiferum ATCC 9817, Hungatella hathewayi 1313, Megasphaera micronuciformis DSM 17226, Micromonas micros ACM 5086, Paraprevotella clara DSM 19731, Peptostreptococcus stomatis W2278, Porphyromonas somerae WAL 6690, Roseburia inulinivorans A2-194, Ruminococcus gnavus VPI C7-9, Shigella dysenteriae CECT 584, Subdoligranulum variabile BI-114, Veillonella alcalescens 259, or a combination thereof, is enriched (increased) in the sample from subject.

In some embodiments, a method as provided herein can include decreasing a population of an enriched (increased) bacterial strain in a subject (e.g., a subject with colorectal cancer). In some embodiments, detecting the decrease in the population of an enriched (increased) bacterial strain comprises determining the bacterial composition in a sample from the subject (e.g., fecal sample or a biopsy sample such as an intestinal biopsy sample or a colorectal biopsy sample). For example, the bacterial composition can be determined in a sample from the subject before administering to the subject an effective amount of a bacterial strain or a composition containing the bacterial strain and after administering to the subject an effective amount of a bacterial strain or a composition containing the bacterial strain. For example, the population of an enriched (increased) bacterial strain can be decreased by at least 1%, at least 2%, at least 3%, at least 4%, at least 5%, at least 6%, at least 7%, at least 10%, at least 20%, at least 25%, at least 30%, at least 35%, at least 40%, or at least 50%, e.g., in a sample from the subject after administration of a bacterial strain or a composition containing the bacterial strain to the subject compared to before administration to the subject of an effective amount of a bacterial strain or a composition containing the bacterial strain. Determining the bacterial composition can include, for example, sequencing one or more nucleic acids from the bacteria. In some embodiments, bacteria can be identified by their 16S rRNA gene sequence.

In some embodiments, the enriched (increased) bacterial strain can be selected from the group consisting of: Alistipes indistinctus DSM 22520, Azotobacter sp. A1 2, Bacteroides asaccharolyticus B440, Bacteroides sp. 2_2_4, Bilophila wadsworthia 3 1 6, Clostridium aldenense RMA9741, Clostridium bolteae DSM 15670, Clostridium lactatifermentans G17, Clostridium sp. LIP1, Desulfovibrio piger DSM 749, Dialister pneumosintes DSM 11619, Eubacterium biforme DSM 3989, Fusobacterium mortiferum ATCC 9817, Hungatella hathewayi 1313, Megasphaera micronuciformis DSM 17226, Micromonas micros ACM 5086, Paraprevotella clara DSM 19731, Peptostreptococcus stomatis W2278, Porphyromonas somerae WAL 6690, Roseburia inulinivorans A2-194, Ruminococcus gnavus VPI C7-9, Shigella dysenteriae CECT 584, Subdoligranulum variabile BI-114, Veillonella alcalescens 259, and a combination thereof.

In some embodiments, Alistipes indistinctus DSM 22520 has a 16S RNA gene that is at least 90% identical to one or more (e.g., one, two, three, four, or all) of SEQ ID NO:120, SEQ ID NO:121, SEQ ID NO:122, SEQ ID NO:123, and SEQ ID NO:124. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, or all) of SEQ ID NO:120, SEQ ID NO:121, SEQ ID NO:122, SEQ ID NO:123, and SEQ ID NO:124.

In some embodiments, Azotobacter sp. A1 2 has a 16S RNA gene that is at least 90% identical to one or more (e.g., one, two, three, four, five, six, or all) of SEQ ID NO:125, SEQ ID NO:126, SEQ ID NO:127, SEQ ID NO:128, SEQ ID NO:129, SEQ ID NO:130, and SEQ ID NO:131. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, or all) of SEQ ID NO:125, SEQ ID NO:126, SEQ ID NO:127, SEQ ID NO:128, SEQ ID NO:129, SEQ ID NO:130, and SEQ ID NO:131.

In some embodiments, Bacteroides asaccharolyticus B440 has a 16S RNA gene that is at least 90% identical to one or more (e.g., one, two, three, four, five, or all) of SEQ ID NO:132, SEQ ID NO:133, SEQ ID NO:134, SEQ ID NO:135, SEQ ID NO:136, and SEQ ID NO:137. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, or all) of SEQ ID NO:132, SEQ ID NO:133, SEQ ID NO:134, SEQ ID NO:135, SEQ ID NO:136, and SEQ ID NO:137.

In some embodiments, Bacteroides sp. 2_2_4 has a 16S RNA gene that is at least 90% identical to one or both of SEQ ID NO:138 and SEQ ID NO:139. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or both of SEQ ID NO:138 and SEQ ID NO:139.

In some embodiments, Bilophila wadsworthia 3 1 6 has a 16S RNA gene that is at least 90% identical to one or more (e.g., one, two, three, four, five, six or all) of SEQ ID NO:140, SEQ ID NO:141, SEQ ID NO:142, SEQ ID NO:143, SEQ ID NO:144, SEQ ID NO:145, and SEQ ID NO:146. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six or all) of SEQ ID NO:140, SEQ ID NO:141, SEQ ID NO:142, SEQ ID NO:143, SEQ ID NO:144, SEQ ID NO:145, and SEQ ID NO:146.

In some embodiments, Clostridium aldenense RMA9741 has a 16S RNA gene that is at least 90% identical to SEQ ID NO:147. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:147.

In some embodiments, Clostridium bolteae DSM 15670 has a 16S RNA gene that is at least 90% identical to one or more (e.g., one, two, three, four, or all) of SEQ ID NO:148, SEQ ID NO:149, SEQ ID NO:150, SEQ ID NO:151, and SEQ ID NO:152. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, or all) of SEQ ID NO:148, SEQ ID NO:149, SEQ ID NO:150, SEQ ID NO:151, and SEQ ID NO:152.

In some embodiments, Clostridium lactatifermentans G17 has a 16S RNA gene that is at least 90% identical to SEQ ID NO:153. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:153.

In some embodiments, Clostridium sp. LIP1 has a 16S RNA gene that is at least 90% identical to SEQ ID NO:154. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:154.

In some embodiments, Desulfovibrio piger DSM 749 has a 16S RNA gene that is at least 90% identical to SEQ ID NO:155. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:155.

In some embodiments, Dialister pneumosintes DSM 11619 has a 16S RNA gene that is at least 90% identical to SEQ ID NO:156. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:156.

In some embodiments, Eubacterium biforme DSM 3989 has a 16S RNA gene that is at least 90% identical to one or more (e.g., one, two, three, or all) of SEQ ID NO:157, SEQ ID NO:158, SEQ ID NO:159, and SEQ ID NO:160. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, or all) of SEQ ID NO:157, SEQ ID NO:158, SEQ ID NO:159, and SEQ ID NO:160.

In some embodiments, Fusobacterium mortiferum ATCC 9817 has a 16S RNA gene that is at least 90% identical to one or more (e.g., one, two, or all) of SEQ ID NO:161, SEQ ID NO:162, and SEQ ID NO:163. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, or all) of SEQ ID NO:161, SEQ ID NO:162, and SEQ ID NO:163.

In some embodiments, Hungatella hathewayi 1313 has a 16S RNA gene that is at least 90% identical to SEQ ID NO:164. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:164.

In some embodiments, Megasphaera micronuciformis DSM 17226 has a 16S RNA gene that is at least 90% identical to SEQ ID NO:165. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:165.

In some embodiments, Micromonas micros ACM 5086 has a 16S RNA gene that is at least 90% identical to one or more (e.g., one, two, three, four, five, or all) of SEQ ID NO:166, SEQ ID NO:167, SEQ ID NO:168, SEQ ID NO:169, SEQ ID NO:170, and SEQ ID NO:171. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, or all) of SEQ ID NO:166, SEQ ID NO:167, SEQ ID NO:168, SEQ ID NO:169, SEQ ID NO:170, and SEQ ID NO:171.

In some embodiments, Paraprevotella clara DSM 19731 has a 16S RNA gene that is at least 90% identical to one or both of SEQ ID NO:172 and SEQ ID NO:173. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or both of SEQ ID NO:172 and SEQ ID NO:173.

In some embodiments, Peptostreptococcus stomatis W2278 has a 16S RNA gene that is at least 90% identical to one or more (e.g., one, two, or all) of SEQ ID NO:174, SEQ ID NO:175, and SEQ ID NO:176. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, or all) of SEQ ID NO:174, SEQ ID NO:175, and SEQ ID NO:176.

In some embodiments, Porphyromonas somerae WAL 6690 has a 16S RNA gene that is at least 90% identical to one or both of SEQ ID NO:177 and SEQ ID NO:178. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or both of SEQ ID NO:177 and SEQ ID NO:178.

In some embodiments, Roseburia inulinivorans A2-194 has a 16S RNA gene that is at least 90% identical to SEQ ID NO:179. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:179.

In some embodiments, Ruminococcus gnavus VPI C7-9 has a 16S RNA gene that is at least 90% identical to one or more (e.g., one, two, three, four, five, or all) of SEQ ID NO:180, SEQ ID NO:181, SEQ ID NO:182, SEQ ID NO:183, SEQ ID NO:184, and SEQ ID NO:185. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, or all) of SEQ ID NO:180, SEQ ID NO:181, SEQ ID NO:182, SEQ ID NO:183, SEQ ID NO:184, and SEQ ID NO:185.

In some embodiments, Shigella dysenteriae CECT 584 has a 16S RNA gene that is at least 90% identical to SEQ ID NO:186. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:186.

In some embodiments, Subdoligranulum variabile BI-114 has a 16S RNA gene that is at least 90% identical to one or both of SEQ ID NO:187 and SEQ ID NO:188. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or both of SEQ ID NO:187 and SEQ ID NO:188.

In some embodiments, Veillonella alcalescens 259 has a 16S RNA gene that is at least 90% identical to one or more (e.g., one, two, three, or all) of SEQ ID NO:189, SEQ ID NO:190, SEQ ID NO:191, and SEQ ID NO:192. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, or all) of SEQ ID NO:189, SEQ ID NO:190, SEQ ID NO:191, and SEQ ID NO:192.

In some embodiments, decreasing the population of an enriched (increased) bacterial strain can include administering a bacteriophage to the subject. See, for example, Sabino et al. Aliment Pharmacol Ther. 51(1):53-63, 2020. In some embodiments, decreasing the population of an enriched (increased) bacterial strain can include administering to the subject a composition comprising an effective amount of a bacterial strain (e.g., a bacterial strain selected from the group consisting of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, and a combination thereof; or a bacterial strain selected from the group consisting of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, and a combination thereof).

In some embodiments, methods provided herein can include administering the composition that includes an effective amount of one or more bacterial strains to the subject at least once per day. For example, the composition can be administered two, three, four, or more times per day. In some embodiments, an effective amount of the bacterial strain is administered in one dose, e.g., once per day. In some embodiments, an effective amount of the bacterial strain is administered in more than one dose, e.g., more than once per day. In some embodiments, the method comprises administering the composition to the subject daily, every other day, every three days, or once a week.

In some embodiments, an effective amount of a bacterial strain (e.g., Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, or a combination thereof) in a composition described herein can include at least about 1×10³ CFUs of the bacterial strain. For example, an effective amount of a bacterial strain can be at least about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, or about 1×10¹⁴ CFUs of the bacterial strain. In some embodiments, the effective amount of a bacterial strain in a composition described herein comprises about 1×10³ to about 1×10¹⁵ CFUs of the bacterial strain (e.g., about 1×10³ to about 1×10⁶, about 1×10³ to about 1×10⁸, about 1×10³ to about 1×10¹⁰, about 1×10³ to about 1×10¹², about 1×10³ to about 1×10¹⁴, about 1×10⁷ to about 1×10¹², about 1×10¹³ to about 1×10¹⁵, about 1×10¹¹ to about 1×10¹⁵, about 1×10⁹ to about 1×10¹⁵, about 1×10⁷ to about 1×10¹⁵, or about 1×10⁵ to about 1×10¹⁵ CFUs of the bacterial strain).

In some embodiments, methods provided herein can include administering a composition comprising a bacterial strain as described herein in combination with one or more other treatments of colorectal cancer and/or in combination with adjunct therapies such as a therapeutic agent. In some embodiments, the treatment of colorectal cancer can be one or more of surgery, ablation, radiation therapy, embolization, or a combination thereof. In some embodiments, the treatment of colorectal cancer and/or adjunct therapy can be a therapeutic agent such as chemotherapeutic agent, targeted therapy, or immunotherapy. The composition comprising a bacterial strain and any other treatments and/or adjunct therapies can be administered together (e.g., in the same formulation), or the composition comprising the bacterial strain can be administered concurrently with, prior to, or subsequent to, the one or more other treatments or adjunct therapies.

In some embodiments, the therapeutic agent administered in combination with a composition comprising a bacterial strain as described herein includes a chemotherapeutic agent, targeted therapy, immunotherapy, or a combination thereof. In some embodiments, the chemotherapeutic agent includes aflibercept, capecitabine, fluorouracil, irinotecan, leucovorin, oxaliplatin, trifluridine, tipiracil, or a combination thereof. In some embodiments, the targeted therapy includes bevacizumab, cetuximab, ziv-aflibercept, panitumumab, regorafenib, or a combination thereof. In some embodiments, the combination of therapeutic agents includes FOLFOX (a combination of folinic acid, fluorouracil, and oxaliplatin), FOLFIRI (a combination of folinic acid, fluorouracil and irinotecan hydrochloride), CAPDX (a combination of capecitabine and oxaliplatin), FOLFIRI-bevacizumab, FOLFIRI-cetuximab, or XELOX (a combination of capecitabine and oxaliplatin), or a combination thereof. In some embodiments, the immunotherapy includes a checkpoint inhibitor. In some embodiments, the checkpoint inhibitor targets one or more of: CTLA-4, PD-1, PD-L1, BTLA, LAG-3, A2AR, TIM-3, B7-H3, VISTA, and IDO. In some embodiments, the checkpoint inhibitor is selected from the group consisting of: ipilimumab, nivolumab, pembrolizumab, pidilizumab, atezolizumab, avelumab, durvalumab, cemiplimab, and a combination thereof. In some embodiments, the immunotherapy includes a cellular therapy (e.g., as CAR T-cell therapy). In some embodiments, an effective amount of the therapeutic agent is administered in combination with a composition comprising a bacterial strain as described herein.

In some embodiments, a prebiotic and/or probiotic can be administered in combination with a composition comprising a bacterial strain as described herein. Non-limiting examples of a probiotic include one of more of Bifidobacteria (e.g., B. animalis, B. breve, B. lactis, B. longum, B. longum, or B. infantis), Lactobacillus (e.g., L. acidophilus, L. reuteri, L. bulgaricus, L. lactis, L. casei, L. rhamnosus, L. plantarum, L. paracasei, or L. delbreuckii/bulgaricus), Saccharomyces boulardii, E. coli Nissle 1917, and Streptococcus thermophiles. Non-limiting examples of a prebiotic include a fructooligosaccharide (e.g., oligofructose, inulin, or an inulin-type fructan), a galactooligosaccharide, an amino acid, or an alcohol. See, for example, Ramirez-Farias et al. (2008. Br. J Nutr. 4:1-10) and Pool-Zobel and Sauer (2007. J Nutr. 137:2580-2584).

In some embodiments, an effective amount of the treatment of atopic dermatitis and/or adjunct therapy is administered in combination with a composition comprising a bacterial strain as described herein.

In some embodiments, methods provided herein can include monitoring the subject after treatment with a composition described herein to determine if one or more symptoms have been alleviated, if the severity of one or more symptoms has been reduced, or if progression of the disease has been delayed or inhibited in the subject. There are numerous scores and clinical markers that can be utilized to assess the efficacy of administering a composition that includes bacterial strain as described herein in treating colorectal cancer.

As an example, tumor or lesion size can be monitored. Any procedure that allows an assessment of the tumor or lesion size can be used. Non-limiting examples include digital rectal exam, an endoscopy (e.g., a colonoscopy), and imaging (e.g., PET, MRI, ERUS, DRE, CT). See, for example, McKeown et al. J Cancer. 2014; 5(1): 31-43. In some embodiments, tumor burden can be assessed using RECIST (e.g., RECIST version 1 or version 1.1). See, for example, Eisenhauer et al., Eur. J. Cancer. 45(2):228-47 (2009).

In some embodiments, treatment of colorectal cancer in a subject can be assessed using one or more indexes or biomarkers selected from the group consisting of: CEA (carcinoembryonic antigen), DNA methylation such as CpG island methylator phenotype (CIMP), ΔTAp73, thymidylate synthase, and circulating tumor cells (CTCs). In some embodiments, circulating tumor cells (CTCs) are detected by CEA mRNA, human telomerase reverse transcriptase, cytokeratin-19, and/or cytokeratin-20. For example, an improvement in one or more of the above indexes or biomarkers after administering a bacterial strain, or a composition thereof, as described herein to the subject indicates treatment of the colorectal cancer.

In some embodiments, compositions provided herein can include one or more excipients and can be formulated for any of a number of delivery systems suitable for administration to a subject (e.g., probiotic or LBP delivery systems). Non-limiting examples of an excipient include a buffering agent, a diluent, a preservative, a stabilizer, a binding agent, a filler, a lubricant, a dispersion enhancer, a disintegrant, a lubricant, a disintegrant, a wetting agent, a glidant, a flavoring agent, a sweetener, and a coloring agent. For example, in some embodiments, tablets or capsules can be prepared by conventional means with excipients such as binding agents, fillers, lubricants, disintegrants, or wetting agents. Any of the compositions described herein can be administered to a subject to treat colorectal cancer as described herein.

In some embodiments, a composition as described herein can be formulated for oral delivery. In some embodiments, the composition can be formulated as a tablet, a chewable tablet, a capsule, a stick pack, a powder, effervescent powder, or a liquid. In some embodiments, a composition can include coated beads that contain the bacterial strain. In some embodiments, a powder comprising the bacterial strain can be suspended or dissolved in a drinkable liquid such as water for administration. In some embodiments, the composition is a solid composition.

In some embodiments, a composition described herein can be formulated for various immediate and controlled release profiles of the bacterial strain. For example, a controlled release formulation can include a controlled release coating disposed over the bacterial strain. In some embodiments, the controlled release coating is an enteric coating, a semi-enteric coating, a delayed release coating, or a pulsed release coating. In some embodiments, a coating can be suitable if it provides an appropriate lag in active release (i.e., release of the bacterial strain). For example, in some embodiments, the composition can be formulated as a tablet that includes a coating (e.g., an enteric coating).

In some embodiments, the composition can be formulated for topical delivery. In some embodiments, the composition can be in the form of a paste, gel, cream, spray, suppository, mousse, emollient, ointment, foam, or suspension.

In some embodiments, the bacterial strain in the composition is a culture of a single strain of organism. In some embodiments, the composition comprises a bacterial strain that is isolated. In some embodiments, the bacterial strain is isolated and cultured in vitro to increase the number or concentration of the bacterial strain. Increasing the number or concentration of the bacterial strain can be useful, for example, to enhance the efficacy of a composition comprising the bacterial strain.

In some embodiments, an effective amount of the bacterial strain in a composition described herein comprises at least about 1×10³ CFU of the bacterial strain. For example, at least about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, or about 1×10¹⁴ CFUs of the bacterial strain. In some embodiments, the effective amount of a bacterial strain in a composition described herein comprises about 1×10³ to about 1×10¹⁵ CFUs of the bacterial strain. For example, about 1×10³ to about 1×10⁶, about 1×10³ to about 1×10⁸, about 1×10³ to about 1×10¹⁰, about 1×10³ to about 1×10¹², about 1×10³ to about 1×10¹⁴, about 1×10⁷ to about 1×10¹², about 1×10¹³ to about 1×10¹⁵, about 1×10¹¹ to about 1×10¹⁵, about 1×10⁹ to about 1×10¹⁵, about 1×10⁷ to about 1×10¹⁵, or about 1×10⁵ to about 1×10¹⁵ CFUs of the bacterial strain.

In some embodiments, the composition can include one or more biologically pure strains (e.g., two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, or twenty-two bacterial strains). For example, the composition can include biologically pure Bacteroides eggerthii 1 2 48FAA, biologically pure Bacteroides eggerthii CCUG 9559, biologically pure Bacteroides goldsteinii ATCC BAA 1180, biologically pure Bacteroides pectinophilus N3, biologically pure Bacteroides plebeius M12, biologically pure Bacteroides vulgatus 8482, biologically pure Barnesiella intestinihominis DSM 21032, biologically pure Bifidobacterium stercoris EG1, biologically pure Clostridium sp. 40, biologically pure Clostridium spiroforme CCM 6168, biologically pure Eubacterium eligens DSM 3376, biologically pure Eubacterium hallii DSM 3353, biologically pure Megamonas funiformis DSM 19343, biologically pure Megasphaera elsdenii LC1, biologically pure Parabacteroides johnsonii M-165, biologically pure Butyricimonas virosa MT12, biologically pure Clostridium citroniae DSM 19261, biologically pure Lactobacillus ruminis RF3, biologically pure Methanosphaera stadtmanae MCB-3, biologically pure Ruminococcus sp. 18P13, biologically pure Slackia piriformis DSM 22477, biologically pure Streptococcus hongkongensis HKU30, or any combination thereof.

In some embodiments, the composition is a solid composition that includes at least 1×10³ CFUs of a bacterial strain (e.g., a biologically pure strain) and one or more excipients. Identifying characteristics of suitable strains, including homology to 16S rRNA sequences are described above.

In some embodiments, each member of the same bacterial strain has a 16S rRNA gene sequence with at least about 90%, about 91%, about 92%, about 93%, about 94%, about 95%, about 96%, about 97%, about 98%, about 99%, about 99.5%, about 99.6%, about 99.7%, about 99.8%, or about 99.9% sequence identity to the 16S rRNA gene sequence of each other member of the same bacterial strain.

In some embodiments, a bacterial strain in a composition described herein is preserved. Methods for preserving bacterial strains can include lyophilization and cryopreservation, optionally in the presence of a protectant. Non-limiting examples of protectants include sucrose, inulin, and glycerol. In some embodiments, a composition can include a lyophilized or cryopreserved bacterial strain such as Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, or a combination thereof, and an optional protectant. In some embodiments, a composition can include a lyophilized or cryopreserved bacterial strain such as Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, or a combination thereof, and an optional protectant.

In some embodiments, wherein the bacterial strain is a combination of two or more of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, and Parabacteroides johnsonii M-165, one or more of Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, and Parabacteroides johnsonii M-165 are lyophilized or cryopreserved.

In some embodiments, the composition is a live bacterial product (LBP). In some embodiments, the bacterial strain in the composition is viable. The viable bacterial strain may be, for example, cryopreserved and/or lyophilized. In some embodiments, a composition for delivery of live bacterial strains (e.g., Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, or a combination thereof), can be formulated to maintain viability of the bacterial strain. In some embodiments, the composition comprises elements that protect the bacterial strain from the acidic environment of the stomach (e.g., an enteric coating).

In some embodiments, wherein the bacterial strain is a combination of two or more of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, and Parabacteroides johnsonii M-165, one or more of Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, and Parabacteroides johnsonii M-165 are viable.

In some embodiments, the bacterial strain in the composition can be non-viable. In some embodiments, the non-viable bacterial strain is heat-killed, irradiated, or lysed.

In some embodiments, wherein the bacterial strain is a combination of two or more of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, and Parabacteroides johnsonii M-165, one or more of Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, and Parabacteroides johnsonii M-165 are non-viable (e.g., heat-killed, irradiated, or lysed).

In some embodiments, the bacterial strain as described herein may be used in prophylactic applications. For example, in a prophylactic application, a bacterial strain or a composition described herein can be administered to a subject susceptible to, or otherwise at risk of, a particular disease in an amount that is sufficient to at least partially reduce the risk of developing a disease. One of ordinary skill in the art will appreciate that the precise amounts of the bacterial strain administered may depend on a number of subject specific factors such as the subject's state of health and/or weight.

Also provided herein are methods of identifying a subject as having colorectal cancer that include: (a) identifying a subject having a sample that has: (i) an increased level of one or more (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, or 24) bacterial species selected from the group consisting of: Alistipes indistinctus, Azotobacter sp., Bacteroides asaccharolyticus, Bacteroides sp., Bilophila wadsworthia, Clostridium aldenense, Clostridium bolteae, Clostridium lactatifermentans, Clostridium sp., Desulfovibrio piger, Dialister pneumosintes, Eubacterium biforme, Fusobacterium mortiferum, Hungatella hathewayi, Megasphaera micronuciformis, Micromonas micros, Paraprevotella clara, Peptostreptococcus stomatis, Porphyromonas somerae, Roseburia inulinivorans, Ruminococcus gnavus, Shigella dysenteriae, Subdoligranulum variabile, Veillonella alcalescens; and/or (ii) a decreased level of one or more (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, or 22) bacterial species selected from the group consisting of: Bacteroides eggerthii, Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, and Streptococcus hongkongensis, as having colorectal cancer; or (b) identifying a subject having a sample that does not have: (i) an increased level of one or more (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, or 24) bacterial species selected from the group consisting of: Alistipes indistinctus, Azotobacter sp., Bacteroides asaccharolyticus, Bacteroides sp., Bilophila wadsworthia, Clostridium aldenense, Clostridium bolteae, Clostridium lactatifermentans, Clostridium sp., Desulfovibrio piger, Dialister pneumosintes, Eubacterium biforme, Fusobacterium mortiferum, Hungatella hathewayi, Megasphaera micronuciformis, Micromonas micros, Paraprevotella clara, Peptostreptococcus stomatis, Porphyromonas somerae, Roseburia inulinivorans, Ruminococcus gnavus, Shigella dysenteriae, Subdoligranulum variabile, Veillonella alcalescens; and/or (ii) a decreased level of one or more (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, or 22) bacterial species selected from the group consisting of: Bacteroides eggerthii, Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, and Streptococcus hongkongensis, as not having colorectal cancer.

Also provided herein are methods of diagnosing a subject as having colorectal cancer that include: (a) identifying a subject having a sample that has: (i) an increased level of one or more (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, or 24) bacterial species selected from the group consisting of: Alistipes indistinctus, Azotobacter sp., Bacteroides asaccharolyticus, Bacteroides sp., Bilophila wadsworthia, Clostridium aldenense, Clostridium bolteae, Clostridium lactatifermentans, Clostridium sp., Desulfovibrio piger, Dialister pneumosintes, Eubacterium biforme, Fusobacterium mortiferum, Hungatella hathewayi, Megasphaera micronuciformis, Micromonas micros, Paraprevotella clara, Peptostreptococcus stomatis, Porphyromonas somerae, Roseburia inulinivorans, Ruminococcus gnavus, Shigella dysenteriae, Subdoligranulum variabile, Veillonella alcalescens; and/or (ii) a decreased level of one or more (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, or 22) bacterial species selected from the group consisting of: Bacteroides eggerthii, Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, and Streptococcus hongkongensis, as having colorectal cancer; or (b) identifying a subject having a sample that does not have: (i) an increased level of one or more (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, or 24) bacterial species selected from the group consisting of: Alistipes indistinctus, Azotobacter sp., Bacteroides asaccharolyticus, Bacteroides sp., Bilophila wadsworthia, Clostridium aldenense, Clostridium bolteae, Clostridium lactatifermentans, Clostridium sp., Desulfovibrio piger, Dialister pneumosintes, Eubacterium biforme, Fusobacterium mortiferum, Hungatella hathewayi, Megasphaera micronuciformis, Micromonas micros, Paraprevotella clara, Peptostreptococcus stomatis, Porphyromonas somerae, Roseburia inulinivorans, Ruminococcus gnavus, Shigella dysenteriae, Subdoligranulum variabile, Veillonella alcalescens; and/or (ii) a decreased level of one or more (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, or 22) bacterial species selected from the group consisting of: Bacteroides eggerthii, Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, and Streptococcus hongkongensis, as not having colorectal cancer.

The invention will be further described in the following examples, which do not limit the scope of the invention described in the claims.

### EXAMPLES

### Example 1. Identification of Bacterial Strains Associated with Colorectal Cancer

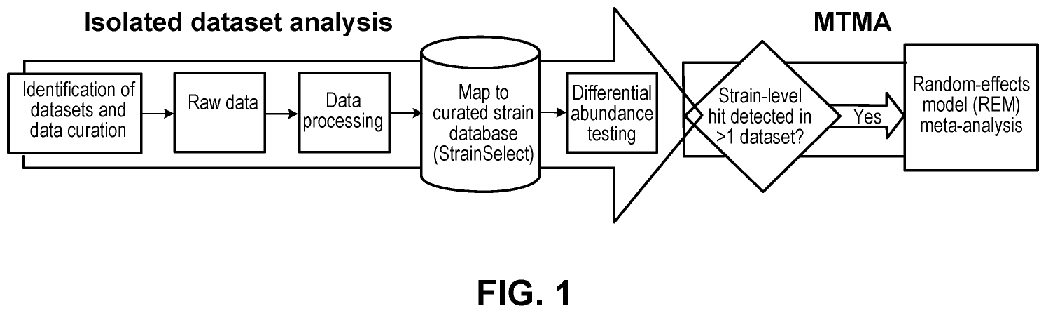

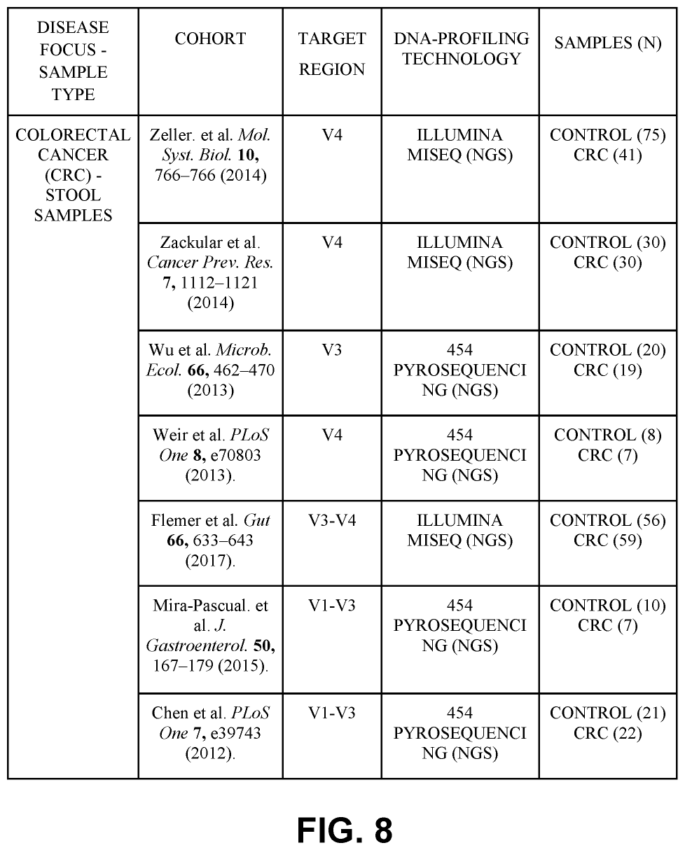

A multiple-technology meta-analysis (MTMA) approach was applied to datasets, which led to the identification of strains unique to colorectal cancer. To overcome challenges of comparing clinical variables across datasets from multiple institutes, a controlled vocabulary of hierarchically organized terms and manually re-annotated metadata from public datasets using this vocabulary was created. FIG. 8 provides the details regarding the cohorts, datasets, and contrasts analyzed to identify differentially abundant (DA) taxa correlating with disease progression. Statistical analysis of each dataset was performed using the workflow described in FIG. 1. Strain-level annotation was achieved using StrainSelect, a database containing sequence information of bacterial and archaeal strains connected to genome identifiers, which facilitated comparative analysis of taxa abundances at a strain-level across datasets

### Methods

### Procurement of Raw Data and Metadata Curation

Fastq/Fasta files and metadata were procured from public repositories. Metadata stored with raw data, such as NCBI's RunInfo table associated with the SRA Run Selector, and/or metadata published in tables in the primary text or supplementary files of the publication, were retrieved and manually re-annotated using a controlled vocabulary of hierarchically organized terms. An in-house database was created to store all study-related data and facilitate appropriate metadata annotation of all datasets via manual curation. Clinical metadata was stored in this database as a series of label:value pairs attached to the biospecimen from which the data files were generated.

### Processing, Strain Annotation and Statistical Analyses of Raw Data

16S rRNA Sequencing Datasets (16S-NGS and 16S-Sanger)

For NGS datasets, paired-end reads were merged and aligned to the StrainSelect database version 2014 (SS14) using USEARCH (usearch_global) (see, Edgar. Bioinformatics 26, 2460-2461 (2010)). All sequences matching a unique strain at an identity ≥99% were assigned a strain-level annotation. To ensure specificity of these strain matches, a difference of ≥0.25% between the identity of the best match and the second best match was required e.g., 99.75 vs. 99.5. StrainSelect is a repository of strain identifiers obtained from gene sequencing, genome sequencing, draft genomes, and metagenomic assemblies of known prokaryotic strains. Distinct strain matches counted to generate strain-level abundances. Remaining sequences were quality-filtered, chimera-filtered and clustered at ≥97% similarity via UPARSE (Allali et al. doi:10.1186/s12866-017-1101-8) to generated de-novo OTUs. OTU abundances were generated by aligning and counting all non-strain sequences against OTU representatives.

Downstream of a 5% prevalence filter, DESeq2 (Wood and Salzberg. Genome Biol. 15, R46 (2014)) was used to calculate significant differences in isolated analysis across all bins (OTUs and strains) and adjusted p-values were determined with the Benjamini-Hochberg correction. Significant results were determined as adjusted p-values <0.05. Log 2 fold change and standard error were calculated via DESeq2 and applied to subsequent analyses.

### Multi-Technology Meta-Analysis

Log 2 fold change and standard errors pertaining to per-dataset statistical results in each disease area were integrated in MTMA using a Random effects model (REM), generated using the metafor R package. Only bins with strain-level annotations in each dataset, and only those strains observed in at least two datasets, post prevalence filtering, were retained for REM analysis. False discovery correction for REM generated p-values was achieved using the Benjamini-Hochberg method. Differences are deemed to be statistically significant at adjusted (Benjamini-Hochberg corrected) p-values <0.05 in both isolated dataset analysis and MTMA.

### Results

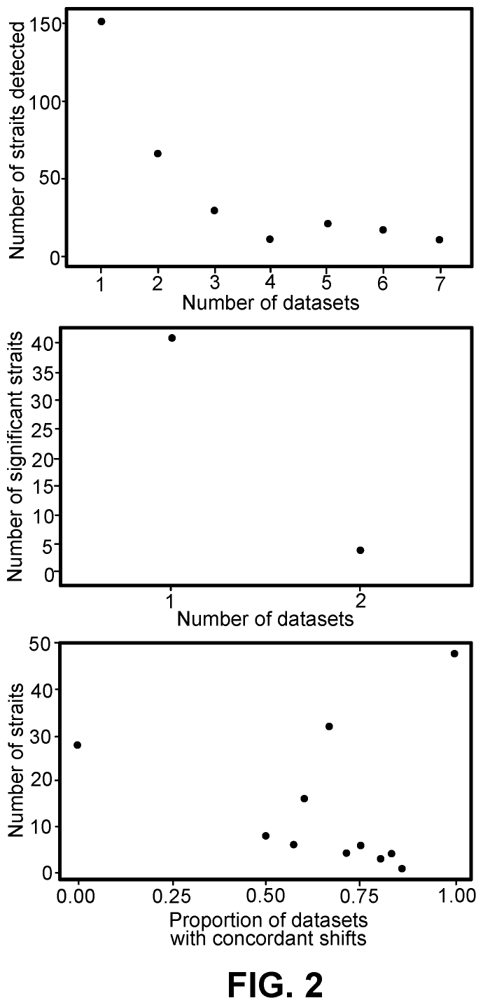

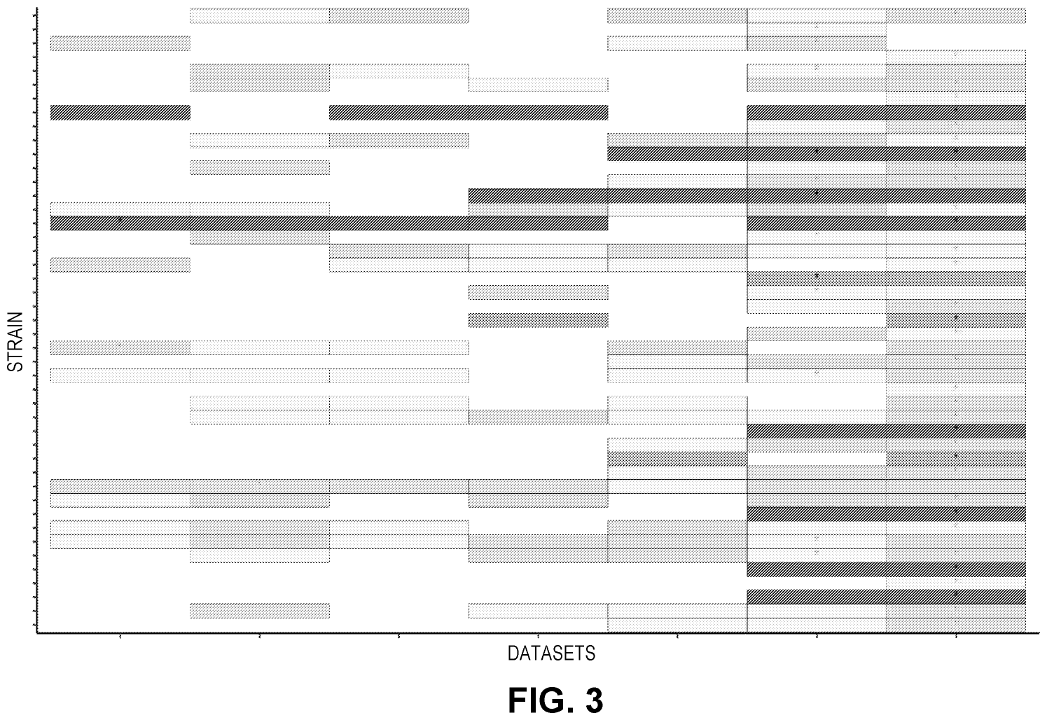

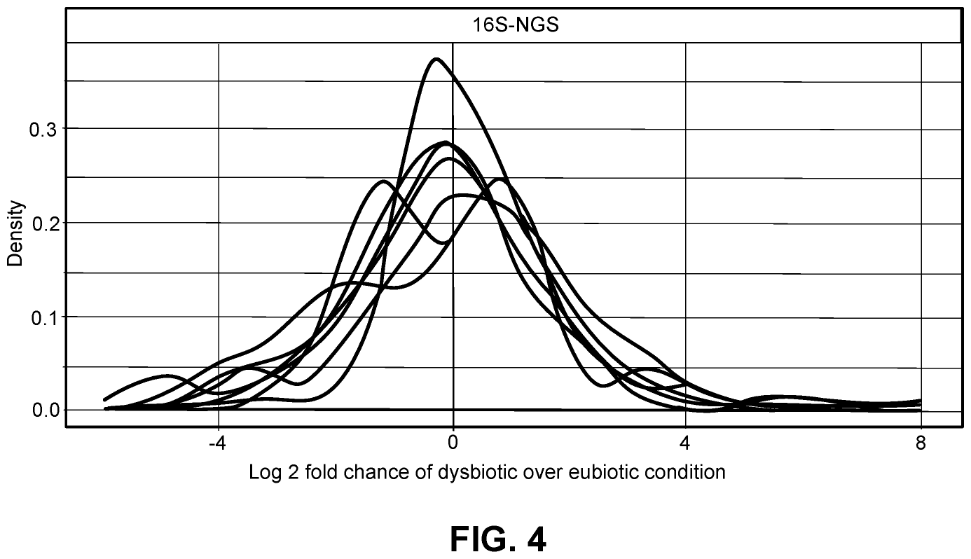

Identification of differentially abundant strains across cohorts from a simple comparison of isolated datasets was limited as less than 3.5% of the strains were detected across all datasets (FIG. 2, top panel). Further, while isolated analysis identified 45 strains as significantly differentially abundant within a disease, these strain-disease associations were mostly cohort-specific (FIG. 2, middle panel) and in many cases not supported in trend, i.e., a strain being consistently associated with either homeostasis or dysbiosis, across other datasets (FIG. 3; light-shaded rows). Variation was also observed in the magnitude of differential abundance derived from the cohorts (log 2 fold change; FIG. 4).

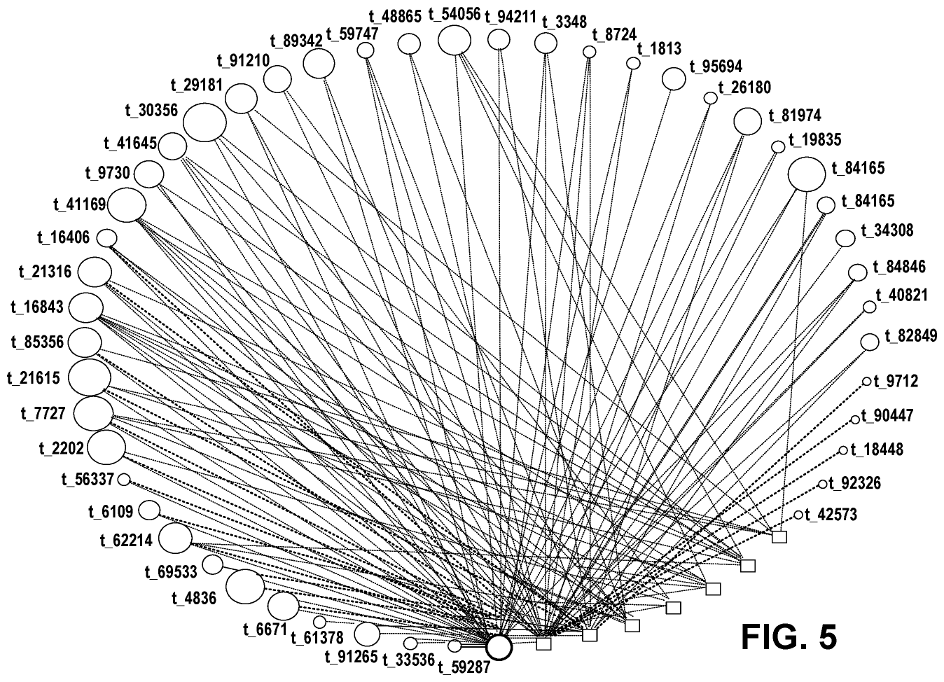

Strain-level results were integrated from isolated analyses in each disease via MTMA as described in FIG. 1. Significant associations were identified only when the direction of differential abundance of the strain was supported by multiple datasets (FIG. 5; blue strains connected to MTMA nodes via thick-solid lines). The associations in isolated analyses that were not supported in trend by other datasets were not significant by MTMA (FIG. 5; dark-green circles). Several strain-disease associations identified in MTMA were not identified in isolated analyses of the datasets (FIG. 5; purple dots). Thus, MTMA corroborates findings from isolated analysis if supported across datasets but eliminates if discordant, and MTMA identifies novel disease-strain associations that isolated analyses failed to detect.

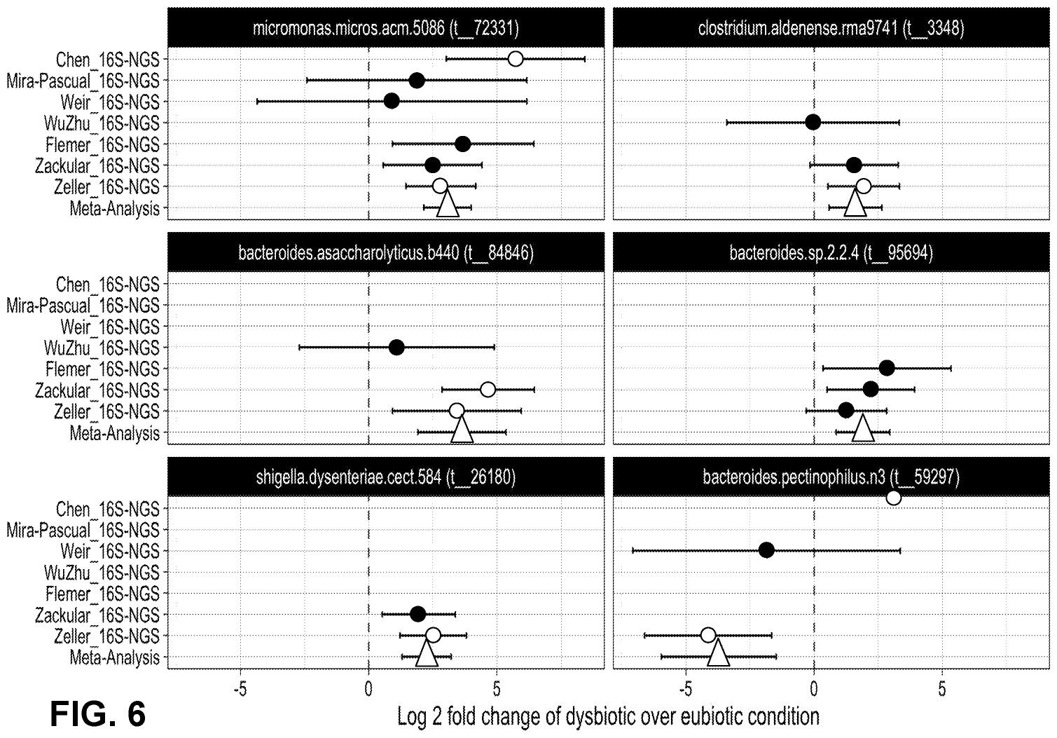

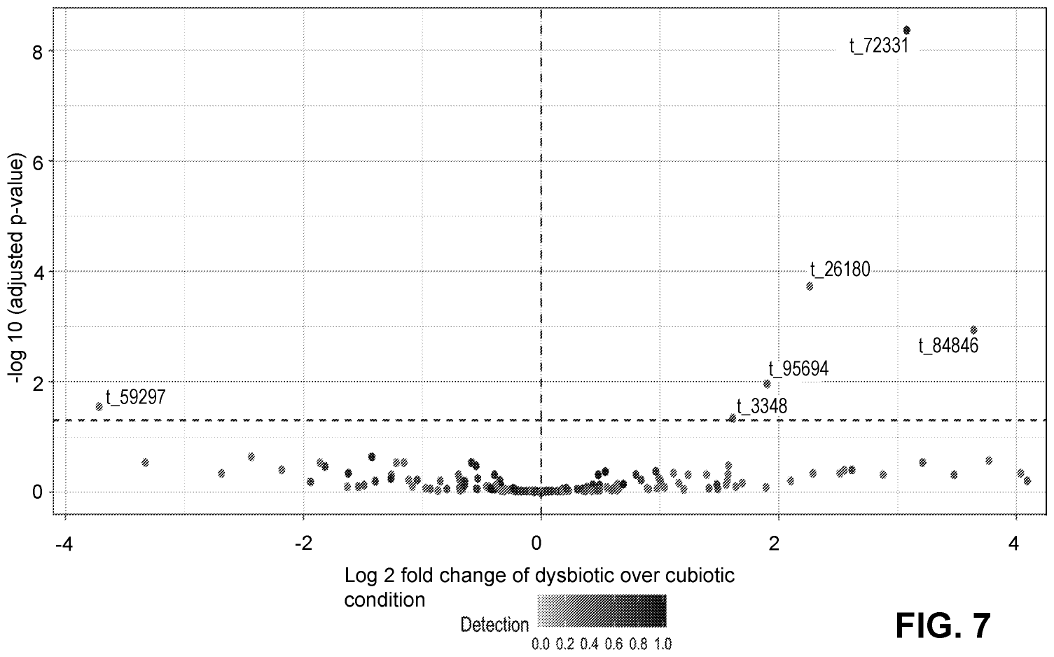

Enrichment (increase) of disease-associated bacteria in colorectal cancer (FIG. 6), many of which were classified as belonging to pathogenic species, indicated that colonization by pathogens as opposed to a lack of homeostasis could be a key driver in these diseases (FIG. 7).

MTMA can enable synthesis of existing knowledge of the microbiome, and the approach as shown in FIG. 1 can facilitate comparative analysis of taxa abundances at a strain-level across datasets generated with different DNA-profiling technologies. Harnessing the MTMA framework, with its ability to integrate datasets across DNA-profiling technologies and pinpoint specific strains, can allow for identification of robust microbiome modulators of disease by integrating the growing body of evidence on the role played by microbiome in disease.

### OTHER EMBODIMENTS

It is to be understood that while the invention has been described in conjunction with the detailed description thereof, the foregoing description is intended to illustrate and not limit the scope of the invention which is defined by the scope of the appended claims. Other aspects, advantages, and modification are within the scope of the following claims.

What is claimed is:

1. A method of identifying a subject as having colorectal cancer, the method comprising:
(a) identifying a subject having a sample that has:
(i) an increased level of one or more bacterial species selected from the group consisting of: Alistipes indistinctus, Azotobacter sp., Bacteroides asaccharolyticus, Bacteroides sp., Bilophila wadsworthia, Clostridium aldenense, Clostridium bolteae, Clostridium lactatifermentans, Clostridium sp., Desulfovibrio piger, Dialister pneumosintes, Eubacterium biforme, Fusobacterium mortiferum, Hungatella hathewayi, Megasphaera micronuciformis, Micromonas micros, Paraprevotella clara, Peptostreptococcus stomatis, Porphyromonas somerae, Roseburia inulinivorans, Ruminococcus gnavus, Shigella dysenteriae, Subdoligranulum variabile, and Veillonella alcalescens; and/or
(ii) a decreased level of one or more bacterial species selected from the group consisting of: Bacteroides eggerthii, Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, and Streptococcus hongkongensis, as having colorectal cancer; or
(b) identifying a subject having a sample that does not have:
(i) an increased level of one or more bacterial species selected from the group consisting of: Alistipes indistinctus, Azotobacter sp., Bacteroides asaccharolyticus, Bacteroides sp., Bilophila wadsworthia, Clostridium aldenense, Clostridium bolteae, Clostridium lactatifermentans, Clostridium sp., Desulfovibrio piger, Dialister pneumosintes, Eubacterium biforme, Fusobacterium mortiferum, Hungatella hathewayi, Megasphaera micronuciformis, Micromonas micros, Paraprevotella clara, Peptostreptococcus stomatis, Porphyromonas somerae, Roseburia inulinivorans, Ruminococcus gnavus, Shigella dysenteriae, Subdoligranulum variabile, and Veillonella alcalescens; and/or
(ii) a decreased level of one or more bacterial species selected from the group consisting of: Bacteroides eggerthii, Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, and Streptococcus hongkongensis, as not having colorectal cancer.

2. A method of diagnosing a subject as having colorectal cancer, the method comprising:
(a) identifying a subject having a sample that has:
(i) an increased level of one or more bacterial species selected from the group consisting of: Alistipes indistinctus, Azotobacter sp., Bacteroides asaccharolyticus, Bacteroides sp., Bilophila wadsworthia, Clostridium aldenense, Clostridium bolteae, Clostridium lactatifermentans, Clostridium sp., Desulfovibrio piger, Dialister pneumosintes, Eubacterium biforme, Fusobacterium mortiferum, Hungatella hathewayi, Megasphaera micronuciformis, Micromonas micros, Paraprevotella clara, Peptostreptococcus stomatis, Porphyromonas somerae, Roseburia inulinivorans, Ruminococcus gnavus, Shigella dysenteriae, Subdoligranulum variabile, and Veillonella alcalescens; and/or
(ii) a decreased level of one or more bacterial species selected from the group consisting of: Bacteroides eggerthii, Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, and Streptococcus hongkongensis, as having colorectal cancer; or
(b) identifying a subject having a sample that does not have:
(i) an increased level of one or more bacterial species selected from the group consisting of: Alistipes indistinctus, Azotobacter sp., Bacteroides asaccharolyticus, Bacteroides sp., Bilophila wadsworthia, Clostridium aldenense, Clostridium bolteae, Clostridium lactatifermentans, Clostridium sp., Desulfovibrio piger, Dialister pneumosintes, Eubacterium biforme, Fusobacterium mortiferum, Hungatella hathewayi, Megasphaera micronuciformis, Micromonas micros, Paraprevotella clara, Peptostreptococcus stomatis, Porphyromonas somerae, Roseburia inulinivorans, Ruminococcus gnavus, Shigella dysenteriae, Subdoligranulum variabile, Veillonella alcalescens; and/or
(ii) a decreased level of one or more bacterial species selected from the group consisting of: Bacteroides eggerthii, Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, and Streptococcus hongkongensis, as not having colorectal cancer.

3. A method for treating a subject in need thereof, the method comprising administering to the subject a composition comprising an effective amount of a bacterial strain selected from the group consisting of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, and a combination thereof.

4. The method of claim 3, wherein the subject has colorectal cancer.

5. A method for treating colorectal cancer in a subject, the method comprising administering to the subject a composition comprising an effective amount of a bacterial species selected from the group consisting of: Bacteroides eggerthii, Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, Streptococcus hongkongensis, and a combination thereof.

6. A method for treating colorectal cancer in a subject, the method comprising:
(a) detecting a dysbiosis associated with colorectal cancer in a sample from the subject; and
(b) administering to the subject a composition comprising an effective amount of a bacterial species selected from the group consisting of: Bacteroides eggerthii, Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, Streptococcus hongkongensis, and a combination thereof.

7. The method of claim 6, wherein the sample is a fecal sample.

8. The method of claim 6, wherein the sample is a biopsy sample.

9. The method of claim 8, wherein the biopsy sample is a colorectal biopsy sample.

10. The method of any one of claims 6-9, wherein detecting the dysbiosis associated with colorectal cancer comprises determining bacterial gene expression in the sample from the subject.

11. The method of any one of claims 6-10, wherein detecting the dysbiosis associated with colorectal cancer comprises determining bacterial composition in the sample from the subject.

12. The method of claim any one of claims 6-11, wherein detecting the dysbiosis associated with colorectal cancer comprises determining that Alistipes indistinctus, Azotobacter sp., Bacteroides asaccharolyticus, Bacteroides sp., Bilophila wadsworthia, Clostridium aldenense, Clostridium bolteae, Clostridium lactatifermentans, Clostridium sp., Desulfovibrio piger, Dialister pneumosintes, Eubacterium biforme, Fusobacterium mortiferum, Hungatella hathewayi, Megasphaera micronuciformis, Micromonas micros, Paraprevotella clara, Peptostreptococcus stomatis, Porphyromonas somerae, Roseburia inulinivorans, Ruminococcus gnavus, Shigella dysenteriae, Subdoligranulum variabile, Veillonella alcalescens, or a combination thereof, is increased in the sample from subject.

13. The method of claim any one of claims 6-12, wherein detecting the dysbiosis associated with colorectal cancer comprises determining that Bacteroides eggerthii, Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, Streptococcus hongkongensis, or a combination thereof, is decreased in the sample from subject.

14. The method of claim 13, wherein Bacteroides eggerthii, Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, Streptococcus hongkongensis, or a combination thereof, is decreased in the gastrointestinal tract of the subject.

15. A method for treating a subject in need thereof, the method comprising decreasing a population of an increased bacterial strain in the subject, wherein the increased bacterial species is selected from the group consisting of: Alistipes indistinctus, Azotobacter sp., Bacteroides asaccharolyticus, Bacteroides sp., Bilophila wadsworthia, Clostridium aldenense, Clostridium bolteae, Clostridium lactatifermentans, Clostridium sp., Desulfovibrio piger, Dialister pneumosintes, Eubacterium biforme, Fusobacterium mortiferum, Hungatella hathewayi, Megasphaera micronuciformis, Micromonas micros, Paraprevotella clara, Peptostreptococcus stomatis, Porphyromonas somerae, Roseburia inulinivorans, Ruminococcus gnavus, Shigella dysenteriae, Subdoligranulum variabile, Veillonella alcalescens, and a combination thereof.

16. The method of claim 15, wherein the subject has colorectal cancer.

17. The method of claim 15 or 16, wherein decreasing the population of an increased bacterial strain comprises administering to the subject a bacteriophage.

18. The method of any one of claims 15-17, wherein decreasing the population of an increased bacterial species comprises administering to the subject a composition comprising an effective amount of a bacterial species selected from the group consisting of: Bacteroides eggerthii, Bacteroides eggerthii, Bacteroides goldsteinii, Bacteroides pectinophilus, Bacteroides plebeius, Bacteroides vulgatus, Barnesiella intestinihominis, Bifidobacterium stercoris, Clostridium sp., Clostridium spiroforme, Eubacterium eligens, Eubacterium hallii, Megamonas funiformis, Megasphaera elsdenii, Parabacteroides johnsonii, Butyricimonas virosa, Clostridium citroniae, Lactobacillus ruminis, Methanosphaera stadtmanae, Ruminococcus sp., Slackia piriformis, Streptococcus hongkongensis, and a combination thereof.

19. The method of any one of claims 1, 2, 4-14, and 16-18, wherein the colorectal cancer is adenocarcinoma, a carcinoid tumor, a gastrointestinal stromal tumor, or lymphoma.

20. The method of any one of claims 1-14, 18, and 19, wherein the bacterial species Bacteroides eggerthii comprises the strain Bacteroides eggerthii 1 2 48FAA.

21. The method of any one of claims 1-14 and 18-20, wherein the bacterial species Bacteroides eggerthii comprises the strain Bacteroides eggerthii CCUG 9559.

22. The method of any one of claims 1-14 and 18-21, wherein the bacterial species Bacteroides goldsteinii comprises the strain Bacteroides goldsteinii ATCC BAA 1180.

23. The method of any one of claims 1-14 and 18-22, wherein the bacterial species Bacteroides pectinophilus comprises the strain Bacteroides pectinophilus N3.

24. The method of any one of claims 1-14 and 18-23, wherein the bacterial species Bacteroides plebeius comprises the strain Bacteroides plebeius M12.

25. The method of any one of claims 1-14 and 18-24, wherein the bacterial species Bacteroides vulgatus comprises the strain Bacteroides vulgatus 8482.

26. The method of any one of claims 1-14 and 18-25, wherein the bacterial species Barnesiella intestinihominis comprises the strain Barnesiella intestinihominis DSM 21032.

27. The method of any one of claims 1-14 and 18-26, wherein the bacterial species Bifidobacterium stercoris comprises the strain Bifidobacterium stercoris EG1.

28. The method of any one of claims 1-14 and 18-27, wherein the bacterial species Clostridium sp. comprises the strain Clostridium sp. 40.

29. The method of any one of claims 1-14 and 18-28, wherein the bacterial species Clostridium spiroforme comprises the strain Clostridium spiroforme CCM 6168.

30. The method of any one of claims 1-14 and 18-29, wherein the bacterial species Eubacterium eligens comprises the strain Eubacterium eligens DSM 3376.

31. The method of any one of claims 1-14 and 18-30, wherein the bacterial species Eubacterium hallii comprises the strain Eubacterium hallii DSM 3353.

32. The method of any one of claims 1-14 and 18-31, wherein the bacterial species Megamonas funiformis comprises the species Megamonas funiformis DSM 19343.

33. The method of any one of claims 1-14 and 18-32, wherein the bacterial species Megasphaera elsdenii comprises the strain Megasphaera elsdenii LC1.

34. The method of any one of claims 1-14 and 18-33, wherein the bacterial species Parabacteroides johnsonii comprises the strain Parabacteroides johnsonii M-165.

35. The method of any one of claims 1-14 and 18-34, wherein the bacterial species Butyricimonas virosa comprises the strain Butyricimonas virosa MT12.

36. The method of any one of claims 1-14 and 18-35, wherein the bacterial species Clostridium citroniae comprises the strain Clostridium citroniae DSM 19261.

37. The method of any one of claims 1-14 and 18-36, wherein the bacterial species Lactobacillus ruminis comprises the strain Lactobacillus ruminis RF3.

38. The method of any one of claims 1-14 and 18-37, wherein the bacterial species Methanosphaera stadtmanae comprises the strain Methanosphaera stadtmanae MCB-3.

39. The method of any one of claims 1-14 and 18-38, wherein the bacterial species Ruminococcus sp. comprises the strain Ruminococcus sp. 18P13.

40. The method of any one of claims 1-14 and 18-39, wherein the bacterial species Slackia piriformis comprises the strain Slackia piriformis DSM 22477.

41. The method of any one of claims 1-14 and 18-40, wherein the bacterial species Streptococcus hongkongensis comprises the strain Streptococcus hongkongensis HKU30.

42. The method of any one of claims 3-14 and 18-41, wherein the bacterial species improves intestinal barrier function of the subject.

43. The method of any one of claims 20-42, wherein the Bacteroides eggerthii 1 2 48FAA has a 16S RNA gene that is at least 95% identical to SEQ ID NO:1.

44. The method of any one of claims 20-42, wherein the Bacteroides eggerthii 1 2 48FAA has a 16S RNA gene that is at least 95% identical to SEQ ID NO:2.

45. The method of any one of claims 20-42, wherein the Bacteroides eggerthii 1 2 48FAA has a 16S RNA gene that is at least 95% identical to SEQ ID NO:3.

46. The method of any one of claims 20-42, wherein the Bacteroides eggerthii 1 2 48FAA has a 16S RNA gene that is at least 95% identical to SEQ ID NO:4.

47. The method of any one of claims 20-42, wherein the Bacteroides eggerthii 1 2 48FAA has a 16S RNA gene that is at least 95% identical to SEQ ID NO:5.

48. The method of any one of claims 20-42, wherein the Bacteroides eggerthii 1 2 48FAA has a 16S RNA gene that is at least 95% identical to SEQ ID NO:6.

49. The method of any one of claims 21-48, wherein the Bacteroides eggerthii CCUG 9559 has a 16S RNA gene that is at least 95% identical to SEQ ID NO:7.

50. The method of any one of claims 21-48, wherein the Bacteroides eggerthii CCUG 9559 has a 16S RNA gene that is at least 95% identical to SEQ ID NO:8.

51. The method of any one of claims 21-48, wherein the Bacteroides eggerthii CCUG 9559 has a 16S RNA gene that is at least 95% identical to SEQ ID NO:9.

52. The method of any one of claims 21-48, wherein the Bacteroides eggerthii CCUG 9559 has a 16S RNA gene that is at least 95% identical to SEQ ID NO:10.

53. The method of any one of claims 21-48, wherein the Bacteroides eggerthii CCUG 9559 has a 16S RNA gene that is at least 95% identical to SEQ ID NO:11.

54. The method of any one of claims 21-48, wherein the Bacteroides eggerthii CCUG 9559 has a 16S RNA gene that is at least 95% identical to SEQ ID NO:12.

55. The method of any one of claims 22-54, wherein the Bacteroides goldsteinii ATCC BAA 1180 has a 16S RNA gene that is at least 95% identical to SEQ ID NO:13.

56. The method of any one of claims 22-54, wherein the Bacteroides goldsteinii ATCC BAA 1180 has a 16S RNA gene that is at least 95% identical to SEQ ID NO:14.

57. The method of any one of claims 22-54, wherein the Bacteroides goldsteinii ATCC BAA 1180 has a 16S RNA gene that is at least 95% identical to SEQ ID NO:15.

58. The method of any one of claims 22-54, wherein the Bacteroides goldsteinii ATCC BAA 1180 has a 16S RNA gene that is at least 95% identical to SEQ ID NO:16.

59. The method of any one of claims 22-54, wherein the Bacteroides goldsteinii ATCC BAA 1180 has a 16S RNA gene that is at least 95% identical to SEQ ID NO:17.

60. The method of any one of claims 22-54, wherein the Bacteroides goldsteinii ATCC BAA 1180 has a 16S RNA gene that is at least 95% identical to SEQ ID NO:18.

61. The method of any one of claims 22-54, wherein the Bacteroides goldsteinii ATCC BAA 1180 has a 16S RNA gene that is at least 95% identical to SEQ ID NO:19.

62. The method of any one of claims 23-61, wherein the Bacteroides pectinophilus N3 has a 16S RNA gene that is at least 95% identical to SEQ ID NO:20.

63. The method of any one of claims 23-61, wherein the Bacteroides pectinophilus N3 has a 16S RNA gene that is at least 95% identical to SEQ ID NO:21.

64. The method of any one of claims 23-61, wherein the Bacteroides pectinophilus N3 has a 16S RNA gene that is at least 95% identical to SEQ ID NO:22.

65. The method of any one of claims 23-61, wherein the Bacteroides pectinophilus N3 has a 16S RNA gene that is at least 95% identical to SEQ ID NO:23.

66. The method of any one of claims 23-61, wherein the Bacteroides pectinophilus N3 has a 16S RNA gene that is at least 95% identical to SEQ ID NO:24.

67. The method of any one of claims 23-61, wherein the Bacteroides pectinophilus N3 has a 16S RNA gene that is at least 95% identical to SEQ ID NO:25.

68. The method of any one of claims 24-67, wherein the Bacteroides plebeius M12 has a 16S RNA gene that is at least 95% identical to SEQ ID NO:26.

69. The method of any one of claims 24-67, wherein the Bacteroides plebeius M12 has a 16S RNA gene that is at least 95% identical to SEQ ID NO:27.

70. The method of any one of claims 24-67, wherein the Bacteroides plebeius M12 has a 16S RNA gene that is at least 95% identical to SEQ ID NO:28.

71. The method of any one of claims 25-70, wherein the Bacteroides vulgatus 8482 has a 16S RNA gene that is at least 95% identical to SEQ ID NO:29.

72. The method of any one of claims 25-71, Bacteroides vulgatus 8482 has a 16S RNA gene that is at least 95% identical to SEQ ID NO:30.

73. The method of any one of claims 25-71, Bacteroides vulgatus 8482 has a 16S RNA gene that is at least 95% identical to SEQ ID NO:31.

74. The method of any one of claims 25-71, Bacteroides vulgatus 8482 has a 16S RNA gene that is at least 95% identical to SEQ ID NO:32.

75. The method of any one of claims 25-71, Bacteroides vulgatus 8482 has a 16S RNA gene that is at least 95% identical to SEQ ID NO:33.

76. The method of any one of claims 25-71, Bacteroides vulgatus 8482 has a 16S RNA gene that is at least 95% identical to SEQ ID NO:34.

77. The method of any one of claims 25-71, Bacteroides vulgatus 8482 has a 16S RNA gene that is at least 95% identical to SEQ ID NO:35.

78. The method of any one of claims 25-71, Bacteroides vulgatus 8482 has a 16S RNA gene that is at least 95% identical to SEQ ID NO:36.

79. The method of any one of claims 25-71, Bacteroides vulgatus 8482 has a 16S RNA gene that is at least 95% identical to SEQ ID NO:37.

80. The method of any one of claims 25-71, Bacteroides vulgatus 8482 has a 16S RNA gene that is at least 95% identical to SEQ ID NO:38.

81. The method of any one of claims 25-71, Bacteroides vulgatus 8482 has a 16S RNA gene that is at least 95% identical to SEQ ID NO:39.

82. The method of any one of claims 25-71, Bacteroides vulgatus 8482 has a 16S RNA gene that is at least 95% identical to SEQ ID NO:40.

83. The method of any one of claims 26-82, wherein the Barnesiella intestinihominis DSM 21032 has a 16S RNA gene that is at least 95% identical to SEQ ID NO:41.

84. The method of any one of claims 26-82, wherein the Barnesiella intestinihominis DSM 21032 has a 16S RNA gene that is at least 95% identical to SEQ ID NO:42.

85. The method of any one of claims 26-82, wherein the Barnesiella intestinihominis DSM 21032 has a 16S RNA gene that is at least 95% identical to SEQ ID NO:43.

86. The method of any one of claims 26-82, wherein the Barnesiella intestinihominis DSM 21032 has a 16S RNA gene that is at least 95% identical to SEQ ID NO:44.

87. The method of any one of claims 26-82, wherein the Barnesiella intestinihominis DSM 21032 has a 16S RNA gene that is at least 95% identical to SEQ ID NO:45.

88. The method of any one of claims 26-82, wherein the Barnesiella intestinihominis DSM 21032 has a 16S RNA gene that is at least 95% identical to SEQ ID NO:46.

89. The method of any one of claims 26-82, wherein the Barnesiella intestinihominis DSM 21032 has a 16S RNA gene that is at least 95% identical to SEQ ID NO:47.

90. The method of any one of claims 26-82, wherein the Barnesiella intestinihominis DSM 21032 has a 16S RNA gene that is at least 95% identical to SEQ ID NO:48.

91. The method of any one of claims 26-82, wherein the Barnesiella intestinihominis DSM 21032 has a 16S RNA gene that is at least 95% identical to SEQ ID NO:49.

92. The method of any one of claims 26-82, wherein the Barnesiella intestinihominis DSM 21032 has a 16S RNA gene that is at least 95% identical to SEQ ID NO:50.

93. The method of any one of claims 27-92, wherein the Bifidobacterium stercoris EG1 has a 16S RNA gene that is at least 95% identical to SEQ ID NO:51.

94. The method of any one of claims 27-92, wherein the Bifidobacterium stercoris EG1 has a 16S RNA gene that is at least 95% identical to SEQ ID NO:52.

95. The method of any one of claims 27-92, wherein the Bifidobacterium stercoris EG1 has a 16S RNA gene that is at least 95% identical to SEQ ID NO:53.

96. The method of any one of claims 27-92, wherein the Bifidobacterium stercoris EG1 has a 16S RNA gene that is at least 95% identical to SEQ ID NO:54.

97. The method of any one of claims 28-96, wherein the Clostridium sp. 40 has a 16S RNA gene that is at least 95% identical to SEQ ID NO:57.

98. The method of any one of claims 29-97, wherein the Clostridium spiroforme CCM 6168 has a 16S RNA gene that is at least 95% identical to SEQ ID NO:58.

99. The method of any one of claims 29-97, wherein the Clostridium spiroforme CCM 6168 has a 16S RNA gene that is at least 95% identical to SEQ ID NO:59.

100. The method of any one of claims 29-97, wherein the Clostridium spiroforme CCM 6168 has a 16S RNA gene that is at least 95% identical to SEQ ID NO:60.

101. The method of any one of claims 29-97, wherein the Clostridium spiroforme CCM 6168 has a 16S RNA gene that is at least 95% identical to SEQ ID NO:61.

102. The method of any one of claims 29-97, wherein the Clostridium spiroforme CCM 6168 has a 16S RNA gene that is at least 95% identical to SEQ ID NO:62.

103. The method of any one of claims 29-97, wherein the Clostridium spiroforme CCM 6168 has a 16S RNA gene that is at least 95% identical to SEQ ID NO:63.

104. The method of any one of claims 30-103, wherein the Eubacterium eligens DSM 3376 has a 16S RNA gene that is at least 95% identical to SEQ ID NO:64.

105. The method of any one of claims 30-103, wherein the Eubacterium eligens DSM 3376 has a 16S RNA gene that is at least 95% identical to SEQ ID NO:65.

106. The method of any one of claims 30-103, wherein the Eubacterium eligens DSM 3376 has a 16S RNA gene that is at least 95% identical to SEQ ID NO:66.

107. The method of any one of claims 30-103, wherein the Eubacterium eligens DSM 3376 has a 16S RNA gene that is at least 95% identical to SEQ ID NO:67.

108. The method of any one of claims 30-103, wherein the Eubacterium eligens DSM 3376 has a 16S RNA gene that is at least 95% identical to SEQ ID NO:68.

109. The method of any one of claims 30-103, wherein the Eubacterium eligens DSM 3376 has a 16S RNA gene that is at least 95% identical to SEQ ID NO:69.

110. The method of any one of claims 30-103, wherein the Eubacterium eligens DSM 3376 has a 16S RNA gene that is at least 95% identical to SEQ ID NO:70.

111. The method of any one of claims 31-110, wherein the Eubacterium hallii DSM 3353 has a 16S RNA gene that is at least 95% identical to SEQ ID NO:71.

112. The method of any one of claims 31-110, wherein the Eubacterium hallii DSM 3353 has a 16S RNA gene that is at least 95% identical to SEQ ID NO:72.

113. The method of any one of claims 32-112, wherein the Megamonas funiformis DSM 19343 has a 16S RNA gene that is at least 95% identical to SEQ ID NO:80.

114. The method of any one of claims 32-112, wherein the Megamonas funiformis DSM 19343 has a 16S RNA gene that is at least 95% identical to SEQ ID NO:81.

115. The method of any one of claims 32-112, wherein the Megamonas funiformis DSM 19343 has a 16S RNA gene that is at least 95% identical to SEQ ID NO:82.

116. The method of any one of claims 32-112, wherein the Megamonas funiformis DSM 19343 has a 16S RNA gene that is at least 95% identical to SEQ ID NO:83.

117. The method of any one of claims 32-112, wherein the Megamonas funiformis DSM 19343 has a 16S RNA gene that is at least 95% identical to SEQ ID NO:84.

118. The method of any one of claims 32-112, wherein the Megamonas funiformis DSM 19343 has a 16S RNA gene that is at least 95% identical to SEQ ID NO:85.

119. The method of any one of claims 32-112, wherein the Megamonas funiformis DSM 19343 has a 16S RNA gene that is at least 95% identical to SEQ ID NO:86.

120. The method of any one of claims 33-119, wherein the Megasphaera elsdenii LC1 has a 16S RNA gene that is at least 95% identical to SEQ ID NO:87.

121. The method of any one of claims 33-119, wherein the Megasphaera elsdenii LC1 has a 16S RNA gene that is at least 95% identical to SEQ ID NO:88.

122. The method of any one of claims 33-119, wherein the Megasphaera elsdenii LC1 has a 16S RNA gene that is at least 95% identical to SEQ ID NO:89.

123. The method of any one of claims 33-119, wherein the Megasphaera elsdenii LC1 has a 16S RNA gene that is at least 95% identical to SEQ ID NO:90.

124. The method of any one of claims 33-119, wherein the Megasphaera elsdenii LC1 has a 16S RNA gene that is at least 95% identical to SEQ ID NO:91.

125. The method of any one of claims 33-119, wherein the Megasphaera elsdenii LC1 has a 16S RNA gene that is at least 95% identical to SEQ ID NO:92.

126. The method of any one of claims 33-119, wherein the Megasphaera elsdenii LC1 has a 16S RNA gene that is at least 95% identical to SEQ ID NO:93.

127. The method of any one of claims 33-119, wherein the Megasphaera elsdenii LC1 has a 16S RNA gene that is at least 95% identical to SEQ ID NO:94.

128. The method of any one of claims 33-119, wherein the Megasphaera elsdenii LC1 has a 16S RNA gene that is at least 95% identical to SEQ ID NO:95.

129. The method of any one of claims 34-128, wherein the Parabacteroides johnsonii M 165 has a 16S RNA gene that is at least 95% identical to SEQ ID NO:104.

130. The method of any one of claims 34-128, wherein the Parabacteroides johnsonii M 165 has a 16S RNA gene that is at least 95% identical to SEQ ID NO:105.

131. The method of any one of claims 34-128, wherein the Parabacteroides johnsonii M 165 has a 16S RNA gene that is at least 95% identical to SEQ ID NO:106.

132. The method of any one of claims 34-128, wherein the Parabacteroides johnsonii M 165 has a 16S RNA gene that is at least 95% identical to SEQ ID NO:107.

133. The method of any one of claims 35-132, wherein the Butyricimonas virosa MT12 has a 16S RNA gene that is at least 95% identical to SEQ ID NO:55.

134. The method of any one of claims 36-133, wherein the Clostridium citroniae DSM 19261 has a 16S RNA gene that is at least 95% identical to SEQ ID NO:56.

135. The method of any one of claims 37-134, wherein the Lactobacillus ruminis RF3 has a 16S RNA gene that is at least 95% identical to SEQ ID NO:73.

136. The method of any one of claims 37-134, wherein the Lactobacillus ruminis RF3 has a 16S RNA gene that is at least 95% identical to SEQ ID NO:74.

137. The method of any one of claims 37-134, wherein the Lactobacillus ruminis RF3 has a 16S RNA gene that is at least 95% identical to SEQ ID NO:75.

138. The method of any one of claims 37-134, wherein the Lactobacillus ruminis RF3 has a 16S RNA gene that is at least 95% identical to SEQ ID NO:76.

139. The method of any one of claims 37-134, wherein the Lactobacillus ruminis RF3 has a 16S RNA gene that is at least 95% identical to SEQ ID NO:77.

140. The method of any one of claims 37-134, wherein the Lactobacillus ruminis RF3 has a 16S RNA gene that is at least 95% identical to SEQ ID NO:78.

141. The method of any one of claims 37-134, wherein the Lactobacillus ruminis RF3 has a 16S RNA gene that is at least 95% identical to SEQ ID NO:79.

142. The method of any one of claims 38-141, wherein the Methanosphaera stadtmanae MCB-3 has a 16S RNA gene that is at least 95% identical to SEQ ID NO:96.

143. The method of any one of claims 38-141, wherein the Methanosphaera stadtmanae MCB-3 has a 16S RNA gene that is at least 95% identical to SEQ ID NO:97.

144. The method of any one of claims 38-141, wherein the Methanosphaera stadtmanae MCB-3 has a 16S RNA gene that is at least 95% identical to SEQ ID NO:98.

145. The method of any one of claims 38-141, wherein the Methanosphaera stadtmanae MCB-3 has a 16S RNA gene that is at least 95% identical to SEQ ID NO:99.

146. The method of any one of claims 38-141, wherein the Methanosphaera stadtmanae MCB-3 has a 16S RNA gene that is at least 95% identical to SEQ ID NO:100.

147. The method of any one of claims 38-141, wherein the Methanosphaera stadtmanae MCB-3 has a 16S RNA gene that is at least 95% identical to SEQ ID NO:101.

148. The method of any one of claims 38-141, wherein the Methanosphaera stadtmanae MCB-3 has a 16S RNA gene that is at least 95% identical to SEQ ID NO:102.

149. The method of any one of claims 38-141, wherein the Methanosphaera stadtmanae MCB-3 has a 16S RNA gene that is at least 95% identical to SEQ ID NO:103.

150. The method of any one of claims 39-149, wherein the Ruminococcus sp. 18P13 has a 16S RNA gene that is at least 95% identical to SEQ ID NO:108.

151. The method of any one of claims 39-149, wherein the Ruminococcus sp. 18P13 has a 16S RNA gene that is at least 95% identical to SEQ ID NO:109.

152. The method of any one of claims 39-149, wherein the Ruminococcus sp. 18P13 has a 16S RNA gene that is at least 95% identical to SEQ ID NO:110.

153. The method of any one of claims 39-149, wherein the Ruminococcus sp. 18P13 has a 16S RNA gene that is at least 95% identical to SEQ ID NO:111.

154. The method of any one of claims 40-153, wherein the Slackia piriformis DSM 22477 has a 16S RNA gene that is at least 95% identical to SEQ ID NO:112.

155. The method of any one of claims 40-153, wherein the Slackia piriformis DSM 22477 has a 16S RNA gene that is at least 95% identical to SEQ ID NO:113.

156. The method of any one of claims 40-153, wherein the Slackia piriformis DSM 22477 has a 16S RNA gene that is at least 95% identical to SEQ ID NO:114.

157. The method of any one of claims 40-153, wherein the Slackia piriformis DSM 22477 has a 16S RNA gene that is at least 95% identical to SEQ ID NO:115.

158. The method of any one of claims 40-153, wherein the Slackia piriformis DSM 22477 has a 16S RNA gene that is at least 95% identical to SEQ ID NO:116.

159. The method of any one of claims 40-153, wherein the Slackia piriformis DSM 22477 has a 16S RNA gene that is at least 95% identical to SEQ ID NO:117.

160. The method of any one of claims 41-159, wherein the Streptococcus hongkongensis HKU30 has a 16S RNA gene that is at least 95% identical to SEQ ID NO:118.

161. The method of any one of claims 41-159, wherein the Streptococcus hongkongensis HKU30 has a 16S RNA gene that is at least 95% identical to SEQ ID NO:119.

162. The method of any one of claims 3-14 and 18-161, wherein the bacterial species in the composition is viable.

163. The method of any one of claims 3-14 and 18-162, wherein the bacterial strain is lyophilized.

164. The method of any one of claims 3-14 and 18-163, wherein the composition further comprises one or more cryopreservants.

165. The method of any one of claims 3-14 and 18-164, wherein the effective amount of the bacterial strain comprises at least about 1×10³ colony forming units (CFU) of the bacterial strain.

166. The method of any one of claims 3-14 and 18-165, wherein the effective amount of the bacterial strain comprises about 1×10⁴ to about 1×10¹⁵ CFU of the bacterial strain.

167. The method of any one of claims 3-14 and 18-166, wherein the effective amount of the bacterial strain comprises about 1×10⁶ to about 1×10¹⁰ CFU of the bacterial strain.

168. The method of any one of claims 3-14 and 18-161, wherein the bacterial strain in the composition is non-viable.

169. The method of claim 168, wherein the non-viable bacterial strain is heat-killed, irradiated, or lysed.

170. The method of any one of claims 3-14 and 18-169, wherein the method comprises administering the composition to the subject once, twice, or three times per day.

171. The method of any one of claims 3-14 and 18-170, wherein the composition is formulated for oral administration.

172. The method of any one of claims 3-14 and 18-170, wherein the composition is formulated for rectal administration.

173. The method of any one of claims 3-14 and 18-172, wherein the composition is formulated as a tablet, a capsule, a powder, or a liquid.

174. The method of any one of claims 3-14 and 18-173, wherein the composition is formulated as a tablet.

175. The method of claim 174, wherein the tablet is coated.

176. The method of claim 175, wherein the coating comprises an enteric coating.

177. The method of any one of claims 3-176, wherein the method further comprises administering another treatment of colorectal cancer and/or other adjunct therapy to the subject.

178. The method of claim 177, wherein the composition comprising the bacterial strain treatment and the treatment for colorectal cancer and/or adjunct therapy are administered simultaneously.

179. The method of claim 177, wherein the composition comprising the bacterial strain treatment and the treatment for colorectal cancer and/or adjunct therapy are administered sequentially.

180. The method of any one of claims 177-179, wherein the treatment for colorectal cancer and/or adjunct therapy comprises a probiotic.

181. The method of any one of claims 177-179, wherein the treatment for colorectal cancer and/or adjunct therapy comprises surgery, radiation therapy, ablation, embolization, or a combination thereof.

182. The method of any one of claims 177-179, wherein the treatment for colorectal cancer and/or adjunct therapy comprises a therapeutic agent.

183. The method of claim 182, wherein the therapeutic agent comprises a chemotherapeutic agent, targeted therapy, immunotherapy, a cell therapy, or a combination thereof.

184. The method of claim 183, wherein the chemotherapeutic agent comprises aflibercept, capecitabine, fluorouracil, irinotecan, leucovorin, oxaliplatin, trifluridine, tipiracil, or a combination thereof.

185. The method of claim 183, wherein the targeted therapy comprises bevacizumab, cetuximab, ziv-aflibercept, panitumumab, regorafenib, or a combination thereof.

186. The method of any one of claims 183-185, wherein the combination of therapeutic agents is selected from the group consisting of: FOLFOX, FOLFIRI, CAPDX, FOLFIRI-bevacizumab, FOLFIRI-cetuximab, or XELOX, and a combination thereof.

187. The method of claim 183, wherein the immunotherapy comprises a checkpoint inhibitor.

188. The method of claim 187, wherein the checkpoint inhibitor targets one or more of: CTLA-4, PD-1, PD-L1, BTLA, LAG-3, A2AR, TIM-3, B7-H3, VISTA, and IDO.

189. The method of any one of claims 183-188, wherein the composition comprising the bacterial strain further comprises the therapeutic agent.

190. The method of claim 183, wherein the cell therapy is a CAR T-cell therapy.

191. The method of any one of claims 1-190, wherein the subject is a human.

192. A method for treating a subject in need thereof, the method comprising administering to the subject a composition comprising an effective amount of a bacterial strain selected from the group consisting of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, and a combination thereof.

193. The method of claim 192, wherein the subject has colorectal cancer.

194. A method for treating colorectal cancer in a subject, the method comprising administering to the subject a composition comprising an effective amount of a bacterial strain selected from the group consisting of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, and a combination thereof.

195. A method for treating colorectal cancer in a subject, the method comprising:
(a) detecting a dysbiosis associated with colorectal cancer in a sample from the subject; and
(b) administering to the subject a composition comprising an effective amount of a bacterial strain selected from the group consisting of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, and a combination thereof.

196. The method of claim 195, wherein the sample is a fecal sample.

197. The method of claim 195, wherein the sample is a biopsy sample.

198. The method of claim 197, wherein the biopsy sample is a colorectal biopsy sample.

199. The method of any one of claims 195-198, wherein detecting the dysbiosis associated with colorectal cancer comprises determining bacterial gene expression in the sample from the subject.

200. The method of any one of claims 195-199, wherein detecting the dysbiosis associated with colorectal cancer comprises determining bacterial composition in the sample from the subject.

201. The method of claim any one of claims 195-200, wherein detecting the dysbiosis associated with colorectal cancer comprises determining that Alistipes indistinctus DSM 22520, Azotobacter sp. A1 2, Bacteroides asaccharolyticus B440, Bacteroides sp. 2_2_4, Bilophila wadsworthia 3 1 6, Clostridium aldenense RMA9741, Clostridium bolteae DSM 15670, Clostridium lactatifermentans G17, Clostridium sp. LIP1, Desulfovibrio piger DSM 749, Dialister pneumosintes DSM 11619, Eubacterium biforme DSM 3989, Fusobacterium mortiferum ATCC 9817, Hungatella hathewayi 1313, Megasphaera micronuciformis DSM 17226, Micromonas micros ACM 5086, Paraprevotella clara DSM 19731, Peptostreptococcus stomatis W2278, Porphyromonas somerae WAL 6690, Roseburia inulinivorans A2-194, Ruminococcus gnavus VPI C7-9, Shigella dysenteriae CECT 584, Subdoligranulum variabile BI-114, Veillonella alcalescens 259, or a combination thereof, is increased in the sample from subject.

202. The method of claim any one of claims 195-201, wherein detecting the dysbiosis associated with colorectal cancer comprises determining that Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, or a combination thereof, is decreased in the sample from subject.

203. The method of claim 202, wherein Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, or a combination thereof, is decreased in the gastrointestinal tract of the subject.

204. A method for treating a subject in need thereof, the method comprising decreasing a population of an increased bacterial strain in the subject, wherein the increased bacterial strain is selected from the group consisting of: Alistipes indistinctus DSM 22520, Azotobacter sp. A1 2, Bacteroides asaccharolyticus B440, Bacteroides sp. 2_2_4, Bilophila wadsworthia 3 1 6, Clostridium aldenense RMA9741, Clostridium bolteae DSM 15670, Clostridium lactatifermentans G17, Clostridium sp. LIP1, Desulfovibrio piger DSM 749, Dialister pneumosintes DSM 11619, Eubacterium biforme DSM 3989, Fusobacterium mortiferum ATCC 9817, Hungatella hathewayi 1313, Megasphaera micronuciformis DSM 17226, Micromonas micros ACM 5086, Paraprevotella clara DSM 19731, Peptostreptococcus stomatis W2278, Porphyromonas somerae WAL 6690, Roseburia inulinivorans A2-194, Ruminococcus gnavus VPI C7-9, Shigella dysenteriae CECT 584, Subdoligranulum variabile BI-114, Veillonella alcalescens 259, and a combination thereof.

205. The method of claim 204, wherein the subject has colorectal cancer.

206. The method of claim 204 or 205, wherein decreasing the population of an increased bacterial strain comprises administering to the subject a bacteriophage.

207. The method of any one of claims 204-206, wherein decreasing the population of an increased bacterial strain comprises administering to the subject a composition comprising an effective amount of a bacterial strain selected from the group consisting of: Bacteroides eggerthii 1 2 48FAA, Bacteroides eggerthii CCUG 9559, Bacteroides goldsteinii ATCC BAA 1180, Bacteroides pectinophilus N3, Bacteroides plebeius M12, Bacteroides vulgatus 8482, Barnesiella intestinihominis DSM 21032, Bifidobacterium stercoris EG1, Clostridium sp. 40, Clostridium spiroforme CCM 6168, Eubacterium eligens DSM 3376, Eubacterium hallii DSM 3353, Megamonas funiformis DSM 19343, Megasphaera elsdenii LC1, Parabacteroides johnsonii M-165, Butyricimonas virosa MT12, Clostridium citroniae DSM 19261, Lactobacillus ruminis RF3, Methanosphaera stadtmanae MCB-3, Ruminococcus sp. 18P13, Slackia piriformis DSM 22477, Streptococcus hongkongensis HKU30, and a combination thereof.

208. The method of any one of claims 192-203 and 205-207, wherein the colorectal cancer is adenocarcinoma, a carcinoid tumor, a gastrointestinal stromal tumor, or lymphoma.

209. The method of any one of claims 192-203 and 207-208, wherein the bacterial strain comprises Bacteroides eggerthii 1 2 48FAA.

210. The method of any one of claims 192-203 and 207-209, wherein the bacterial strain comprises Bacteroides eggerthii CCUG 9559.

211. The method of any one of claims 192-203 and 207-210, wherein the bacterial strain comprises Bacteroides goldsteinii ATCC BAA 1180.

212. The method of any one of claims 192-203 and 207-211, wherein the bacterial strain comprises Bacteroides pectinophilus N3.

213. The method of any one of claims 192-203 and 207-212, wherein the bacterial strain comprises Bacteroides plebeius M12.

214. The method of any one of claims 192-203 and 207-213, wherein the bacterial strain comprises Bacteroides vulgatus 8482.

215. The method of any one of claims 192-203 and 207-214, wherein the bacterial strain comprises Barnesiella intestinihominis DSM 21032.

216. The method of any one of claims 192-203 and 207-215, wherein the bacterial strain comprises Bifidobacterium stercoris EG1.

217. The method of any one of claims 192-203 and 207-216, wherein the bacterial strain comprises Clostridium sp. 40.

218. The method of any one of claims 192-203 and 207-217, wherein the bacterial strain comprises Clostridium spiroforme CCM 6168.

219. The method of any one of claims 192-203 and 207-218, wherein the bacterial strain comprises Eubacterium eligens DSM 3376.

220. The method of any one of claims 192-203 and 207-219, wherein the bacterial strain comprises Eubacterium hallii DSM 3353.

221. The method of any one of claims 192-203 and 207-220, wherein the bacterial strain comprises Megamonas funiformis DSM 19343.

222. The method of any one of claims 192-203 and 207-221, wherein the bacterial strain comprises Megasphaera elsdenii LC1.

223. The method of any one of claims 192-203 and 207-222, wherein the bacterial strain comprises Parabacteroides johnsonii M-165.

224. The method of any one of claims 192-203 and 207-223, wherein the bacterial strain comprises Butyricimonas virosa MT12.

225. The method of any one of claims 192-203 and 207-224, wherein the bacterial strain comprises Clostridium citroniae DSM 19261.

226. The method of any one of claims 192-203 and 207-225, wherein the bacterial strain comprises Lactobacillus ruminis RF3.

227. The method of any one of claims 192-203 and 207-226, wherein the bacterial strain comprises Methanosphaera stadtmanae MCB-3.

228. The method of any one of claims 192-203 and 207-227, wherein the bacterial strain comprises Ruminococcus sp. 18P13.

229. The method of any one of claims 192-203 and 207-228, wherein the bacterial strain comprises Slackia piriformis DSM 22477.

230. The method of any one of claims 192-203 and 207-229, wherein the bacterial strain comprises Streptococcus hongkongensis HKU30.

231. The method of any one of claims 192-203 and 207-230, wherein the bacterial strain improves intestinal barrier function of the subject.

232. The method of any one of claims 192-203 and 207-231, wherein the Bacteroides eggerthii 1 2 48FAA has a 16S RNA gene that is at least 95% identical to SEQ ID NO:1.

233. The method of any one of claims 192-203 and 207-232, wherein the Bacteroides eggerthii 1 2 48FAA has a 16S RNA gene that is at least 95% identical to SEQ ID NO:2.

234. The method of any one of claims 192-203 and 207-233, wherein the Bacteroides eggerthii 1 2 48FAA has a 16S RNA gene that is at least 95% identical to SEQ ID NO:3.

235. The method of any one of claims 192-203 and 207-234, wherein the Bacteroides eggerthii 1 2 48FAA has a 16S RNA gene that is at least 95% identical to SEQ ID NO:4.

236. The method of any one of claims 192-203 and 207-235, wherein the Bacteroides eggerthii 1 2 48FAA has a 16S RNA gene that is at least 95% identical to SEQ ID NO:5.

237. The method of any one of claims 192-203 and 207-236, wherein the Bacteroides eggerthii 1 2 48FAA has a 16S RNA gene that is at least 95% identical to SEQ ID NO:6.

238. The method of any one of claims 192-203 and 207-237, wherein the Bacteroides eggerthii CCUG 9559 has a 16S RNA gene that is at least 95% identical to SEQ ID NO:7.

239. The method of any one of claims 192-203 and 207-238, wherein the Bacteroides eggerthii CCUG 9559 has a 16S RNA gene that is at least 95% identical to SEQ ID NO:8.

240. The method of any one of claims 192-203 and 207-239, wherein the Bacteroides eggerthii CCUG 9559 has a 16S RNA gene that is at least 95% identical to SEQ ID NO:9.

241. The method of any one of claims 192-203 and 207-240, wherein the Bacteroides eggerthii CCUG 9559 has a 16S RNA gene that is at least 95% identical to SEQ ID NO:10.

242. The method of any one of claims 192-203 and 207-241, wherein the Bacteroides eggerthii CCUG 9559 has a 16S RNA gene that is at least 95% identical to SEQ ID NO:11.

243. The method of any one of claims 192-203 and 207-242, wherein the Bacteroides eggerthii CCUG 9559 has a 16S RNA gene that is at least 95% identical to SEQ ID NO:12.

244. The method of any one of claims 192-203 and 207-243, wherein the Bacteroides goldsteinii ATCC BAA 1180 has a 16S RNA gene that is at least 95% identical to SEQ ID NO:13.

245. The method of any one of claims 192-203 and 207-244, wherein the Bacteroides goldsteinii ATCC BAA 1180 has a 16S RNA gene that is at least 95% identical to SEQ ID NO:14.

246. The method of any one of claims 192-203 and 207-245, wherein the Bacteroides goldsteinii ATCC BAA 1180 has a 16S RNA gene that is at least 95% identical to SEQ ID NO:15.

247. The method of any one of claims 192-203 and 207-246, wherein the Bacteroides goldsteinii ATCC BAA 1180 has a 16S RNA gene that is at least 95% identical to SEQ ID NO:16.

248. The method of any one of claims 192-203 and 207-247, wherein the Bacteroides goldsteinii ATCC BAA 1180 has a 16S RNA gene that is at least 95% identical to SEQ ID NO:17.

249. The method of any one of claims 192-203 and 207-248, wherein the Bacteroides goldsteinii ATCC BAA 1180 has a 16S RNA gene that is at least 95% identical to SEQ ID NO:18.

250. The method of any one of claims 192-203 and 207-249, wherein the Bacteroides goldsteinii ATCC BAA 1180 has a 16S RNA gene that is at least 95% identical to SEQ ID NO:19.

251. The method of any one of claims 192-203 and 207-250, wherein the Bacteroides pectinophilus N3 has a 16S RNA gene that is at least 95% identical to SEQ ID NO:20.

252. The method of any one of claims 192-203 and 207-251, wherein the Bacteroides pectinophilus N3 has a 16S RNA gene that is at least 95% identical to SEQ ID NO:21.

253. The method of any one of claims 192-203 and 207-252, wherein the Bacteroides pectinophilus N3 has a 16S RNA gene that is at least 95% identical to SEQ ID NO:22.

254. The method of any one of claims 192-203 and 207-253, wherein the Bacteroides pectinophilus N3 has a 16S RNA gene that is at least 95% identical to SEQ ID NO:23.

255. The method of any one of claims 192-203 and 207-254, wherein the Bacteroides pectinophilus N3 has a 16S RNA gene that is at least 95% identical to SEQ ID NO:24.

256. The method of any one of claims 192-203 and 207-255, wherein the Bacteroides pectinophilus N3 has a 16S RNA gene that is at least 95% identical to SEQ ID NO:25.

257. The method of any one of claims 192-203 and 207-256, wherein the Bacteroides plebeius M12 has a 16S RNA gene that is at least 95% identical to SEQ ID NO:26.

258. The method of any one of claims 192-203 and 207-257, wherein the Bacteroides plebeius M12 has a 16S RNA gene that is at least 95% identical to SEQ ID NO:27.

259. The method of any one of claims 192-203 and 207-258, wherein the Bacteroides plebeius M12 has a 16S RNA gene that is at least 95% identical to SEQ ID NO:28.

260. The method of any one of claims 192-203 and 207-259, wherein the Bacteroides vulgatus 8482 has a 16S RNA gene that is at least 95% identical to SEQ ID NO:29.

261. The method of any one of claims 192-203 and 207-260, Bacteroides vulgatus 8482 has a 16S RNA gene that is at least 95% identical to SEQ ID NO:30.

262. The method of any one of claims 192-203 and 207-261, Bacteroides vulgatus 8482 has a 16S RNA gene that is at least 95% identical to SEQ ID NO:31.

263. The method of any one of claims 192-203 and 207-262, Bacteroides vulgatus 8482 has a 16S RNA gene that is at least 95% identical to SEQ ID NO:32.

264. The method of any one of claims 192-203 and 207-263, Bacteroides vulgatus 8482 has a 16S RNA gene that is at least 95% identical to SEQ ID NO:33.

265. The method of any one of claims 192-203 and 207-264, Bacteroides vulgatus 8482 has a 16S RNA gene that is at least 95% identical to SEQ ID NO:34.

266. The method of any one of claims 192-203 and 207-265, Bacteroides vulgatus 8482 has a 16S RNA gene that is at least 95% identical to SEQ ID NO:35.

267. The method of any one of claims 192-203 and 207-266, Bacteroides vulgatus 8482 has a 16S RNA gene that is at least 95% identical to SEQ ID NO:36.

268. The method of any one of claims 192-203 and 207-267, Bacteroides vulgatus 8482 has a 16S RNA gene that is at least 95% identical to SEQ ID NO:37.

269. The method of any one of claims 192-203 and 207-268, Bacteroides vulgatus 8482 has a 16S RNA gene that is at least 95% identical to SEQ ID NO:38.

270. The method of any one of claims 192-203 and 207-269, Bacteroides vulgatus 8482 has a 16S RNA gene that is at least 95% identical to SEQ ID NO:39.

271. The method of any one of claims 192-203 and 207-270, Bacteroides vulgatus 8482 has a 16S RNA gene that is at least 95% identical to SEQ ID NO:40.

272. The method of any one of claims 192-203 and 207-271, wherein the Barnesiella intestinihominis DSM 21032 has a 16S RNA gene that is at least 95% identical to SEQ ID NO:41.

273. The method of any one of claims 192-203 and 207-272, wherein the Barnesiella intestinihominis DSM 21032 has a 16S RNA gene that is at least 95% identical to SEQ ID NO:42.

274. The method of any one of claims 192-203 and 207-273, wherein the Barnesiella intestinihominis DSM 21032 has a 16S RNA gene that is at least 95% identical to SEQ ID NO:43.

275. The method of any one of claims 192-203 and 207-274, wherein the Barnesiella intestinihominis DSM 21032 has a 16S RNA gene that is at least 95% identical to SEQ ID NO:44.

276. The method of any one of claims 192-203 and 207-275, wherein the Barnesiella intestinihominis DSM 21032 has a 16S RNA gene that is at least 95% identical to SEQ ID NO:45.

277. The method of any one of claims 192-203 and 207-276, wherein the Barnesiella intestinihominis DSM 21032 has a 16S RNA gene that is at least 95% identical to SEQ ID NO:46.

278. The method of any one of claims 192-203 and 207-277, wherein the Barnesiella intestinihominis DSM 21032 has a 16S RNA gene that is at least 95% identical to SEQ ID NO:47.

279. The method of any one of claims 192-203 and 207-278, wherein the Barnesiella intestinihominis DSM 21032 has a 16S RNA gene that is at least 95% identical to SEQ ID NO:48.

280. The method of any one of claims 192-203 and 207-279, wherein the Barnesiella intestinihominis DSM 21032 has a 16S RNA gene that is at least 95% identical to SEQ ID NO:49.

281. The method of any one of claims 192-203 and 207-280, wherein the Barnesiella intestinihominis DSM 21032 has a 16S RNA gene that is at least 95% identical to SEQ ID NO:50.

282. The method of any one of claims 192-203 and 207-281, wherein the Bifidobacterium stercoris EG1 has a 16S RNA gene that is at least 95% identical to SEQ ID NO:51.

283. The method of any one of claims 192-203 and 207-282, wherein the Bifidobacterium stercoris EG1 has a 16S RNA gene that is at least 95% identical to SEQ ID NO:52.

284. The method of any one of claims 192-203 and 207-283, wherein the Bifidobacterium stercoris EG1 has a 16S RNA gene that is at least 95% identical to SEQ ID NO:53.

285. The method of any one of claims 192-203 and 207-284, wherein the Bifidobacterium stercoris EG1 has a 16S RNA gene that is at least 95% identical to SEQ ID NO:54.

286. The method of any one of claims 192-203 and 207-285, wherein the Clostridium sp. 40 has a 16S RNA gene that is at least 95% identical to SEQ ID NO:57.

287. The method of any one of claims 192-203 and 207-286, wherein the Clostridium spiroforme CCM 6168 has a 16S RNA gene that is at least 95% identical to SEQ ID NO:58.

288. The method of any one of claims 192-203 and 207-287, wherein the Clostridium spiroforme CCM 6168 has a 16S RNA gene that is at least 95% identical to SEQ ID NO:59.

289. The method of any one of claims 192-203 and 207-288, wherein the Clostridium spiroforme CCM 6168 has a 16S RNA gene that is at least 95% identical to SEQ ID NO:60.

290. The method of any one of claims 192-203 and 207-289, wherein the Clostridium spiroforme CCM 6168 has a 16S RNA gene that is at least 95% identical to SEQ ID NO:61.

291. The method of any one of claims 192-203 and 207-290, wherein the Clostridium spiroforme CCM 6168 has a 16S RNA gene that is at least 95% identical to SEQ ID NO:62.

292. The method of any one of claims 192-203 and 207-291, wherein the Clostridium spiroforme CCM 6168 has a 16S RNA gene that is at least 95% identical to SEQ ID NO:63.

293. The method of any one of claims 192-203 and 207-292, wherein the Eubacterium eligens DSM 3376 has a 16S RNA gene that is at least 95% identical to SEQ ID NO:64.

294. The method of any one of claims 192-203 and 207-293, wherein the Eubacterium eligens DSM 3376 has a 16S RNA gene that is at least 95% identical to SEQ ID NO:65.

295. The method of any one of claims 192-203 and 207-294, wherein the Eubacterium eligens DSM 3376 has a 16S RNA gene that is at least 95% identical to SEQ ID NO:66.

296. The method of any one of claims 192-203 and 207-295, wherein the Eubacterium eligens DSM 3376 has a 16S RNA gene that is at least 95% identical to SEQ ID NO:67.

297. The method of any one of claims 192-203 and 207-296, wherein the Eubacterium eligens DSM 3376 has a 16S RNA gene that is at least 95% identical to SEQ ID NO:68.

298. The method of any one of claims 192-203 and 207-297, wherein the Eubacterium eligens DSM 3376 has a 16S RNA gene that is at least 95% identical to SEQ ID NO:69.

299. The method of any one of claims 192-203 and 207-298, wherein the Eubacterium eligens DSM 3376 has a 16S RNA gene that is at least 95% identical to SEQ ID NO:70.

300. The method of any one of claims 192-203 and 207-299, wherein the Eubacterium hallii DSM 3353 has a 16S RNA gene that is at least 95% identical to SEQ ID NO:71.

301. The method of any one of claims 192-203 and 207-300, wherein the Eubacterium hallii DSM 3353 has a 16S RNA gene that is at least 95% identical to SEQ ID NO:72.

302. The method of any one of claims 192-203 and 207-301, wherein the Megamonas funiformis DSM 19343 has a 16S RNA gene that is at least 95% identical to SEQ ID NO:80.

303. The method of any one of claims 192-203 and 207-302, wherein the Megamonas funiformis DSM 19343 has a 16S RNA gene that is at least 95% identical to SEQ ID NO:81.

304. The method of any one of claims 192-203 and 207-303, wherein the Megamonas funiformis DSM 19343 has a 16S RNA gene that is at least 95% identical to SEQ ID NO:82.

305. The method of any one of claims 192-203 and 207-304, wherein the Megamonas funiformis DSM 19343 has a 16S RNA gene that is at least 95% identical to SEQ ID NO:83.

306. The method of any one of claims 192-203 and 207-305, wherein the Megamonas funiformis DSM 19343 has a 16S RNA gene that is at least 95% identical to SEQ ID NO:84.

307. The method of any one of claims 192-203 and 207-306, wherein the Megamonas funiformis DSM 19343 has a 16S RNA gene that is at least 95% identical to SEQ ID NO:85.

308. The method of any one of claims 192-203 and 207-307, wherein the Megamonas funiformis DSM 19343 has a 16S RNA gene that is at least 95% identical to SEQ ID NO:86.

309. The method of any one of claims 192-203 and 207-308, wherein the Megasphaera elsdenii LC1 has a 16S RNA gene that is at least 95% identical to SEQ ID NO:87.

310. The method of any one of claims 192-203 and 207-309, wherein the Megasphaera elsdenii LC1 has a 16S RNA gene that is at least 95% identical to SEQ ID NO:88.

311. The method of any one of claims 192-203 and 207-310, wherein the Megasphaera elsdenii LC1 has a 16S RNA gene that is at least 95% identical to SEQ ID NO:89.

312. The method of any one of claims 192-203 and 207-311, wherein the Megasphaera elsdenii LC1 has a 16S RNA gene that is at least 95% identical to SEQ ID NO:90.

313. The method of any one of claims 192-203 and 207-312, wherein the Megasphaera elsdenii LC1 has a 16S RNA gene that is at least 95% identical to SEQ ID NO:91.

314. The method of any one of claims 192-203 and 207-313, wherein the Megasphaera elsdenii LC1 has a 16S RNA gene that is at least 95% identical to SEQ ID NO:92.

315. The method of any one of claims 192-203 and 207-314, wherein the Megasphaera elsdenii LC1 has a 16S RNA gene that is at least 95% identical to SEQ ID NO:93.

316. The method of any one of claims 192-203 and 207-315, wherein the Megasphaera elsdenii LC1 has a 16S RNA gene that is at least 95% identical to SEQ ID NO:94.

317. The method of any one of claims 192-203 and 207-316, wherein the Megasphaera elsdenii LC1 has a 16S RNA gene that is at least 95% identical to SEQ ID NO:95.

318. The method of any one of claims 192-203 and 207-317, wherein the Parabacteroides johnsonii M 165 has a 16S RNA gene that is at least 95% identical to SEQ ID NO:104.

319. The method of any one of claims 192-203 and 207-318, wherein the Parabacteroides johnsonii M 165 has a 16S RNA gene that is at least 95% identical to SEQ ID NO:105.

320. The method of any one of claims 192-203 and 207-319, wherein the Parabacteroides johnsonii M 165 has a 16S RNA gene that is at least 95% identical to SEQ ID NO:106.

321. The method of any one of claims 192-203 and 207-320, wherein the Parabacteroides johnsonii M 165 has a 16S RNA gene that is at least 95% identical to SEQ ID NO:107.

322. The method of any one of claims 192-203 and 207-321, wherein the Butyricimonas virosa MT12 has a 16S RNA gene that is at least 95% identical to SEQ ID NO:55.

323. The method of any one of claims 192-203 and 207-322, wherein the Clostridium citroniae DSM 19261 has a 16S RNA gene that is at least 95% identical to SEQ ID NO:56.

324. The method of any one of claims 192-203 and 207-323, wherein the Lactobacillus ruminis RF3 has a 16S RNA gene that is at least 95% identical to SEQ ID NO:73.

325. The method of any one of claims 192-203 and 207-324, wherein the Lactobacillus ruminis RF3 has a 16S RNA gene that is at least 95% identical to SEQ ID NO:74.

326. The method of any one of claims 192-203 and 207-325, wherein the Lactobacillus ruminis RF3 has a 16S RNA gene that is at least 95% identical to SEQ ID NO:75.

327. The method of any one of claims 192-203 and 207-326, wherein the Lactobacillus ruminis RF3 has a 16S RNA gene that is at least 95% identical to SEQ ID NO:76.

328. The method of any one of claims 192-203 and 207-327, wherein the Lactobacillus ruminis RF3 has a 16S RNA gene that is at least 95% identical to SEQ ID NO:77.

329. The method of any one of claims 192-203 and 207-328, wherein the Lactobacillus ruminis RF3 has a 16S RNA gene that is at least 95% identical to SEQ ID NO:78.

330. The method of any one of claims 192-203 and 207-329, wherein the Lactobacillus ruminis RF3 has a 16S RNA gene that is at least 95% identical to SEQ ID NO:79.

331. The method of any one of claims 192-203 and 207-330, wherein the Methanosphaera stadtmanae MCB-3 has a 16S RNA gene that is at least 95% identical to SEQ ID NO:96.

332. The method of any one of claims 192-203 and 207-331, wherein the Methanosphaera stadtmanae MCB-3 has a 16S RNA gene that is at least 95% identical to SEQ ID NO:97.

333. The method of any one of claims 192-203 and 207-332, wherein the Methanosphaera stadtmanae MCB-3 has a 16S RNA gene that is at least 95% identical to SEQ ID NO:98.

334. The method of any one of claims 192-203 and 207-333, wherein the Methanosphaera stadtmanae MCB-3 has a 16S RNA gene that is at least 95% identical to SEQ ID NO:99.

335. The method of any one of claims 192-203 and 207-334, wherein the Methanosphaera stadtmanae MCB-3 has a 16S RNA gene that is at least 95% identical to SEQ ID NO:100.

336. The method of any one of claims 192-203 and 207-335, wherein the Methanosphaera stadtmanae MCB-3 has a 16S RNA gene that is at least 95% identical to SEQ ID NO:101.

337. The method of any one of claims 192-203 and 207-336, wherein the Methanosphaera stadtmanae MCB-3 has a 16S RNA gene that is at least 95% identical to SEQ ID NO:102.

338. The method of any one of claims 192-203 and 207-337, wherein the Methanosphaera stadtmanae MCB-3 has a 16S RNA gene that is at least 95% identical to SEQ ID NO:103.

339. The method of any one of claims 192-203 and 207-338, wherein the Ruminococcus sp. 18P13 has a 16S RNA gene that is at least 95% identical to SEQ ID NO:108.

340. The method of any one of claims 192-203 and 207-339, wherein the Ruminococcus sp. 18P13 has a 16S RNA gene that is at least 95% identical to SEQ ID NO:109.

341. The method of any one of claims 192-203 and 207-340, wherein the Ruminococcus sp. 18P13 has a 16S RNA gene that is at least 95% identical to SEQ ID NO:110.

342. The method of any one of claims 192-203 and 207-341, wherein the Ruminococcus sp. 18P13 has a 16S RNA gene that is at least 95% identical to SEQ ID NO:111.

343. The method of any one of claims 192-203 and 207-342, wherein the Slackia piriformis DSM 22477 has a 16S RNA gene that is at least 95% identical to SEQ ID NO:112.

344. The method of any one of claims 192-203 and 207-343, wherein the Slackia piriformis DSM 22477 has a 16S RNA gene that is at least 95% identical to SEQ ID NO:113.

345. The method of any one of claims 192-203 and 207-344, wherein the Slackia piriformis DSM 22477 has a 16S RNA gene that is at least 95% identical to SEQ ID NO:114.

346. The method of any one of claims 192-203 and 207-345, wherein the Slackia piriformis DSM 22477 has a 16S RNA gene that is at least 95% identical to SEQ ID NO:115.

347. The method of any one of claims 192-203 and 207-346, wherein the Slackia piriformis DSM 22477 has a 16S RNA gene that is at least 95% identical to SEQ ID NO:116.

348. The method of any one of claims 192-203 and 207-347, wherein the Slackia piriformis DSM 22477 has a 16S RNA gene that is at least 95% identical to SEQ ID NO:117.

349. The method of any one of claims 192-203 and 207-348, wherein the Streptococcus hongkongensis HKU30 has a 16S RNA gene that is at least 95% identical to SEQ ID NO:118.

350. The method of any one of claims 192-203 and 207-349, wherein the Streptococcus hongkongensis HKU30 has a 16S RNA gene that is at least 95% identical to SEQ ID NO:119.

351. The method of any one of claims 192-203 and 207-350, wherein the bacterial strain in the composition is viable.

352. The method of any one of claims 192-203 and 207-351, wherein the bacterial strain is lyophilized.

353. The method of any one of claims 192-203 and 207-352, wherein the composition further comprises one or more cryopreservants.

354. The method of any one of claims 192-203 and 207-353, wherein the effective amount of the bacterial strain comprises at least about 1×10³ colony forming units (CFU) of the bacterial strain.

355. The method of any one of claims 192-203 and 207-354, wherein the effective amount of the bacterial strain comprises about 1×10⁴ to about 1×10¹⁵ CFU of the bacterial strain.

356. The method of any one of claims 192-203 and 207-355, wherein the effective amount of the bacterial strain comprises about 1×10⁶ to about 1×10¹⁰ CFU of the bacterial strain.

357. The method of any one of claims 192-203 and 207-350, wherein the bacterial strain in the composition is non-viable.

358. The method of claim 357, wherein the non-viable bacterial strain is heat-killed, irradiated, or lysed.

359. The method of any one of claims 192-203 and 207-358, wherein the method comprises administering the composition to the subject once, twice, or three times per day.

360. The method of any one of claims 192-203 and 207-359, wherein the composition is formulated for oral administration.

361. The method of any one of claims 192-203 and 207-359, wherein the composition is formulated for rectal administration.

362. The method of any one of claims 192-203 and 207-361, wherein the composition is formulated as a tablet, a capsule, a powder, or a liquid.

363. The method of any one of claims 192-203 and 207-361, wherein the composition is formulated as a tablet.

364. The method of claim 363, wherein the tablet is coated.

365. The method of claim 364, wherein the coating comprises an enteric coating.

366. The method of any one of claims 192-203 and 207-365, wherein the method further comprises administering another treatment of colorectal cancer and/or other adjunct therapy to the subject.

367. The method of claim 366, wherein the composition comprising the bacterial strain treatment and the treatment for colorectal cancer and/or adjunct therapy are administered simultaneously.

368. The method of claim 366, wherein the composition comprising the bacterial strain treatment and the treatment for colorectal cancer and/or adjunct therapy are administered sequentially.

369. The method of any one of claims 366-368, wherein the treatment for colorectal cancer and/or adjunct therapy comprises a probiotic.

370. The method of any one of claims 366-368, wherein the treatment for colorectal cancer and/or adjunct therapy comprises surgery, radiation therapy, ablation, embolization, or a combination thereof.

371. The method of any one of claims 366-368, wherein the treatment for colorectal cancer and/or adjunct therapy comprises a therapeutic agent.

372. The method of claim 371, wherein the therapeutic agent comprises a chemotherapeutic agent, targeted therapy, immunotherapy, a cell therapy, or a combination thereof.

373. The method of claim 372, wherein the chemotherapeutic agent comprises aflibercept, capecitabine, fluorouracil, irinotecan, leucovorin, oxaliplatin, trifluridine, tipiracil, or a combination thereof.

374. The method of claim 372, wherein the targeted therapy comprises bevacizumab, cetuximab, ziv-aflibercept, panitumumab, regorafenib, or a combination thereof.

375. The method of claim 372, wherein the combination of therapeutic agents is selected from the group consisting of: FOLFOX, FOLFIRI, CAPDX, FOLFIRI-bevacizumab, FOLFIRI-cetuximab, or XELOX, and a combination thereof.

376. The method of claim 372, wherein the immunotherapy comprises a checkpoint inhibitor.

377. The method of claim 376, wherein the checkpoint inhibitor targets one or more of: CTLA-4, PD-1, PD-L1, BTLA, LAG-3, A2AR, TIM-3, B7-H3, VISTA, and IDO.

378. The method of any one of claims 371-377, wherein the composition comprising the bacterial strain further comprises the therapeutic agent.

379. The method of claim 372, wherein the cell therapy is a CAR T-cell therapy.

380. The method of any one of claims 192-379, wherein the subject is a human.


SEQUENCE LISTING (192 sequences)

SEQ ID NO: 1 - DNA, 1531 bp, Bacteroides eggerthii
  misc_feature: Bacteroides eggerthii 1 2 48FAA
ttacaatgaagagtttgatcctggctcaggatgaacgctagctacaggcttaacacatgcaagtcgaggggcagcatgattgaagcttgcttcaatcgatggcgaccggcgcacgggtgagtaacacgtatccaacctgccgataactcggggatagcctttcgaaagaaagattaatacccgatagtatagtatttccgcatggtttcactattaaagaatttcggttatcgatggggatgcgttccattagatagttggcggggtaacggcccaccaagtcaacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtggagtatgcatactcctttgtatgtaccgtatgaataaggatcggctaactccgtgccagcagccgcggtaatacggaggatccgagcgttatccggatttattgggtttaaagggagcgtaggcgggtgcttaagtcagttgtgaaagtttgcggctcaaccgtaaaattgcagttgatactgggtaccttgagtgcagcataggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgttggcgatacacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagtggaatgtagtggaaacattacagccttcgggccgctgtgaaggtgctgcatggttgtcgtcagctcgtgccgtgaggtgtcggcttaagtgccataacgagcgcaacccttatctatagttactatcaggtcatgctgaggactctatggagactgccgtcgtaagatgtgaggaaggtggggatgacgtcaaatcagcacggcccttacgtccggggctacacacgtgttacaatggggggtacagaaggcagctacctggcgacaggatgctaatccctaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcaaggagcgtcctagggtaaaactggtgattggggctaagtcgtaacaaggtagccgtaccggaaggtgcggctggaacacctcctttct

SEQ ID NO: 2 - DNA, 1539 bp, Bacteroides eggerthii
  misc_feature: Bacteroides eggerthii 1 2 48FAA
cttttacaatgaagagtttgatcctggctcaggatgaacgctagctacaggcttaacacatgcaagtcgaggggcagcatgattgaagcttgcttcaatcgatggcgaccggcgcacgggtgagtaacacgtatccaacctgccgataactcggggatagcctttcgaaagaaagattaatacccgatagtatagtatttccgcatggtttcactattaaagaatttcggttatcgatggggatgcgttccattagatagttggcggggtaacggcccaccaagtcaacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtggagtatgcatactcctttgtatgtaccgtatgaataaggatcggctaactccgtgccagcagccgcggtaatacggaggatccgagcgttatccggatttattgggtttaaagggagcgtaggcgggtgcttaagtcagttgtgaaagtttgcggctcaaccgtaaaattgcagttgatactgggtaccttgagtgcagcataggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgttggcgatacacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagtggaatgtagtggaaacattacagccttcgggccgctgtgaaggtgctgcatggttgtcgtcagctcgtgccgtgaggtgtcggcttaagtgccataacgagcgcaacccttatctatagttactatcaggtcatgctgaggactctatggagactgccgtcgtaagatgtgaggaaggtggggatgacgtcaaatcagcacggcccttacgtccggggctacacacgtgttacaatggggggtacagaaggcagctacctggcgacaggatgctaatccctaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcaaggagcgtcctagggtaaaactggtgattggggctaagtcgtaacaaggtagccgtaccggaaggtgcggctggaacacctcctttctggagc

SEQ ID NO: 3 - DNA, 1539 bp, Bacteroides eggerthii
  misc_feature: Bacteroides eggerthii 1 2 48FAA
cttttacaatgaagagtttgatcctggctcaggatgaacgctagctacaggcttaacacatgcaagtcgaggggcagcatgattgaagcttgcttcaatcgatggcgaccggcgcacgggtgagtaacacgtatccaacctgccgataactcggggatagcctttcgaaagaaagattaatacccgatagcatagtttttccgcatggtttcactattaaagaatttcggttatcgatggggatgcgttccattagatagttggcggggtaacggcccaccaagtcaacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtggagtatgcatactcctttgtatgtaccgtatgaataaggatcggctaactccgtgccagcagccgcggtaatacggaggatccgagcgttatccggatttattgggtttaaagggagcgtaggcgggtgcttaagtcagttgtgaaagtttgcggctcaaccgtaaaattgcagttgatactgggcgccttgagtgcagcataggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgttggcgatacacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagcggaatgtagtggaaacattacagccttcgggccgctgtgaaggtgctgcatggttgtcgtcagctcgtgccgtgaggtgtcggcttaagtgccataacgagcgcaacccttatctatagttactatcaggtcatgctgaggactctatggagactgccgtcgtaagatgtgaggaaggtggggatgacgtcaaatcagcacggcccttacgtccggggctacacacgtgttacaatggggggtacagaaggcagctacctggcgactggatgctaatccctaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcaaggagcgtcctagggtaaaactggtgattggggctaagtcgtaacaaggtagccgtaccggaaggtgcggctggaacacctcctttctggagc

SEQ ID NO: 4 - DNA, 1539 bp, Bacteroides eggerthii; identical to SEQ ID NO: 2
  misc_feature: Bacteroides eggerthii 1 2 48FAA

SEQ ID NO: 5 - DNA, 1539 bp, Bacteroides eggerthii; identical to SEQ ID NO: 2
  misc_feature: Bacteroides eggerthii 1 2 48FAA

SEQ ID NO: 6 - DNA, 1531 bp, Bacteroides eggerthii; identical to SEQ ID NO: 1
  misc_feature: Bacteroides eggerthii 1 2 48FAA

SEQ ID NO: 7 - DNA, 1516 bp, Bacteroides eggerthii
  misc_feature: Bacteroides eggerthii CCUG 9559
gtttgatcctggctcaggatgaacgctagctacaggcttaacacatgcaagtcgaggggcagcatgattgaagcttgcttcaatcgatggcgaccggcgcacgggtgagtaacacgtatccaacctgccgataactcggggatagcctttcgaaagaaagattaatacccgatagtatagtttttccgcatggtttcactattaaagaatttcggttatcgatggggatgcgttccattagatagttggcggggtaacggcccaccaagtcaacgatggataggggttctgagaggaaggtcccccacattggaactgagacacggtccaaactcctacgggaggcagcagtgaggaatattggccaatggacgagagtctgaaccagccaagtagcgtgaaggatgactgccctatggttttaaacttcttttatacgggaataaagtggagtatgcatactcctttgtatgtaccgtatgaataaggatcggctaactccgtgccagcagccgcggtaatacggaggatccgagcgttatccggatttattgggtttaaagggagcgtaggcgggtgcttaagtcagttgtgaaagtttgcggctcaaccgtaaaattgcagttgatactgggcgccttgagtgcagcataggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgttggcgatacacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagcggaatgtagtggaaacattacagccttcgggccgctgtgaaggtgctgcatggttgtcgtcagctcgtgccgtgaggtgtcggcttaagtgccataacgagcgcaacccttatctatagttactatcaggtcatgctgaggactctatggagactgccgtcgtaagatgtgaggaaggtggggatgacgtcaaatcagcacggcccttacgtccggggctacacacgtgttacaatggggggtacagaaggcagctacctggcgacaggatgctaatcccgaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaccgcaggagcgtcctagggtaaaactggtgattggggctaagtcgtaacaaggtagccgtaccggaaggtgcggctggaacacctcctttct

SEQ ID NO: 8 - DNA, 1539 bp, Bacteroides eggerthii
  misc_feature: Bacteroides eggerthii CCUG 9559
cttttacaatgaagagtttgatcctggctcaggatgaacgctagctacaggcttaacacatgcaagtcgaggggcagcatgattgaagcttgcttcaatcgatggcgaccggcgcacgggtgagtaacacgtatccaacctgccgataactcggggatagcctttcgaaagaaagattaatacccgatagtatagtttttccgcatggtttcactattaaagaatttcggttatcgatggggatgcgttccattagatagttggcggggtaacggcccaccaagtcaacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtggagtatgcatactcctttgtatgtaccgtatgaataaggatcggctaactccgtgccagcagccgcggtaatacggaggatccgagcgttatccggatttattgggtttaaagggagcgtaggcgggtgcttaagtcagttgtgaaagtttgcggctcaaccgtaaaattgcagttgatactgggcgccttgagtgcagcataggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgttggcgatacacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagcggaatgtagtggaaacattacagccttcgggccgctgtgaaggtgctgcatggttgtcgtcagctcgtgccgtgaggtgtcggcttaagtgccataacgagcgcaacccttatctatagttactatcaggtcatgctgaggactctatggagactgccgtcgtaagatgtgaggaaggtggggatgacgtcaaatcagcacggcccttacgtccggggctacacacgtgttacaatggggggtacagaaggcagctacctggcgacaggatgctaatcccgaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcaaggagcgtcctagggtaaaactggtgattggggctaagtcgtaacaaggtagccgtaccggaaggtgcggctggaacacctcctttctggagc

SEQ ID NO: 9 - DNA, 1527 bp, Bacteroides eggerthii
  misc_feature: Bacteroides eggerthii CCUG 9559
tacaatgaagagtttgatcctggctcaggatgaacgctagctacaggcttaacacatgcaagtcgaggggcagcatgattgaagcttgcttcaatcgatggcgaccggcgcacgggtgagtaacacgtatccaacctgccgataactcggggatagcctttcgaaagaaagattaatacccgatagtatagtttttccgcatggtttcactattaaagaatttcggttatcgatggggatgcgttccattagatagttggcggggtaacggcccaccaagtcaacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtggagtatgcatactcctttgtatgtaccgtatgaataaggatcggctaactccgtgccagcagccgcggtaatacggaggatccgagcgttatccggatttattgggtttaaagggagcgtaggcgggtgcttaagtcagttgtgaaagtttgcggctcaaccgtaaaattgcagttgatactgggcgccttgagtgcagcataggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgttggcgatacacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagcggaatgtagtggaaacattacagccttcgggccgctgtgaaggtgctgcatggttgtcgtcagctcgtgccgtgaggtgtcggcttaagtgccataacgagcgcaacccttatctatagttactatcaggtcatgctgaggactctatggagactgccgtcgtaagatgtgaggaaggtggggatgacgtcaaatcagcacggcccttacgtccggggctacacacgtgttacaatggggggtacagaaggcagctacctggcgacaggatgctaatcccgaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcaaggagcgtcctagggtaaaactggtgattggggctaagtcgtaacaaggtagccgtaccggaaggtgcggctggaacacctcctt

SEQ ID NO: 10 - DNA, 1527 bp, Bacteroides eggerthii; identical to SEQ ID NO: 9
  misc_feature: Bacteroides eggerthii CCUG 9559

SEQ ID NO: 11 - DNA, 1421 bp, Bacteroides eggerthii
  misc_feature: Bacteroides eggerthii CCUG 9559
ttgatcatggctcaggatgaacgttagctacaggacttacacatgcaagtcgaggggcagcatgattgaagcttgcttcaatcgatggcgaccggcgcacgggtgagtaacacgtatccaacctgccgataactcggggatagcctttcgaaagaaagattaatacccgatagtatagtttttccgcatggtttcattattaaagaatttcggttatcgatggggatgcgttccattagatagttggcggggtaacggcccaccaagtcaacgatggataggggttctgagaggaaggtcccccacattggaactgagacacggtccaaattcctacgggaggcagcagtgaggaatattggtcaatggacgagagtctgaaccagccaagtagcgtgaaggatgactgccctatgggttgtaaacttcttttatacgggaataaagtggagtatgcatactcctttgtatgtaccgtatgaataaggatcggctaactccgtgccagcagccgcggtaatacggaggatccgagcgttatccggatttattgggtttaaagggagcgtaggcgggtgcttaagtcagttgtgaaagtttgcggctcaaccgtaaaattgcagttgatactgggcgccttgagtgcagcataggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgttggcgatacacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagcggaatgtagtggaaacattacagccttcggccgctgtgaagtgctgcatggttgtcgtcagctcgtgccgtgaggtgtcggcttaagtgccataacgagcgcaacccttatctatagttactatcaggtcatgctgaggactctatggagactgccgtcgtaagatgtgaggaaggtggggatgacgtcaaatcagcacggcccttacgtccggggctacacacgtgttacaatggggggtacagaaggcagctacctggcgacaggatgctaatcccgaaaacctctctcagttcggattggagtctgcaacccgactccatgaagctggattcgmtagtaatcgcgcatcagccacggcgcggtgaatacgttcccgggccttgtacacaccgcccgtcaagccatgaaagccgggggtacctgaagtacgta

SEQ ID NO: 12 - DNA, 1488 bp, Bacteroides eggerthii
  misc_feature: Bacteroides eggerthii CCUG 9559
agagtttgatcctggctcaggatgaacgctagctacaggcttaacacatgcaagtcgaggggcagcatgattgaagcttgcttcaatcgatggcgaccggcgcacgggtgagtaacacgtatccaacctgccgataactcggggatagcctttcgaaagaaagattaatacccgatagtatagtttttccgcatggtttcactattaaagaatttcggttatcgatggggatgcgttccattagatagttggcggggtaacggcccaccaagtcaacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtggagtatgcatactcctttgtatgtaccgtatgaataaggatcggctaactccgtgccagcagccgcggtaatacggaggatccgagcgttatccggatttattgggtttaaagggagcgtaggcgggtgcttaagtcagttgtgaaagtttgcggctcaaccgtaaaattgcagttgatactgggcgccttgagtgcagcataggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgttggcgatacacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagcggaatgtagtggaaacattacagccttcgggccgctgtgaaggtgctgcatggttgtcgtcagctcgtgccgtgaggtgtcggcttaagtgccataacgagcgcaacccttatctatagttactatcaggtcatgctgaggactctatggagactgccgtcgtaagatgtgaggaaggtggggatgacgtcaaatcagcacggcccttacgtccggggctacacacgtgttacaatggggggtacagaaggcagctacctggcgacaggatgctaatcccgaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcaaggagcgtcctagggtaaaactggtgattggggctaagtcgtaacaaggtaacc

SEQ ID NO: 13 - DNA, 1386 bp, Bacteroides goldsteinii
  misc_feature: Bacteroides goldsteinii ATCC BAA 1180
gtcgaggggcagcacggatgtagcaatacattggtggcgaccggcgcacgggtgagtaacgcgtatgcaacctgcctatcagaggggaataacccggcgaaagtcggactaataccgcataaaacaggggttccacatggaaatatttgttaaagaattatcgctgatagatgggcatgcgttccattagatagttggtgaggtaacggctcaccaagtccacgatggataggggttctgagaggaaggtcccccacactggtactgagacacggaccagactcctacgggaggcagcagtgaggaatattggtcaatgggcgagagcctgaaccagccaagtcgcgtgaaggatgaaggatctatggtttgtaaacttcttttatatgggaataaagtgaggaacgtgttcctttttgtatgtaccatatgaataagcatcggctaactccgtgccagcagccgcggtaatacggaggatgcgagcgttatccggatttattgggtttaaagggtgcgtaggtggttaattaagtcagcggtgaaagtttgtggctcaaccataaaattgccgttgaaactggttgacttgagtatatttgaggtaggcggaatgcgtggtgtagcggtgaaatgcatagatatcacgcagaactccgattgcgaaggcagcttactaaactataactgacactgaagcacgaaagcgtggggatcaaacaggattagataccctggtagtccacgcagtaaacgatgattactagctgtttgcgatacacagtaagcggcacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgcattatgacagctctggaaacacattctctagtaatagcaatttgcgaggtgctgcatggttgtcgtcagctcgtgccgtgaggtgtcggcttaagtgccataacgagcgcaacccttatcactagttactaacaggtcaagctgaggactctagtgagactgccagcgtaagctgtgaggaaggtggggatgacgtcaaatcagcacggcccttacatccggggcgacacacgtgttacaatggtggggacaaagggcagctacctggcgacaggatgctaatctccaaaccccatctcagttcggatcgaagtctgcaacccgacttcgtgaagctggattcgctagtaatcgcgcatcagccatggcgcggtgaatacgttcccgggccttgtacacaccgcccgtcaagccatgggagttgggggtacctaaagtccgtaaccgcaagga

SEQ ID NO: 14 - DNA, 1518 bp, Bacteroides goldsteinii
  misc_feature: Bacteroides goldsteinii ATCC BAA 1180
gtttgatcctggctcaggatgaacgctagcgacaggcttaacacatgcaagtcgaggggcagcacgatgtagcaatacattggtggcgaccggcgcacgggtgagtaacgcgtatgcaacctacctatcagaggggaataacccggcgaaagtcggactaataccgcataaaacaggggttccacatggaaatatttgttaaagaattatcgctgatagatgggcatgcgttccattagatagttggtgaggtaacggctcaccaagtccacgatggataggggttctgagaggaaggtcccccacactggtactgagacacggaccagactcctacgggaggcagcagtgaggaatattggtcaatgggcgagagcctgaaccagccaagtcgcgtgaaggatgaaggatctatggtttgtaaacttcttttatatgggaataaagtgaggaacgtgttcctttttgtatgtaccatatgaataagcatcggctaactccgtgccagcagccgcggtaatacggaggatgcgagcgttatccggatttattgggtttaaagggtgcgtaggtggttaattaagtcagcggtgaaagtttgtggctcaaccataaaattgccgttgaaactggttgacttgagtatatttgaggtaggcggaatgcgtggtgtagcggtgaaatgcatagatatcacgcagaactccgattgcgaaggcagcttactaaactataactgacactgaagcacgaaagcgtggggatcaaacaggattagataccctggtagtccacgcagtaaacgatgattactagctgtttgcgatacacagtaagcggcacagcgaaagcgttaagtaatccacctggggagacgccggcaacggtgaaactcaaaggaattgacgggggcccgcacaagcggaggaacatgtggtttaattcgatgatacgcgaggaaccttacccgggtttgaacgcatattgacagctctggaaacagagtctctagtaatagcaatttgcgaggtgctgcatggttgtcgtcagctcgtgccgtgaggtgtcggcttaagtgccataacgagcgcaacccttatcactagttactaacaggtcatgctgaggactctagtgagactgccagcgtaagctgtgaggaaggtggggatgacgtcaaatcagcacggcccttacatccggggcgacacacgtgttacaatggtggggacaaagggcagctaccgtgtgagcggatgcgaatctccaaaccccatctcagttcggatcgaagtctgcaacccgacttcgtgaagctggattcgctagtaatcgcgcatcagccatggcgcggtgaatacgttcccgggccttgtacacaccacccgtcaagccatgggagttgggggtacctaaagtccgtaaccgcaaggatcggcctagggtaaaaccgatgactggggctaagtcgtaacaaggtagccgtaccggaaggtgcggctggaacacctcctttct

SEQ ID NO: 15 - DNA, 1542 bp, Bacteroides goldsteinii
  misc_feature: Bacteroides goldsteinii ATCC BAA 1180
caatttaaacaacgaagagtttgatcctggctcaggatgaacgctagcgacaggcttaacacatgcaagtcgaggggcagcacgatgtagcaatacattggtggcgaccggcgcacgggtgagtaacgcgtatgcaacctacctatcagaggggaataacccggcgaaagtcggactaataccgcataaaacaggggttccacatggaaatatttgttaaagaattatcgctgatagatgggcatgcgttccattagatagttggtgaggtaacggctcaccaagtccacgatggataggggttctgagaggaaggtcccccacactggtactgagacacggaccagactcctacgggaggcagcagtgaggaatattggtcaatgggcgagagcctgaaccagccaagtcgcgtgaaggatgaaggatctatggtttgtaaacttcttttatatgggaataaagtgaggaacgtgttcctttttgtatgtaccatatgaataagcatcggctaactccgtgccagcagccgcggtaatacggaggatgcgagcgttatccggatttattgggtttaaagggtgcgtaggtggttaattaagtcagcggtgaaagtttgtggctcaaccataaaattgccgttgaaactggttgacttgagtatatttgaggtaggcggaatgcgtggtgtagcggtgaaatgcatagatatcacgcagaactccgattgcgaaggcagcttactaaactataactgacactgaagcacgaaagcgtggggatcaaacaggattagataccctggtagtccacgcagtaaacgatgattactagctgtttgcgatacacagtaagcggcacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgcatattgacagctctggaaacagagtctctagtaatagcaatttgcgaggtgctgcatggttgtcgtcagctcgtgccgtgaggtgtcggcttaagtgccataacgagcgcaacccttatcactagttactaacaggtcatgctgaggactctagtgagactgccagcgtaagctgtgaggaaggtggggatgacgtcaaatcagcacggcccttacatccggggcgacacacgtgttacaatggtggggacaaagggcagctaccgtgtgagcggatgcaaatctccaaaccccatctcagttcggatcgaagtctgcaacccgacttcgtgaagctggattcgctagtaatcgcgcatcagccatggcgcggtgaatacgttcccgggccttgtacacaccgcccgtcaagccatgggagttgggggtacctaaagtccgtaaccgcaaggatcggcctagggtaaaaccgatgactggggctaagtcgtaacaaggtagccgtaccggaaggtgcggctggaacacctcctttctggagc

SEQ ID NO: 16 - DNA, 1542 bp, Bacteroides goldsteinii
  misc_feature: Bacteroides goldsteinii ATCC BAA 1180
caatttaaacaacgaagagtttgatcctggctcaggatgaacgctagcgacaggcttaacacatgcaagtcgaggggcagcacgatgtagcaatacattggtggcgaccggcgcacgggtgagtaacgcgtatgcaacctacctatcagaggggaataacccggcgaaagtcggactaataccgcataaaacaggggttccacatggaaatatttgttaaagaattatcgctgatagatgggcatgcgttccattagatagttggtgaggtaacggctcaccaagtccacgatggataggggttctgagaggaaggtcccccacactggtactgagacacggaccagactcctacgggaggcagcagtgaggaatattggtcaatgggcgagagcctgaaccagccaagtcgcgtgaaggatgaaggatctatggtttgtaaacttcttttatatgggaataaagtgaggaacgtgttcctttttgtatgtaccatatgaataagcatcggctaactccgtgccagcagccgcggtaatacggaggatgcgagcgttatccggatttattgggtttaaagggtgcgtaggtggttaattaagtcagcggtgaaagtttgtggctcaaccataaaattgccgttgaaactggttgacttgagtatatttgaggtaggcggaatgcgtggtgtagcggtgaaatgcatagatatcacgcagaactccgattgcgaaggcagcttactaaactataactgacactgaagcacgaaagcgtggggatcaaacaggattagataccctggtagtccacgcagtaaacgatgattactagctgtttgcgatacacagtaagcggcacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgcatattgacagctctggaaacagagtctctagtaatagcaatttgcgaggtgctgcatggttgtcgtcagctcgtgccgtgaggtgtcggcttaagtgccataacgagcgcaacccttatcactagttactaacaggtcatgctgaggactctagtgagactgccagcgtaagctgtgaggaaggtggggatgacgtcaaatcagcacggcccttacatccggggcgacacacgtgttacaatggtggggacaaagggcagctaccgtgtgagcggatgctaatctccaaacctcatctcagttcggatcgaagtctgcaacccgacttcgtgaagctggattcgctagtaatcgcgcatcagccatggcgcggtgaatacgttcccgggccttgtacacaccgcccgtcaagccatgggagttgggggtacctaaagtccgtaaccgcaaggatcggcctagggtaaaaccgatgactggggctaagtcgtaacaaggtagccgtaccggaaggtgcggctggaacacctcctttctggagc

SEQ ID NO: 17 - DNA, 1523 bp, Bacteroides goldsteinii
  misc_feature: Bacteroides goldsteinii ATCC BAA 1180
caatttaaacaacgaagagtttgatcctggctcaggatgaacgctagcgacaggcttaacacatgcaagtcgaggggcagcacgatgtagcaatacattggtggcgaccggcgcacgggtgagtaacgcgtatgcaacctacctatcagaggggaataacccggcgaaagtcggactaataccgcataaaacaggggttccacatggaaatatttgttaaagaattatcgctgatagatgggcatgcgttccattagatagttggtgaggtaacggctcaccaagtccacgatggataggggttctgagaggaaggtcccccacactggtactgagacacggaccagactcctacgggaggcagcagtgaggaatattggtcaatgggcgagagcctgaaccagccaagtcgcgtgaaggatgaaggatctatggtttgtaaacttcttttatatgggaataaagtgaggaacgtgttcctttttgtatgtaccatatgaataagcatcggctaactccgtgccagcagccgcggtaatacggaggatgcgagcgttatccggatttattgggtttaaagggtgcgtaggtggttaattaagtcagcggtgaaagtttgtggctcaaccataaaattgccgttgaaactggttgacttgagtatatttgaggtaggcggaatgcgtggtgtagcggtgaaatgcatagatatcacgcagaactccgattgcgaaggcagcttactaaactataactgacactgaagcacgaaagcgtggggatcaaacaggattagataccctggtagtccacgcagtaaacgatgattactagctgtttgcgatacacagtaagcggcacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgcatattgacagctctggaaacagagtctctagtaatagcaatttgcgaggtgctgcatggttgtcgtcagctcgtgccgtgaggtgtcggcttaagtgccataacgagcgcaacccttatcactagttactaacaggtcatgctgaggactctagtgagactgccagcgtaagctgtgaggaaggtggggatgacgtcaaatcagcacggcccttacatccggggcgacacacgtgttacaatggtggggacaaagggcagctaccgtgtgagcggatgctaatctccaaacctcatctcagttcggatcgaagtctgcaacccgacttcgtgaagctggattcgctagtaatcgcgcatcagccatggcgcggtgaatacgttcccgggccttgtacacaccgcccgtcaagccatgggagttgggggtacctaaagtccgtaaccgcaaggatcggcctagggtaaaaccgatgactggggctaagtcgtaacaaggtagccgtaccggaaggtgcggctgg

SEQ ID NO: 18 - DNA, 1542 bp, Bacteroides goldsteinii; identical to SEQ ID NO: 15
  misc_feature: Bacteroides goldsteinii ATCC BAA 1180

SEQ ID NO: 19 - DNA, 1476 bp, Bacteroides goldsteinii
  misc_feature: Bacteroides goldsteinii ATCC BAA 1180
tggctcaggatgaacgctagcgacaggcttaacacatgcaagtcgaggggcagcacgatgtagcaatacattggtggcgaccggcgcacgggtgagtaacgcgtatgcaacctacctatcagaggggaataacccggcgaaagtcggactaataccgcataaaacaggggttccacatggaaatatttgttaaagaattatcgctgatagatgggcatgcgttccattagatagttggtgaggtaacggctcaccaagtccacgatggataggggttctgagaggaaggtcccccacactggtactgagacacggaccagactcctacgggaggcagcagtgaggaatattggtcaatgggcgagagcctgaaccagccaagtcgcgtgaaggatgaaggatctatggtttgtaaacttcttttatatgggaataaagtgaggaacgtgttcctttttgtatgtaccatatgaataagcatcggctaactccgtgccagcagccgcggtaatacggaggatgcgagcgttatccggatttattgggtttaaagggtgcgtaggtggttaattaagtcagcggtgaaagtttgtggctcaaccataaaattgccgttgaaactggttgacttgagtatatttgaggtaggcggaatgcgtggtgtagcggtgaaatgcatagatatcacgcagaactccgattgcgaaggcagcttactaaactataactgacactgaagcacgaaagcgtggggatcaaacaggattagataccctggtagtccacgcagtaaacgatgattactagctgtttgcgatacacagtaagcggcacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgcatattgacagctctggaaacagagtctctagtaatagcaatttgcgaggtgctgcatggttgtcgtcagctcgtgccgtgaggtgtcggcttaagtgccataacgagcgcaacccttatcactagttactaacaggtcatgctgaggactctagtgagactgccagcgtaagctgtgaggaaggtggggatgacgtcaaatcagcacggcccttacatccggggcgacacacgtgttacaatggtggggacaaagggcagctaccgtgtgagcggatgctaatctccaaacctcatctcagttcggatcgaagtctgcaacccgacttcgtgaagctggattcgctagtaatcgcgcatcagccatggcgcggtgaatacgttcccgggccttgtacacaccgcccgtcaagccatgggagttgggggtacctaaagtccgtaaccgcaaggatcggcctagggtaaaaccgatgactggggctaagtcgtaacaaggtaacc

SEQ ID NO: 20 - DNA, 1542 bp, Bacteroides pectinophilus
  misc_feature: Bacteroides pectinophilus N3
tttttatgagagtttgatcctggctcaggatgaacgctggcggcgtgcttaacacatgcaagtcgaacgaagcacttaatgagatttcttcggattgattattttgtgactgagtggcggacgggtgagtaacgcgtgggtaacctgccccagaccgggggataacagtcggaaacgactgctaataccgcataagcgcacagtgccgcatggcacggtgtgaaaaactccggtggtctgggatggacccgcgtccgattagttagttggcggggtaacggcccaccaagaccgcgatcggtagccgacctgagagggtgaccggccacattgggactgagacacggcccagactcctacgggaggcagcagtggggaatattgcacaatggaggaaactctgatgcagcgacgccgcgtgagcgaagaagtatttcggtatgtaaagctctgtcagcagggaagataatgacggtacctgaccaagaagccccggctaactacgtgccagcagccgcggtaatacgtagggggcaagcgttatccggatttactgggtgtaaagggagtgtaggtggctgtgcaagtcaggagtgaaagcccggggctcaaccccgggactgctcttgaaactgcgcagcttgagtgcaggaggggcaggcggaattcctagtgtagcggtgaaatgcgtagatattaggaggaacaccggtggcgaaggcggcctgctggactgtaactgacactgaggctcgaaagcgtggggagcaaacaggattagataccctggtagtccacgccgtaaacgatgaatactaggtgtcggggctcataagagcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaggtcttgacatcctcctgaacggaaggtaatgcttccggtccttcgggacaggagagacaggtggtgcatggttgtcgtcagctcgtgtcgtgagatgttgggttaagtcccgcaacgagcgcaacccctatcctcagtagccagcagtccggctgggcactctgtggagactgccggggataacccggaggaaggtggggatgacgtcaaatcatcatgccccttatgacctgggctacacacgtgctacaatggcgctcaacaaagggaagcaatgccgcgaggccgagcaaatcccaaaaacagcgtcccagttcggactgtagtctgcaacccgactacacgaagctggaatcgctagtaatcgcgaatcagaatgtcgcggtgaatacgttcccgggtcttgtacacaccgcccgtcacaccatgggagtcggaaatgcccgaagtcagtgacccaaccgcaaggagggagctgccgaaggcaggtctggtaactggggtgaagtcgtaacaaggtagccgtatcggaaggtgcggctggatcacctcctttctaaggaa

SEQ ID NO: 21 - DNA, 1542 bp, Bacteroides pectinophilus
  misc_feature: Bacteroides pectinophilus N3
tttttatgagagtttgatcctggctcaggatgaacgctggcggcgtgcttaacacatgcaagtcgaacgaagcacttaatgagatttcttcggattgattattttgtgactgagtggcggacgggtgagtaacgcgtgggtaacctgccccagaccgggggataacagtcggaaacgactgctaataccgcataagcgcacagtgccgcatggcacagtgtgaaaaactccggtggtctgggatggacccgcgtccgattagttagttggcggggtaacggcccaccaagaccgcgatcggtagccgacctgagagggtgaccggccacattgggactgagacacggcccagactcctacgggaggcagcagtggggaatattgcacaatggaggaaactctgatgcagcgacgccgcgtgagcgaagaagtatttcggtatgtaaagctctgtcagcagggaagataatgacggtacctgaccaagaagccccggctaactacgtgccagcagccgcggtaatacgtagggggcaagcgttatccggatttactgggtgtaaagggagtgtaggtggctgtgcaagtcaggagtgaaagcccggggctcaaccccgggactgctcttgaaactgcgcagcttgagtgcaggaggggcaggcggaattcctagtgtagcggtgaaatgcgtagatattaggaggaacaccggtggcgaaggcggcctgctggactgtaactgacactgaggctcgaaagcgtggggagcaaacaggattagataccctggtagtccacgccgtaaacgatgaatactaggtgtcggggctcataagagcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaggtcttgacatcctcctgaacggaaggtaatgcttccggtccttcgggacaggagagacaggtggtgcatggttgtcgtcagctcgtgtcgtgagatgttgggttaagtcccgcaacgagcgcaacccctatcctcagtagccagcagtccggctgggcactctgtggagactgccggggataacccggaggaaggtggggatgacgtcaaatcatcatgccccttatgacctgggctacacacgtgctacaatggcgctcaacaaagggaagcaatgccgcgaggccgagcaaatcccaaaaacagcgtcccagttcggactgtagtctgcaacccgactacacgaagctggaatcgctagtaatcgcgaatcagaatgtcgcggtgaatacgttcccgggtcttgtacacaccgcccgtcacaccatgggagtcggaaatgcccgaagtcagtgacccaaccgcaaggagggagctgccgaaggcaggtctggtaactggggtgaagtcgtaacaaggtagccgtatcggaaggtgcggctggatcacctcctttctaaggaa

SEQ ID NO: 22 - DNA, 1530 bp, Bacteroides pectinophilus
  misc_feature: Bacteroides pectinophilus N3
tgagagtttgatcctggctcaggatgaacgctggcggcgtgcttaacacatgcaagtcgaacgaagcacttaatgagatttcttcggattgattattttgtgactgagtggcggacgggtgagtaacgcgtgggtaacctgccccagaccgggggataacagtcggaaacgactgctaataccgcataagcgcacagtgccgcatggcacagtgtgaaaaactccggtggtctgggatggacccgcgtccgattagttagttggcggggtaacggcccaccaagaccgcgatcggtagccgacctgagagggtgaccggccacattgggactgagacacggcccagactcctacgggaggcagcagtggggaatattgcacaatggaggaaactctgatgcagcgacgccgcgtgagcgaagaagtatttcggtatgtaaagctctgtcagcagggaagataatgacggtacctgaccaagaagccccggctaactacgtgccagcagccgcggtaatacgtagggggcaagcgttatccggatttactgggtgtaaagggagtgtaggtggctgtgcaagtcaggagtgaaagcccggggctcaaccccgggactgctcttgaaactgcgcagcttgagtgcaggaggggcaggcggaattcctagtgtagcggtgaaatgcgtagatattaggaggaacaccggtggcgaaggcggcctgctggactgtaactgacactgaggctcgaaagcgtggggagcaaacaggattagataccctggtagtccacgccgtaaacgatgaatactaggtgtcggggctcataagagcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaggtcttgacatcctcctgaacggaaggtaatgcttccggtccttcgggacaggagagacaggtggtgcatggttgtcgtcagctcgtgtcgtgagatgttgggttaagtcccgcaacgagcgcaacccctatcctcagtagccagcagtccggctgggcactctgtggagactgccggggataacccggaggaaggtggggatgacgtcaaatcatcatgccccttatgacctgggctacacacgtgctacaatggcgctcaacaaagggaagcaatgccgcgaggccgagcaaatcccaaaaacagcgtcccagttcggactgtagtctgcaacccgactacacgaagctggaatcgctagtaatcgcgaatcagaatgtcgcggtgaatacgttcccgggtcttgtacacaccgcccgtcacaccatgggagtcggaaatgcccgaagtcagtgacccaaccgcaaggagggagctgccgaaggcaggtctggtaactggggtgaagtcgtaacaaggtagccgtatcggaaggtgcggctggatcacctcctttct

SEQ ID NO: 23 - DNA, 1530 bp, Bacteroides pectinophilus
  misc_feature: Bacteroides pectinophilus N3
tgagagtttgatcctggctcaggatgaacgctggcggcgtgcttaacacatgcaagtcgaacgaagcacttaatgagatttcttcggattgattattttgtgactgagtggcggacgggtgagtaacgcgtgggtaacctgccccagaccgggggataacagtcggaaacgactgctaataccgcataagcgcacagtgccgcatggcacggtgtgaaaaactccggtggtctgggatggacccgcgtccgattagttagttggcggggtaacggcccaccaagaccgcgatcggtagccgacctgagagggtgaccggccacattgggactgagacacggcccagactcctacgggaggcagcagtggggaatattgcacaatggaggaaactctgatgcagcgacgccgcgtgagcgaagaagtatttcggtatgtaaagctctgtcagcagggaagataatgacggtacctgaccaagaagccccggctaactacgtgccagcagccgcggtaatacgtagggggcaagcgttatccggatttactgggtgtaaagggagtgtaggtggctgtgcaagtcaggagtgaaagcccggggctcaaccccgggactgctcttgaaactgcgcagcttgagtgcaggaggggcaggcggaattcctagtgtagcggtgaaatgcgtagatattaggaggaacaccggtggcgaaggcggcctgctggactgtaactgacactgaggctcgaaagcgtggggagcaaacaggattagataccctggtagtccacgccgtaaacgatgaatactaggtgtcggggctcataagagcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaggtcttgacatcctcctgaacggaaggtaatgcttccggtccttcgggacaggagagacaggtggtgcatggttgtcgtcagctcgtgtcgtgagatgttgggttaagtcccgcaacgagcgcaacccctatcctcagtagccagcagtccggctgggcactctgtggagactgccggggataacccggaggaaggtggggatgacgtcaaatcatcatgccccttatgacctgggctacacacgtgctacaatggcgctcaacaaagggaagcaatgccgcgaggccgagcaaatcccaaaaacagcgtcccagttcggactgtagtctgcaacccgactacacgaagctggaatcgctagtaatcgcgaatcagaatgtcgcggtgaatacgttcccgggtcttgtacacaccgcccgtcacaccatgggagtcggaaatgcccgaagtcagtgacccaaccgcaaggagggagctgccgaaggcaggtctggtaactggggtgaagtcgtaacaaggtagccgtatcggaaggtgcggctggatcacctcctttct

SEQ ID NO: 24 - DNA, 1530 bp, Bacteroides pectinophilus; identical to SEQ ID NO: 22
  misc_feature: Bacteroides pectinophilus N3

SEQ ID NO: 25 - DNA, 1530 bp, Bacteroides pectinophilus; identical to SEQ ID NO: 23
  misc_feature: Bacteroides pectinophilus N3

SEQ ID NO: 26 - DNA, 1489 bp, Bacteroides plebeius
  misc_feature: Bacteroides plebeius M12
agagtttgatcctggctcaggatgaacgctagctacaggcttaacacatgcaagtcgaggggcagcgggattgaagcttgcttcaattgccggcgaccggcgcacgggtgagtaacgcgtatccaaccttccgtacactcagggatagcctttcgaaagaaagattaatacctgatggtatgatgagattgcatgatagcatcattaaagatttatcggtgtacgatggggatgcgttccattaggtagtaggcggggtaacggcccacctagcctacgatggataggggttctgagaggaaggtcccccacattggaactgagacacggtccaaactcctacgggaggcagcagtgaggaatattggtcaatggacgagagtctgaaccagccaagtagcgtgaaggatgaaggtcctacggattgtaaacttcttttataagggaataaaacctcccacgtgtgggagcttgtatgtaccttatgaataagcatcggctaactccgtgccagcagccgcggtaatacggaggatgcgagcgttatccggatttattgggtttaaagggagcgcagacgggtcgttaagtcagctgtgaaagtttggggctcaaccttaaaattgcagttgatactggcgtccttgagtgcggttgaggtgtgcggaattcgtggtgtagcggtgaaatgcttagatatcacgaagaactccgattgcgaaggcagcacactaagccgtaactgacgttcatgctcgaaagtgtgggtatcaaacaggattagataccctggtagtccacacggtaaacgatggatactcgctgttggcgatatacagtcagcggcttagcgaaagcgttaagtatcccacctggggagtacgccggcaacggtgaaactcaaaggaattgacgggggcccgcacaagcggaggaacatgtggtttaattcgatgatacgcgaggaaccttacccgggcttaaattgcaaaggaatgatctggaaacaggtcagtcttcggacctttgtgaaggtgctgcatggttgtcgtcagctcgtgccgtgaggtgtcggcttaagtgccataacgagcgcaaccctcgccgccagttactaacaggtaatgctgaggactctggcgggactgccatcgtaagatgtgaggaaggtggggatgacgtcaaatcagcacggcccttacgtccggggctacacacgtgttacaatgggcggtacagaaggcagctacctggcgacaggatgccaatcccgaaagccgctctcagttcggactggagtctgcaacccgactccacgaagctggattcgctagtaatcgcgcatcagccacggcgcggtgaatacgttcccgggccttgtacacaccgcccgtcaagccatgaaagccgggggtacctgaagtgcgtaaccgcaaggagcgccctagggtaaaaccggtaattggggctaagtcgtaacaaggtaacc

SEQ ID NO: 27 - DNA, 1128 bp, Bacteroides plebeius
  misc_feature: Bacteroides plebeius M12
ttttacaatgaagagtttgatcctggctcaggatgaacgctagctacaggcttaacacatgcaagtcgaggggcagcgggattgaagcttgcttcaattgccggcgaccggcgcacgggtgagtaacgcgtatccaaccttccgtacactcagggatagcctttcgaaagaaagattaatacctgatggtatgatgagattgcatgatagcatcattaaagatttatcggtgtacgatggggatgcgttccattaggtagtaggcggggtaacggcccacctagcctacgatggataggggttctgagaggaaggtcccccacattggaactgagacacggtccaaactcctacgggaggcagcagtgaggaatattggtcaatggacgagagtctgaaccagccaagtagcgtgaaggatgaaggtcctacggattgtaaacttcttttataagggaataaaacctcccacgtgtgggagcttgtatgtaccttatgaataagcatcggctaactccgtgccagcagccgcggtaatacggaggatgcgagcgttatccggatttattgggtttaaagggagcgcagacgggtcgttaagtcagctgtgaaagtttggggctcaaccttaaaattgcagttgatactggcgtccttgagtgcggttgaggtgtgcggaattcgtggtgtagcggtgaaatgcttagatatcacgaagaactccgattgcgaaggcagcacactaagccgtaactgacgttcatgctcgaaagtgtgggtatcaaacaggattagataccctggtagtccacacggtaaacgatggatactcgctgttggcgatatacagtcagcggcttagcgaaagcgttaagtatcccacctggggagtacgccggcaacggtgaaactcaaaggaattgacgggggcccgcacaagcggaggaacatgtggtttattcgatgatacgcgaggaaccttacccgggcttaattgcaaaggaatgatctgaaacagtcagtcttcggactttgtgaggtgctgcatgtgtcgtcagctcgtgccgtgagtgtcgcttaagtgcaataacgagcgcatccctccgccgccagttacctaacaggtaa

SEQ ID NO: 28 - DNA, 1315 bp, Bacteroides plebeius
  misc_feature: Bacteroides plebeius M12
ttttacaatgaagagtttgatcctggctcaggatgaacgctagctacaggcttaacacatgcaagtcgaggggcagcgggattgaagcttgcttcaattgccggcgaccggcgcacgggtgagtaacgcgtatccaaccttccgtacactcagggatagcctttcgaaagaaagattaatacctgatggtatgatgagattgcatgatagcatcattaaagatttatcggtgtacgatggggatgcgttccattaggtagtaggcggggtaacggcccacctagcctacgatggataggggttctgagaggaaggtcccccacattggaactgagacacggtccaaactcctacgggaggcagcagtgaggaatattggtcaatggacgagagtctgaaccagccaagtagcgtgaaggatgaaggtcctacggattgtaaacttcttttataagggaataaaacctcccacgtgtgggagcttgtatgtaccttatgaataagcatcggctaactccgtgccagcagccgcggtaatacggaggatgcgagcgttatccggatttattgggtttaaagggagcgcagacgggtcgttaagtcagctgtgaaagtttggggctcaaccttaaaattgcagttgatactggcgtccttgagtgcggttgaggtgtgcggaattcgtggtgtagcggtgaaatgcttagatatcacgaagaactccgattgcgaaggcagcacactaagccgtaactgacgttcatgctcgaaagtgtgggtatcaaacaggattagataccctggtagtccacacggtaaacgatggatactcgctgttggcgatatacagtcagcggcttagcgaaagcgttaagtatcccacctggggagtacgccggcaacggtgaaactcaaaggaattgacgggggcccgcacaagcggaggaacatgtggtttaattcgatgatacgcgaggaaccttacccgggcttaaattgcaaaggaatgatctggaaacaggtcagtcttcggacctttgtgaaggtgctgcatggttgtcgtcagctcgtgccgtgaggtgtcggcttaagtgccataacgagcgcaaccctcgccgccagttactaacaggtaatgctgaggactctggcgggactgccatcgtagatgtgaggaaggtggggatgacgtcaatcagcacggcccttacgtccggggctacacacgtgttacaatgggcggtacagagcagctactgcgacagatgcatccgaagccgcttctcagttcggactggagttctgcaacccgact

SEQ ID NO: 29 - DNA, 1531 bp, Bacteroides vulgatus
  misc_feature: Bacteroides vulgatus 8482
  misc_feature (33)..(33): n is a, c, g, or t
  misc_feature (95)..(95): n is a, c, g, or t
  misc_feature (97)..(97): n is a, c, g, or t
  misc_feature (393)..(393): n is a, c, g, or t
  misc_feature (479)..(479): n is a, c, g, or t
  misc_feature (542)..(542): n is a, c, g, or t
  misc_feature (742)..(742): n is a, c, g, or t
  misc_feature (930)..(930): n is a, c, g, or t
  misc_feature (1202)..(1202): n is a, c, g, or t
  misc_feature (1253)..(1253): n is a, c, g, or t
  misc_feature (1497)..(1497): n is a, c, g, or t
  misc_feature (1508)..(1516): n is a, c, g, or t
ttacaatgaagagtttgatcctggctcaggatnaacgctagctacaggcttaacacatgcaagtcgaggggcagcatggtcttagcttgctaagncnatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagccngaaccagccaagtagcgtgaaggatgactgccctatgggttgtaaacttcttttataaaggaataaagtcgggtatggatacccgnttgcatgtactttatgaataaggatcggctaactccgtgccagcagccgcggtaatacggagnatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctnagctgcaactgacattgaggctcgaaagtgtgggtatcaaacaggattagataccctggtagtccacacggtaaacgatgaatactcgctgtttgcgatatactgcaagcggccaagcgaaagcgttaagtattccacctggggagtacgccggcaacggtgaaactcaaaggaattgacgggggccngcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttatgctgaggactctgacaagactgccatcgtaagatgtgaggaaggtggggatgacgtcaaatcagcacngcccttacgtccggggctacacacgtgttacaatggggggtacagagggcngctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagtcatgggagccgggggtacctgaagtgcgtaaccgcgaggagcgccctagggtaaaactggtgactggggctaagtcgtaacaaggtagcngtaccggaagnnnnnnnnngaacacctcctttct

SEQ ID NO: 30 - DNA, 1393 bp, Bacteroides vulgatus
  misc_feature: Bacteroides vulgatus 8482
caggcttaccacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaagatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatactgcaagcggccaagcgaaagcgttaagtattccacctggggagtacgccggcaacggtgaaactcaaaggaattgacgggggcccgcacaagcggaggaacatgtggtttaattcgatgatacgcgaggaacttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggtcccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgta

SEQ ID NO: 31 - DNA, 1520 bp, Bacteroides vulgatus
  misc_feature: Bacteroides vulgatus 8482
gtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggtcatgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaaggtgcggctggaacacctcctttct

SEQ ID NO: 32 - DNA, 1532 bp, Bacteroides vulgatus
  misc_feature: Bacteroides vulgatus 8482
ttacaatgaagagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatatttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatactgcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttttgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaaggtgcggctggaacacctcctttct

SEQ ID NO: 33 - DNA, 1532 bp, Bacteroides vulgatus; identical to SEQ ID NO: 32
  misc_feature: Bacteroides vulgatus 8482

SEQ ID NO: 34 - DNA, 1538 bp, Bacteroides vulgatus
  misc_feature: Bacteroides vulgatus 8482
tattacaatgaagagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatactgcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaaggtgcggctggaacacctcctttctggag

SEQ ID NO: 35 - DNA, 1538 bp, Bacteroides vulgatus; identical to SEQ ID NO: 34
  misc_feature: Bacteroides vulgatus 8482

SEQ ID NO: 36 - DNA, 1538 bp, Bacteroides vulgatus
  misc_feature: Bacteroides vulgatus 8482
tattacaatgaagagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggtcatgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaaggtgcggctggaacacctcctttctggag

SEQ ID NO: 37 - DNA, 1538 bp, Bacteroides vulgatus
  misc_feature: Bacteroides vulgatus 8482
tattacaatgaagagtttgatcctagctcaggatgaacgctagctacaggcttaacacatgcaagtcgaggggcagcatggtcttagcttgctaaggccgatggcgaccggcgcacgggtgagtaacacgtatccaacctgccgtctactcttggatagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggtcatgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaaggtgcggctggaacacctcctttctggag

SEQ ID NO: 38 - DNA, 1538 bp, Bacteroides vulgatus
  misc_feature: Bacteroides vulgatus 8482
tattacaatgaagagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaggaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttctgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatcccaaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaaggtgcggctggaacacctcctttctggag

SEQ ID NO: 39 - DNA, 1538 bp, Bacteroides vulgatus
  misc_feature: Bacteroides vulgatus 8482
tattacaatgaagagtttgatcctggctcaggatgaacgctagctacaggcttaacacatgcaagtcgaggggcagcatggtcttagcttgctaagactgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgcaggcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggtttcgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatcccaaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaaggtgcggctggaacacctcctttctggag

SEQ ID NO: 40 - DNA, 1489 bp, Bacteroides vulgatus
  misc_feature: Bacteroides vulgatus 8482
agagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttatgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtaacc

SEQ ID NO: 41 - DNA, 1484 bp, Barnesiella intestinihominis
  misc_feature: Barnesiella intestinihominis DSM 21032
gatgaacgctagcgacaggcctaacacatgcaagtcgaggggcagcggggaggtagcaatacctttgccggcgaccggcgcacgggtgagtaacacgtatgcaatccacctgtaacagggggataacccggagaaatccggactaataccccataatatgggcgctccgcatggagagttcattaaagagagcaattttggttacagacgagcatgcgctccattagccagttggcggggtaacggcccaccaaagcgacgatggataggggttctgagaggaaggtcccccacattggaactgagacacggtccaaactcctacgggaggcagcagtgaggaatattggtcaatggtcggcagactgaaccagccaagtcgcgtgagggaagacggccctacgggttgtaaacctcttttgtcggagagtaaagtacgctacgtgtagcgtattgcaagtatccgaagaaaaagcatcggctaactccgtgccagcagccgcggtaatacggaggatgcgagcgttatccggatttattgggtttaaagggtgcgtaggcggcacgccaagtcagcggtgaaatttccgggctcaacccggagtgtgccgttgaaactggcgagctagagtacacaagaggcaggcggaatgcgtggtgtagcggtgaaatgcatagatatcacgcagaaccccgattgcgaaggcagcctgctagggtgaaacagacgctgaggcacgaaagcgtgggtatcgaacaggattagataccctggtagtccacgcagtaaacgatgaatactaactgtttgcgatacaatgtaagcggtacagcgaaagcgttaagtattccacctggggagtacgccggcaacggtgaaactcaaaggaattgacgggggcccgcacaagcggaggaacatgtggtttaattcgatgatacgcgaggaaccttacccgggctcaaacgcagggggaatgtcggtgaaagccggcagctagtaatagtcacctgcgaggtgctgcatggttgtcgtcagctcgtgccgtgaggtgtcggcttaagtgccataacgagcgcaacccctatcgacagttactaacgggtgaagccgaggactctgtcgagactgccggcgcaagccgcgaggaaggtggggatgacgtcaaatcagcacggcccttacgtccggggcgacacacgtgttacaatggcaggtacagaaggcagccagtcagcaatgacgcgcgaatcccgaaaacctgtctcagttcggattggagtctgcaacccgactccatgaagctggattcgctagtaatcgcgcatcagccatggcgcggtgaatacgttcccgggccttgtacacaccgcccgtcaagccatggaagccgggagtacctgaagcatgcaaccgcaaggagcgtacgaaggtaataccggtaactggggctaagtcgtaacaaggtagccgtaccggaagg

SEQ ID NO: 42 - DNA, 1536 bp, Barnesiella intestinihominis
  misc_feature: Barnesiella intestinihominis DSM 21032
atacaacgaagagtttgatcctggctcaggatgaacgctagcgacaggcctaacacatgcaagtcgaggggcagcggggaggtagcaatacctttgccggcgaccggcgcacgggtgagtaacacgtatgcaatccacctgtaacagggggataacccggagaaatccggactaataccccataatatgggcgctccgcatggagagttcattaaagagagcaattttggttacagacgagcatgcgctccattagccagttggcggggtaacggcccaccaaagcgacgatggataggggttctgagaggaaggtcccccacattggaactgagacacggtccaaactcctacgggaggcagcagtgaggaatattggtcaatggtcggcagactgaaccagccaagtcgcgtgagggaagacggccctacgggttgtaaacctcttttgtcggagagtaaagtacgctacgtgtagcgtattgcaagtatccgaagaaaaagcatcggctaactccgtgccagcagccgcggtaatacggaggatgcgagcgttatccggatttattgggtttaaagggtgcgtaggcggcacgccaagtcagcggtgaaatttccgggctcaacccggagtgtgccgttgaaactggcgagctagagtacacaagaggcaggcggaatgcgtggtgtagcggtgaaatgcatagatatcacgcagaaccccgattgcgaaggcagcctgctagggtgaaacagacgctgaggcacgaaagcgtgggtatcgaacaggattagataccctggtagtccacgcagtaaacgatgaatactaactgtttgcgatacaatgtaagcggtacagcgaaagcgttaagtattccacctggggagtacgccggcaacggtgaaactcaaaggaattgacgggggcccgcacaagcggaggaacatgtggtttaattcgatgatacgcgaggaaccttacccgggctcaaacgcagggggaatgtcggtgaaagccggcagctagtaatagtcacctgcgaggtgctgcatggttgtcgtcagctcgtgccgtgaggtgtcggcttaagtgccataacgagcgcaacccctatcgacagttactaacgggtgaagccgaggactctgtcgagactgccggcgcaagccgcgaggaaggtggggatgacgtcaaatcagcacggcccttacgtccggggcgacacacgtgttacaatggcaggtacagaaggcagccagtcagcaatgacgcgcgaatcccgaaaacctgtctcagttcggattggagtctgcaacccgactccatgaagctggattcgctagtaatcgcgcatcagccatggcgcggtgaatacgttcccgggccttgtacacaccgcccgtcaagccatggaagccgggagtacctgaagcatgcaaccgcaaggagcgtacgaaggtaataccggtaactggggctaagtcgtaacaaggtagccgtaccggaaggtgcggctggaacacctcctttct

SEQ ID NO: 43 - DNA, 1536 bp, Barnesiella intestinihominis; identical to SEQ ID NO: 42
  misc_feature: Barnesiella intestinihominis DSM 21032

SEQ ID NO: 44 - DNA, 1536 bp, Barnesiella intestinihominis
  misc_feature: Barnesiella intestinihominis DSM 21032
atacaacgaagagtttgatcctggctcaggatgaacgctagcgacaggcctaacacatgcaagtcgaggggcagcggggaggtagcaatacctttgccggcgaccggcgcacgggtgagtaacacgtatgcaatccacctgtaacagggggataacccggagaaatccggactaataccccataatatgggcgctccgcatggagagttcattaaagagagcaattttggttacagacgagcatgcgctccattagccagttggcggggtaacggcccaccaaagcgacgatggataggggttctgagaggaaggtcccccacattggaactgagacacggtccaaactcctacgggaggcagcagtgaggaatattggtcaatggtcggcagactgaaccagccaagtcgcgtgagggaagacggccctacgggttgtaaacctcttttgtcggagagtaaagtacgctacgtgtagcgtattgcaagtatccgaagaaaaagcatcggctaactccgtgccagcagccgcggtaatacggaggatgcgagcgttatccggatttattgggtttaaagggtgcgtaggcggcacgccaagtcagcggtgaaatttccgggctcaacccggagtgtgccgttgaaactggcgagctagagtgcacaagaggcaggcggaatgcgtggtgtagcggtgaaatgcatagatatcacgcagaaccccgattgcgaaggcagcctgctagggtgaaacagacgctgaggcacgaaagcgtgggtatcgaacaggattagataccctggtagtccacgcagtaaacgatgaatactaactgtttgcgatacaatgtaagcggtacagcgaaagcgttaagtattccacctggggagtacgccggcaacggtgaaactcaaaggaattgacgggggcccgcacaagcggaggaacatgtggtttaattcgatgatacgcgaggaaccttacccgggctcaaacgcagggggaatgtcggtgaaagccggcagctagtaatagtcacctgcgaggtgctgcatggttgtcgtcagctcgtgccgtgaggtgtcggcttaagtgccataacgagcgcaacccctatcgacagttactaacgggtgaagccgaggactctgtcgagactgccggcgcaagccgcgaggaaggtggggatgacgtcaaatcagcacggcccttacgtccggggcgacacacgtgttacaatggcaggtacagaaggcagccagtcagcaatgacgcgcgaatcccgaaaacctgtctcagttcggattggagtctgcaacccgactccatgaagctggattcgctagtaatcgcgcatcagccatggcgcggtgaatacgttcccgggccttgtacacaccgcccgtcaagccatggaagccgggagtacctgaagcatgcaaccgcaaggagcgtacgaaggtaataccggtaactggggctaagtcgtaacaaggtagccgtaccggaaggtgcggctggaacacctcctttct

SEQ ID NO: 45 - DNA, 1536 bp, Barnesiella intestinihominis; identical to SEQ ID NO: 42
  misc_feature: Barnesiella intestinihominis DSM 21032

SEQ ID NO: 46 - DNA, 1540 bp, Barnesiella intestinihominis
  misc_feature: Barnesiella intestinihominis DSM 21032
atacaacgaagagtttgatcctggctcaggatgaacgctagcgacaggcctaacacatgcaagtcgaggggcagcggggaggtagcaatacctttgccggcgaccggcgcacgggtgagtaacacgtatgcaatccacctgtaacagggggataacccggagaaatccggactaataccccataatatgggcgctccgcatggagagttcattaaagagagcaattttggttacagacgagcatgcgctccattagccagttggcggggtaacggcccaccaaagcgacgatggataggggttctgagaggaaggtcccccacattggaactgagacacggtccaaactcctacgggaggcagcagtgaggaatattggtcaatggtcggcagactgaaccagccaagtcgcgtgagggaagacggccctacgggttgtaaacctcttttgtcggagagtaaagtacgctacgtgtagcgtattgcaagtatccgaagaaaaagcatcggctaactccgtgccagcagccgcggtaatacggaggatgcgagcgttatccggatttattgggtttaaagggtgcgtaggcggcacgccaagtcagcggtgaaatttccgggctcaacccggagtgtgccgttgaaactggcgagctagagtacacaagaggcaggcggaatgcgtggtgtagcggtgaaatgcatagatatcacgcagaaccccgattgcgaaggcagcctgctagggtgaaacagacgctgaggcacgaaagcgtgggtatcgaacaggattagataccctggtagtccacgcagtaaacgatgaatactaactgtttgcgatacaatgtaagcggtacagcgaaagcgttaagtattccacctggggagtacgccggcaacggtgaaactcaaaggaattgacgggggcccgcacaagcggaggaacatgtggtttaattcgatgatacgcgaggaaccttacccgggctcaaacgcagggggaatgtcggtgaaagccggcagctagtaatagtcacctgcgaggtgctgcatggttgtcgtcagctcgtgccgtgaggtgtcggcttaagtgccataacgagcgcaacccctatcgacagttactaacgggtgaagccgaggactctgtcgagactgccggcgcaagccgcgaggaaggtggggatgacgtcaaatcagcacggcccttacgtccggggcgacacacgtgttacaatggcaggtacagaaggcagccagtcagcaatgacgcgcgaatcccgaaaacctgtctcagttcggattggagtctgcaacccgactccatgaagctggattcgctagtaatcgcgcatcagccatggcgcggtgaatacgttcccgggccttgtacacaccgcccgtcaagccatggaagccgggagtacctgaagcatgcaaccgcaaggagcgtacgaaggtaataccggtaactggggctaagtcgtaacaaggtagccgtaccggaaggtgcggctggaacacctcctttctggag

SEQ ID NO: 47 - DNA, 1540 bp, Barnesiella intestinihominis; identical to SEQ ID NO: 46
  misc_feature: Barnesiella intestinihominis DSM 21032

SEQ ID NO: 48 - DNA, 1540 bp, Barnesiella intestinihominis
  misc_feature: Barnesiella intestinihominis DSM 21032
atacaacgaagagtttgatcctggctcaggatgaacgctagcgacaggcctaacacatgcaagtcgaggggcagcggggaggtagcaatacctttgccggcgaccggcgcacgggtgagtaacacgtatgcaatccacctgtaacagggggataacccggagaaatccggactaataccccataatatgggcgctccgcatggagagttcattaaagagagcaattttggttacagacgagcatgcgctccattagccagttggcggggtaacggcccaccaaagcgacgatggataggggttctgagaggaaggtcccccacattggaactgagacacggtccaaactcctacgggaggcagcagtgaggaatattggtcaatggtcggcagactgaaccagccaagtcgcgtgagggaagacggccctacgggttgtaaacctcttttgtcggagagtaaagtacgctacgtgtagcgtattgcaagtatccgaagaaaaagcatcggctaactccgtgccagcagccgcggtaatacggaggatgcgagcgttatccggatttattgggtttaaagggtgcgtaggcggcacgccaagtcagcggtgaaatttccgggctcaacccggagtgtgccgttgaaactggcgagctagagtgcacaagaggcaggcggaatgcgtggtgtagcggtgaaatgcatagatatcacgcagaaccccgattgcgaaggcagcctgctagggtgaaacagacgctgaggcacgaaagcgtgggtatcgaacaggattagataccctggtagtccacgcagtaaacgatgaatactaactgtttgcgatacaatgtaagcggtacagcgaaagcgttaagtattccacctggggagtacgccggcaacggtgaaactcaaaggaattgacgggggcccgcacaagcggaggaacatgtggtttaattcgatgatacgcgaggaaccttacccgggctcaaacgcagggggaatgtcggtgaaagccggcagctagtaatagtcacctgcgaggtgctgcatggttgtcgtcagctcgtgccgtgaggtgtcggcttaagtgccataacgagcgcaacccctatcgacagttactaacgggtgaagccgaggactctgtcgagactgccggcgcaagccgcgaggaaggtggggatgacgtcaaatcagcacggcccttacgtccggggcgacacacgtgttacaatggcaggtacagaaggcagccagtcagcaatgacgcgcgaatcccgaaaacctgtctcagttcggattggagtctgcaacccgactccatgaagctggattcgctagtaatcgcgcatcagccatggcgcggtgaatacgttcccgggccttgtacacaccgcccgtcaagccatggaagccgggagtacctgaagcatgcaaccgcaaggagcgtacgaaggtaataccggtaactggggctaagtcgtaacaaggtagccgtaccggaaggtgcggctggaacacctcctttctggag

SEQ ID NO: 49 - DNA, 1540 bp, Barnesiella intestinihominis; identical to SEQ ID NO: 46
  misc_feature: Barnesiella intestinihominis DSM 21032

SEQ ID NO: 50 - DNA, 1481 bp, Barnesiella intestinihominis
  misc_feature: Barnesiella intestinihominis DSM 21032
tggctcaggatgaacgctagcgacaggcctaacacatgcaagtcgaggggcagcggggaggtagcaatacctttgccggcgaccggcgcacgggtgagtaacacgtatgcaatccacctgtaacagggggataacccggagaaatccggactaataccccataatatgggcgctccgcatggagagttcattaaagagagcaattttggttacagacgagcatgcgctccattagccagttggcggggtaacggcccaccaaagcgacgatggataggggttctgagaggaaggtcccccacattggaactgagacacggtccaaactcctacgggaggcagcagtgaggaatattggtcaatggtcggcagactgaaccagccaagtcgcgtgagggaagacggccctacgggttgtaaacctcttttgtcggagagtaaagtacgctacgtgtagcgtattgcaagtatccgaagaaaaagcatcggctaactccgtgccagcagccgcggtaatacggaggatgcgagcgttatccggatttattgggtttaaagggtgcgtaggcggcacgccaagtcagcggtgaaatttccgggctcaacccggagtgtgccgttgaaactggcgagctagagtacacaagaggcaggcggaatgcgtggtgtagcggtgaaatgcatagatatcacgcagaaccccgattgcgaaggcagcctgctagggtgaaacagacgctgaggcacgaaagcgtgggtatcgaacaggattagataccctggtagtccacgcagtaaacgatgaatactaactgtttgcgatacaatgtaagcggtacagcgaaagcgttaagtattccacctggggagtacgccggcaacggtgaaactcaaaggaattgacgggggcccgcacaagcggaggaacatgtggtttaattcgatgatacgcgaggaaccttacccgggctcaaacgcagggggaatgtcggtgaaagccggcagctagtaatagtcacctgcgaggtgctgcatggttgtcgtcagctcgtgccgtgaggtgtcggcttaagtgccataacgagcgcaacccctatcgacagttactaacgggtgaagccgaggactctgtcgagactgccggcgcaagccgcgaggaaggtggggatgacgtcaaatcagcacggcccttacgtccggggcgacacacgtgttacaatggcaggtacagaaggcagccagtcagcaatgacgcgcgaatcccgaaaacctgtctcagttcggattggagtctgcaacccgactccatgaagctggattcgctagtaatcgcgcatcagccatggcgcggtgaatacgttcccgggccttgtacacaccgcccgtcaagccatggaagccgggagtacctgaagcatgcaaccgcaaggagcgtacgaaggtaataccggtaactggggctaagtcgtaacaaggtaacc

SEQ ID NO: 51 - DNA, 1538 bp, Bifidobacterium stercoris
  misc_feature: Bifidobacterium stercoris EG1
tttttgtggagggttcgattctggctcaggatgaacgctggcggcgtgcttaacacatgcaagtcgaacgggatcccaggagcttgctcctgggtgagagtggcgaacgggtgagtaatgcgtgaccgacctgccccatacaccggaatagctcctggaaacgggtggtaatgccggatgctccgacatgacgcatgtcgtgtcgggaaagattcatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccataaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatacggtttcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacaacgggatgcgacactgtgaggtggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccaacctttttggggggagccgtctaaggtgagactcgtgattgggactaagtcgtaacaaggtagccgtaccggaaggtgcggctggatcacctcctttctacggaga

SEQ ID NO: 52 - DNA, 1538 bp, Bifidobacterium stercoris
  misc_feature: Bifidobacterium stercoris EG1
tttttgtggagggttcgattctggctcaggatgaacgctggcggcgtgcttaacacatgcaagtcgaacgggatcccaggagcttgctcctgggtgagagtggcgaacgggtgagtaatgcgtgaccgacctgccccatacaccggaatagctcctggaaacgggtggtaatgccggatgctccgacatgacgcatgtcgtgtcgggaaagattcatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatacggtctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacctcgtgagggggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccaacctttttggggggagccgtctaaggtgagactcgtgattgggactaagtcgtaacaaggtagccgtaccggaaggtgcggctggatcacctcctttctacggaga

SEQ ID NO: 53 - DNA, 1538 bp, Bifidobacterium stercoris; identical to SEQ ID NO: 52
  misc_feature: Bifidobacterium stercoris EG1

SEQ ID NO: 54 - DNA, 1416 bp, Bifidobacterium stercoris
  misc_feature: Bifidobacterium stercoris EG1
  misc_feature (1408)..(1408): n is a, c, g, or t
gatgaacgctggcggcgtgcttaccatgcaagtcgaacgggatcccaggagcttgctcctgggtgagagtggcgaacgggtgagtaatgcgtgaccgacctgccccatacaccggaatagctcctggaaacgggtggtaatgccggatgctccgacatgacgcatgtcgtgtcgggaaagattcatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaccttacctgggcttgacatgttcccgacagccgtagagatacggtctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacctcgtgagggggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccaaccttttnggggggag

SEQ ID NO: 55 - DNA, 1486 bp, Butyricimonas virosa
  misc_feature: Butyricimonas virosa MT12
agagtttgatcctggctcaggatgaacgctagcgacaggcttaacacatgcaagtcgaggggcagcacggtgtagcaatacactggtggcgaccggcgcacgggtgagtaacacgtgtgcaaccaaccccgtaccgggagataacccgcggaaacgtggactaacatcccatgatactcgagagccgcatggctctcgatttaaaattccggtggtacgggacgggcacgcgcgacattaggtagttggcggggtaacggcccaccaagccgacgatgtctaggggttctgagaggaaggtcccccacactggaactgagacacggtccagactcctacgggaggcagcagtgaggaatattggtcaatgggcgagagcctgaaccagccaagtcgcgtgagggaagaatggtctatggcctgtaaacctcttttgtcagggaagaataaggatgacgagtcattcgatgccagtacttgacgaataagcatcggctaactccgtgccagcagccgcggtaatacgggggatgcgagcgttatccggatttattgggtttaaagggcgcgtaggcgggacgtcaagtcagcggtaaaagactgcagctaaactgtagcacgccgttgaaactggcgccctggagacgagacgagggaggcggaacaagtgaagtagcggtgaaatgcatagatatcacttggaaccccgatagcgaaggcagcttcccaggctcgttctgacgctgatgcgcgagagcgtgggtagcgaacaggattagataccctggtagtccacgccgtaaacgatgctcactggatcttggcgatacactgccagggttcaagcgaaagtattaagtgagccacctggggagtacgtcggcaacgatgaaactcaaaggaattgacgggggcccgcacaagcggaggaacatgtggtttaattcgatgatacgcgaggaaccttacccgggtttaaatgtagattgcatgaggtggaaacgcttcttcccttcggggctatttacaaggtgctgcatggttgtcgtcagctcgtgccgtgaggtgtcgggttaagtcccataacgagcgcaacccctatcgccagttgccatcggttgaagccgggcactctgtcgagactgccaccgtaaggtgcgaggaaggcggggatgacgtcaaatcagcacggcccttacacccggggcgacacacgtgttacaatggccggtacagagggcagccacggggtgacccggagcgaatctctaaagccggtcgtagttcggactggagtctgcaacccgactccacgaagttggattcgctagtaatcgcgcatcagccatggcgcggtgaatacgttcccgggccttgtacacaccgcccgtcaagccatggaagccgggagtacctgaagatcgtgaccgcgaggaacgggctagggtaataccggtaactggggctaagtcgtaacaaggtaacc

SEQ ID NO: 56 - DNA, 1419 bp, Clostridium citroniae
  misc_feature: Clostridium citroniae DSM 19261
cgtgcctaacacatgcaagtcgaacgaagcaatcagaatgaaattttcggatggatttctgggtgactgagtggcggacgggtgagtaacgcgtggataacctgcctcacactgggggataacagttagaaatggctgctaataccgcataagcgcacagtaccgcatggtacggtgtgaaaaactccggtggtgtgagatggatccgcgtctgattagttagttggcggggtaacggcccaccaagacagcgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaactgacggtacctgactaagaagccccggctaactacgtgccagcagccgcggtaatacgtagggggcaagcgttatccggatttactgggtgtaaagggagcgtagacggcgaagcaagtctggagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggtcagtaaagtgacctttccttcgggacaggggagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcgaccctgcgaaggcaagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagggagctgccgaagg

SEQ ID NO: 57 - DNA, 1409 bp, Clostridium
  misc_feature: Clostridium sp. 40
ctggcggcgtgcctaacacatgcaagtcgagcgattctcttcggagaagagcggcggacgggtgagtaacgcgtgggtaacctgcctcatacacatggataacataccgaaaggtatgctaatacaggataacataagagattcgcatgtttctcttatcaaagctccggcggtatgagatggacccgcgtctgattagctagttggtaaggtaatggcttaccaaggcgacgatcagtagccgacctgagagggtgatcggccacattggaactgagacacggtccaaactcctacgggaggcagcagtggggaatattgcacaatgggcgaaagcctgatgcagcaacgccgcgtgagtgatgaaggccttcgggtcgtaaaactctgtcctcaaggaagataatgacggtacttgaggaggaagccccggctaactacgtgccagcagccgcggtaatacgtagggggctagcgttatccggatttactgggcgtaaagggtgcgtaggtggtttcttaagtcaggagtgaaaggctacggcttaaccgtagtaagctcttgaaactgggaaacttgagtgcaggagaggaaagtggaattcctagtgtagcggtgaaatgcgtagatattaggaggaacaccagtagcgaaggcggctttctggactgtaactgacactgaggcacgaaagcgtggggagcaaacaggattagataccctggtagtccacgccgtaaacgatgagtactaggtgtcgggggttacccccctcggtgccgcagctaacgcattaagtactccgcctggggagtacgctcgcaagagtgaaactcaaaggaattgacggggacccgcacaagtagcggagcatgtggtttaattcgaagcaacgcgaagaaccttacctaagcttgacatccttttgaycgatgcctaatcgcatttttcccttcggggacagaagtgacaggtggtgcatggttgtcgtcagctcgtgtcgtgagatgttgggttaagtcccgcaacgagcgcaacccttgcctttagttgccagcattaagttgggcactctagagggactgccagggataacctggaggaaggtggggatgacgtcaaatcatcatgccccttatgcttagggctacacacgtgctacaatgggtggtacagagggcagccaagtcgtgaggccgagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccacggaagttgggggcgcccgaagccacttagctaacccttttgggaagcgagtgtcgaaggtgaaatcaat

SEQ ID NO: 58 - DNA, 1388 bp, Clostridium spiroforme
  misc_feature: Clostridium spiroforme CCM 6168
gatcctggctcaggatgaacgctggcggcgtgcctaacacatgcaagtcgagcgagcagcaatgctcgagtggcgaacgggtgagtaatacataagtaacctgccctagacagggggataactgctggaaacggcagctaagaccgcataggtatggacactgcatggtgaccatattaaaatgccaaggcactggtagaggatggacttatggcgcattagctggttggtgaggtaacagctcaccaaggcgacgatgcgtagccgacctgagagggtgaccggccacactgggactgagacacggcccagactcctacgggaggcagcagtagggaattttcggcaatggggggaaccctgaccgagcaacgccgcgtgaaggaagaaggaattcgttctgtaaacttctgttataaaggaagaacggcggatatagggaatgatatccgagtgacggtactttatgagaaagccacggctaactacgtgccagcagccgcggtaatacgtaggtggcgagcgttatccggaattattgggcgtaaagagggagcaggcggcggcagaggtctgtggtgaaagactgaagcttaacttcagtaagccatagaaaccgggctgctagagtgcaggagaggatcgtggaattccatgtgtagcggtgaaatgcgtagatatatggaggaacaccagtggcgaaggcgacggtctggcctgtaactgacgcacattcccgaaagcgtggggagcaaataggattagataccctagtagtccacgccgtaaacgatgagtactaagtgttgggagtcaaatttcagtgctgcagttaacgcaataagtactccgcctgagtagtacgttcgcaagaatgaaactcaaaggaattgacgggggccgcacaagcggtggagcatgtggtttaattcgaagcaacgcgaagaaccttaccaggtcttgacatccggataaagacctcgagagatgaggggatagatatatccgagacaggtggtgcatggttgtcgtcagctcgtgtcgtgagatgttgggttaagtcccgcaacgagcgcaacccttgtcgctagttaccatcattaagttggggactctagcgagactgccagtgacaagctggaggaaggcggggatgacgtcaaatcatcatgccccttatgacctgggctacacacgtgctacaatggatggagcagagggaagcgaagccgcgaggtgaagcaaaacccataaaaccattctcagttcggattgtagtctgcaactcgactacatgaagttggaatcgctagtaatcgcgaatcagcatgtcgcggtgaatacgttcccgggccttgtacacaccgcccgtcacaccatgagagttg

SEQ ID NO: 59 - DNA, 1512 bp, Clostridium spiroforme
  misc_feature: Clostridium spiroforme CCM 6168
gagagtttgatcctggctcaggatgaacgctggcggctgcctaatacatgcaagtcgaacgcgagcagcaatgctcgagtggcgaacgggtgagtaatacataagtaacctgccctagacagggggataactgctggaaaccagctaagaccgcataggtatggacactgcatggtgaccatattaaaagtgccaaggcactggtagaggatggacttatggcgcattagctggttggtgaggtaacagctcaccaaggcgacgatgcgtagccgacctgagagggtgaccggccacactgggactgagacacggcccagactcctacgggaggcagcagtagggaattttcggcaatggggggaaccctgaccgagcaacgccgcgtgaaggaagaaggaattcgttctgtaaacttctgttataaaggaagaacggcggatatagggaatgatatccgagtgacggtactttatgagaaagccacggctaactacgtgccagcagccgcggtaatacgtaggtggcgagcgttatccggaattattgggcgtaaagagggagcaggcggcggcagaggtctgtggtgaaagactgaagcttabacttcagtaagccatagaaaccgggctgctagagtgcaggagaggatcgtggaattcctgtgtagcggtgaaatgcgtagatatatggaggaacaccagtggcgaaggcgacggtctggcctgtaactgacgctcattcccgaaagcgtggggagcaaataggattagataccctagtagtccacgccgtaaacgatgagtactaagtgttgggagtcaaatttcagtgctgcagttaacgcaataagtactccgcctgagtagtacgttcgcaagaatgaaactcaaaggaattgacgggggccgcacaagcggtggagcatgtggtttaattcgaagcaacgcgaagaaccttaccaggtcttgacatccggataaagacctcagagatgaggggatagatatatccgagacaggtggtgcatggttgtcgtcagctcgtgtcgtgagatgttgggttaagtcccgcaacgagcgcaacccttgtcgctagttaccatcattaagttggggactctagcgagactgccagtgacaagctggaggaaggcggggatgacgtcaaatcatcatgccccttatgacctgggctacacacgtgctacaatggatggagcagagggaagcgaagccgcgaggtgaagcaaaacccataaaaccattctcagttcggattgtagtctgcaactcgactacatgaagttggaatcgctagtaatcgcgaatcagcatgtcgcggtgaatacgttctcgggccttgtacacaccgcccgtcacaccatgagagttgataacacccgaagccggtggcctaaccgcaaggagggagctgtctaaggtgggattgatgattggggtgaagtcgtaacaaggtatccctacgggaacgtgcggctggatcacctcc

SEQ ID NO: 60 - DNA, 1380 bp, Clostridium spiroforme
  misc_feature: Clostridium spiroforme CCM 6168
tggagagtttgatcctggctcaggatgaacgctggcggcgtgcctaatacatgcaagtcgaacgcgagcagcaatgctcgagtggcgaacgggtgagtaatacataagtaacccgccctagacagggggataactgctggaaacggcagctaagaccgcataggtatggacactgcatggtgaccatattaaaagtgccaaggcactggtagaggatggacttatggcgcattagctggttggtgaggtaacggctcaccaaggcgacgatgcgtagccgacctgagagggtgaccggccacactgggactgagacacggcccagactcctacgggaggcagcagtagggaattttcggcaatggggggaaccctgaccgagcaacgccgcgtgaaggaagaaggaattcgttctgtaaacttctgttataaaggaagaacggcggatatagggaatgatatccgagtgacggtactttatgagaaagccacggctaactacgtgccagcagccgcggtaatacgtaggtggcgagcgttatccggaattattgggcgtaaagagggagcaggcggcggcagaggtctatggtgaaagactgaagcttaacttcagtaagccatagaaaccgggctgctagagtgcaggagaggatcgtggaattccatgtgtagcggtgaaatgcgtagatatatggaggaacaccagtggcgaaggcgacggtctggcctgtaactgacgctcattcccgaaagcgtggggagcaaataggattagataccctagtagtccacgccgtaaacgatgagtactaagtgttgggagtcaaatttcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatccggataaagacctcagagatgaggggatagatatatccgagacaggtggtgcatggttgtcgtcagctcgtgtcgtgagatgttgggttaagtcccgcaacgagcgcaacccttgtcgctagttaccatcattaagttggggactctagcgagactgccagtgacaagctggaggaaggcggggatgacgtcaaatcatcatgccccttatgacctgcgctacacacgtgctacaatgtatggagcagagggaagcgaagccgcgaggtgaagcaaacccataaaccattctcagttcggattgtagtctgcaactcgactacatgagtgtatcgctagtatcgcgatcagcatgtcgcgtgaatacgttctcggccttgtacacaccccgcccgtca

SEQ ID NO: 61 - DNA, 1386 bp, Clostridium spiroforme
  misc_feature: Clostridium spiroforme CCM 6168
ggacaatggagagtttgatcctggctcaggatgaacgctggcggcgtgcctaatacatgcaagtcgaacgcgagcagcaatgctcgagtggcgaacgggtgagtaatacataagtaacccgccctagacagggggataactgctggaaacggcagctaagaccgcataggtatggacactgcatggtgaccatattaaaagtgccaaggcactggtagaggatggacttatggcgcattagctggttggtgaggtaacggctcaccaaggcgacgatgcgtagccgacctgagagggtgaccggccacactgggactgagacacggcccagactcctacgggaggcagcagtagggaattttcggcaatggggggaaccctgaccgagcaacgccgcgtgaaggaagaaggaattcgttctgtaaacttctgttataaaggaagaacggcggatatagggaatgatatccgagtgacggtactttatgagaaagccacggctaactacgtgccagcagccgcggtaatacgtaggtggcgagcgttatccggaattattgggcgtaaagagggagcaggcggcggcagaggtctatggtgaaagactgaagcttaacttcagtaagccatagaaaccgggctgctagagtgcaggagaggatcgtggaattccatgtgtagcggtgaaatgcgtagatatatggaggaacaccagtggcgaaggcgacggtctggcctgtaactgacgctcattcccgaaagcgtggggagcaaataggattagataccctagtagtccacgccgtaaacgatgagtactaagtgttgggagtcaaatttcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatccggataaagacctcagagatgaggggatagatatatccgagacaggtggtgcatggttgtcgtcagctcgtgtcgtgagatgttgggttaagtcccgcaacgagcgcaacccttgtcgctagttaccatcattaagttggggactctagcgagactgccagtgacaagctggaggaaggcggggatgacgtcaaatcatcatgccccttatgacctgcgctacacacgtgctacaatgtatggagcagagggaagcgaagccgcgaggtgaagcaaacccataaaccattctcagttcggattgtagtctgcaactcgactacatgagtgtatcgctagtatcgcgatcagcatgtcgcgtgaatacgttctcggccttgtacacaccccgcccgtca

SEQ ID NO: 62 - DNA, 1400 bp, Clostridium spiroforme
  misc_feature: Clostridium spiroforme CCM 6168
aatggagagtttgatcctggctcaggatgaacgctggcggcgtgcctaatacatgcaagtcgaacgcgagcagcaatgctcgagtggcgaacgggtgagtaatacataagtaacccgccctagacagggggataactgctggaaacggcagctaagaccgcataggtatggacactgcatggtgaccatattaaaagtgccaaggcactggtagaggatggacttatggcgcattagctggttggtgaggtaacggctcaccaaggcgacgatgcgtagccgacctgagagggtgaccggccacactgggactgagacacggcccagactcctacgggaggcagcagtagggaattttcggcaatggggggaaccctgaccgagcaacgccgcgtgaaggaagaaggaattcgttctgtaaacttctgttataaaggaagaacggcggatatagggaatgatatccgagtgacggtactttatgagaaagccacggctaactacgtgccagcagccgcggtaatacgtaggtggcgagcgttatccggaattattgggcgtaaagagggagcaggcggcggcagaggtctatggtgaaagactgaagcttaacttcagtaagccatagaaaccgggctgctagagtgcaggagaggatcgtggaattccatgtgtagcggtgaaatgcgtagatatatggaggaacaccagtggcgaaggcgacggtctggcctgtaactgacgctcattcccgaaagcgtggggagcaaataggattagataccctagtagtccacgccgtaaacgatgagtactaagtgttgggagtcaaatttcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatccggataaagacctcagagatgaggggatagatatatccgagacaggtggtgcatggttgtcgtcagctcgtgtcgtgagatgttgggttaagtcccgcaacgagcgcaacccttgtcgctagttaccatcattaagttggggactctagcgagactgccagtgacaagctggaggaaggcggggatgacgtcaaatcatcatgccccttatgacctgcgctacacacgtgctacaatgtatggagcagagggaagcgaagccgcgaggtgaagcaaacccataaaccattctcagttcggattgtagtctgcaactcgactacatgagtgtatcgctagtatcgcgatcagcatgtcgcgtgaatacgttctcggccttgtacacaccccgcccgtcaacaccattgagaagttgg

SEQ ID NO: 63 - DNA, 1305 bp, Clostridium spiroforme
  misc_feature: Clostridium spiroforme CCM 6168
aatggagagtttgatcctggctcaggatgaacgctggcggcgtgcctaatacatgcaagtcgaacgcgagcagcaatgctcgagtggcgaacgggtgagtaatacataagtaacctgccctagacagggggataactgctggaaacggcagctaagaccgcataggtatggacactgcatggtgaccatattaaaagtgccaaggcactggtagaggatggacttatggcgcattagctggttggtgaggtaacagctcaccaaggcgacgatgcgtagccgacctgagagggtgaccggccacactgggactgagacacggcccagactcctacgggaggcagcagtagggaattttcggcaatggggggaaccctgaccgagcaacgccgcgtgaaggaagaaggaattcgttctgtaaacttctgttataaaggaagaacggcggatatagggaatgatatccgagtgacggtactttatgagaaagccacggctaactacgtgccagcagccgcggtaatacgtaggtggcgagcgttatccggaattattgggcgtaaagagggagcaggcggcggcagaggtctgtggtgaaagactgaagcttaacttcagtaagccatagaaaccgggctgctagagtgcaggagaggatcgtggaattccatgtgtagcggtgaaatgcgtagatatatggaggaacaccagtggcgaaggcgacggtctggcctgtaactgacgctcattcccgaaagcgtggggagcaaataggattagataccctagtagtccacgccgtaaacgatgagtactaagtgttgggagtcaaatttcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatccggataaagacctcagagatgaggggatagatatatccgagacaggtggtgcatggttgtcgtcagctcgtgtcgtgagatgttgggttaagtcccgcaacgagcgcaacccttgtcgctagttaccatcattaagttggggactctagcgagactgccagtgacaagctggaggaaggcggggatgacgtcaaatcatcatgccccttatgacctggctacacacgtgctacaatgatggagcagagggaagcgaagccgcgagtgagcaaacccataaaccattctcagtcggattgtagtctgcactcgactacatga

SEQ ID NO: 64 - DNA, 1474 bp, Eubacterium eligens
  misc_feature: Eubacterium eligens DSM 3376
  misc_feature (534)..(534): n is a, c, g, or t
  misc_feature (601)..(601): n is a, c, g, or t
  misc_feature (615)..(617): n is a, c, g, or t
  misc_feature (921)..(921): n is a, c, g, or t
  misc_feature (955)..(956): n is a, c, g, or t
  misc_feature (1183)..(1183): n is a, c, g, or t
  misc_feature (1389)..(1389): n is a, c, g, or t
  misc_feature (1466)..(1466): n is a, c, g, or t
tgagagtttgatcctggctcaggatgaacgctggcggcgtgcttaacacatgcaagtcgaacgaagcatttacgaacagattayttcggtatgaagttcctttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcnagcgttatccggatttactgggtgtaaagggagtgtaggtggccatgcaagtcagaagtgaaaatcnggggctyaaccccnnnactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggcccataagggcttcggtgccgcagcaaacgcaataagtattccacctggggagtacgttcgcaagaatgaaactcaaaggaattgacggggaccngcacaagcggtggagcatgtggtttaattcgaannaacgcgaagaaccttaccaagtcttgacatcccactgaccggacagtaatgtgtcctttcttcgggacartrgagacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcnaatcatcatgccccttatgacttgggctacacacgtgctacaatggcgtaaacaaagtgaagcgaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgnccgtcacaccatgggagtcgaaaatgcccgaagtcggtgacctaacgtaagaaggagccgccgaaggcaggtttganaactgggg

SEQ ID NO: 65 - DNA, 1535 bp, Eubacterium eligens
  misc_feature: Eubacterium eligens DSM 3376
ctttttatgagagtttgatcctggctcaggatgaacgctggcggcgtgcttaacacatgcaagtcgaacgaagcatttagaacagattacttcggtttgaagttctttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggcccataagggcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcctcttgaccggtcagtaatgtggcctttccttcgggacaagagagacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcgaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgtaagaaggagccgccgaaggcaggtttgataactggggtgaagtcgtaacaaggtagccgtatcggaaggtgcggctggatcacctcctttct

SEQ ID NO: 66 - DNA, 1540 bp, Eubacterium eligens
  misc_feature: Eubacterium eligens DSM 3376
tttttatgagagtttgatcctggctcaggatgaacgctggcggcgtgcttaacacatgcaagtcgaacgaagcatttagaacagattacttcggtttgaagttctttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggcccataagggcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcctcttgaccggtcagtaatgtggcctttccttcgggacaagagagacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcgaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgtaagaaggagccgccgaaggcaggtttgataactggggtgaagtcgtaacaaggtagccgtatcggaaggtgcggctggatcacctcctttctaaggaa

SEQ ID NO: 67 - DNA, 1541 bp, Eubacterium eligens
  misc_feature: Eubacterium eligens DSM 3376
tttttatgagagtttgatcctggctcaggatgaacgctggcggcgtgcttaacacatgcaagtcgaacgaagcatttagaacagattatttcggtatgaagttctttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggcccaaaagggcttcggtgccgcagcaaacgcaataagtattccacctggggagtacgttcgcaagaatgaaactcaaaggaattgacggggacccgcacaagcggtggagcatgtggtttaattcgaagcaacgcgaagaaaccttaccaagtcttgacatcccactgaccggacagtaatgtgtcctttccttcgggacagtggtgacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcgaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgtaagaaggagccgccgaaggcaggtttgataactggggtgaagtcgtaacaaggtagccgtatcggaaggtgcggctggatcacctcctttctaaggaa

SEQ ID NO: 68 - DNA, 1540 bp, Eubacterium eligens
  misc_feature: Eubacterium eligens DSM 3376
tttttatgagagtttgatcctggctcaggatgaacgctggcggcgtgcttaacacatgcaagtcgaacgaagcatttagaacagattatttcggtatgaagttctttatgactgagtggcggacgggtgagtaacgcgtgggtaacctgccttgtactgggggatagcagctggaaacggctggtaataccgcataagcgcacaatgttgcatgacatagtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggcttaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggcccataagggcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgaccggacagtaatgtgtcctttccttcgggacagtggtgacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcgaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgaaagaaggagccgccgaaggcaggtttgataactggggtgaagtcgtaacaaggtagccgtatcggaaggtgcggctggatcacctcctttctaaggaa

SEQ ID NO: 69 - DNA, 1540 bp, Eubacterium eligens
  misc_feature: Eubacterium eligens DSM 3376
tttttatgagagtttgatcctggctcaggatgaacgctggcggcgtgcttaacacatgcaagtcgaacgaagcatttagaacagattatttcggtatgaagttctttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtgaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggcccataagggcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgaccggacagtaatgtgtcctttccttcgggacagtggagacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcaaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgcaagaaggagccgccgaaggcaggtttgataactggggtgaagtcgtaacaaggtagccgtatcggaaggtgcggctggatcacctcctttctaaggaa

SEQ ID NO: 70 - DNA, 1540 bp, Eubacterium eligens
  misc_feature: Eubacterium eligens DSM 3376
tttttatgagagtttgatcctggctcaggatgaacgctggcggcgtgcttaacacatgcaagtcgaacgaagcatttgcgacagattttttcggaatgaagttgcttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggcccataagggcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgaccggacagtaatgtgtcctttccttcgggacagtggagacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcgaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgtaagaaggagccgccgaaggcaggtttgataactggggtgaagtcgtaacaaggtagccgtatcggaaggtgcggctggatcacctcctttctaaggaa

SEQ ID NO: 71 - DNA, 1424 bp, Eubacterium hallii
  misc_feature: Eubacterium hallii DSM 3353
gagtggcggacgggtgagtaacgcgtgggtaacctgccctgtacagggggataacagctggaaacggctgctaataccgcataagcgcacgaggagacatctccttgtgtgaaaaactccggtggtacaggatgggcccgcgtctgattagctggttggcagggtaacggcctaccaaggcaacgatcagtagccggtctgagaggatgaacggccacattggaactgagacacggtccaaactcctacgggaggcagcagtggggaatattgcacaatgggggaaaccctgatgcagcaacgccgcgtgagtgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggtgcgtaggtggcagtgcaagtcagatgtgaaaggccggggctcaaccccggagctgcatttgaaactgctcggctagagtacaggagaggcaggcggaattcctagtgtagcggtgaaatgcgtagatattaggaggaacaccagtggcgaaggcggcctgctggactgttactgacactgaggcacgaaagcgtggggagcaaacaggattagataccctggtagtccacgccgtaaacgatgaatactaggtgtcggggccgtataggcttcggtgccgccgctaacgcagtaagtattccacctggggagtacgttcgcaagaatgaaactcaaaggaattgacggggacccgcacaagcggtggagcatgtggtttaattcgaagcaacgcgaagaaccttaccaggtcttgacatccttctgaccgcaccttaatcggtgctttccttcgggacagaagagacaggtggtgcatggttgtcgtcagctcgtgtcgtgagatgttgggttaagtcccgcaacgagcgcaacccctatcttcagtagccagcaggtaaggctgggcactctggagagactgccagggataacctggaggaaggtggggacgacgtcaaatcatcatgccccttatgatctgggcgacacacgtgctacaatggcggtcacagagtgaggcgaacccgcgagggggagcaaaccacaaaaaggccgtcccagttcggactgtagtctgcaacccgactacacgaagctggaatcgctagtaatcgcgaatcagaatgtcgcggtgaatacgttcccgggtcttgtacacaccgcccgtcacaccatgggagtcggaaatgcccgaagccagtgacccaaccttttggagggagctgtcgaaggtggagccggtaactggggtgaagtcgtaacaaggtagccgtatcggaaggtgcggctggatcacctcctttct

SEQ ID NO: 72 - DNA, 1424 bp, Eubacterium hallii; identical to SEQ ID NO: 71
  misc_feature: Eubacterium hallii DSM 3353

SEQ ID NO: 73 - DNA, 1567 bp, Lactobacillus ruminis
  misc_feature: Lactobacillus ruminis RF3
tataaattgagagtttgatcctggctcaggacgaacgctggcggcgtgcctaatacatgcaagtcgaacgaagctttctttcaccgaatgcttgcattcaccgaaagaagcttagtggcgaacgggtgagtaacacgtaggcaacctgcccaaaagagggggataacacttggaaacaggtgctaataccgcataaccatgaacaccgcatgatgtttatgtaaaagacggcttttgctgtcacttttggatgggcctgcggcgtattaacttgttggtggggtaatggcctaccaaggtgatgatacgtagccgaactgagaggttgatcggccacattgggactgagacacggcccaaactcctacgggaggcagcagtagggaatcttccacaatggacgaaagtctgatggagcaacgccgcgtgaatgaagaaggccttcgggtcgtaaaattctgttgtcagagaagaacatgcgtgagagtaactgttcacgtattgacggtatctgaccagaaagccacggctaactacgtgccagcagccgcggtaatacgtaggtggcaagcgttgtccggatttattgggcgtaaagggaacgcaggcggtcttttaagtctgatgtgaaagccttcggcttaaccgaagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgagtgctaagtgttggagggtttccgcccttcagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcttctgacaattccagagatggaacgttcccttcggggacagaatgacaggtggtgcatggttgtcgtcagctcgtgtcgtgagatgttgggttaagtcccgcaacgagcgcaacccttattgtcagttgccatcattaagttgggcactctggcgagactgccggtgacaaaccggaggaaggtggggatgacgtcaaatcatcatgccccttatgacctgggctacacacgtgctacaatggacggtacaacgagtcgctaactcgcgagggcaagctaatctcttaaagccgttctcagttcggattgcaggctgcaactcgcctgcatgaagtcggaatcgctagtaatcgcgaatcagcatgtcgcggtgaatacgttcccgggccttgtacacaccgcccgtcacaccatgagagtttgtaacacccaaagtcggtggggtaaccttttggagccagccgcctaaggtgggacagatgattggggtgaagtcgtaacaaggtagccgtaggagaacctgcggctggatcacctcctttct

SEQ ID NO: 74 - DNA, 1572 bp, Lactobacillus ruminis
  misc_feature: Lactobacillus ruminis RF3
ataaattgagagtttgatcctggctcaggacgaacgctggcggcgtgcctaatacatgcaagtcgaacgaagctttctttcaccgaatgcttgcattcaccgaaagaagcttagtggcgaacgggtgagtaacacgtaggcaacctgcccaaaagagggggataacacttggaaacaggtgctaataccgcataaccatgaacaccgcatgatgtttatgtaaaagacggcttttgctgtcacttttggatgggcctgcggcgtattaacttgttggtggggtaatggcctaccaaggtgatgatacgtagccgaactgagaggttgatcggccacattgggactgagacacggcccaaactcctacgggaggcagcagtagggaatcttccacaatggacgaaagtctgatggagcaacgccgcgtgaatgaagaaggccttcgggtcgtaaaattctgttgtcagagaagaacatgcgtgagagtaactgttcacgtattgacggtatctgaccagaaagccacggctaactacgtgccagcagccgcggtaatacgtaggtggcaagcgttgtccggatttattgggcgtaaagggaacgcaggcggtcttttaagtctgatgtgaaagccttcggcttaaccgaagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgagtgctaagtgttggagggtttccgcccttcagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcttctgacaattccagagatggaacgttcccttcggggacagaatgacaggtggtgcatggttgtcgtcagctcgtgtcgtgagatgttgggttaagtcccgcaacgagcgcaacccttattgtcagttgccatcattaagttgggcactctggcgagactgccggtgacaaaccggaggaaggtggggatgacgtcaaatcatcatgccccttatgacctgggctacacacgtgctacaatggacggtacaacgagtcgctaactcgcgagggcaagctaatctcttaaagccgttctcagttcggattgcaggctgcaactcgcctgcatgaagtcggaatcgctagtaatcgcgaatcagcatgtcgcggtgaatacgttcccgggccttgtacacaccgcccgtcacaccatgagagtttgtaacacccaaagtcggtggggtaaccttttggagccagccgcctaaggtgggacagatgattggggtgaagtcgtaacaaggtagccgtaggagaacctgcggctggatcacctcctttctaaggaa

SEQ ID NO: 75 - DNA, 1572 bp, Lactobacillus ruminis
  misc_feature: Lactobacillus ruminis RF3
ataaattgagagtttgatcctggctcaggacgaacgctggcggcgtgcctaatacatgcaagtcgaacgaagctttctttcaccgaatgcttgcattcaccgaaagaagcttagtggcgaacgggtgagtaacacgtaggcaacctgcccaaaagagggggataacacttggaaacaggtgctaataccgcataaccatgaacaccgcatgatgtttatgtaaaagacggcttttgctgtcacttttggatgggcctgcggcgtattaacttgttggtggggtaatggcctaccaaggtgatgatacgtagccgaactgagaggttgatcggccacattgggactgagacacggcccaaactcctacgggaggcagcagtagggaatcttccacaatggacgaaagtctgatggagcaacgccgcgtgaatgaagaaggccttcgggtcgtaaaattctgttgtcagagaagaacgtgcgtgagagtaactgttcacgtattgacggtatctgaccagaaagccacggctaactacgtgccagcagccgcggtaatacgtaggtggcaagcgttgtccggatttattgggcgtaaagggaacgcaggcggtcttttaaatctgatgtgaaagccttcggcttaaccgaagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgagtgctaagtgttggagggtttccgcccttcagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcttctgacaattccagagatggaacgttcccttcggggacagaatgacaggtggtgcatggttgtcgtcagctcgtgtcgtgagatgttgggttaagtcccgcaacgagcgcaacccttattgtcagttgccatcattaagttgggcactctggcgagactgccggtgacaaaccggaggaaggtggggatgacgtcaaatcatcatgccccttatgacctgggctacacacgtgctacaatggacggtacaacgagtcgctaactcgcgagggcaagctaatctcttaaagccgttctcagttcggattgcaggctgcaactcgcctgcatgaagtcggaatcgctagtaatcgcgaatcagcatgtcgcggtgaatacgttcccgggccttgtacacaccgcccgtcacaccatgagagtttgtaacacccaaagtcggtggggtaaccttttggagccagccgcctaaggtgggacagatgattggggtgaagtcgtaacaaggtagccgtaggagaacctgcggctggatcacctcctttctaaggaa

SEQ ID NO: 76 - DNA, 1572 bp, Lactobacillus ruminis
  misc_feature: Lactobacillus ruminis RF3
ataaattgagagtttgatcctggctcaggacgaacgctggcggcgtgcctaatacatgcaagtcgaacgaagctttctttcaccgaatgcttgcattcaccgaaagaagcttagtggcgaacgggtgagtaacacgtaggcaacctgcccaaaagagggggataacacttggaaacaggtgctaataccgcataaccatgaacaccgcatgatgttcatgtaaaagacggcttttgctgtcacttttggatgggcctgcggcgtattaacttgttggtggggtaatggcctaccaaggtgatgatacgtagccgaactgagaggttgatcggccacattgggactgagacacggcccaaactcctacgggaggcagcagtagggaatcttccacaatggacgaaagtctgatggagcaacgccgcgtgaatgaagaaggccttcgggtcgtaaaattctgttgtcagagaagaacgtgcgtgagagtaactgttcacgtattgacggtatctgaccagaaagccacggctaactacgtgccagcagccgcggtaatacgtaggtggcaagcgttgtccggatttattgggcgtaaagggaacgcaggcggtcttttaagtctgatgtgaaagccttcggcttaaccgaagtagtgcattggaaactggaagacttgagtgcataagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgagtgctaagtgttggagggtttccgcccttcagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcttctgacaattccagagatggaacgttcccttcggggacagaatgacaggtggtgcatggttgtcgtcagctcgtgtcgtgagatgttgggttaagtcccgcaacgagcgctacccttattgtcagttgccatcattaagttgggcactctggcgagactgccggtgacaaaccggaggaaggtggggatgacgtcaaatcatcatgccccttatgacctgggctacacacgtgctacaatggacggtacaacgagtcgctaactcgtgagggcaagctaatctcttaaagccgttctcagttcggattgcaggctgcgactcgcctgcatgaagtcggaatcgctagtaatcgcgaatcagcatgtcgcggtgaatacgttcccgggccttgtacacaccgcccgtcacaccatgagagtttgtaacacccaaagtcggtggggtaaccttttggagccagccgcctaaggtgggacagatgattggggtgaagtcgtaacaaggtagccgtaggagaacctgcggctggatcacctcctttctaaggaa

SEQ ID NO: 77 - DNA, 1572 bp, Lactobacillus ruminis
  misc_feature: Lactobacillus ruminis RF3
ataaattgagagtttgatcctggctcaggacgaacgctggcggcgtgcctaatacatgcaagtcgaacgaagctttctttcaccgaatgcttgcattcaccgaaagaagcttagtggcgaacgggtgagtaacacgtaggcaacctgcccaaaagagggggataacacttggaaacaggtgctaataccgcataaccatgaacaccgcatgatgttcatgtaaaagacggcttttgctgtcacttttggatgggcctgcggcgtattaacttgttggtggggtaatggcctaccaaggtgatgatacgtagccgaactgagaggttgatcggccacattgggactgagacacggcccaaactcctacgggaggcagcagtagggaatcttccacaatggacgaaagtctgatggagcaacgccgcgtgaatgaagaaggccttcgggtcgtaaaattctgttgtcagagaagaacgtgcgtgagagtaactgttcacgtattgacggtatctgaccagaaagccacggctaactacgtgccagcagccgcggtaatacgtaggtggcaagcgttgtccggatttattgggcgtaaagggaacgcaggcggtcttttaagtctgatgtgaaagccttcggcttaaccgaagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgagtgctaagtgttggagggtttccgcccttcagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcttctgacaattccagagatggaacgttcccttcggggacagaatgacaggtggtgcatggttgtcgtcagctcgtgtcgtgagatgttgggttaagtcccgcaacgagcgcaacccttattgtcagttgccatcattaagttgggcactctggcgagactgccggtgacaaaccggaggaaggtggggatgacgtcaaatcatcatgccccttatgacctgggctacacacgtgctacaatggacggtacaacgagtcgctaactcgcgagggcaagctaatctcttaaagccgttctcagttcggattgcaggctgcaactcgcctgcatgaagtcggaatcgctagtaatcgcgaatcagcatgtcgcggtgaatacgttcccgggccttgtacacaccgcccgtcacaccatgagagtttgtaacacccaaagtcggtggggtaaccttttggagccagccgcctaaggtgggacagatgattggggtgaagtcgtaacaaggtagccgtaggagaacctgcggctggatcacctcctttctaaggaa

SEQ ID NO: 78 - DNA, 1572 bp, Lactobacillus ruminis
  misc_feature: Lactobacillus ruminis RF3
ataaattgagagtttgatcctggctcaggacgaacgctggcggcgtgcctaatacatgcaagtcgaacgaagctttctttcaccgaatgcttgcattcaccgaaagaagcttagtggcgaacgggtgagtaacacgtaggcaacctgcccaaaagagggggataacacttggaaacaggtgctaataccgcataaccatgaacaccgcatgatgttcatgtaaaagacggcttttgctgtcacttttggatgggcctgcggcgtattaacttgttggtggggtaatggcctaccaaggtgatgatacgtagccgaactgagaggttgatcggccacattgggactgagacacggcccaaactcctacgggaggcagcagtagggaatcttccacaatggacgaaagtctgatggagcaacgccgcgtgaatgaagaaggccttcgggtcgtaaaattctgttgtcagagaagaacgtgcgtgagagtaactgttcacgtattgacggtatctgaccagaaagccacggctaactacgtgccagcagccgcggtaatacgtaggtggcaagcgttgtccggatttattgggcgtaaagggaacgcaggcggtcttttaaatctgatgtgaaagccttcggcttaaccgaagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgagtgctaagtgttggagggtttccgcccttcagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcttctgacaattccagagatggaacgttcccttcggggacagaatgacaggtggtgcatggttgtcgtcagctcgtgtcgtgagatgttgggttaagtcccgcaacgagcgcaacccttattgtcagttgccatcattaagttgggcactctggcgagactgccggtgacaaaccggaggaaggtggggatgacgtcaaatcatcatgccccttatgacctgggctacacacgtgctacaatggacggtacaacgagtcgctaactcgcgagggcaagctaatctcttaaagccgttctcagttcggattgcaggctgcaactcgcctgcatgaagtcggaatcgctagtaatcgcgaatcagcatgtcgcggtgaatacgttcccgggccttgtacacaccgcccgtcacaccatgagagtttgtaacacccaaagtcggtggggtaaccatttggagccagccgcctaaggtgggacagatgattggggtgaagtcgtaacaaggtagccgtaggagaacctgcggctggatcacctcctttctaaggaa

SEQ ID NO: 79 - DNA, 1572 bp, Lactobacillus ruminis; identical to SEQ ID NO: 78
  misc_feature: Lactobacillus ruminis RF3

SEQ ID NO: 80 - DNA, 1331 bp, Megamonas funiformis
  misc_feature: Megamonas funiformis DSM 19343
agcttccgcttgaagatgagcttgcgtctgattagctagttggtgagggtaaaggcccaccaaggcgacgatcagtagccggtctgagaggatgaacggccacattgggactgagacacggcccagactcctacgggaggcagcagtggggaatcttccgcaatgggcgaaagcctgacggagcaacgccgcgtgaacgatgaaggtcttaggatcgtaaagttctgttgttagggacgaaggataaggattataatacagtctttgtttgacggtacctaacgaggaagccacggctaactacgtgccagcagccgcggtaatacgtaggcggcaagcgttgtccggaattattgggcgtaaagggagcgcaggcgggaaactaagcggatcttaaaagtgcggggctcaaccccgtgatggggtccgaactggttttcttgagtgcaggagaggaaagcggaattcccagtgtagcggtgaaatgcgtagatattgggaagaacaccagtggcgaaggcggctttctggactgtaactgacgctgaggctcgaaagctagggtagcgaacgggattagataccccggtagtcctagccgtaaacgatggatactaggtgtgggaggtatcgactccttccgtgccggagttaacgcaataagtatcccgcctggggagtacggccgcaaggttgaaactcaaaggaattgacgggggcccgcacaagcggtggagtatgtggtttaattcgacgcaacgcgaagaaccttaccaagacttgacattgattgaaagacttagagataagttccttctcttcggagaacaagaaaacaggtggtgcatggctgtcgtcagctcgtgtcgtgagatgttgggttaagtcccgcaacgagcgcaacccctatactatgttgccagcatttcggatgggaactcatagtagactgccgcggacaacgcggaggaaggcggggatgacgtcaagtcatcatgccccttacgtcttgggctacacacgtactacaatgggatgaacagagggaagcgaaatcgcgaggtggagcggatccctaaaagcatctctcagttcggattgtaggctgaaactcgcctacatgaagtcggaatcgctagtaatcgcaggtcagcatactgcggtgaatacgttcccgggccttgtacacaccgcccgtcacaccacgaaagtcattcacacccgaagccggctaagggcctatggtaccgaccgtctaaggtgggggcgatgattggggtgaagtcgtaacaaggtagccgtatcggaaggtgcggctggatcacctcctt

SEQ ID NO: 81 - DNA, 1559 bp, Megamonas funiformis
  misc_feature: Megamonas funiformis DSM 19343
tttttttggagagtttgatcctggctcaggacgaacgctggcggcgtgcttaacacatgcaagtcgaacggggggtttatttcggtaaacaccaagtggcgaacgggtgagtaacgcgtaagcaatctaccttcaagatggggacaacacttcgaaaggggtgctaataccgaatgaatgtaagagtatcgcatgagacacttactaaaggaggcctctgacataagcttccgcttgaagatgagcttgcgtctgattagctagttggtgagggtaaaggcccaccaaggcgacgatcagtagccggtctgagaggatgaacggccacattgggactgagacacggcccagactcctacgggaggcagcagtggggaatcttccgcaatgggcgaaagcctgacggagcaacgccgcgtgaacgatgaaggtcttaggatcgtaaagttctgttgttagggacgaaggataagaatcataataaagtctttatttgacggtacctaacgaggaagccacggctaactacgtgccagcagccgcggtaatacgtaggcggcaagcgttgtccggaattattgggcgtaaagggagcgcaggcgggaaactaagcggatcttaaaagtgcggggctcaaccccgtgatggggtccgaactggttttcttgagtgcaggagaggaaagcggaattcccagtgtagcggtgaaatgcgtagatattgggaagaacaccagtggcgaaggcggctttctggactgtaactgacgctgaggctcgaaagctagggtagcgaacgggattagataccccggtagtcctagccgtaaacgatggatactaggtgtgggaggtatcgactccttccgtgccggagttaacgcaataagtatcccgcctggggagtacggccgcaaggttgaaactcaaaggaattgacgggggcccgcacaagcggtggagtatgtggtttaattcgacgcaacgcgaagaaccttaccaagacttgacattgattgaaagacttagagataagttccttctcttcggagaacaagaaaacaggtggtgcatggctgtcgtcagctcgtgtcgtgagatgttgggttaagtcccgcaacgagcgcaacccctatactatgttgccagcattacggatgggaactcatagtagactgccgcggacaacgcggaggaaggcggggatgacgtcaagtcatcatgccccttacgtcttgggctacacacgtactacaatgggatgaacagagggaagcgaaatcgcgaggtggagcggatccctaaaagcatctctcagttcggattgtaggctgaaactcgcctacatgaagtcggaatcgctagtaatcgcaggtcagcatactgcggtgaatacgttcccgggccttgtacacaccgcccgtcacaccacgaaagtcattcacacccgaagccggctaagggcctatggtaccgaccgtctaaggggggggcgatgattggggtgaagtcgtaacaaggtagccgtatcggaaggtgcggctggatcacctcctttct

SEQ ID NO: 82 - DNA, 1559 bp, Megamonas funiformis
  misc_feature: Megamonas funiformis DSM 19343
tttttttggagagtttgatcctggctcaggacgaacgctggcggcgtgcttaacacatgcaagtcgaacggggtgtttatttcggtaaacaccaagtggcgaacgggtgagtaacgcgtaagcaatctaccttcaagatggggacaacacttcgaaaggggtgctaataccgaatgaatgtaagagtatcgcatgagacacttactaaaggaggcctctgacataagcttccgcttgaagatgagcttgcgtctgattagctagttggtgagggtaaaggcccaccaaggcgacgatcagtagccggtctgagaggatgaacggccacattgggactgagacacggcccagactcctacgggaggcagcagtggggaatcttccgcaatgggcgaaagcctgacggagcaacgccgcgtgaacgatgaaggtcttaggatcgtaaagttctgttgttagggacgaagggtaaggattataatacggtttttgtttgacggtacctaacgaggaagccacggctaactacgtgccagcagccgcggtaatacgtaggcggcaagcgttgtccggaattattgggcgtaaagggagcgcaggcgggaaactaagcggatcttaaaagtgcggggctcaaccccgtgatggggtccgaactggttttcttgagtgcaggagaggaaagcggaattcccagtgtagcggtgaaatgcgtagatattgggaagaacaccagtggcgaaggcggctttctggactgtaactgacgctgaggctcgaaagctagggtagcgaacgggattagataccccggtagtcctagccgtaaacgatggatactaggtgtgggaggtatcgactccttccgtgccggagttaacgcaataagtatcccgcctggggagtacggccgcaaggttgaaactcaaaggaattgacgggggcccgcacaagcggtggagtatgtggtttaattcgacgcaacgcgaagaaccttaccaagacttgacattgattgaaagacttagagataagttccttctcttcggagaacaagaaaacaggtggtgcatggctgtcgtcagctcgtgtcgtgagatgttgggttaagtcccgcaacgagcgcaacccctatactatgttgccagcatttcggatgggaactcatagtagactgccgcggacaacgcggaggaaggcggggatgacgtcaagtcatcatgccccttacgtcttgggctacacacgtactacaatgggatgaacagagggaagcgaaatcgcgaggtggagcggatccctaaaagcatctctcagttcggattgtaggctgaaactcgcctacatgaagtcggaatcgctagtaatcgcaggtcagcatactgcggtgaatacgttcccgggccttgtacacaccgcccgtcacaccacgaaagtcattcacacccgaagccggctaagggcctatggtaccgaccgtctagggtgggggcgatgattggggtgaagtcgtaacaaggtagccgtatcggaaggtgcggctggatcacctcctttct

SEQ ID NO: 83 - DNA, 1559 bp, Megamonas funiformis; identical to SEQ ID NO: 82
  misc_feature: Megamonas funiformis DSM 19343

SEQ ID NO: 84 - DNA, 1559 bp, Megamonas funiformis; identical to SEQ ID NO: 81
  misc_feature: Megamonas funiformis DSM 19343

SEQ ID NO: 85 - DNA, 1564 bp, Megamonas funiformis
  misc_feature: Megamonas funiformis DSM 19343
ttttttggagagtttgatcctggctcaggacgaacgctggcggcgtgcttaacacatgcaagtcgaacggggggtttatttcggtaaacaccaagtggcgaacgggtgagtaacgcgtaagcaatctaccttcaagatggggacaacacttcgaaaggggtgctaataccgaatgaatgtaagagtatcgcatgagacacttactaaaggaggcctctgacataagcttccgcttgaagatgagcttgcgtctgattagctagttggtgagggtaaaggcccaccaaggcgacgatcagtagccggtctgagaggatgaacggccacattgggactgagacacggcccagactcctacgggaggcagcagtggggaatcttccgcaatgggcgaaagcctgacggagcaacgccgcgtgaacgatgaaggtcttaggatcgtaaagttctgttgttagggacgaaggataagaatcataataaagtctttatttgacggtacctaacgaggaagccacggctaactacgtgccagcagccgcggtaatacgtaggcggcaagcgttgtccggaattattgggcgtaaagggagcgcaggcgggaaactaagcggatcttaaaagtgcggggctcaaccccgtgatggggtccgaactggttttcttgagtgcaggagaggaaagcggaattcccagtgtagcggtgaaatgcgtagatattgggaagaacaccagtggcgaaggcggctttctggactgtaactgacgctgaggctcgaaagctagggtagcgaacgggattagataccccggtagtcctagccgtaaacgatggatactaggtgtgggaggtatcgactccttccgtgccggagttaacgcaataagtatcccgcctggggagtacggccgcaaggttgaaactcaaaggaattgacgggggcccgcacaagcggtggagtatgtggtttaattcgacgcaacgcgaagaaccttaccaagacttgacattgattgaaagacttagagataagttccttctcttcggagaacaagaaaacaggtggtgcatggctgtcgtcagctcgtgtcgtgagatgttgggttaagtcccgcaacgagcgcaacccctatactatgttgccagcattacggatgggaactcatagtagactgccgcggacaacgcggaggaaggcggggatgacgtcaagtcatcatgccccttacgtcttgggctacacacgtactacaatgggatgaacagagggaagcgaaatcgcgaggtggagcggatccctaaaagcatctctcagttcggattgtaggctgaaactcgcctacatgaagtcggaatcgctagtaatcgcaggtcagcatactgcggtgaatacgttcccgggccttgtacacaccgcccgtcacaccacgaaagtcattcacacccgaagccggctaagggcctatggtaccgaccgtctaaggggggggcgatgattggggtgaagtcgtaacaaggtagccgtatcggaaggtgcggctggatcacctcctttctaaggag

SEQ ID NO: 86 - DNA, 1564 bp, Megamonas funiformis; identical to SEQ ID NO: 85
  misc_feature: Megamonas funiformis DSM 19343

SEQ ID NO: 87 - DNA, 1417 bp, Megasphaera elsdenii
  misc_feature: Megasphaera elsdenii LC1
agagtttgatcctggctcaggacgaacgctggcggcgtgcttaacacatgcaagtcgaacgagaagagatgagaagcttgcttcttatcgattcgagtggcaaacgggtgagtaacgcgtaagcaacctgcccttcagatggggacaacagctggaaacggctgctaataccgaatacgttctttttgtcgcatggcagagggaagaaagggaggctcttcggagctttcgctgaaggaggggcttgcgtctgattagctagttggaggggtaacggcccaccaaggcgacgatcagtagccggtctgagaggatgaacggccacattgggactgagacacggcccagactcctacgggaggcagcagtaggggaatcttccgcaatggacgaaagtctgacggagcaacgccgcgtgaacgatgacggccttcgggttgtaaagttctgttatacgggacgaatggcgtacgggtcaatacccgttacgagtgacggtaccgtaagagaaagccacggctaactacgtgccagcagccgcggtaatacgtaggtggcaagcgttgtccggaattattgggcgtaaagggcgcgcaggcggcgtcgtaagtcggtcttaaaagtgcggggcttaaccccgtgagggaccgaaactgcgatgctagagtatcggagaggaaagcggaattcctagtgtagcggtgaaatgcgtagatattaggaggaacaccagtggcgaaagcggctttctggacgacaactgacgctgaggcgcgaaagccaggggagcaaacgggattagataccccggtagtcctggccgtaaacgatggatactaggtgtaggaggtatcgaccccttctgtgccggagttaacgcaataagtatcccgcctggggagtacggccgcaaggctgaaactcaaaggaattgacgggggcccgcacaagcggtggagtatgtggtttaattcgacgcaacgcgaagaaccttaccaagccttgacattgattgctatggatagagatatccagttcctcttcggaggacaagaaaacaggtggtgcacggctgtcgtcagctcgtgtcgtgagatgttgggttaagtcccgcaacgagcgcaacccctatcttctgttaccagcgagttaagtcggggactcaggagagactgccgcagacaatgcggaggaaggcggggatgacgtcaagtcatcatgccccttatggcttgggctacacacgtactacaatggctcttaatagagggaagcgaaggagcgatccggagcaaaccccaaaaacagagtcccagttcggattgcaggctgcaactcgcctgcatgaagcaggaatcgctagtaatcgcaggtcagcatactgcggtgaatacgttcccgggccttgtacacaccg

SEQ ID NO: 88 - DNA, 1568 bp, Megasphaera elsdenii
  misc_feature: Megasphaera elsdenii LC1
atatcatggagagtttgatcctggctcaggacgaacgctggcggcgtgcttaacacatgcaagtcgaacgagaagagatgagaagcttgcttcttatcaattcgagtggcaaacgggtgagtaacgcgtaagcaacctgcccttcagatggggacaacagctggaaacggctgctaataccgaatacgttctttttgtcgcatggcagagggaagaaagggaggctcttcggagctttcgctgaaggaggggcttgcgtctgattagctagttggaggggtaacggcccaccaaggcgacgatcagtagccggtctgagaggatgaacggccacattgggactgagacacggcccagactcctacgggaggcagcagtggggaatcttccgcaatggacgaaagtctgacggagcaacgccgcgtgaacgatgacggccttcgggttgtaaagttctgttatacgggacgaatggcgtagcggtcaatacccgttacgagtgacggtaccgtaagagaaagccacggctaactacgtgccagcagccgcggtaatacgtaggtggcaagcgttgtccggaattattgggcgtaaagggcgcgcaggcggcgtcgtaagtcggtcttaaaagtgcggggcttaaccccgtgaggggaccgaaactgcgatgctagagtatcggagaggaaagcggaattcctagtgtagcggtgaaatgcgtagatattaggaggaacaccagtggcgaaagcggctttctggacgacaactgacgctgaggcgcgaaagccaggggagcaaacgggattagataccccggtagtcctggccgtaaacgatggatactaggtgtaggaggtatcgaccccttctgtgccggagttaacgcaataagtatcccgcctggggagtacggccgcaaggctgaaactcaaaggaattgacgggggcccgcacaagcggtggagtatgtggtttaattcgacgcaacgcgaagaaccttaccaagccttgacattgattgctatggatagagatatccagttcctcttcggaggacaagaaaacaggtggtgcacggctgtcgtcagctcgtgtcgtgagatgttgggttaagtcccgcaacgagcgcaacccctatcttctgttaccagcggttcggccggggactcaggagagactgccgcagacaatgcggaggaaggcggggatgacgtcaagtcatcatgccccttatggcttgggctacacacgtactacaatggctcttaatagagggaagcgaaggagcgatccggagcaaaccccaaaaacagagtcccagttcggattgcaggctgcaactcgcctgcatgaagcaggaatcgctagtaatcgcaggtcagcatactgcggtgaatacgttcccgggccttgtacacaccgcccgtcacaccacgaaagtcattcacacccgaagccggtgaggtaaccttttggagccagccgtcgaaggtgggggcgatgattggggtgaagtcgtaacaaggtagccgtatcggaaggtgcggctggatcacctcctttct

SEQ ID NO: 89 - DNA, 1568 bp, Megasphaera elsdenii
  misc_feature: Megasphaera elsdenii LC1
atggagagtttgatcctggctcaggacgaacgctggcggcgtgcttaacacatgcaagtcgaacgagaagagatgagaagcttgcttcttatcaattcgagtggcaaacgggtgagtaacgcgtaagcaacctgcccttcagatggggacaacagctggaaacggctgctaataccgaatacgttctttttgtcgcatggcagagggaagaaagggaggctcttcggagctttcgctgaaggaggggcttgcgtctgattagctagttggaggggtaacggcccaccaaggcgacgatcagtagccggtctgagaggatgaacggccacattgggactgagacacggcccagactcctacgggaggcagcagtggggaatcttccgcaatggacgaaagtctgacggagcaacgccgcgtgaacgatgacggccttcgggttgtaaagttctgttatacgggacgaatggcgtagcggtcaatacccgttacgagtgacggtaccgtaagagaaagccacggctaactacgtgccagcagccgcggtaatacgtaggtggcaagcgttgtccggaattattgggcgtaaagggcgcgcaggcggcgtcgtaagtcggtcttaaaagtgcggggcttaaccccgtgaggggaccgaaactgcgatgctagagtatcggagaggaaagcggaattcctagtgtagcggtgaaatgcgtagatattaggaggaacaccagtggcgaaagcggctttctggacgacaactgacgctgaggcgcgaaagccaggggagcaaacgggattagataccccggtagtcctggccgtaaacgatggatactaggtgtaggaggtatcgaccccttctgtgccggagttaacgcaataagtatcccgcctggggagtacggccgcaaggctgaaactcaaaggaattgacgggggcccgcacaagcggtggagtatgtggtttaattcgacgcaacgcgaagaaccttaccaagccttgacattgattgctatggatagagatatccagttcctcttcggaggacaagaaaacaggtggtgcacggctgtcgtcagctcgtgtcgtgagatgttgggttaagtcccgcaacgagcgcaacccctatcttctgttaccagcggttcggccggggactcaggagagactgccgcagacaatgcggaggaaggcggggatgacgtcaagtcatcatgccccttatggcttgggctacacacgtactacaatggctcttaatagagggaagcgaaggagcgatccggagcaaaccccaaaaacagagtcccagttcggattgcaggctgcaactcgcctgcatgaagcaggaatcgctagtaatcgcaggtcagcatactgcggtgaatacgttcccgggccttgtacacaccgcccgtcacaccacgaaagtcattcacacccgaagccggtgaggtaaccttttggagccagccgtcgaaggtgggggcgatgattggggtgaagtcgtaacaaggtagccgtatcggaaggtgcggctggatcacctcctttctaggga

SEQ ID NO: 90 - DNA, 1568 bp, Megasphaera elsdenii; identical to SEQ ID NO: 89
  misc_feature: Megasphaera elsdenii LC1

SEQ ID NO: 91 - DNA, 1568 bp, Megasphaera elsdenii; identical to SEQ ID NO: 89
  misc_feature: Megasphaera elsdenii LC1

SEQ ID NO: 92 - DNA, 1568 bp, Megasphaera elsdenii; identical to SEQ ID NO: 89
  misc_feature: Megasphaera elsdenii LC1

SEQ ID NO: 93 - DNA, 1568 bp, Megasphaera elsdenii; identical to SEQ ID NO: 89
  misc_feature: Megasphaera elsdenii LC1

SEQ ID NO: 94 - DNA, 1568 bp, Megasphaera elsdenii; identical to SEQ ID NO: 89
  misc_feature: Megasphaera elsdenii LC1

SEQ ID NO: 95 - DNA, 1568 bp, Megasphaera elsdenii; identical to SEQ ID NO: 89
  misc_feature: Megasphaera elsdenii LC1

SEQ ID NO: 96 - DNA, 1477 bp, Methanosphaera stadtmanae
  misc_feature: Methanosphaera stadtmanae MCB-3
aatccgtttgatcctggcggaagctactgctattgggattcgattaagccatgcaagtcgaatgaacctagttcatggcatacggctcagtaacacgtggataacttacccttaggaccgggataactttgggaaactgaagataatactggatattaggttatgcctggaatggtttacctttgaaatgtatttttcgcctaaggatagatctgcggctgattaggtcgttggtggggtaatggcccaccaagccgatgatcggtacgggttgtgagagcaagagcccggagatggaacctgagacaaggttccagaccctacggggtgcagcaggcgcgaaacctccgcaatgtacgcaagtgcgacggggggatcccaagtgttattcttaacagaataacttttcattagtgtaaaaagcttttagaataagagctgggcaagaccggtgccagccgccgcggtaacaccggcagctcaagtggtagctgtttttattgggcctaaagcgttcgtagccggtttgataagtctttggtgaaagcttgtagcttaactataagaattgctgaagatactgtcagacttgaagtcgggagaggttagaggtactaccggggtaggggtgaaatcctataatcctgggaggaccacctgtggcgaaggcgtctaactggaacgatcttgacggtgagtaacgaaagccaggggcgcgaaccggattagatacccgggtagtcctggccgtaaacgatgtggacttggtgttggaatggcctcgagttgttccggtgccgaagggaagctgttaagtccaccgcctgggaagtacggtcgcaagactgaaacttaaaggaattggcgggggagcaccacaacgcgtggagcctgcggtttaattggattcaacgccggacatctcaccaggagcgacagcagaatgataatcaggttgatgaccttatttgactagctgagaggaggtgcatggccgccgtcagctcgtactgtgaagcgtcctgttaagtcaggcaacgagcgagacccacgcccttagttaccaacacaatctctggattggtgggcacactagggggaccgccagtgataaactggaggaaggagtggacgacggtaggtccgtatgccccgaatctcctgggctacacgcgggctacaatggctatggcaatgggtttctgcactgaaaagtgatggtaatctcctaaacatagtcttagttcggattgagggctgtaactcgccctcatgaagctggaatgcgtagtaatcgcatgtcacaaccgtgcggtgaatacgtccctgctccttgcacacaccgcccgtcacaccacccaaaaagggtttggatgaggccataattttcaaatgttattgtcgaatctaggttttttgaggagggtgaagtcgtaacaaggtagccgtaggggaacctgcggctggatcacctcct

SEQ ID NO: 97 - DNA, 1477 bp, Methanosphaera stadtmanae; identical to SEQ ID NO: 96
  misc_feature: Methanosphaera stadtmanae MCB-3

SEQ ID NO: 98 - DNA, 1267 bp, Methanosphaera stadtmanae
  misc_feature: Methanosphaera stadtmanae MCB-3
gctcagtaacacgtggataacttacccttaggaccgggataactttgggaaactgaagataatactggatattaggttatgcctggaatggtttacctttgaaatgtatttttcgcctaaggatagatctgcggctgattaggtcgttggtggggtaatggcccaccaagccgatgatcggtacgggttgtgagagcaagagcccggagatggaacctgagacaaggttccagaccctacggggtgcagcaggcgcgaaacctccgcaatgtacgcaagtgcgacggggggatcccaagtgttattcttaacagaataacttttcattagtgtaaaaagcttttagaataagagctgggcaagaccggtgccagccgccgtggtaacaccggcagctcaagtggtagctgtttttattgggcctaaagcgttcgtagccggtttgataagtctttggtgaaagcttgtagcttaactataagaattgctgaagatactgtcagacttgaagtcgggagaggttagaggtactaccggggtaggggtgaaatcctataatcctgggaggaccacctgtggcgaaggcgtctaactggaacgatcttgacggtgagtaacgaaagccaggggcgcgaaccggattagatacccgggtagtcctggccgtaaacgatgtggacttggtgttggaatggcctcgagttgttccggtgccgaagggaagctgttaagtccaccgcctgggaagtacggtcgcaagactgaaacttaaaggaattggcgggggagcaccacaacgcgtggagcctgcggtttaattggattcaacgccggacatctcaccaggagcgacagcagaatgataatcaggttgatgaccttatttgactagctgagaggaggtgcatggccgccgtcagctcgtactgtgaagcgtcctgttaagtcaggcaacgagcgagacccacgcccttagttaccaacacaatctctggattggtgggcacactagggggaccgccagtgataaactggaggaaggagtggacgacggtaggtccgtatgccccgaatctcctgggctacacgcgggctacaatggctatggcaatgggtttctgcactgaaaagtgatggtaatctcctaaacatagtcttagttcggattgagggctgtaactcgccctcatgaagctggaatgcgtagtaatcgcatgtcacaaccgtgcggtgaatacgtccctgctccttgcacacaccgcccgtc

SEQ ID NO: 99 - DNA, 1490 bp, Methanosphaera stadtmanae
  misc_feature: Methanosphaera stadtmanae MCB-3
aatagtgtgtaaatccgtttgatcctggcggaagctactgctattgggattcgattaagccatgcaagtcgaatgaacctagttcatggcatacggctcagtaacacgtggataacttacccttaggaccgggataactttgggaaactgaagataatactggatattaggttatgcctggaatggtttacctttgaaatgtatttttcgcctaaggatagatctgcggctgattaggtcgttggtggggtaatggcccaccaagccgatgatcggtacgggttgtgagagcaagagcccggagatggaacctgagacaaggttccagaccctacggggtgcagcaggcgcgaaacctccgcaatgtacgcaagtgcgacggggggatcccaagtgttattcttaacagaataacttttcattagtgtaaaaagcttttagaataagagctgggcaagaccggtgccagccgccgcggtaacaccggcagctcaagtggtagctgtttttattgggcctaaagcgttcgtagccggtttgataagtctttggtgaaagcttgtagcttaactataagaattgctgaagatactgtcagacttgaagtcgggagaggttagaggtactaccggggtaggggtgaaatcctataatcctgggaggaccacctgtggcgaaggcgtctaactggaacgatcttgacggtgagtaacgaaagccaggggcgcgaaccggattagatacccgggtagtcctggccgtaaacgatgtggacttggtgttggaatggcctcgagttgttccggtgccgaagggaagctgttaagtccaccgcctgggaagtacggtcgcaagactgaaacttaaaggaattggcgggggagcaccacaacgcgtggagcctgcggtttaattggattcaacgccggacatctcaccaggagcgacagcagaatgataatcaggttgatgaccttatttgactagctgagaggaggtgcatggccgccgtcagctcgtactgtgaagcgtcctgttaagtcaggcaacgagcgagacccacgcccttagttaccaacacaatctctggattggtgggcacactagggggaccgccagtgataaactggaggaaggagtggacgacggtaggtccgtatgccccgaatctcctgggctacacgcgggctacaatggctatggcaatgggtttctgcactgaaaagtgatggtaatctcctaaacatagtcttagttcggattgagggctgtaactcgccctcatgaagctggaatgcgtagtaatcgcatgtcacaaccgtgcggtgaatacgtccctgctccttgcacacaccgcccgtcacaccacccaaaaagggtttggatgaggccataattttcaaatgttattgtcgaatctaggttttttgaggagggtgaagtcgtaacaaggtagccgtaggggaacctgcggctggatcacctcctaa

SEQ ID NO: 100 - DNA, 1491 bp, Methanosphaera stadtmanae
  misc_feature: Methanosphaera stadtmanae MCB-3
aatagtgtgtaaatccgtttgatcctggcggaagctactgctattgggattcgattaagccatgcaagtcgaatgaacctagttcatggcatacggctcagtaacacgtggataacttacccttaggaccgggataactttgggaaactgaagataatactggatattaggttatgcctggaatggtttacctttgaaatgtatttttcgcctaaggatagatctgcggctgattaggtcgttggtggggtaatggcccaccaagccgatgatcggtacgggttgtgagagcaagagcccggagatggaacctgagacaaggttccagaccctacggggtgcagcaggcgcgaaacctccgcaatgtacgcaagtgcgacggggggatcccaagtgttattcttaacagaataacttttcattagtgtaaaaagcttttagaataagagctgggcaagaccggtgccagccgccgcggtaacaccggcagctcaagtggtagctgtttttattgggcctaaagcgttcgtagccggtttgataagtctttggtgaaagcttgtagcttaactataagaattgctgaagatactgtcagacttgaagtcgggagaggttagaggtactaccggggtaggggtgaaatcctataatcctgggaggaccacctgtggcgaaggcgtctaactggaacgatcttgacggtgagtaacgaaagccaggggcgcgaaccggattagatacccgggtagtcctggccgtaaacgatgtggacttggtgttggaatggcctcgagttgttccggtgccgaagggaagctgttaagtccaccgcctgggaagtacggtcgcaagactgaaacttaaaggaattggcgggggagcaccacaacgcgtggagcctgcggtttaattggattcaacgccggacatctcaccaggagcgacagcagaatgataatcaggttgatgaccttatttgactagctgagaggaggtgcatggccgccgtcagctcgtactgtgaagcgtcctgttaagtcaggcaacgagcgagacccacgcccttagttaccaacacaatctctggattggtgggcacactagggggaccgccagtgataaactggaggaaggagtggacgacggtaggtccgtatgccccgaatctcctgggctacacgcgggctacaatggctatggcaatgggtttctgcactgaaaagtgatggtaatctcctaaacatagtcttagttcggattgagggctgtaactcgccctcatgaagctggaatgcgtagtaatcgcatgtcacaaccgtgcggtgaatacgtccctgctccttgcacacaccgcccgtcacaccacccaaaaagggtttggatgaggccataattttcaaatgttattgtcgaatctaggttttttgaggagggtgaagtcgtaacaaggtagccgtaggggaacctgcggctggatcacctcctaac

SEQ ID NO: 101 - DNA, 1491 bp, Methanosphaera stadtmanae; identical to SEQ ID NO: 100
  misc_feature: Methanosphaera stadtmanae MCB-3

SEQ ID NO: 102 - DNA, 1491 bp, Methanosphaera stadtmanae; identical to SEQ ID NO: 100
  misc_feature: Methanosphaera stadtmanae MCB-3

SEQ ID NO: 103 - DNA, 1425 bp, Methanosphaera stadtmanae
  misc_feature: Methanosphaera stadtmanae MCB-3
  misc_feature (590)..(590): n is a, c, g, or t
  misc_feature (894)..(896): n is a, c, g, or t
aatccgtttgatcctggcggaagctactgctattgggattcgattaagccatgcaagtcgaatgaacctagttcatggcatacggctcagtaacacgtggataacctacccttaggaccgggataactttgggaaactgaagataatactggatattaggttatgcctggaatggtttacctttgaaatgtatttttcgcctaaggatagatctgcggctgattaggtcgttggtggggtaatggcccaccaagccgatgatcggtacgggttgtgagagcaagagcccggagatggaacctgagacaaggttccagaccctacggggtgcagcaggcgcgaaacctccgcaatgtacgcaagtgcgacggggggatcccaagtgttattcttaacagaataacttttcattagtgtaaaaagcttttagaataagagctgggcaagaccggtgccagccgccgcggtaacaccggcagctcaagtggtagctgtttttattgggcctaaagcgttcgtagccggtttgataagtctttggtgaaagcttgtagcttaactataagaattgctgaagatactgtcagacntgaagtcgggagaggttagaggtactaccggggtaggggtgaaatcctataatcctgggaggaccacctgtggcgaaggcgtctaactggaacgatcttgacggtgagtaacgaaagccaggggcgcgaaccggattagatacccgggtagtcctggccgtaaacgatgtggacttggtgttggaatggcctcgagttgttccggtgccgaagggaagctgttaagtccaccgcctgggaagtacggtcgcaagactgaaacttaaaggaattggcgggggagcaccacaacgcgtggagcctnnngtttaattggattcaacgccggacatctcaccaggagcgacagcagaatgataatcaggttgatgaccttatttgactagctgagaggaggtgcatggccgccgtcagctcgtaccgtgaagcgtcctgttaagtcaggcaacgagcgagacccacgcccttagttaccaacacaatctctggattggtgggcacactagggggaccgccagtgataaactggaggaaggagtggacgacggtaggtcagtatgccccgaatctcctgggctacacgcgggctacaatggctatggcaatgggtttctgcactgaaaagtgatggtaatctcctaaacatagtcttagttcggattgagggctgtaactcgccctcatgaagctggaatgcgtagtaatcgcatgtcacaaccgtgcggtgaatacgtccctgctccttgcacacaccgcccgtcacaccacccaaaaagggtttggatgaggccataattttcaaatgttattgtcgaatctaggttttttgaggagg

SEQ ID NO: 104 - DNA, 1492 bp, Parabacteroides johnsonii
  misc_feature: Parabacteroides johnsonii M-165
agagtttgatcctggctcaggatgaacgctagcgacaggcttaacacatgcaagtcgaggggcagcatggtaagtagcaatacttattgatggcgaccggcgcacgggtgagtaacgcgtatgcaacttacctatcagagggggatagcccggcgaaagtcggattaatactccataaaacaggggttccgcatgggactatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggatgaaggatctatggtttgtaaacttcttttataggggaataaagtgtgggacgtgttccattttgtatgtaccctatgaataagcatcggctaactccgtgccagcagccgcggtaatacggaggatgcgagcgttatccggatttattgggtttaaagggtgcgtaggtggtaatttaagtcagcggtgaaagtttgtggctcaaccataaaattgccgttgaaactgggttacttgagtgtgtttgaggtaggcggaatgcgtggtgtagcggtgaaatgcatagatatcacgcagaactccaattgcgaaggcagcttactaaaccataactgacactgaagcacgaaagcgtgggtatcaaacaggattagataccctggtagtccacgcagtaaacgatgattactaggagtttgcgatacacagtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtcagaccgaccttgaaagaggtcttctagcaatagctgattacgaggtgctgcatggttgtcgtcagctcgtgccgtgaggtgtcggcttaagtgccataacgagcgcaacccttatcactagttactaacaggtcaagctgaggactctggtgagactgccagcgtaagctgtgaggaaggtggggatgacgtcaaatcagcacggcccttacatccggggcgacacacgtgttacaatggcatggacaaagggcagctacctggcgacaggatgctaatctctaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaaggatcggcctagggtaaaactggtgactggggctaagtcgtaacaaggtaacc

SEQ ID NO: 105 - DNA, 1546 bp, Parabacteroides johnsonii
  misc_feature: Parabacteroides johnsonii M-165
caatttcaacaacgaagagtttgatcctggctcaggatgaacgctagcgacaggcttaacacatgcaagtcgaggggcagcatggtaagtagcaatacttattgatggcgaccggcgcacgggtgagtaacgcgtatgcaacttacctatcagagggggatagcccggcgaaagtcggattaatactccataaaacaggggttccgcatgggactatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggatgaaggatctatggtttgtaaacttcttttataggggaataaagtgtgggacgtgttccattttgtatgtaccctatgaataagcatcggctaactccgtgccagcagccgcggtaatacggaggatgcgagcgttatccggatttattgggtttaaagggtgcgtaggtggtaatttaagtcagcggtgaaagtttgtggctcaaccataaaattgccgttgaaactgggttacttgagtgtgtttgaggtaggcggaatgcgtggtgtagcggtgaaatgcatagatatcacgcagaactccaattgcgaaggcagcttactaaaccataactgacactgaagcacgaaagcgtgggtatcaaacaggattagataccctggtagtccacgcagtaaacgatgattactaggagtttgcgatacacagtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtcagaccgaccttgaaagaggttttctagcaatagctgattacgaggtgctgcatggttgtcgtcagctcgtgccgtgaggtgtcggcttaagtgccataacgagcgcaacccttatcactagttactaacaggttaagctgaggactctggtgagactgccagcgtaagctgtgaggaaggtggggatgacgtcaaatcagcacggcccttacatccggggcgacacacgtgttacaatggcatggacaaagggcagctacctggcgacaggatgctaatctctaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaaggatcggcctagggtaaaactggtgactggggctaagtcgtaacaaggtagccgtaccggaaggtgcggctggaacacctcctttctggagc

SEQ ID NO: 106 - DNA, 1530 bp, Parabacteroides johnsonii
  misc_feature: Parabacteroides johnsonii M-165
acaacgaagagtttgatcctggctcaggatgaacgctagcgacaggcttaacacatgcaagtcgaggggcagcatggtaagtagcaatacttattgatggcgaccggcgcacgggtgagtaacgcgtatgcaacttacctatcagagggggatagcccggcgaaagtcggattaatactccataaaacaggggttccgcatgggactatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggatgaaggatctatggtttgtaaacttcttttataggggaataaagtgtgggacgtgttccattttgtatgtaccctatgaataagcatcggctaactccgtgccagcagccgcggtaatacggaggatgcgagcgttatccggatttattgggtttaaagggtgcgtaggtggtaatttaagtcagcggtgaaagtttgtggctcaaccataaaattgccgttgaaactgggttacttgagtgtgtttgaggtaggcggaatgcgtggtgtagcggtgaaatgcatagatatcacgcagaactccaattgcgaaggcagcttactaaaccataactgacactgaagcacgaaagcgtgggtatcaaacaggattagataccctggtagtccacgcagtaaacgatgattactaggagtttgcgatacacagtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtcagaccgaccttgaaagaggttttctagcaatagctgattacgaggtgctgcatggttgtcgtcagctcgtgccgtgaggtgtcggcttaagtgccataacgagcgcaacccttatcactagttactaacaggttaagctgaggactctggtgagactgccagcgtaagctgtgaggaaggtggggatgacgtcaaatcagcacggcccttacatccggggcgacacacgtgttacaatggcatggacaaagggcagctacctggcgacaggatgctaatctctaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaaggatcggcctagggtaaaactggtgactggggctaagtcgtaacaaggtagccgtaccggaaggtgcggctggaacacctcctt

SEQ ID NO: 107 - DNA, 1530 bp, Parabacteroides johnsonii; identical to SEQ ID NO: 106
  misc_feature: Parabacteroides johnsonii M-165

SEQ ID NO: 108 - DNA, 1368 bp, Ruminococcus
  misc_feature: Ruminococcus sp. 18P13
tcgaacggagataaagacttcggtttttatcttagtggcggacggttgagtaacacgtgagcaacctgcctttgagagagggatagcttctggaaacggatggtaatacctcataacatagcggtaccgcatgatactgctatcaaagatttatcgctcagagatgggctcgcgtctgattagctagatggtgaggtaacggttcaccatggcgacgatcagtagccggactgagaggttgaacggccacattgggactgagacacggcccagactcctacgggaggcagcagtgcggaatattgcacaatgacgcaagcctgatgcagcgatgccgcgtggaggaagaaggttttcggattgtaaactcctgtcttaagggacgataatgacggtaccttaggaggaagctccggctaactacgtgccagcagccgcggtaatacgtagggagcgagcgttgtccggaattactgggtgtaaagggagcgtaggcgggattgcaagtcagatgtgaaaactatgggcttaacccatagactgcatttgaaactgtagttcttgagtgaagtagaggtaagcggaattcctagtgtagcggtgaaatgcgtagatattaggaggaacatcggtggcgaaggcggcttactgggcttttactgacgctgaggctcgaaagcgtggggagcaaacaggattagataccctggtagtccacgctgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaaaaaccttaccaggtcttgacatcgagtgaatgatctagagatagatcagtccttcgggacacaaagacaggtggtgcatggttgtcgtcagctcgtgtcgtgagatgttgggttaagtcccgcaacgagcgcaacccttacctttagttgctacgcaagagcactctagagggactgccgttgacaaaacggaggaaggtggggatgacgtcaaatcatcatgccccttatgacctgggctacacacgtactacaatggcaatgaacagagggaagcaatacagtgatgtggagcaaatccccaaaaattgtcccagttcagattgtaggctgcaactcgcctacatgaagtcggaattgctagtaatctgcagctcagcatgctgcggtgaatacgttcccgggccttgtacacgccgcccgtcacaccatgggagtcggtaacacccgaagccagtagcctaaccgcaaggagggcgctgt

SEQ ID NO: 109 - DNA, 1510 bp, Ruminococcus
  misc_feature: Ruminococcus sp. 18P13
actttatgaagagtttgatcctggctcaggacgaacgctggcggcacgcctaacacatgcaagtcgaacggagataaagacttcggtttttatcttagtggcggacgggtgagtaacacgtgagcaacctgcctctgagagagggatagcttctggaaacggatggtaatacctcataacatagcggtaccgcatgatactgctatcaaagatttatcgctcagagatgggctcgcgtctgattagctagatggtgaggtaacggctcaccatggcgacgatcagtagccggactgagaggttgaacggccacattgggactgagacacggcccagactcctacgggaggcagcagtggggaatattgcacaatgggcgcaagcctgatgcagcgatgccgcgtggaggaagaaggttttcggattgtaaactcctgtcttaagggacgataatgacggtaccttaggaggaagctccggctaactacgtgccagcagccgcggtaatacgtagggagcgagcgttgtccggaattactgggtgtaaagggagcgtaggcgggattgcaagtcagatgtgaaaactatgggcttaacccatagactgcatttgaaactgtagttcttgagtgaagtagaggtaagcggaattcctagtgtagcggtgaaatgcgtagatattaggaggaacatcggtggcgaaggcggcttactgggcttttactgacgctgaggctcgaaagcgtggggagcaaacaggattagataccctggtagtccacgctgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaaaaaccttaccaggtcttgacatcgagtgaatgatctagagatagatcagtccttcgggacacaaagacaggtggtgcatggttgtcgtcagctcgtgtcgtgagatgttgggttaagtcccgcaacgagcgcaacccttacctttagttgctacgcaagagcactctagagggactgccgttgacaaaacggaggaaggtggggatgacgtcaaatcatcatgccccttatgacctgggctacacacgtactacaatggcaatgaacagagggaagcaatacagtgatgtggagcaaatccccaaaaattgtcccagttcagattgtaggctgcaactcgcctacatgaagtcggaattgctagtaatcgcagatcagcatgctgcggtgaatacgttcccgggccttgtacacaccgcccgtcacaccatgggagtcggtaacacccgaagccagtagcctaaccgcaaggagggcgctgtcgaaggtgggattgatgactggggtgaagtcgtaacaaggtagccgtatcggaaggtgcggctggatcacctcctttct

SEQ ID NO: 110 - DNA, 1494 bp, Ruminococcus
  misc_feature: Ruminococcus sp. 18P13
agagtttgatcctggctcaggacgaacgctggcggcacgcctaacacatgcaagtcgaacggagataaagacttcggtttttatcttagtggcggacgggtgagtaacacgtgagcaacctgcctctgagagagggatagcttctggaaacggatggtaatacctcataacatagcggtaccgcatgatactgctatcaaagatttatcgctcagagatgggctcgcgtctgattagctagatggtgaggtaacggctcaccatggcgacgatcagtagccggactgagaggttgaacggccacattgggactgagacacggcccagactcctacgggaggcagcagtggggaatattgcacaatgggcgcaagcctgatgcagcgatgccgcgtggaggaagaaggttttcggattgtaaactcctgtcttaagggacgataatgacggtaccttaggaggaagctccggctaactacgtgccagcagccgcggtaatacgtagggagcgagcgttgtccggaattactgggtgtaaagggagcgtaggcgggattgcaagtcagatgtgaaaactatgggcttaacccatagactgcatttgaaactgtagttcttgagtgaagtagaggtaagcggaattcctagtgtagcggtgaaatgcgtagatattaggaggaacatcggtggcgaaggcggcttactgggcttttactgacgctgaggctcgaaagcgtggggagcaaacaggattagataccctggtagtccacgctgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaaaaaccttaccaggtcttgacatcgagtgaatgatctagagatagatcagtccttcgggacacaaagacaggtggtgcatggttgtcgtcagctcgtgtcgtgagatgttgggttaagtcccgcaacgagcgcaacccttacctttagttgctacgcaagagcactctagagggactgccgttgacaaaacggaggaaggtggggatgacgtcaaatcatcatgccccttatgacctgggctacacacgtactacaatggcaatgaacagagggaagcaatacagtgatgtggagcaaatccccaaaaattgtcccagttcagattgtaggctgcaactcgcctacatgaagtcggaattgctagtaatcgcagatcagcatgctgcggtgaatacgttcccgggccttgtacacaccgcccgtcacaccatgggagtcggtaacacccgaagccagtagcctaaccgcaaggagggcgctgtcgaaggtgggattgatgactggggtgaagtcgtaacaaggtagccgtatcggaaggtgcggctggatcacct

SEQ ID NO: 111 - DNA, 1508 bp, Ruminococcus
  misc_feature: Ruminococcus sp. 18P13
tttatgaagagtttgatcctggctcaggacgaacgctggcggcacgcctaacacatgcaagtcgaacggagataaagacttcggtttttatcttagtggcggacgggtgagtaacacgtgagcaacctgcctctgagagagggatagcttctggaaacggatggtaatacctcataacatagcggtaccgcatgatactgctatcaaagatttatcgctcagagatgggctcgcgtctgattagctagatggtgaggtaacggctcaccatggcgacgatcagtagccggactgagaggttgaacggccacattgggactgagacacggcccagactcctacgggaggcagcagtggggaatattgcacaatgggcgcaagcctgatgcagcgatgccgcgtggaggaagaaggttttcggattgtaaactcctgtcttaagggacgataatgacggtaccttaggaggaagctccggctaactacgtgccagcagccgcggtaatacgtagggagcgagcgttgtccggaattactgggtgtaaagggagcgtaggcgggattgcaagtcagatgtgaaaactatgggcttaacccatagactgcatttgaaactgtagttcttgagtgaagtagaggtaagcggaattcctagtgtagcggtgaaatgcgtagatattaggaggaacatcggtggcgaaggcggcttactgggcttttactgacgctgaggctcgaaagcgtggggagcaaacaggattagataccctggtagtccacgctgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaaaaaccttaccaggtcttgacatcgagtgaatgatctagagatagatcagtccttcgggacacaaagacaggtggtgcatggttgtcgtcagctcgtgtcgtgagatgttgggttaagtcccgcaacgagcgcaacccttacctttagttgctacgcaagagcactctagagggactgccgttgacaaaacggaggaaggtggggatgacgtcaaatcatcatgccccttatgacctgggctacacacgtactacaatggcaatgaacagagggaagcaatacagtgatgtggagcaaatccccaaaaattgtcccagttcagattgtaggctgcaactcgcctacatgaagtcggaattgctagtaatcgcagatcagcatgctgcggtgaatacgttcccgggccttgtacacaccgcccgtcacaccatggagtcggtaacacccgaagccagtagcctaaccgcaaggagggcgctgtcgaaggtgggattgatgactggggtgaagtcgtaacaaggtagccgtatcggaaggtgcggctggatcacctcctttcta

SEQ ID NO: 112 - DNA, 1515 bp, Slackia piriformis
  misc_feature: Slackia piriformis DSM 22477
ttcgaacggagagtttgatcctggctcaggatgaacgctggcggcgtgcctaacacatgcaagtcgaacggttaaggcgccttcgggcgcgcatagagtggcgaacgggtgagtaacacgtgaccaacctgccccctcctccgggacaacctcgggaaaccgaggctaataccggatggtcccgccgggccgcatggccgggcggggaaagcccaggcgggaggggatggggtcgcggcccatcaggtagtaggcggggtgacggcccacctagctgacgacgggtagccgcgctgagaggcggaccggccacattgggactgagacacggcccagactcctacgggaggcagcagtggggaattttgcgcaatgggggaaaccctgacgcagcaacgccgcgtgcgggacgaaggccttcgggtcgtaaaccgctttcagcagggaagaagaatgacggtacctgcagaagaagccccggctaactacgtgccagcagccgcggtaatacgtagggggcgagcgttatccggattcattgggcgtaaagcgcgcgtaggcgtcccttcaagcggcaccgtcgaagccgggggctcaacccccggaagcgggccgaactggggggatcgagtgcggtaggggaaggcggaattcccggtgtagcggtgaaatgcgcagatatcgggaagaacaccgacggcgaaggcagccttctgggccgccactgacgctgaggcgcgaaagctgggggagcgaacaggattagataccctggtagtcccagccgtaaacgatgggcgctaggtgtggggggagatgtccctccgtgccgcagccaacgcattaagcgccccgcctggggagtacggccgcaaggctaaaactcaaaggaattgacgggggcccgcacaagcagcggagcatgtggcttaattcgaagcaacgcgaagaaccttaccagggcttgacatcaccgtgagccgccggagacggcggggccgaaaggagcggtgacaggtggtgcatggctgtcgtcagctcgtgtcgtgagatgttgggttaagtcccgcaacgagcgcaacccccgccgcgtgttgccagcattgagttgggcactcgcgcgggactgccggcgtcaagccggaggaaggtggggacgacgtcaagtcatcatgcccctcatgccctgggctgcacacgtgctacaatggccggtacagcgggctgcgacgccgcgaggcggagcgaatcccacaaagccggccccagttcggaccgcaggctgcaacccgcctgcgcgaagccggagttgctagtaatcgcggatcagcatgccgcggtgaatacgttcccgggccttgtacacaccgcccgtcacaccacccgagtcgtctgcacccgaagccgccggccgaacccgcaaggggcggaggcgtcgaaggtgtggagggtgaggggggtgaagtcgtaacaaggtagccgtaccggaaggtgcggctggatcacctcctttct

SEQ ID NO: 113 - DNA, 1515 bp, Slackia piriformis; identical to SEQ ID NO: 112
  misc_feature: Slackia piriformis DSM 22477

SEQ ID NO: 114 - DNA, 1521 bp, Slackia piriformis
  misc_feature: Slackia piriformis DSM 22477
ttcgaacggagagtttgatcctggctcaggatgaacgctggcggcgtgcctaacacatgcaagtcgaacggttaaggcgccttcgggcgcgcatagagtggcgaacgggggagtaacacgtgaccaacctgccccctcctccgggacaacctcgggaaaccgaggctaataccggatggtcccgccgggccgcatggccgggcggggaaagcccaggcgggaggggatggggtcgcggcccatcaggtagtaggcggggtgacggcccacctagctgacgacgggtagccgcgctgagaggcggaccggccacattgggactgagacacggcccagactcctacgggaggcagcagtggggaattttgcgcaatgggggaaaccctgacgcagcaacgccgcgtgcgggacgaaggccttcgggtcgtaaaccgctttcagcagggaagaagaatgacggtacctgcagaagaagccccggctaactacgtgccagcagccgcggtaatacgtagggggcgagcgttatccggattcattgggcgtaaagcgcgcgtaggcgtcccttcaagcggcaccgtcgaagccgggggctcaacccccggaagcgggccgaactggggggatcgagtgcggtaggggaaggcggaattcccggtgtagcggtgaaatgcgcagatatcgggaagaacaccgacggcgaaggcagccttctgggccgccactgacgctgaggcgcgaaagctgggggagcgaacaggattagataccctggtagtcccagccgtaaacgatgggcgctaggtgtggggggagatgtccctccgtgccgcagccaacgcattaagcgccccgcctggggagtacggccgcaaggctaaaactcaaaggaattgacgggggcccgcacaagcagcggagcatgtggcttaattcgaagcaacgcgaagaaccttaccagggcttgacatcaccgtgagccgccggagacggcggggccgaaaggagcggtgacaggtggtgcatggctgtcgtcagctcgtgtcgtgagatgttgggttaagtcccgcaacgagcgcaacccccgccgcgtgttgccagcattgagttgggcactcgcgcgggactgccggcgtcaagccggaggaagggggggacgacgtcaagtcatcatgcccctcatgccctgggctgcacacgtgctacaatggccggtacagcgggctgcgacgccgcgaggcggagcgaatcccacaaagccggccccagttcggaccgcaggctgcaacccgcctgcgcgaagccggagttgctagtaatcgcggatcagcatgccgcggtgaatacgttcccgggccttgtacacaccgcccgtcacaccacccgagtcgtctgcacccgaagccgccggccgaacccgcaaggggcggaggcgtcgaaggtgtggagggggaggggggtgaagtcgtaacaaggtagccgtaccggaaggtgcggctggatcacctcctttctagggag

SEQ ID NO: 115 - DNA, 1521 bp, Slackia piriformis
  misc_feature: Slackia piriformis DSM 22477
ttcgaacggagagtttgatcctggctcaggatgaacgctggcggcgtgcctaacacatgcaagtcgaacggttaaggcgccttcgggcgcgcatagagtggcgaacgggtgagtaacacgtgaccaacctgccccctcctccgggacaacctcgggaaaccgaggctaataccggatggtcccgccgggccgcatggccgggcggggaaagcccaggcgggaggggatggggtcgcggcccatcaggtagtaggcggggtgacggcccacctagctgacgacgggtagccgcgctgagaggcggaccggccacattgggactgagacacggcccagactcctacgggaggcagcagtggggaattttgcgcaatgggggaaaccctgacgcagcaacgccgcgtgcgggacgaaggccttcgggtcgtaaaccgctttcagcagggaagaagaatgacggtacctgcagaagaagccccggctaactacgtgccagcagccgcggtaatacgtagggggcgagcgttatccggattcattgggcgtaaagcgcgcgtaggcgtcccttcaagcggcaccgtcgaagccgggggctcaacccccggaagcgggccgaactggggggatcgagtgcggtaggggaaggcggaattcccggtgtagcggtgaaatgcgcagatatcgggaagaacaccgacggcgaaggcagccttctgggccgccactgacgctgaggcgcgaaagctgggggagcgaacaggattagataccctggtagtcccagccgtaaacgatgggcgctaggtgtggggggagatgtccctccgtgccgcagccaacgcattaagcgccccgcctggggagtacggccgcaaggctaaaactcaaaggaattgacgggggcccgcacaagcagcggagcatgtggcttaattcgaagcaacgcgaagaaccttaccagggcttgacatcaccgtgagccgccggagacggcggggccgaaaggagcggtgacaggtggtgcatggctgtcgtcagctcgtgtcgtgagatgttgggttaagtcccgcaacgagcgcaacccccgccgcgtgttgccagcattgagttgggcactcgcgcgggactgccggcgtcaagccggaggaaggtggggacgacgtcaagtcatcatgcccctcatgccctgggctgcacacgtgctacaatggccggtacagcgggctgcgacgccgcgaggcggagcgaatcccacaaagccggccccagttcggaccgcaggctgcaacccgcctgcgcgaagccggagttgctagtaatcgcggatcagcatgccgcggtgaatacgttcccgggccttgtacacaccgcccgtcacaccacccgagtcgtctgcacccgaagccgccggccgaacccgcaaggggcggaggcgtcgaaggtgtggagggtgaggggggtgaagtcgtaacaaggtagccgtaccggaaggtgcggctggatcacctcctttctagggag

SEQ ID NO: 116 - DNA, 1521 bp, Slackia piriformis; identical to SEQ ID NO: 115
  misc_feature: Slackia piriformis DSM 22477

SEQ ID NO: 117 - DNA, 1472 bp, Slackia piriformis
  misc_feature: Slackia piriformis DSM 22477
agagtttgatcctggctcaggatgaacgctggcggcgtgcctaacacatgcaagtcgaacggttaaggcgccttcgggcgcgcatagagtggcgaacgggtgagtaacacgtgaccaacctgccccctcctccgggacaacctcgggaaaccgaggctaataccggatggtcccgccgggccgcatggccgggcggggaaagcccaggcgggaggggatggggtcgcggcccatcaggtagtaggcggggtgacggcccacctagctgacgacgggtagccgcgctgagaggcggaccggccacattgggactgagacacggcccagactcctacgggaggcagcagtggggaattttgcgcaatgggggaaaccctgacgcagcaacgccgcgtgcgggacgaaggccttcgggtcgtaaaccgctttcagcagggaagaagaatgacggtacctgcagaagaagccccggctaactacgtgccagcagccgcggtaatacgtagggggcgagcgttatccggattcattgggcgtaaagcgcgcgtaggcgtcccttcaagcggcaccgtcgaagccgggggctcaacccccggaagcgggccgaactggggggatcgagtgcggtaggggaaggcggaattcccggtgtagcggtgaaatgcgcagatatcgggaagaacaccgacggcgaaggcagccttctgggccgccactgacgctgaggcgcgaaagctgggggagcgaacaggattagataccctggtagtcccagccgtaaacgatgggcgctaggtgtggggggagatgtccctccgtgccgcagccaacgcattaagcgccccgcctggggagtacggccgcaaggctaaaactcaaaggaattgacgggggcccgcacaagcagcggagcatgtggcttaattcgaagcaacgcgaagaaccttaccagggcttgacatcaccgtgagccgccggagacggcggggccgaaaggagcggtgacaggtggtgcatggctgtcgtcagctcgtgtcgtgagatgttgggttaagtcccgcaacgagcgcaacccccgccgcgtgttgccagcattgagttgggcactcgcgcgggactgccggcgtcaagccggaggaaggtggggacgacgtcaagtcatcatgcccctcatgccctgggctgcacacgtgctacaatggccggtacagcgggctgcgacgccgcgaggcggagcgaatcccacaaagccggccccagttcggaccgcaggctgcaacccgcctgcgcgaagccggagttgctagtaatcgcggatcagcatgccgcggtgaatacgttcccgggccttgtacacaccgcccgtcacaccacccgagtcgtctgcacccgaagccgccggccgaacccgcaaggggcggaggcgtcgaaggtgtggagggtgaggggggtgaagtcgtaacaaggtaacc

SEQ ID NO: 118 - DNA, 1414 bp, Streptococcus hongkongensis
  misc_feature: Streptococcus hongkongensis HKU30
agtagaacgctgaggactggtgcttgcactagtctaaggagttgcgaacgggtgagtaacgcgtaggtaacctacctcatagcgggggataactattggaaacgatagctaataccgcatgacactggagtacacatgtacttgagttaaaaggagcaattgcttcactatgagatggacctgcgttgtattagctagttggtgaggtaatggctcaccaaggcgacgatacatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgttagagaagaacgatgatgggagtggaaaatccatcatgtgacggtaactaaccagaaagggacggctaactacgtgccagcagccgcggtaatacgtaggtctcgagcgttgtccggatttattgggcgtaaagcgagcgcaggcggtttgataagtctgaagttaaaggctgtggctcaaccatagttcgctttggaaactgtcaaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttaggccctttccggggcttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccttctgaccgtcctagagataggattttccttagggacagaagtgacaggtggtgcatggttgtcgtcagctcgtgtcgtgagatgttgggttaagtcccgcaacgagcgcaacccctattgttagttgccatcattaagttgggcactctagcgagactgccggtaataaaccggaggaaggtggggatgacgtcaaatcatcatgccccttatgacctgggctacacacgtgctacaatggttggtacaacgagtcgcaagccggtgacggcaagctaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaacctattaggagccagccgcc

SEQ ID NO: 119 - DNA, 1414 bp, Streptococcus hongkongensis
  misc_feature: Streptococcus hongkongensis HKU30
agtagaacgctgaggactggtgcttgcactagtctaaggagttgcgaacgggtgagtaacgcgtaggtaacctacctcatagcgggggataactattggaaacgatagctaataccgcatgacactggagtacacatgtacttgagttaaaaggagcaattgcttcactatgagatggacctgcgttgtattagctagttggtgaggtaatggctcaccaaggcgacgatacatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgttagagaagaacgatgatgggagtggaaaatccatcatgtgacggtaactaaccagaaagggacggctaactacgtgccagcagccgcggtaatacgtaggtctcgagcgttgtccggatttattgggcgtaaagcgagcgcaggcggtttgataagtctgaagttaaaggctgtggctcaaccatagttcgctttggaaactgtcaaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttaggccctttccggggcttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccttctgaccgtcctagagataggattttccttagggacagaagtgacaggtggtgcatggttgtcgtcagctcgtgtcgtgagatgttgggttaagtcccgcaacgagcgcaacccctattgttagttgccatcattaagttgggcactctagcgagactgccggtaataaaccggaggaaggtggggatgacgtcaaatcatcatgccccttatgacctgggctacacacgtgctacaatggttggtacaacgagtcgcaagccggtgacggcaagctaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttataggagccagccgcc

SEQ ID NO: 120 - DNA, 1532 bp, Alistipes indistinctus
  misc_feature: Alistipes indistinctus DSM 22520
tacaatggagagtttgatcctggctcaggataaacgctagcggcaggcctaacacatgcaagtcgaggggcagcgggtggagtatttcggtactcctgccggcgaccggcgcacgggtgcgtaacgcgtatgcaacctacctttaacagggggataatccgaagaaatttggtctaataccccataatatcatttaaggcatcttagatggttgaaaattccgatggttagagatgggcatgcgttgtattagctagttggtgaggtaacggctcaccaaggctacgatacatagggggactgagaggttttccccccacactggtactgagacacggaccagactcctacgggaggcagcagtgaggaatattggtcaatggacgcaagtctgaaccagccatgccgcgtgcaggatgaaggtgctatgcattgtaaactgcttttgtacgagggtaaatgcaggtacgtgtacctgtttgaaagtatcgtacgaataagggtcggctaactccgtgccagcagccgcggtaatacggaggacccgagcgttatccggatttattgggtttaaagggtgcgtaggcggattagtaagttagaggtgaaagctcgatgctcaacatcgaaattgcctctgatactgttagtctagagtatagttgcggaaggcggaatgtgtggtgtagcggtgaaatgcttagatatcacacagaacaccgattgcgaaggcagctttccaagctattactgacgctgatgcacgaaagcgtggggagcgaacaggattagataccctggtagtccacgccgtaaacgatgataactcgttgcaggcgatacacagtctgtgacttagcgaaagcgttaagttatccacctggggagtacgttcgcaagaatgaaactcaaaggaattgacgggggcccgcacaagcggaggaacatgtggtttaattcgatgatacgcgaggaaccttacccgggcttgaaagttagcgacggatcctgaaaggggtcttctcttcggagcgcgaaactaggtgctgcatggttgtcgtcagctcgtgccgtgaggtgtcgggttaagtcccataacgagcgcaacccctactgttagttaccagcacgtcaaggtgggcactctagcaggactgccggtgtaagccgagaggaaggtggggatgacgtcaaatcagcacggcccttacgtccggggcgacacacgtgttacaatggtcggtacagagggtcgctaccccgtgaggggatgccaatctcgaaagccgatctcagttcggattggaggctgaaactcgcctccatgaagttggattcgctagtaatcgcgcatcagccatggcgcggtgaatacgttcccgggccttgtacacaccgcccgtcaagccatgggagttgggggtgcctgaagtacgtgaccgcaaggagcgtcctagggcaaaaccgatgactggggctaagtcgtaacaaggtagccgtaccggaaggtgcggctggaacacctcctttct

SEQ ID NO: 121 - DNA, 1532 bp, Alistipes indistinctus; identical to SEQ ID NO: 120
  misc_feature: Alistipes indistinctus DSM 22520

SEQ ID NO: 122 - DNA, 1535 bp, Alistipes indistinctus
  misc_feature: Alistipes indistinctus DSM 22520
tacaatggagagtttgatcctggctcaggataaacgctagcggcaggcctaacacatgcaagtcgaggggcagcgggtggagtatttcggtactcctgccggcgaccggcgcacgggtgcgtaacgcgtatgcaacctacctttaacagggggataatccgaagaaatttggtctaataccccataatatcatttaaggcatcttagatggttgaaaattccgatggttagagatgggcatgcgttgtattagctagttggtgaggtaacggctcaccaaggctacgatacatagggggactgagaggttttccccccacactggtactgagacacggaccagactcctacgggaggcagcagtgaggaatattggtcaatggacgcaagtctgaaccagccatgccgcgtgcaggatgaaggtgctatgcattgtaaactgcttttgtacgagggtaaatgcaggtacgtgtacctgtttgaaagtatcgtacgaataagggtcggctaactccgtgccagcagccgcggtaatacggaggacccgagcgttatccggatttattgggtttaaagggtgcgtaggcggattagtaagttagaggtgaaagctcgatgctcaacatcgaaattgcctctgatactgttagtctagagtatagttgcggaaggcggaatgtgtggtgtagcggtgaaatgcttagatatcacacagaacaccgattgcgaaggcagctttccaagctattactgacgctgatgcacgaaagcgtggggagcgaacaggattagataccctggtagtccacgccgtaaacgatgataactcgttgcaggcgatacacagtctgtgacttagcgaaagcgttaagttatccacctggggagtacgttcgcaagaatgaaactcaaaggaattgacgggggcccgcacaagcggaggaacatgtggtttaattcgatgatacgcgaggaaccttacccgggcttgaaagttagcgacggatcctgaaaggggtcttctcttcggagcgcgaaactaggtgctgcatggttgtcgtcagctcgtgccgtgaggtgtcgggttaagtcccataacgagcgcaacccctactgttagttaccagcacgtcaaggtgggcactctagcaggactgccggtgtaagccgagaggaaggtggggatgacgtcaaatcagcacggcccttacgtccggggcgacacacgtgttacaatggtcggtacagagggtcgctaccccgtgaggggatgccaatctcgaaagccgatctcagttcggattggaggctgaaactcgcctccatgaagttggattcgctagtaatcgcgcatcagccatggcgcggtgaatacgttcccgggccttgtacacaccgcccgtcaagccatgggagttgggggtgcctgaagtacgtgaccgcaaggagcgtcctagggcaaaaccgatgactggggctaagtcgtaacaaggtagccgtaccggaaggtgcggctggaacacctcctttctgga

SEQ ID NO: 123 - DNA, 1482 bp, Alistipes indistinctus
  misc_feature: Alistipes indistinctus DSM 22520
gataaacgctagcggcaggcctaacacatgcaagtcgaggggcagcgggtggagtatttcggtactcctgccggcgaccggcgcacgggtgcgtaacgcgtatgcaacctacctttaacagggggataatccgaagaaatttggtctaataccccataatatcatttaaggcatcttagatggttgaaaattccgatggttagagatgggcatgcgttgtattagctagttggtgaggtaacggctcaccaaggctacgatacatagggggactgagaggttttccccccacactggtactgagacacggaccagactcctacgggaggcagcagtgaggaatattggtcaatggacgcaagtctgaaccagccatgccgcgtgcaggatgaaggtgctatgcattgtaaactgcttttgtacgagggtaaatgcaggtacgtgtacctgtttgaaagtatcgtacgaataagggtcggctaactccgtgccagcagccgcggtaatacggaggacccgagcgttatccggatttattgggtttaaagggtgcgtaggcggattagtaagttagaggtgaaagctcgatgctcaacatcgaaattgcctctgatactgttagtctagagtatagttgcggaaggcggaatgtgtggtgtagcggtgaaatgcttagatatcacacagaacaccgattgcgaaggcagctttccaagctattactgacgctgatgcacgaaagcgtggggagcgaacaggattagataccctggtagtccacgccgtaaacgatgataactcgttgcaggcgatacacagtctgtgacttagcgaaagcgttaagttatccacctggggagtacgttcgcaagaatgaaactcaaaggaattgacgggggcccgcacaagcggaggaacatgtggtttaattcgatgatacgcgaggaaccttacccgggcttgaaagttagcgacggatcctgaaaggggtcttctcttcggagcgcgaaactaggtgctgcatggttgtcgtcagctcgtgccgtgaggtgtcgggttaagtcccataacgagcgcaacccctactgttagttaccagcacgtcaaggtgggcactctagcaggactgccggtgtaagccgagaggaaggtggggatgacgtcaaatcagcacggcccttacgtccggggcgacacacgtgttacaatggtcggtacagagggtcgctaccccgtgaggggatgccaatctcgaaagccgatctcagttcggattggaggctgaaactcgcctccatgaagttggattcgctagtaatcgcgcatcagccatggcgcggtgaatacgttcccgggccttgtacacaccgcccgtcaagccatgggagttgggggtgcctgaagtacgtgaccgcaaggagcgtcctagggcaaaaccgatgactggggcctaagtcgtaacaaggtagccgtaccggaagg

SEQ ID NO: 124 - DNA, 1490 bp, Alistipes indistinctus
  misc_feature: Alistipes indistinctus DSM 22520
agagtttgatcctggctcaggataaacgctagcggcaggcctaacacatgcaagtcgaggggcagcgggtggagtatttcggtactcctgccggcgaccggcgcacgggtgcgtaacgcgtatgcaacctacctttaacagggggataatccgaagaaatttggtctaataccccataatatcatttaaggcatcttagatggttgaaaattccgatggttagagatgggcatgcgttgtattagctagttggtgaggtaacggctcaccaaggctacgatacatagggggactgagaggttttccccccacactggtactgagacacggaccagactcctacgggaggcagcagtgaggaatattggtcaatggacgcaagtctgaaccagccatgccgcgtgcaggatgaaggtgctatgcattgtaaactgcttttgtacgagggtaaatgcaggtacgtgtacctgtttgaaagtatcgtacgaataagggtcggctaactccgtgccagcagccgcggtaatacggaggacccgagcgttatccggatttattgggtttaaagggtgcgtaggcggattagtaagttagaggtgaaagctcgatgctcaacatcgaaattgcctctgatactgttagtctagagtatagttgcggaaggcggaatgtgtggtgtagcggtgaaatgcttagatatcacacagaacaccgattgcgaaggcagctttccaagctattactgacgctgatgcacgaaagcgtggggagcgaacaggattagataccctggtagtccacgccgtaaacgatgataactcgttgcaggcgatacacagtctgtgacttagcgaaagcgttaagttatccacctggggagtacgttcgcaagaatgaaactcaaaggaattgacgggggcccgcacaagcggaggaacatgtggtttaattcgatgatacgcgaggaaccttacccgggcttgaaagttagcgacggatcctgaaaggggtcttctcttcggagcgcgaaactaggtgctgcatggttgtcgtcagctcgtgccgtgaggtgtcgggttaagtcccataacgagcgcaacccctactgttagttaccagcacgtcaaggtgggcactctagcaggactgccggtgtaagccgagaggaaggtggggatgacgtcaaatcagcacggcccttacgtccggggcgacacacgtgttacaatggtcggtacagagggtcgctaccccgtgaggggatgccaatctcgaaagccgatctcagttcggattggaggctgaaactcgcctccatgaagttggattcgctagtaatcgcgcatcagccatggcgcggtgaatacgttcccgggccttgtacacaccgcccgtcaagccatgggagttgggggtgcctgaagtacgtgaccgcaaggagcgtcctagggcaaaaccgatgactggggctaagtcgtaacaaggtaacc

SEQ ID NO: 125 - DNA, 1465 bp, Azotobacter
  misc_feature: Azotobacter sp. A1 2
  misc_feature (46)..(46): n is a, c, g, or t
  misc_feature (50)..(50): n is a, c, g, or t
  misc_feature (63)..(63): n is a, c, g, or t
  misc_feature (66)..(66): n is a, c, g, or t
atgaacgctagcggcaggcttaacacatgcaagtcgaggggcagcngggngtagcaatactcngcnggcgaccggcgcacgggtgcgtaacgcgtatgcaacctacctttaacaggggcataacactgagaaattggtactaattccccataacattcgagaaggcatcttcttgggttaaaaactccggtggttaaagatgggcatgcgttgtattagctagttggtgaggtaacggctcaccaaggcaacgatacatagggggactgagaggttaaccccccacattggtactgagacacggaccaaactcctacgggaggcagcagtgaggaatattggtcaatggacgcaagtctgaaccagccatgccgcgtgcaggaagacggctctatgagttgtaaactgcttttgtactagggtaaacgcttacgtgtaggagcctgaaagtatagtacgaataaggatcggctaactccgtgccagcagccgcggtaatacggaggatccaagcgttatccggatttattgggtttaaagggtgcgtaggcggtttgataagttagaggtgaaataccggggctcaactccggaactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagtgacgattctggaaacaggatttcccttcggggcacgaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggccgctaccccgcgaggggatgccaatctcgaaagcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaagttcgtgaccgcaaggagcgacctagggcaaaaccggtgactggggctaagtcgtaacaaggtagccgta

SEQ ID NO: 126 - DNA, 1445 bp, Azotobacter
  misc_feature: Azotobacter sp. A1 2
gatgaacgctagcggcaggcttaacacatgcaagtcgaggggcagcggggagtagcaatactccgccggcgaccggcgcacgggtgcgtaacgcgtatgcaacctacctttaacaggggcataacactgagaaattggtactaattccccataacattcgagaaggcatcttcttgggttaaaaactccggtggttaaagatgggcatgcgttgtattagctagttggtgaggtaacggctcaccaaggcaacgatacatagggggactgagaggttaaccccccacattggtactgagacacggaccaaactcctacgggaggcagcagtgaggaatattggtcaatggacgcaagtctgaaccagccatgccgcgtgcaggaagacggctctatgagttgtaaactgcttttgtactagggtaaacgcttctacgtgtaggagcctgaaagtatagtacgaataaggatcggctaactccgtgccagcagccgcggtaatacggaggatccaagcgttatccggatttattgggtttaaagggtgcgtaggcggtttgataagttagaggtgaaataccggggctcaactccggaactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagtgacgattctggaaacaggatttcccttcggggcacgaaactaggtgctgcatggttgttgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggccgctaccccgcgaggggatgccaatctcgaaggcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaagttcgtgaccgcaaggagcgacctagggcaaaaccggtgactggggc

SEQ ID NO: 127 - DNA, 1445 bp, Azotobacter
  misc_feature: Azotobacter sp. A1 2
gatgaacgctagcggcaggcttaacacatgcaagtcgaggggcagcacgaggtagcaatactttggtggcgaccggcgcacgggtgcgtaacgcgtatgcaacctacctttaacaggggcataacactgagaaattggtactaattccccataacattcgagaaggcatcttcttgggttaaaaactccggtggttaaagatgggcatgcgttgtattagctagttggtgaggtaacggctcaccaaggcaacgatacatagggggactgagaggttaaccccccacattggtactgagacacggaccaaactcctacgggaggcagcagtgaggaatattggtcaatggacgcaagtctgaaccagccatgccgcgtgcaggaagacggctctatgagttgtaaactgcttttgtactagggtaaacgctcttacgtgtaggagcctgaaagtatagtacgaataaggatcggctaactccgtgccagcagccgcggtaatacggaggatccaagcgttatccggatttattgggtttaaagggtgcgtaggcggtttgataagttagaggtgaaataccggggctcaactccggaactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagtgacgattctggaaacaggatttcccttcggggcacgaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggccgctaccccgcgaggggatgccaatctcgaaagcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaagttcgtgaccgcaaggagcgacctagggcaaaaccggtgactggggc

SEQ ID NO: 128 - DNA, 1528 bp, Azotobacter
  misc_feature: Azotobacter sp. A1 2
atacaatggagagtttgatcctggctcaggatgaacgctagcggcaggcttaacacatgcaagtcgaggggcagcacgaggtagcaatactttggtggcgaccggcgcacgggtgcgtaacgcgtatgcaacctacctttaacaggggcataacactgagaaattggtactaattccccataacattcgagaaggcatcttcttgggttaaaaactccggtggttaaagatgggcatgcgttgtattagctagttggtgaggtaacggctcaccaaggcaacgatacatagggggactgagaggttaaccccccacattggtactgagacacggaccaaactcctacgggaggcagcagtgaggaatattggtcaatggacgcaagtctgaaccagccatgccgcgtgcaggaagacggctctatgagttgtaaactgcttttgtactagggtaaacgctcttacgtgtaggagcctgaaagtatagtacgaataaggatcggctaactccgtgccagcagccgcggtaatacggaggatccaagcgttatccggatttattgggtttaaagggtgcgtaggcggtttgataagttagaggtgaaataccggggctcaactccggaactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagtgacgattctggaaacaggatttcccttcggggcacgaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggccgctaccccgcgaggggatgccaatctcgaaagcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaagttcgtgaccgcaaggagcgacctagggcaaaaccggtgactggggctaagtcgtaacaaggtagccgtaccggaaggtgcggctggaacacctcctttct

SEQ ID NO: 129 - DNA, 1532 bp, Azotobacter
  misc_feature: Azotobacter sp. A1 2
tacaatggagagtttgatcctggctcaggatgaacgctagcggcaggcttaacacatgcaagtcgaggggcagcacgaggtagcaatactttggtggcgaccggcgcacgggtgcgtaacgcgtatgcaacctacctttaacaggggcataacactgagaaattggtactaattccccataacattcgagaaggcatcttcttgggttaaaaactccggtggttaaagatgggcatgcgttgtattagctagttggtgaggtaacggctcaccaaggcaacgatacatagggggactgagaggttaaccccccacattggtactgagacacggaccaaactcctacgggaggcagcagtgaggaatattggtcaatggacgcaagtctgaaccagccatgccgcgtgcaggaagacggctctatgagttgtaaactgcttttgtactagggtaaacgctcttacgtgtaggagcctgaaagtatagtacgaataaggatcggctaactccgtgccagcagccgcggtaatacggaggatccaagcgttatccggatttattgggtttaaagggtgcgtaggcggtttgataagttagaggtgaaataccggggctcaactccggaactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagtgacgattctggaaacaggatttcccttcggggcacgaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggccgctaccccgcgaggggatgccaatctcgaaagcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaagttcgtgaccgcaaggagcgacctagggcaaaaccggtgactggggctaagtcgtaacaaggtagccgtaccggaaggtgcggctggaacacctcctttctggagc

SEQ ID NO: 130 - DNA, 1531 bp, Azotobacter
  misc_feature: Azotobacter sp. A1 2
tacaatggagagtttgatcctggctcaggatgaacgctagcggcaggcttaacacatgcaagtcgaggggcagcggggagtagcaatactccgccggcgaccggcgcacgggtgcgtaacgcgtatgcaacctacctttaacaggggcataacactgagaaattggtactaattccccataacattcgagaaggcatcttcttgggttaaaaactccggtggttaaagatgggcatgcgttgtattagctagttggtgaggtaacggctcaccaaggcaacgatacatagggggactgagaggttaaccccccacattggtactgagacacggaccaaactcctacgggaggcagcagtgaggaatattggtcaatggacgcaagtctgaaccagccatgccgcgtgcaggaagacggctctatgagttgtaaactgcttttgtactagggtaaacgcttctacgtgtaggagcctgaaagtatagtacgaataaggatcggctaactccgtgccagcagccgcggtaatacggaggatccaagcgttatccggatttattgggtttaaagggtgcgtaggcggtttgataagttagaggtgaaataccggggctcaactccggaactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagtgacgattctggaaacaggatttcccttcggggcacgaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggccgctaccccgcgaggggatgccaatctcgaaagcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaagttcgtgaccgcaaggagcgacctagggcaaaaccggtgactggggctaagtcgtaacaaggtagccgtaccggaaggtgcggctggaacacctcctttctggag

SEQ ID NO: 131 - DNA, 1485 bp, Azotobacter
  misc_feature: Azotobacter sp. A1 2
agagtttgatcctggctcaggatgaacgctagcggcaggcttaacacatgcaagtcgaggggcagcggggagtagcaatactccgccggcgaccggcgcacgggtgcgtaacgcgtatgcaacctacctttaacaggggcataacactgagaaattggtactaattccccataacattcgagaaggcatcttcttgggttaaaaactccggtggttaaagatgggcatgcgttgtattagctagttggtgaggtaacggctcaccaaggcaacgatacatagggggactgagaggttaaccccccacattggtactgagacacggaccaaactcctacgggaggcagcagtgaggaatattggtcaatggacgcaagtctgaaccagccatgccgcgtgcaggaagacggctctatgagttgtaaactgcttttgtactagggtaaacgcttttacgtgtaggagcctgaaagtatagtacgaataaggatcggctaactccgtgccagcagccgcggtaatacggaggatccaagcgttatccggatttattgggtttaaagggtgcgtaggcggtttgataagttagaggtgaaataccggggctcaactccggaactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagtgacgattctggaaacaggatttcccttcggggcacgaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggccgctaccccgcgaggggatgccaatctcgaaagcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaagttcgtgaccgcaaggagcgacctagggcaaaaccggtgactggggctaagtcgtaacaaggtaacc

SEQ ID NO: 132 - DNA, 1476 bp, Bacteroides asaccharolyticus
  misc_feature: Bacteroides asaccharolyticus B440
  misc_feature (337)..(337): n is a, c, g, or t
  misc_feature (438)..(438): n is a, c, g, or t
  misc_feature (474)..(474): n is a, c, g, or t
  misc_feature (900)..(900): n is a, c, g, or t
  misc_feature (1017)..(1017): n is a, c, g, or t
  misc_feature (1029)..(1030): n is a, c, g, or t
acaatggagagtttgactctggctcaggatgaacgctagcgataggcttaacacatgcaagtcgaggggcagcgagaygtagcaatacgtcgtcggcgaccggcgaatgggtgagtaacacgtatgcaacttacctcttagtggtgaataacccgatgaaagtcggactaatacaccatatactccttagatcccatgagaagaggaggaaagattaatcgctaagagatgggcctgcgttccattagctagttggtaaggtaacggcttaccaaggcaacgatggatagggggactgagaggttgaccccccacattgacactgagatacgggtcnaactcctacgggaggcagcagtgaggaatattggtcaatgggcgagagcctgaaccagccaagtcgcgtgaaggaagactgcccgcaagggttgtaaactncttttgtatgggattaaagtcacctacgtgtaggtntttgcagttaccatacgaataagcatcggctaactccgtgccagcagccgcggtaatacggaggatgcgagcgttatccggaattattgggtttaaagggtgcgtaggttgcaagggaagtcaggggtgaaaagctgtagctcaactatggtcttgcctttgaaactctctagctagagtgtactggaggtacgtggaacgtgtggtgtagcggtgaaatgcatagatatcacacagaactccgattgcgcaggcagcgtactacattacaactgacactgaagcacgaaagcgtgggtatccaacaggattagataccctggtagtccacgcagtaaacgatgaatactagatctatgcgatatacagtatgggtctaagcgaaagcgataagtattccacctggggagtacgccggcaacggtnaaactcaaagagattggcgggggtccgcacaagcggaggaacatgtggtttaattcgatgatacgcgaggaaccttacccgggattgaaatgtagatgcatgaggctgagaggtctnttccttcggggnntctatgtaggtgctgcatggttgtcgtcagctcgtgccgtgaggtgtcggcttaagtgccataacgagcgcaacccgcgtcgatagttactaacgagtcaagtcgaggactctatcgagacagccgtcgtaagacgtgaggaaggagcggatgacgtcaaatcagcacggcccttacatccggggcgacacacgtgttacaatggtagggacagcgagcagccatctggtgacagagagctaatctataaaccctatcccagttcggatcggagtctgcaactcgactctgtgaagctggattcgctagtaatcgcgcatcagccatggcgcggtgaatacgttcccggaccttgcacacaccgcccgtcaagccatgggagtcgggggtacctgaagagcgtgaccgtcacaggagcgcttgagggtaaaactggtgactggggc

SEQ ID NO: 133 - DNA, 1533 bp, Bacteroides asaccharolyticus
  misc_feature: Bacteroides asaccharolyticus B440
taacaatggagagtttgactctggctcaggatgaacgctagcgataggcttaacacatgcaagtcgaggggcagcgagatgtagcaatacgtcgtcggcgaccggcgaatgggtgagtaacacgtatgcaacttacctcttagtggtgaataacccgatgaaagtcggactaatacaccatatactccttagatcccatgagaagaggaggaaagattaatcgctaagagatgggcctgcgttccattagctagttggtaaggtaacggcttaccaaggcaacgatggatagggggactgagaggttgaccccccacattgacactgagatacgggtcaaactcctacgggaggcagcagtgaggaatattggtcaatgggcgagagcctgaaccagccaagtcgcgtgaaggaagactgcccgcaagggttgtaaacttcttttgtatgggattaaagtcacctacgtgtaggtgtttgcagttaccatacgaataagcatcggctaactccgtgccagcagccgcggtaatacggaggatgcgagcgttatccggaattattgggtttaaagggtgcgtaggttgcaagggaagtcaggggtgaaaagctgtagctcaactatggtcttgcctttgaaactctctagctagagtgtactggaggtacgtggaacgtgtggtgtagcggtgaaatgcatagatatcacacagaactccgattgcgcaggcagcgtactacattacaactgacactgaagcacgaaagcgtgggtatccaacaggattagataccctggtagtccacgcagtaaacgatgaatactagatctatgcgatatacagtatgggtctaagcgaaagcgataagtattccacctggggagtacgccggcaacggtgaaactcaaagagattggcgggggtccgcacaagcggaggaacatgtggtttaattcgatgatacgcgaggaaccttacccgggattgaaatgtagatgcatgaggctgagaggtctcttcccttcggggcttctatgtaggtgctgcatggttgtcgtcagctcgtgccgtgaggtgtcggcttaagtgccataacgagcgcaacccgcgtcgatagttactaacgagtcaagtcgaggactctatcgagacagccgtcgtaagacgtgaggaaggagcggatgacgtcaaatcagcacggcccttacatccggggcgacacacgtgttacaatggtagggacagcgagcagccatctggtgacagagagctaatctataaaccctatcccagttcggatcggagtctgcaactcgactctgtgaagctggattcgctagtaatcgcgcatcagccatggcgcggtgaatacgttcccggaccttgcacacaccgcccgtcaagccatgggagtcgggggtacctgaagagcgtgaccgtcacaggagcgcttgagggtaaaactggtgactggggctaagtcgtaacaaggtagccgtaccggaaggtgcggctggaacacctcctttct

SEQ ID NO: 134 - DNA, 1540 bp, Bacteroides asaccharolyticus
  misc_feature: Bacteroides asaccharolyticus B440
taacaatggagagtttgactctggctcaggatgaacgctagcgataggcttaacacatgcaagtcgaggggcagcgagatgtagcaatacgtcgtcggcgaccggcgaatgggtgagtaacacgtatgcaacttacctcttagtggtgaataacccgatgaaagtcggactaatacaccatatactccttagatcccatgagaagaggaggaaagattaatcgctaagagatgggcctgcgttccattagctagttggtaaggtaacggcttaccaaggcaacgatggatagggggactgagaggttgaccccccacattgacactgagatacgggtcaaactcctacgggaggcagcagtgaggaatattggtcaatgggcgagagcctgaaccagccaagtcgcgtgaaggaagactgcccgcaagggttgtaaacttcttttgtatgggattaaagtcacctacgtgtaggtgtttgcagttaccatacgaataagcatcggctaactccgtgccagcagccgcggtaatacggaggatgcgagcgttatccggaattattgggtttaaagggtgcgtaggttgcaagggaagtcaggggtgaaaagctgtagctcaactatggtcttgcctttgaaactctctagctagagtgtactggaggtacgtggaacgtgtggtgtagcggtgaaatgcatagatatcacacagaactccgattgcgcaggcagcgtactacattacaactgacactgaagcacgaaagcgtgggtatccaacaggattagataccctggtagtccacgcagtaaacgatgaatactagatctatgcgatatacagtatgggtctaagcgaaagcgataagtattccacctggggagtacgccggcaacggtgaaactcaaagagattggcgggggtccgcacaagcggaggaacatgtggtttaattcgatgatacgcgaggaaccttacccgggattgaaatgtagatgcatgaggctgagaggtctcttcccttcggggcttctatgtaggtgctgcatggttgtcgtcagctcgtgccgtgaggtgtcggcttaagtgccataacgagcgcaacccgcgtcgatagttactaacgagtcaagtcgaggactctatcgagacagccgtcgtaagacgtgaggaaggagcggatgacgtcaaatcagcacggcccttacatccggggcgacacacgtgttacaatggtagggacagcgagcagccatctggtgacagagagctaatctataaaccctatcccagttcggatcggagtctgcaactcgactctgtgaagctggattcgctagtaatcgcgcatcagccatggcgcggtgaatacgttcccggaccttgcacacaccgcccgtcaagccatgggagtcgggggtacctgaagagcgtgaccgtcacaggagcgcttgagggtaaaactggtgactggggctaagtcgtaacaaggtagccgtaccggaaggtgcggctggaacacctcctttctggagaca

SEQ ID NO: 135 - DNA, 1540 bp, Bacteroides asaccharolyticus; identical to SEQ ID NO: 134
  misc_feature: Bacteroides asaccharolyticus B440

SEQ ID NO: 136 - DNA, 1365 bp, Bacteroides asaccharolyticus
  misc_feature: Bacteroides asaccharolyticus B440
gtttaacacatgcaagtcgaggggcagcgagatgtagcaatacgtcgtcggcgaccggcgaatgggtgagtaacacgtatgcaacttacctcttagtggtgaataacccgatgaaagtcggactaatacaccatatactccttagatcccatgagaagaggaggaaagattaatcgctaagagatgggcctgcgttccattagctagttggtaaggtaacggcttaccaaggcaacgatggatagggggactgagaggttgaccccccacattgacactgagatacgggtcaaactcctacgggaggcagcagtgaggaatattggtcaatgggcgagagcctgaaccagccaagtcgcgtgaaggaagactgcccgcaagggttgtaaacttcttttgtatgggattaaagtcacctacgtgtaggtgtttgcagttaccatacgaataagcatcggctaactccgtgccagcagccgcggtaatacggaggatgcgagcgttatccggaattattgggtttaaagggtgcgtaggttgcaagggaagtcaggggtgaaaagctgtagctcaactatggtcttgcctttgaaactctctagctagagtgtactggaggtacgtggaacgtgtggtgtagcggtgaaatgcatagatatcacacagaactccgattgcgcaggcagcgtactacattacaactgacactgaagcacgaaagcgtgggtatccaacaggattagataccctggtagtccacgcagtaaacgatgaatactagatctatgcgatatacagtatgggtctaagcgaaagcgataagtattccacctggggagtacgccggcaacggtgaaactcaaagagattggcgggggtccgcacaagcggaggaacatgtggtttaattcgatgatacgcgaggaaccttacccgggattgaaatgtagatgcatgaggctgagaggtctcttcccttcggggcttctatgtaggtgctgcatggttgtcgtcagctcgtgccgtgaggtgtcggcttaagtgccataacgagcgcaacccgcgtcgatagttactaacgagtcaagtcgaggactctatcgagacagccgtcgtaagacgtgaggaaggagcggatgacgtcaaatcagcacggcccttacatccggggcgacacacgtgttacaatggtagggacagcgagcagccatctggtgacagagagctaatctataaaccctatcccagttcggatcggagtctgcaactcgactctgtgaagctggattcgctagtaatcgcgcatcagccatggcgcggtgaatacgttcccggaccttgcacacaccgcccgtcaagccatga

SEQ ID NO: 137 - DNA, 1479 bp, Bacteroides asaccharolyticus
  misc_feature: Bacteroides asaccharolyticus B440
ctggctcaggatgaacgctagcgataggcttaacacatgcaagtcgaggggcagcgagatgtagcaatacgtcgtcggcgaccggcgaatgggtgagtaacacgtatgcaacttacctcttagtggtgaataacccgatgaaagtcggactaatacaccatatactccttagatcccatgagaagaggaggaaagattaatcgctaagagatgggcctgcgttccattagctagttggtaaggtaacggcttaccaaggcaacgatggatagggggactgagaggttgaccccccacattgacactgagatacgggtcaaactcctacgggaggcagcagtgaggaatattggtcaatgggcgagagcctgaaccagccaagtcgcgtgaaggaagactgcccgcaagggttgtaaacttcttttgtatgggattaaagtcacctacgtgtaggtgtttgcagttaccatacgaataagcatcggctaactccgtgccagcagccgcggtaatacggaggatgcgagcgttatccggaattattgggtttaaagggtgcgtaggttgcaagggaagtcaggggtgaaaagctgtagctcaactatggtcttgcctttgaaactctctagctagagtgtactggaggtacgtggaacgtgtggtgtagcggtgaaatgcatagatatcacacagaactccgattgcgcaggcagcgtactacattacaactgacactgaagcacgaaagcgtgggtatccaacaggattagataccctggtagtccacgcagtaaacgatgaatactagatctatgcgatatacagtatgggtctaagcgaaagcgataagtattccacctggggagtacgccggcaacggtgaaactcaaagagattggcgggggtccgcacaagcggaggaacatgtggtttaattcgatgatacgcgaggaaccttacccgggattgaaatgtagatgcatgaggctgagaggtctcttcccttcggggcttctatgtaggtgctgcatggttgtcgtcagctcgtgccgtgaggtgtcggcttaagtgccataacgagcgcaacccgcgtcgatagttactaacgagtcaagtcgaggactctatcgagacagccgtcgtaagacgtgaggaaggagcggatgacgtcaaatcagcacggcccttacatccggggcgacacacgtgttacaatggtagggacagcgagcagccatctggtgacagagagctaatctataaaccctatcccagttcggatcggagtctgcaactcgactctgtgaagctggattcgctagtaatcgcgcatcagccatggcgcggtgaatacgttcccggaccttgcacacaccgcccgtcaagccatgggagtcgggggtacctgaagagcgtgaccgtcacaggagcgcttgagggtaaaactggtgactggggctaagtcgtaacaaggtaacc

SEQ ID NO: 138 - DNA, 1537 bp, Bacteroides
  misc_feature: Bacteroides sp. 2_2_4
cttttacaatgaagagtttgatcctggctcaggatgaacgctagctacaggcttaacacatgcaagtcgaggggcagcatttcgctagcaatagtgaagatggcgaccggcgcacgggtgagtaacacgtatccaacctgccgataactcggggatagcctttcgaaagaaagattaatatccgatagtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtgggattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactgggagtcttgagtacagtagaggtgggcggaattcgtggtgtagcggtgaaatgcttagatatcacgaagaactccgattgcgaaggcagctcactagactgtcactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaataatctggaaacaggttagccgcaaggcatttgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggtagccgtaccggaaggtgcggctggaacacctcctttctggagc

SEQ ID NO: 139 - DNA, 1525 bp, Bacteroides
  misc_feature: Bacteroides sp. 2_2_4
tacaatgaagagtttgatcctggctcaggatgaacgctagctacaggcttaacacatgcaagtcgaggggcagcatttcgctagcaatagtgaagatggcgaccggcgcacgggtgagtaacacgtatccaacctgccgataactcggggatagcctttcgaaagaaagattaatatccgatagtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtgggattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactgggagtcttgagtacagtagaggtgggcggaattcgtggtgtagcggtgaaatgcttagatatcacgaagaactccgattgcgaaggcagctcactagactgtcactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaataatctggaaacaggttagccgcaaggcatttgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggtagccgtaccggaaggtgcggctggaacacctcctt

SEQ ID NO: 140 - DNA, 1555 bp, Bilophila wadsworthia
  misc_feature: Bilophila wadsworthia 3 1 6
atcaactggagagtttgattctggctcagattgaacgctggcggcgtgcttaacacatgcaagtcgaacgtgaaagtccttcggggcgagtaaagtggcgcacgggtgagtaacgcgtggataatctacccttaagatggggataacggctggaaacggtcgctaataccgaatacgctcccgattttatcgttggggggaaagatggcctctgcttgcaagctatcgcttaaggatgagtccgcgtcccattagctagttggcggggtaacggcccaccaaggcgacgatgggtagccggtctgagaggatgaccggccacactggaactggaacacggtccagactcctacgggaggcagcagtggggaatattgcgcaatgggcgaaagcctgacgcagcgacgccgcgtgagggatgaaggttctcggatcgtaaacctctgtcaggggggaagaaaccccctcgtgtgaataatgcgagggcttgacggtacccccaaaggaagcaccggctaactccgtgccagcagccgcggtaatacggagggtgcaagcgttaatcggaatcactgggcgtaaagcgcacgtaggcggcttggtaagtcaggggtgaaatcccacagcccaactgtggaactgcctttgatactgccaggcttgagtaccggagagggtggcggaattccaggtgtaggagtgaaatccgtagatatctggaggaacaccggtggcgaaggcggccacctggacggtaactgacgctgaggtgcgaaagcgtgggtagcaaacaggattagataccctggtagtccacgctgtaaacgatgggtgctgggtgctgggatgtatgtctcggtgccgtagctaacgcgataagcaccccgcctggggagtacggtcgcaaggctgaaactcaaagaaattgacgggggcccgcacaagcggtggagtatgtggtttaattcgatgcaacgcgaagaaccttacccaggcttgacatctagggaacccttcggaaatgaaggggtgcccttcggggagccctaagacaggtgctgcatggctgtcgtcagctcgtgccgtgaggtgttgggttaagtcccgcaacgagcgcaacccctatcttcagttgccagcaggtaaggctgggcactctggagagaccgccccggtcaacggggaggaaggtggggacgacgtcaagtcatcatggcccttacgcctggggctacacacgtactacaatggcgcgcacaaagggtagcgagaccgcgaggtggagccaatcccaaaaaacgcgtcccagtccggattggagtctgcaactcgactccatgaagtcggaatcgctagtaattcgagatcagcatgctcgggtgaatgcgttcccgggccttgtacacaccgcccgtcacaccacgaaagtcggttttacccgaagccggtgagctaactcgcaagaggagcagccgtctacggtagggccgatgattggggtgaagtcgtaacaaggtagccgtaggggaacctgcggctggatcacctcctttat

SEQ ID NO: 141 - DNA, 1555 bp, Bilophila wadsworthia; identical to SEQ ID NO: 140
  misc_feature: Bilophila wadsworthia 3 1 6

SEQ ID NO: 142 - DNA, 1554 bp, Bilophila wadsworthia
  misc_feature: Bilophila wadsworthia 3 1 6
caactggagagtttgattctggctcagattgaacgctggcggcgtgcttaacacatgcaagtcgaacgtgaaagtccttcggggcgagtaaagtggcgcacgggtgagtaacgcgtggataatctacccttaagatggggataacggctggaaacggtcgctaataccgaatacgctcccgattttatcattggggggaaagatggcctctgcttgcaagctatcgcttaaggatgagtccgcgtcccattagctagttggcggggtaacggcccaccaaggcgacgatgggtagccggtctgagaggatgaccggccacactggaactggaacacggtccagactcctacgggaggcagcagtggggaatattgcgcaatgggcgaaagcctgacgcagcgacgccgcgtgagggatgaaggttctcggatcgtaaacctctgtcaggggggaagaaaccccctcgtgtgaataatgcgagggcttgacggtacccccaaaggaagcaccggctaactccgtgccagcagccgcggtaatacggagggtgcaagcgttaatcggaatcactgggcgtaaagcgcacgtaggcggcttggtaagtcaggggtgaaatcccacagcccaactgtggaactgcctttgatactgccaggcttgagtaccggagagggtggcggaattccaggtgtaggagtgaaatccgtagatatctggaggaacaccggtggcgaaggcggccacctggacggtaactgacgctgaggtgcgaaagcgtgggtagcaaacaggattagataccctggtagtccacgctgtaaacgatgggtgctgggtgctgggatgtatgtctcggtgccgtagctaacgcgataagcaccccgcctggggagtacggtcgcaaggctgaaactcaaagaaattgacgggggcccgcacaagcggtggagtatgtggtttaattcgatgcaacgcgaagaaccttacccaggcttgacatctagggaacccttcggaaatgaaggggtgcccttcggggagccctaagacaggtgctgcatggctgtcgtcagctcgtgccgtgaggtgttgggttaagtcccgcaacgagcgcaacccctatcttcagttgccagcaggtaaggctgggcactctggagagaccgccccggtcaacggggaggaaggtggggacgacgtcaagtcatcatggcccttacgcctggggctacacacgtactacaatggcgcgcacaaagggtagcgagaccgcgaggtggagccaatcccaaaaaacgcgtcccagtccggattggagtctgcaactcgactccatgaagtcggaatcgctagtaattcgagatcagcatgctcgggtgaatgcgttcccgggccttgtacacaccgcccgtcacaccacgaaagtcggttttacccgaagccggtgagctaactcgcaagaggagcagccgtctacggtagggccgatgattggggtgaagtcgtaacaaggtagccgtaggggaacctgcggctggatcacctcctttata

SEQ ID NO: 143 - DNA, 1554 bp, Bilophila wadsworthia
  misc_feature: Bilophila wadsworthia 3 1 6
caactggagagtttgattctggctcagattgaacgctggcggcgtgcttaacacatgcaagtcgaacgtgaaagtccttcggggcgagtaaagtggcgcacgggtgagtaacgcgtggataatctacccttaagatggggataacggctggaaacggtcgctaataccgaatacgctcccgattttatcgttggggggaaagatggcctctgcttgcaagctatcgcttaaggatgagtccgcgtcccattagctagttggcggggtaacggcccaccaaggcgacgatgggtagccggtctgagaggatgaccggccacactggaactggaacacggtccagactcctacgggaggcagcagtggggaatattgcgcaatgggcgaaagcctgacgcagcgacgccgcgtgagggatgaaggttctcggatcgtaaacctctgtcaggggggaagaaaccccctcgtgtgaataatgcgagggcttgacggtacccccaaaggaagcaccggctaactccgtgccagcagccgcggtaatacggagggtgcaagcgttaatcggaatcactgggcgtaaagcgcacgtaggcggcttggtaagtcaggggtgaaatcccacagcccaactgtggaactgcctttgatactgccaggcttgagtaccggagagggtggcggaattccaggtgtaggagtgaaatccgtagatatctggaggaacaccggtggcgaaggcggccacctggacggtaactgacgctgaggtgcgaaagcgtgggtagcaaacaggattagataccctggtagtccacgctgtaaacgatgggtgctgggtgctgggatgtatgtctcggtgccgtagctaacgcgataagcaccccgcctggggagtacggtcgcaaggctgaaactcaaagaaattgacgggggcccgcacaagcggtggagtatgtggtttaattcgatgcaacgcgaagaaccttacccaggcttgacatctagggaacccttcggaaatgaaggggtgcccttcggggagccctaagacaggtgctgcatggctgtcgtcagctcgtgccgtgaggtgttgggttaagtcccgcaacgagcgcaacccctatcttcagttgccagcaggtaaggctgggcactctggagagaccgccccggtcaacggggaggaaggtggggacgacgtcaagtcatcatggcccttacgcctggggctacacacgtactacaatggcgcgcacaaagggtagcgagaccgcgaggtggagccaatcccaaaaaacgcgtcccagtccggattggagtctgcaactcgactccatgaagtcggaatcgctagtaattcgagatcagcatgctcgggtgaatgcgttcccgggccttgtacacaccgcccgtcacaccacgaaagtcggttttacccgaagccggtgagctaactcgcaagaggagcagccgtctacggtagggccgatgattggggtgaagtcgtaacaaggtagccgtaggggaacctgcggctggatcacctcctttata

SEQ ID NO: 144 - DNA, 1554 bp, Bilophila wadsworthia; identical to SEQ ID NO: 142
  misc_feature: Bilophila wadsworthia 3 1 6

SEQ ID NO: 145 - DNA, 1554 bp, Bilophila wadsworthia; identical to SEQ ID NO: 143
  misc_feature: Bilophila wadsworthia 3 1 6

SEQ ID NO: 146 - DNA, 1551 bp, Bilophila wadsworthia
  misc_feature: Bilophila wadsworthia 3 1 6
tcaactggagagtttgattctggctcagattgaacgctggcggcgtgcttaacacatgcaagtcgaacgtgaaagtccttcggggcgagtaaagtggcgcacgggtgagtaacgcgtggataatctacccttaagatggggataacggctggaaacggtcgctaataccgaatacgctcccgattttatcgttggggggaaagatggcctctgcttgcaagctatcgcttaaggatgagtccgcgtcccattagctagttggcggggtaacggcccaccaaggcgacgatgggtagccggtctgagaggatgaccggccacactggaactggaacacggtccagactcctacgggaggcagcagtggggaatattgcgcaatgggcgaaagcctgacgcagcgacgccgcgtgagggatgaaggttctcggatcgtaaacctctgtcaggggggaagaaaccccctcgtgtgaataatgcgagggcttgacggtacccccaaaggaagcaccggctaactccgtgccagcagccgcggtaatacggagggtgcaagcgttaatcggaatcactgggcgtaaagcgcacgtaggcggcttggtaagtcaggggtgaaatcccacagcccaactgtggaactgcctttgatactgccaggcttgagtaccggagagggtggcggaattccaggtgtaggagtgaaatccgtagatatctggaggaacaccggtggcgaaggcggccacctggacggtaactgacgctgaggtgcgaaagcgtgggtagcaaacaggattagataccctggtagtccacgctgtaaacgatgggtgctgggtgctgggatgtatgtctcggtgccgtagctaacgcgataagcaccccgcctggggagtacggtcgcaaggctgaaactcaaagaaattgacgggggcccgcacaagcggtggagtatgtggtttaattcgatgcaacgcgaagaaccttacccaggcttgacatctagggaacccttcggaaatgaaggggtgcccttcggggagccctaagacaggtgctgcatggctgtcgtcagctcgtgccgtgaggtgttgggttaagtcccgcaacgagcgcaacccctatcttcagttgccagcaggtaaggctgggcactctggagagaccgccccggtcaacggggaggaaggtggggacgacgtcaagtcatcatggcccttacgcctggggctacacacgtactacaatggcgcgcacaaagggtagcgagaccgcgaggtggagccaatcccaaaaaacgcgtcccagtccggattggagtctgcaactcgactccatgaagtcggaatcgctagtaattcgagatcagcatgctcgggtgaatgcgttcccgggccttgtacacaccgcccgtcacaccacgaaagtcggttttacccgaagccggtgagctaactcgcaagaggagcagccgtctacggtagggccgatgattggggtgaagtcgtaacaaggtagccgtaggggaacctgcggctggatcacctcctt

SEQ ID NO: 147 - DNA, 1383 bp, Clostridium aldenense
  misc_feature: Clostridium aldenense RMA9741
gagtggcggacgggtgagtaacgcgtggataacctgcctcacactgggggataacagttagaaatggctgctaataccgcataagcgcacagtaccgcatggtacggtgtgaaaaacccaggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgcaagcctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgacgcaagtctggagtgaaagcccggggcccaaccccgggactgctttggaaactgtgttgctggagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatgctaggtgtcggggggcaaagcccttcggtgccgccgctaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggcacgtaacggtgcccttccttcgggacaggggagacaggtggtgcatggttgtcgtcagctcgtgtcgtgagatgttgggttaagtcccgcaacgagcgcaacccttatccttagtagccagcacgtcgtggtgggcactctagggagactgccagggataacctggaggaaggtggggatgacgtcaaatcatcatgccccttatgatttgggctacacacgtgctacaatggcgtaaacaaagggaggcgaccctgcgaaggcaagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagcaacgcccgaagtcagtgacccaaccttaacaggagggagctgccgaaggcggggcaggtaactggggtgaagtcgtaac

SEQ ID NO: 148 - DNA, 1378 bp, Clostridium bolteae
  misc_feature: Clostridium bolteae DSM 15670
gagtggcggacgggtgagtaacgcgtggataacctgcctcacactgggggataacagttagaaatgactgctaataccgcataagcgcacagtaccgcatggtacagtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtc

SEQ ID NO: 149 - DNA, 1544 bp, Clostridium bolteae
  misc_feature: Clostridium bolteae DSM 15670
ttttaaacatgagagtttgatcctggctcaggatgaacgctggcggcgtgcctaacacatgcaagtcgaacgaagcaattgaaggaagttttcggatggaattcgattgactgagtggcggacgggtgagtaacgcgtggataacctgcctcacactgggggataacagttagaaatgactgctaataccgcataagcgcacagtaccgcatggtacagtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtcgtaacaaggtagccgtatcggaaggtgcggctggatcacctcctttctaaggaa

SEQ ID NO: 150 - DNA, 1529 bp, Clostridium bolteae
  misc_feature: Clostridium bolteae DSM 15670
tgagagtttgatcctggctcaggatgaacgctggcggcgtgcctaacacatgcaagtcgaacgaagcaattgaaggaagttttcggatggaattcgattgactgagtggcggacgggtgagtaacgcgtggataacctgcctcacactgggggataacagttagaaatgactgctaataccgcataagcgcacagtaccgcatggtacagtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtcgtaacaaggtagccgtatcggaaggtgcggctggatcacctcctttct

SEQ ID NO: 151 - DNA, 1498 bp, Clostridium bolteae
  misc_feature: Clostridium bolteae DSM 15670
agagtttgatcctggctcaggatgaacgctggcggcgtgcctaacacatgcaagtcgaacgaagcaattaaaatgaagttttcggatggatttttaattgactgagtggcggacgggtgagtaacgcgtggataacctgcctcacactgggggataacagttagaaatgactgctaataccgcataagcgcacagtgccgcatggtacagtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccgcctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtagagctgggcactctagggagactgccagggataacctggaggaaggtggggatgacgtcaaatcatcatgccccttatgatttgggctacacacgtgctacaatggcgtaaacaaagggaagcaaggcagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtcgtaacaaggtaaccgta

SEQ ID NO: 152 - DNA, 1497 bp, Clostridium bolteae
  misc_feature: Clostridium bolteae DSM 15670
  misc_feature (66)..(67): n is a, c, g, or t
gagtttgattatggctcaggatgaacgctggcggcgtgcttaacacatgcaagtcgaacgaagatnnttagaatgagagcttcggcaggatttttatctatcttagtggcggacgggtgagtaacgtgtgggcaacctgccctgtactggggaataatcattggaaacgatgactaataccgcatgtggtcctcggaaggcatcttctgaggaagaaaggatttattcggtacaggatgggcccgcatctgattagctagttggtgagataacagcccaccaaggcgacgatcagtagccgacctgagagggtgatcggccacattgggactgagacacggcccaaactcctacgggaggcagcagtggggaatattgcacaatgggcgaaagcctgatgcagcaacgccgcgtgaaggatgaagggtttcggctcgtaaacttctatcaatagggaagaaataaatgacggtacctaaataagaagccccggctaactacgtgccagcagccgcggtaatacgtagggggcaagcgttatccggaattactgggtgtaaagggagcgtaggcggcatggtaagccagatgtgaaagccttgggcttaacccgaggattgcatttggaactatcaagctagagtacaggagaggaaagcggaattcctagtgtagcggtgaaatgcgtagatattaggaagaacaccagtggcgaaggcggctttctggactgaaactgacgctgaggctcgaaagcgtggggagcaaacaggattagataccctggtagtccacgccgtaaacgatgagtgctaggtgtcggggaggaatcctcggtgccgcagctaacgcaataagcactccacctggggagtacgaccgcaaggttgaaactcaaaggaattgacgggggcccgcacaagcggtggagcatgtggtttaattcgaagcaacgcgaagaaccttaccaaggcttgacatcccgatgaccgtcctagagataggacttctcttcggagcatcggtgacaggtggtgcatggttgtcgtcagctcgtgtcgtgagatgttgggttaagtcccgcaacgagcgcaacccttatcttcagtagccatcattcagttgggcactctggagagactgccgtggataacacggaggaaggtggggatgacgtcaaatcatcatgccccttatgtcttgggctacacacgtgctacaatggctggtaacaaagtgacgcaaaacggcgacgttaagcaaatcacaaaaacccagtcccagttcggattgtagtctgcaactcgactacatgaagctggaatcgctagtaatcgcgaatcagcatgtcgcggtgaatacgttcccgggccttgtacacaccgcccgtcacaccatgggagttggaagcacccgaagtcggtgacctaaccgtaaggaaggagccgccgaaggtgaagccagtgactggggtgaagtcgtaacaaggtaaccgta

SEQ ID NO: 153 - DNA, 1497 bp, Clostridium lactatifermentans; identical to SEQ ID NO: 152
  misc_feature: Clostridium lactatifermentans G17
  misc_feature (66)..(67): n is a, c, g, or t

SEQ ID NO: 154 - DNA, 1464 bp, Clostridium
  misc_feature: Clostridium sp. LIP1
  misc_feature (43)..(48): n is a, c, g, or t
aacgctggcggcgtgcttaacacatgcaagtcgaacgggaagnnnnnnctgagtggcggacgggtgagtaacgcgtggataacctgcctcacactgggggataacagttagaaatgactgctaataccgcataagcgcacgctaccgcatggtagtgtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggagtgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaggagagacaggtggtgcatggttgtcgtcagctcgtgtcgtgagatgttgggttaagtcccgcaacgagcgcaacccttatccttagtagccagcaggtagagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaagaagggagctgccgaaggcggggcaggtaactggggtgaagtcgtaacaaggtagccgtatcggaaggtgcggctggatca

SEQ ID NO: 155 - DNA, 1542 bp, Desulfovibrio piger
  misc_feature: Desulfovibrio piger DSM 749
agagtttgatcctggctcagattgaacgctggcggcgtgcttaacacatgcaagtcgtacgcgaaagggacttcggtcccgagtaaagtggcgcacgggtgagtaacacgtggataatctgcctctatgatggggataacagttggaaacgactgctaataccgaatacgctcatgatgaactttgtgaggaaaggtggcctctgcttgcaagctatcgcatagagatgagtccgcgtcccattagctagttggtggggtaacggcctaccaaggcaacgatgggtagccgatctgagaggatgatcggccacactggaactgaaacacggtccagactcctacgggaggcagcagtggggaatattgcgcaatgggcgaaagcctgacgcagcgacgccgcgtgagggatgaaggtcttcggatcgtaaacctctgtcagaagggaagaaactagggtgttctaatcatcatcctactgacggtaccttcaaaggaagcaccggctaactccgtgccagcagccgcggtaatacggagggtgcaagcgttaatcggaatcactgggcgtaaagcgcacgtaggctgttatgtaagtcaggggtgaaagcccacggctcaaccgtggaactgcccttgatactgcacgactcgaatccgggagagggtggcggaattccaggtgtaggagtgaaatccgtagatatctggaggaacatcagtggcgaaggcggccacctggaccggtattgacgctgaggtgcgaaagcgtggggagcaaacaggattagataccctggtagtccacgccgtaaacgatggatgctagatgtcgggatgtatgtctcggtgtcgtagttaacgcgttaagcatcccgcctggggagtacggtcgcaaggctgaaactcaaagaaattgacgggggcccgcacaagcggtggagtatgtggtttaattcgatgcaacgcgaagaaccttacctaggtttgacatctggggaaccctcccgaaaatgaggggtgcccttcggggagccccaagacaggtgctgcatggctgtcgtcagctcgtgtcgtgagatgttgggttaagtcccgcaacgagcgcaacccctatgcatagttgccagcaagtaaagttgggcactctatgcagactgcccgggttaaccgggaggaaggtggggacgacgtcaagtcatcatggcccttacacctagggctacacacgtactacaatggcacgcacaaagggcagcgataccgtgaggtggagccaatcccaaaaaacgtgtcccagtccggattgcagtctgcaactcgactgcatgaagtcggaatcgctagtaattcgaggtcagcatactcgggtgaatgcgttcccgggccttgtacacaccgcccgtcacaccacgaaagtcggttttacccgaagccggtgagccaactagcaatagaggcagccgtctacggtagggccgatgattggggtgaagtcgtaacaaggtagccgtaggggaacctgcggctggatcacctcctt

SEQ ID NO: 156 - DNA, 1504 bp, Dialister pneumosintes
  misc_feature: Dialister pneumosintes DSM 11619
cgaacgctggcggcgtgcttaacacatgcaagtcgaacggaaagagatgaaagagcttgctcttttattaatttcagtggcaaacgggtgagtaacacgtaaacaacctgccttaaggatggggataacagacggaaacgactgctaataccgaatacgttctaagcatcgcatggtgcatagaagaaagggtggcctctacaagaaagctatcgccttaagaggggtttgcgactgattaggtagttggtgaggtaacggctcaccaagccgacgatcagtagccggtctgagaggatgaacggccacactggaactgagacacggtccagactcctacgggaggcagcagtggggaatcttccgcaatggacgaaagtctgacggagcaacgccgcgtgaacgaagaaggtcttcggattgtaaagttctgtgattcgggacgaaagggtttgtggtgaataatcattgacattgacggtaccgaaaaagcaagccacggctaactacgtgccagcagccgcggtaatacgtaggtggcaagcgttgtccggaattattgggcgtaaagcgcgcgcaggcggtttcttaagtccatcttaaaagcgtggggctcaaccccatgaggggatggaaactgggaagctggagtatcggagaggaaagtggaattcctagtgtagcggtgaaatgcgtagagattaggaagaacaccggtggcgaaggcgactttctagacgaaaactgacgctgaggcgcgaaagcgtggggagcaaacaggattagataccctggtagtccacgccgtaaacgatggatactaggtgtaggaggtatcgacccctcctgtgccggagttaacgcaataagtatcccgcctgggaagtacgatcgcaagattaaaactcaaaggaattgacgggggcccgcacaagcggtggagtatgtggtttaattcgacgcaacgcgaagaaccttaccaagccttgacattgatcgcaatccatagaaatatggagttcttcttcggaagacgagaaaacaggtggtgcacggctgtcgtcagctcgtgtcgtgagatgttgggttaagtcccgcaacgagcgcaacccctatttcctgttaccagcacgtaaaggtggggactcaggagagaccgccgcggacaacgcggaggaaggcggggatgacgtcaagtcatcatgccccttatggcttgggctacacacgtactacaatgggtgccaacaaagagaagcgaaatcgcgagatggagcggacctcataaacgcacccccagttcagattgcaggctgcaactcgcctgcatgaagtaggaatcgctagtaatcgcgggtcagcataccgcggtgaatacgttcccgggccttgtacacaccgcccgtcacactatgagagttagagacacccgaagccggtgaggtaaccgcaagggaccagccgtctaaggtggagctgatgattggagtgaagtcgtaacaag

SEQ ID NO: 157 - DNA, 1483 bp, Eubacterium biforme
  misc_feature: Eubacterium biforme DSM 3989
  misc_feature (27)..(27): n is a, c, g, or t
  misc_feature (116)..(116): n is a, c, g, or t
  misc_feature (238)..(238): n is a, c, g, or t
  misc_feature (337)..(338): n is a, c, g, or t
  misc_feature (575)..(575): n is a, c, g, or t
  misc_feature (924)..(925): n is a, c, g, or t
  misc_feature (929)..(929): n is a, c, g, or t
  misc_feature (964)..(964): n is a, c, g, or t
  misc_feature (1225)..(1225): n is a, c, g, or t
  misc_feature (1381)..(1381): n is a, c, g, or t
tggagagtttgatcctggctcaggatnaacgctggcggcatgcctaatacatgcaagtcgaacgagaggaaggaaagcttgcttttctgaatctagtggcgaacgggtgagtaacncgtaggtaacctgcccatgtgcccgggataacttctggaaacggatgctaaaaccggataggtagcagacaagcatttgactgctattaaagtggctaaggccatgaacatggatggacctncggtgcattagctagttggtgaggtaacggcccaccaaggcgacgatgcatagccggcctgagagggcggacggccacattgggactgagacacggccnnaactcctacgggaggcagcagtagggaattttcgtcaatggggggaaccctgaacgagcaatgccgcgtgagtgaggaaggtcttcggatcgtaaagctctgttgtaagagaaaaacgacattcatagggaatgatgagtgagtgatggtatcttaccagaaagtcacggctaactacgtgccagcagccgcggtaatacgtaggtggcgagcgttatccggaatgattgggcgtanagggtgcgtaggtggcagaacaagtctggagtaaaaggtatgggctcaacccgtactggctctggaaactgttcagctagagaacagaagaggacggcggaactccatgtgtagcggtaaaatgcgtagatatatggaagaacaccggtggcgaaggcggccgtctggtctgttgctgacactgaagcacgaaagcgtggggagcaaataggattagataccctagtagtccacgccgtaaacgatgagaactaggtgttgggggaataactcagtgccgcagttaacgcaataagttctccgcctggggagtatgcacgcaagtgtgaaactcaaaggaattgacggnngccngcacaagcggtggagtatgtggtttaattcgaagnaacgcgaagaaccttaccaggccttgacatcccttgcagagrtatagagatatatccgaggttaacaaggagacaggtggtgcatggttgtcgtcagctcgtgtcgtgagatgttgggttaagtcccgcaacgagcgcaacccttgtgatatgttactaacattgagttgaggactcatatcagactgccggtgataaaccggaggaaggtggggatgacgtcaaatcatcatgccccttatggcctgggctacacacgtnctacaatggcgtctacaaagagcggcaagcctgtgaaggcaagcgaatctcataaaggacgtctcagttcggattgaagtctgcaacccgacttcatgaagctggaatcgctagtaatcgcgaatcagcatgtcgcggtgaatacgttctcgggcnttgtacacaccgcccgtcaaaccatgggagttggtaatgcccgaagccggtggcataacctttcgaggagtgagccgtcgaaggcaggaccgatgactgggg

SEQ ID NO: 158 - DNA, 1548 bp, Eubacterium biforme
  misc_feature: Eubacterium biforme DSM 3989
caaatggagagtttgatcctggctcaggatgaacgctggcggcatgcctaatacatgcaagtcgaacgagaggaaggaaagcttgcttttctgaatctagtggcgaacgggtgagtaacacgtaggtaacctgcccatgtgcccgggataacttctggaaacggatgctaaaaccggataggtagcagacaagcatttgactgctattaaagtggctaaggccatgaacatggatggacctgcggtgcattagctagttggtgaggtaacggcccaccaaggcgacgatgcatagccggcctgagagggcggacggccacattgggactgagacacggcccaaactcctacgggaggcagcagtagggaattttcgtcaatggggggaaccctgaacgagcaatgccgcgtgagtgaggaaggtcttcggatcgtaaagctctgttgtaagagaaaaacgacattcatagggaatgatgagtgagtgatggtatcttaccagaaagtcacggctaactacgtgccagcagccgcggtaatacgtaggtggcgagcgttatccggaatgattgggcgtaaagggtgcgtaggtggcagaacaagtctggagtaaaaggtatgggctcaacccgtactggctctggaaactgttcagctagagaacagaagaggacggcggaactccatgtgtagcggtaaaatgcgtagatatatggaagaacaccggtggcgaaggcggccgtctggtctgttgctgacactgaagcacgaaagcgtggggagcaaataggattagataccctagtagtccacgccgtaaacgatgagaactaggtgttgggggaataactcagtgccgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatcccttgcagaggtatagagatatatccgaggttatcaaggagacaggtggtgcatggttgtcgtcagctcgtgtcgtgagatgttgggttaagtcccgcaacgagcgcaacccttgtgatatgttactaacattgagttgaggactcatatcagactgccggtgataaaccggaggaaggtggggatgacgtcaaatcatcatgccccttatggcctgggctacacacgtactacaatggcgtctacaaagagcggcaagcctgtgaaggcaagcgaatctcataaaggacgtctcagttcggattgaagtctgcaacccgacttcatgaagctggaatcgctagtaatcgcgaatcagcatgtcgcggtgaatacgttctcgggccttgtacacaccgcccgtcaaaccatgggagttggtaatgcccgaagccggtggcataacctttcgaggagtgagccgtcgaaggcaggaccgatgactggggttaagtcgtaacaaggtatccctacgggaacgtggggatggatcacctcctttctaaggag

SEQ ID NO: 159 - DNA, 1538 bp, Eubacterium biforme
  misc_feature: Eubacterium biforme DSM 3989
tggagagtttgatcctggctcaggatgaacgctggcggcatgcctaatacatgcaagtcgaacgagaggaaggaaagcttgcttttctgaatctagtggcgaacgggtgagtaacacgtaggtaacctgcccatgtgcccgggataacttctggaaacggatgctaaaaccggataggtagcagacaagcatttgactgctattaaagtggctaaggccatgaacatggatggacctgcggtgcattagctagttggtgaggtaacggcccaccaaggcgacgatgcatagccggcctgagagggcggacggccacattgggactgagacacggcccaaactcctacgggaggcagcagtagggaattttcgtcaatggggggaaccctgaacgagcaatgccgcgtgagtgaggaaggtcttcggatcgtaaagctctgttgtaagagaaaaacgacattcatagggaatgatgagtgagtgatggtatcttaccagaaagtcacggctaactacgtgccagcagccgcggtaatacgtaggtggcgagcgttatccggaatgattgggcgtaaagggtgcgtaggtggcagaacaagtctggagtaaaaggtatgggctcaacccgtactggctctggaaactgttcagctagagaacagaagaggacggcggaactccatgtgtagcggtaaaatgcgtagatatatggaagaacaccggtggcgaaggcggccgtctggtctgttgctgacactgaagcacgaaagcgtggggagcaaataggattagataccctagtagtccacgccgtaaacgatgagaactaggtgttgggggaataactcagtgccgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatcccttgcagaggtatagagatatatccgaggttatcaaggagacaggtggtgcatggttgtcgtcagctcgtgtcgtgagatgttgggttaagtcccgcaacgagcgcaacccttgtgatatgttactaacattgagttgaggactcatatcagactgccggtgataaaccggaggaaggtggggatgacgtcaaatcatcatgccccttatggcctgggctacacacgtactacaatggcgtctacaaagagcggcaagcctgtgaaggcaagcgaatctcataaaggacgtctcagttcggattgaagtctgcaacccgacttcatgaagctggaatcgctagtaatcgcgaatcagcatgtcgcggtgaatacgttctcgggccttgtacacaccgcccgtcaaaccatgggagttggtaatgcccgaagccggtggcataacctttcgaggagtgagccgtcgaaggcaggaccgatgactggggttaagtcgtaacaaggtatccctacgggaacgtggggatggatcacctcctttct

SEQ ID NO: 160 - DNA, 1538 bp, Eubacterium biforme; identical to SEQ ID NO: 159
  misc_feature: Eubacterium biforme DSM 3989

SEQ ID NO: 161 - DNA, 1512 bp, Fusobacterium mortiferum
  misc_feature: Fusobacterium mortiferum ATCC 9817
attgaacgaagagtttgatcctggctcaggatgaacgctgacagaatgcttaacacatgcaagtctacttgatccttcgggtgatggtggcggacgggtgagtaacgcgtaaagaacttgccctgcagtctgggacaacatttggaaacgaatgctaataccggatattatgtgagtttcgcatgaaattcatatgaaagctatatgcgctgcaggagagctttgcgtcctattagctagttggtgaggtaacggctcaccaaggcgatgataggtagccggcctgagagggtgaacggccacaaggggactgagacacggcccttactcctacgggaggcagcagtggggaatattggacaatggaccaaaagtctgatccagcaattctgtgtgcacgatgaagtttttcggaatgtaaagtgctttcagttgggacgaagtaagtgacggtaccaacagaagaagcgacggctaaatacgtgccagcagccgcggtaatacgtatgtcgcaagcgttatccggatttattgggcgtaaagcgcgtctaggcggtttggtaagtctgatgtgaaaatgcggggctcaactccgtattgcgttggaaactgtcaaactagagtactggagaggtgggcggaactacaagtgtagaggtgaaattcgtagatatttgtaggaatgccgatggggaagccagcccactggacagatactgacgctaaagcgcgaaagcgtgggtagcaaacaggattagataccctggtagtccacgccgtaaacgatgattactaggtgttgggggtcgaacctcagcgcccaagctaacgcgataagtaatccgcctggggagtacgtacgcaagtatgaaactcaaaggaattgacggggacccgcacaagcggtggagcatgtggtttaattcgacgcaacgcgaggaaccttaccagcgtttgacatcctaagaaattagcagagatgcttttgtgccccttcgggggaacttagtgacaggtggtgcatggctgtcgtcagctcgtgtcgtgagatgttgggttaagtcccgcaacgagcgcaacccctttcgtatgttgccatcattaagttgggcactcatgcgatactgcctgcgatgagcaggaggaaggtggggatgacgtcaagtcatcatgccccttatacgctgggctacacacgtgctacaatgggtagtacagagagtcgcaaacctgcgagggggagctaatctcagaaaactattctcagttcggattgtactctgcaactcgagtacatgaagttggaatcgctagtaatcgcaaatcagctatgttgcggtgaatacgttctcgggtcttgtacacaccgcccgtcacaccacgagagttggttgcacctgaagtagcaggcctaaccgcaaggagggatgctccgagggtgtgattagcgattggggtgaagtcgtaacaaggtatccgtacgggaacgtgcggatggatcacctcctttct

SEQ ID NO: 162 - DNA, 1525 bp, Fusobacterium mortiferum
  misc_feature: Fusobacterium mortiferum ATCC 9817
ttatatagattgaacgaagagtttgatcctggctcaggatgaacgctgacagaatgcttaacacatgcaagtctacttgatccttcgggtgatggtggcggacgggtgagtaacgcgtaaagaacttgccctgcagtctgggacaacatttggaaacgaatgctaataccggatattatgtgagtttcgcatgaaattcatatgaaagctatatgcgctgcaggagagctttgcgtcctattagctagttggtgaggtaacggctcaccaaggcgatgataggtagccggcctgagagggtgaacggccacaaggggactgagacacggcccttactcctacgggaggcagcagtggggaatattggacaatggaccaaaagtctgatccagcaattctgtgtgcacgatgaagtttttcggaatgtaaagtgctttcagttgggacgaagtaagtgacggtaccaacagaagaagcgacggctaaatacgtgccagcagccgcggtaatacgtatgtcgcaagcgttatccggatttattgggcgtaaagcgcgtctaggcggtttggtaagtctgatgtgaaaatgcggggctcaactccgtattgcgttggaaactgtcaaactagagtactggagaggtgggcggaactacaagtgtagaggtgaaattcgtagatatttgtaggaatgccgatggggaagccagcccactggacagatactgacgctaaagcgcgaaagcgtgggtagcaaacaggattagataccctggtagtccacgccgtaaacgatgattactaggtgttgggggtcgaacctcagcgcccaagctaacgcgataagtaatccgcctggggagtacgtacgcaagtatgaaactcaaaggaattgacggggacccgcacaagcggtggagcatgtggtttaattcgacgcaacgcgaggaaccttaccagcgtttgacatcctaagaaattagcagagatgcttttgtgccccttcgggggaacttagtgacaggtggtgcatggctgtcgtcagctcgtgtcgtgagatgttgggttaagtcccgcaacgagcgcaacccctttcgtatgttgccatcattaagttgggcactcatgcgatactgcctgcgatgagcaggaggaaggtggggatgacgtcaagtcatcatgccccttatacgctgggctacacacgtgctacaatgggtagtacagagagtcgcaaacctgcgagggggagctaatctcagaaaactattctcagttcggattgtactctgcaactcgagtacatgaagttggaatcgctagtaatcgcaaatcagctatgttgcggtgaatacgttctcgggtcttgtacacaccgcccgtcacaccacgagagttggttgcacctgaagtagcaggcctaaccgcaaggagggatgctccgagggtgtgattagcgattggggtgaagtcgtaacaaggtatccgtacgggaacgtgcggatggatcacctcctttctaagga

SEQ ID NO: 163 - DNA, 1503 bp, Fusobacterium mortiferum
  misc_feature: Fusobacterium mortiferum ATCC 9817
agagtttgatcctggctcaggatgaacgctgacagaatgcttaacacatgcaagtctacttgatccttcgggtgatggtggcggacgggtgagtaacgcgtaaagaacttgccctgcagtctgggacaacatttggaaacgaatgctaataccggatattatgtgagtttcgcatgaaattcatatgaaagctatatgcgctgcaggagagctttgcgtcctattagctagttggtgaggtaacggctcaccaaggcgatgataggtagccggcctgagagggtgaacggccacaaggggactgagacacggcccttactcctacgggaggcagcagtggggaatattggacaatggaccaaaagtctgatccagcaattctgtgtgcacgatgaagtttttcggaatgtaaagtgctttcagttgggacgaagtaagtgacggtaccaacagaagaagcgacggctaaatacgtgccagcagccgcggtaatacgtatgtcgcaagcgttatccggatttattgggcgtaaagcgcgtctaggcggtttggtaagtctgatgtgaaaatgcggggctcaactccgtattgcgttggaaactgtcaaactagagtactggagaggtgggcggaactacaagtgtagaggtgaaattcgtagatatttgtaggaatgccgatggggaagccagcccactggacagatactgacgctaaagcgcgaaagcgtgggtagcaaacaggattagataccctggtagtccacgccgtaaacgatgattactaggtgttgggggtcgaacctcagcgcccaagctaacgcgataagtaatccgcctggggagtacgtacgcaagtatgaaactcaaaggaattgacggggacccgcacaagcggtggagcatgtggtttaattcgacgcaacgcgaggaaccttaccagcgtttgacatcctaagaaattagcagagatgcttttgtgccccttcgggggaacttagtgacaggtggtgcatggctgtcgtcagctcgtgtcgtgagatgttgggttaagtcccgcaacgagcgcaacccctttcgtatgttgccatcattaagttgggcactcatgcgatactgcctgcgatgagcaggaggaaggtggggatgacgtcaagtcatcatgccccttatacgctgggctacacacgtgctacaatgggtagtacagagagtcgcaaacctgcgagggggagctaatctcagaaaactattctcagttcggattgtactctgcaactcgagtacatgaagttggaatcgctagtaatcgcaaatcagctatgttgcggtgaatacgttctcgggtcttgtacacaccgcccgtcacaccacgagagttggttgcacctgaagtagcaggcctaaccgcaaggagggatgctccgagggtgtgattagcgattggggtgaagtcgtaacaaggtatccgtacgggaacgtgcggatggatcacctcctttct

SEQ ID NO: 164 - DNA, 1493 bp, Hungatella hathewayi
  misc_feature: Hungatella hathewayi 1313
  misc_feature (191)..(191): n is a, c, g, or t
  misc_feature (211)..(211): n is a, c, g, or t
  misc_feature (248)..(248): n is a, c, g, or t
  misc_feature (979)..(979): n is a, c, g, or t
ctcaggatgaacgctggcggcgtgcttaacacatgcaagtcgagcgaagcggtttcaatgaagttttcggatggatttgaaattgacttagcggcggacgggtgagtaacgcgtgggtaacctgccttacactgggggataacagttagaaatgactgctaataccgcataagcgcacagggccgcatggnctggtgtgaaaaactccggnggtgtaagatggacccgcgtctgattaggtagttggnggggtaacggcccaccaagccgacgatcagtagccgacctgagagggtgaccggccacattgggactgagacacggcccaaactcctacgggaggcagcagtggggaatattggacaatgggcgaaagcctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtttagcaagtctgaagtgaaagcccggggctcaaccccggtactgctttggaaactgttagacttgagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgtcggggggcaaagcccttcggtgccgccgcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgaaaacacnttaaccgtgatccctcttcggagcagtggagacaggtggtgcatggttgtcgtcagctcgtgtcgtgagatgttgggttaagtcccgcaacgagcgcaacccttatccttagtagccagcgagtagagtcgggcactctggggagactgccagggataacctggaggaaggtggggatgacgtcaaatcatcatgccccttatgatttgggctacacacgtgctacaatggcgtaaacaaagggaggcaaaggagcgatctggagcaaaccccaaaaataacgtctcagttcggattgcaggctgcaactcgcctgcatgaagctggaatcgctagtaatcgcgaatcagaatgtcgcggtgaatacgttcccgggtcttgtacacaccgcccgtcacaccatgggagttggtaacgcccgaagtcagtgacccaaccgaaaggagggagctgccgaaggcgggactgataactggggtgaagtcgtaacaaggtagccgtatcggaaggtgcg

SEQ ID NO: 165 - DNA, 1408 bp, Megasphaera micronuciformis
  misc_feature: Megasphaera micronuciformis DSM 17226
agcttgctttttctacaatctagtggcaaacgggtgagtaacgcgtaaacaacctgccctacggatggggacaacagctggaaacggctgctaataccgaatacgttccgaaagtcgcatgactgtcggaagaaaggatggcctctatttataagctatcgccggaggaggggtttgcgtctgattagctagttggaggggtaacggcccaccaaggcgacgatcagtagccggtctgagaggatgaacggccacattggaactgagacacggtccagactcctacgggaggcagcagtggggaatcttccgcaatgggcgaaagcctgacggagcaacgccgcgtgagtgaagacggccttcgggttgtaaagctctgttatacgggacgaataatcttgtggttaatacccataagaagtgacggtaccgtaagagaaagccacggctaactacgtgccagcagccgcggtaatacgtaggtggcaagcgttgtccggaattattgggcgtaaagggcgcgcaggcggcttcttaagtctgtcttaaaagtgcggggcttaaccccgtgatgggatggaaactgggaagctcagagtatcggagaggaaagcggaattcctagtgtagcggtgaaatgcgtagatattaggaggaacaccagtggcgaaagcggcttttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacggccgcaaggttgaaactcaaaggaattgacgggggcccgcacaagcggtggagtatgtggtttaattcgacgcaacgcgaagaaccttaccaagccttgacattgatcgcaaggagtagagatactctgttcttcttcggaagacgagaaaacaggtggtgcacggctgtcgtcagctcgtgtcgtgagatgttgggttaagtcccgcaacgagcgcaacccctatcttctgttaccagcacgtaaaggtggggactcaggagagactgccgcagacaatgcggaggaaggcggggatgacgtcaagtcatcatgccccttatggcttgggctacacacgtactacaatggctcttaatagagggaagcgaaggagcgatccggagcaaaccccaaaaacagagtctcagttcggattgtaggctgcaactcgcctacatgaagcaggaatcgctagtaatcgcaggtcagcatactgcggtgaatacgttcccgggccttgtacacaccgcccgtcacaccacgaaagtcattcacacccgaagccggtgaggtaaccgcaaggagcca

SEQ ID NO: 166 - DNA, 1487 bp, Micromonas micros
  misc_feature: Micromonas micros ACM 5086
agagtttgatcctggctcaggacgaacgctggcggcgtgcttaacacatgcaagtcgaacgtgatttttgtggaaattctttcgggaatggaaatgaaatgaaagtggcgaacgggtgagtaacacgtgagcaacctaccttacacagggggatagccgttggaaacgacgattaataccgcatgagaccacagaatcgcatgatataggggtcaaagatttatcggtgtaagaagggctcgcgtctgattagctagttggaagggtaaaggcctaccaaggcgacgatcagtagccggtctgagaggatgaacggccacattggaactgagacacggtccaaactcctacgggaggcagcagtggggaatattgcacaatggggggaaccctgatgcagcgacgccgcgtgagcgaagaaggttttcgaatcgtaaagctctgtcctatgagaagataatgacggtatcataggaggaagccccggctaaatacgtgccagcagccgcggtaatacgtatggggcgagcgttgtccggaattattgggcgtaaagggtacgtaggcggttttttaagtcaggtgtgaaagcgtgaggcttaacctcattaagcacttgaaactggaagacttgagtgaaggagaggaaagtggaattcctagtgtagcggtgaaatgcgtagatattaggaggaataccggtggcgaaggcgactttctggacttttactgacgctcaggtacgaaagcgtggggagcaaacaggattagataccctggtagtccacgccgtaaacgatgaatgctaggtgttgggagtcaaatctcggtgccgaagttaacacattaagcattccgcctggggagtacggtggcaacactgaaactcaaaggaattgacggggacccgcacaagcagcggagcatgtggtttaattcgaagcaacgcgaagaaccttaccaaggcttgacatatagttgagttattgagaaattgataagtccctcgggacaactatacaggtggtgcatggttgtcgtcagctcgtgtcgtgagatgttgggttaagtcccgcaacgagcgcaacccttatcttcagttgccagcacgtagaggtgggaactctggagagactgccgatgacaaatcggaggaaggtggggatgacgtcaaatcatcatgccctttatgtcttgggctacacacgtgctacaatggttggtacaacgagaagcgagatagagatgttaagcgaaactctaaaaaccaatctcagttcggattgtaggctgcaactcgcctacatgaagtcggagttgctagtaatcgcgaatcagaatgtcgcggtgaatgcgttcccgggtcttgtacacaccgcccgtcacaccatgggagttggcaatacccgaagccgccgatctaaccgcaaggaggaaggcgtcgaaggtagggttaatgactggggtgaagtcgtaacaaggtaacc

SEQ ID NO: 167 - DNA, 1536 bp, Micromonas micros
  misc_feature: Micromonas micros ACM 5086
tttaaatagagagtttgatcctggctcaggacgaacgctggcggcgtgcttaacacatgcaagtcgaacgtgatttttgtggaaattctttcgggaatggaaatgaaatgaaagtggcgaacgggtgagtaacacgtgagcaacctaccttacacagggggatagccgttggaaacgacgattaataccgcatgagaccacagaatcgcatgatataggggtcaaagatttatcggtgtaagaagggctcgcgtctgattagctagttggaagggtaaaggcctaccaaggcgacgatcagtagccggtctgagaggatgaacggccacattggaactgagacacggtccaaactcctacgggaggcagcagtggggaatattgcacaatggggggaaccctgatgcagcgacgccgcgtgagcgaagaaggttttcgaatcgtaaagctctgtcctatgagaagataatgacggtatcataggaggaagccccggctaaatacgtgccagcagccgcggtaatacgtatggggcgagcgttgtccggaattattgggcgtaaagggtacgtaggcggttttttaagtcaggtgtgaaagcgtgaggcttaacctcattaagcacttgaaactggaagacttgagtgaaggagaggaaagtggaattcctagtgtagcggtgaaatgcgtagatattaggaggaataccggtggcgaaggcgactttctggacttttactgacgctcaggtacgaaagcgtggggagcaaacaggattagataccctggtagtccacgccgtaaacgatgaatgctaggtgttgggagtcaaatctcggtgccgaagttaacacattaagcattccgcctggggagtacggtggcaacactgaaactcaaaggaattgacggggacccgcacaagcagcggagcatgtggtttaattcgaagcaacgcgaagaaccttaccaaggcttgacatatagttgagttattgagaaattgataagtccctcgggacaactatacaggtggtgcatggttgtcgtcagctcgtgtcgtgagatgttgggttaagtcccgcaacgagcgcaacccttatcttcagttgccagcacgtagaggtgggaactctggagagactgccgatgacaaatcggaggaaggtggggatgacgtcaaatcatcatgccctttatgtcttgggctacacacgtgctacaatggttggtacaacgagaagcgagatagagatgttaagcgaaactctaaaaaccaatctcagttcggattgtaggctgcaactcgcctacatgaagtcggagttgctagtaatcgcgaatcagaatgtcgcggtgaatgcgttcccgggtcttgtacacaccgcccgtcacaccatgggagttggcaatacccgaagccgccgatctaaccgcaaggaggaaggcgtcgaaggtagggttaatgactggggtgaagtcgtaacaaggtagccgtatcggaaggtgcggctggatcacctcctttctaaggag

SEQ ID NO: 168 - DNA, 1521 bp, Micromonas micros
  misc_feature: Micromonas micros ACM 5086
tagagagtttgatcctggctcaggacgaacgctggcggcgtgcttaacacatgcaagtcgaacgtgatttttgtggaaattctttcgggaatggaaatgaaatgaaagtggcgaacgggtgagtaacacgtgagcaacctaccttacacagggggatagccgttggaaacgacgattaataccgcatgagaccacagaatcgcatgatataggggtcaaagatttatcggtgtaagaagggctcgcgtctgattagctagttggaagggtaaaggcctaccaaggcgacgatcagtagccggtctgagaggatgaacggccacattggaactgagacacggtccaaactcctacgggaggcagcagtggggaatattgcacaatggggggaaccctgatgcagcgacgccgcgtgagcgaagaaggttttcgaatcgtaaagctctgtcctatgagaagataatgacggtatcataggaggaagccccggctaaatacgtgccagcagccgcggtaatacgtatggggcgagcgttgtccggaattattgggcgtaaagggtacgtaggcggttttttaagtcaggtgtgaaagcgtgaggcttaacctcattaagcacttgaaactggaagacttgagtgaaggagaggaaagtggaattcctagtgtagcggtgaaatgcgtagatattaggaggaataccggtggcgaaggcgactttctggacttttactgacgctcaggtacgaaagcgtggggagcaaacaggattagataccctggtagtccacgccgtaaacgatgaatgctaggtgttgggagtcaaatctcggtgccgaagttaacacattaagcattccgcctggggagtacggtggcaacactgaaactcaaaggaattgacggggacccgcacaagcagcggagcatgtggtttaattcgaagcaacgcgaagaaccttaccaaggcttgacatatagttgagttattgagaaattgataagtccctcgggacaactatacaggtggtgcatggttgtcgtcagctcgtgtcgtgagatgttgggttaagtcccgcaacgagcgcaacccttatcttcagttgccagcacgtagaggtgggaactctggagagactgccgatgacaaatcggaggaaggtggggatgacgtcaaatcatcatgccctttatgtcttgggctacacacgtgctacaatggttggtacaacgagaagcgagatagagatgttaagcgaaactctaaaaaccaatctcagttcggattgtaggctgcaactcgcctacatgaagtcggagttgctagtaatcgcgaatcagaatgtcgcggtgaatgcgttcccgggtcttgtacacaccgcccgtcacaccatgggagttggcaatacccgaagccgccgatctaaccgcaaggaggaaggcgtcgaaggtagggttaatgactggggtgaagtcgtaacaaggtagccgtatcggaaggtgcggctggatcacctcctt

SEQ ID NO: 169 - DNA, 1349 bp, Micromonas micros
  misc_feature: Micromonas micros ACM 5086
agtggcgaacgggtgagtaacacgtgagcaacctaccttacacagggggatagccgttggaaacgacgattaataccgcatgagaccacagaatcgcatgatataggggtcaaagatttatcggtgtaagaagggctcgcgtctgattagctagttggaagggtaaaggcctaccaaggcgacgatcagtagccggtctgagaggatgaacggccacattggaactgagacacggtccaaactcctacgggaggcagcagtggggaatattgcacaatggggggaaccctgatgcagcgacgccgcgtgagcgaagaaggttttcgaatcgtaaagctctgtcctatgagaagataatgacggtatcataggaggaagccccggctaaatacgtgccagcagccgcggtaatacgtatggggcgagcgttgtccggaattattgggcgtaaagggtacgtaggcggttttttaagtcaggtgtgaaagcgtgaggcttaacctcattaagcacttgaaactggaagacttgagtgaaggagaggaaagtggaattcctagtgtagcggtgaaatgcgtagatattaggaggaataccggtggcgaaggcgactttctggacttttactgacgctcaggtacgaaagcgtggggagcaaacaggattagataccctggtagtccacgccgtaaacgatgaatgctaggtgttgggagtcaaatctcggtgccgaagttaacacattaagcattccgcctggggagtacggtggcaacactgaaactcaaaggaattgacggggacccgcacaagcagcggagcatgtggtttaattcgaagcaacgcgaagaaccttaccaaggcttgacatatagttgagttattgagaaattgataagtccctcgggacaactatacaggtggtgcatggttgtcgtcagctcgtgtcgtgagatgttgggttaagtcccgcaacgagcgcaacccttatcttcagttgccagcacgtagaggtgggaactctggagagactgccgatgacaaatcggaggaaggtggggatgacgtcaaatcatcatgccctttatgtcttgggctacacacgtgctacaatggttggtacaacgagaagcgagatagagatgttaagcgaaactctaaaaaccaatctcagttcggattgtaggctgcaactcgcctacatgaagtcggagttgctagtaatcgcgaatcagaatgtcgcggtgaatgcgttcccgggtcttgtacacaccgcccgtcacaccatgggagttggcaatacccgaagccgccgatctaaccgcaaggaggaaggcgtcgaaggtagg

SEQ ID NO: 170 - DNA, 1530 bp, Micromonas micros
  misc_feature: Micromonas micros ACM 5086
tttaaatagagagtttgatcctggctcaggacgaacgctggcggcgtgcttaacacatgcaagtcgaacgtgatttttgtggaaattctttcgggaatggaaatgaaatgaaagtggcgaacgggtgagtaacacgtgagcaacctaccttacacagggggatagccgttggaaacgacgattaataccgcatgagaccacagaatcgcatgatataggggtcaaagatttatcggtgtaagaagggctcgcgtctgattagctagttggaagggtaaaggcctaccaaggcgacgatcagtagccggtctgagaggatgaacggccacattggaactgagacacggtccaaactcctacgggaggcagcagtggggaatattgcacaatggggggaaccctgatgcagcgacgccgcgtgagcgaagaaggttttcgaatcgtaaagctctgtcctatgagaagataatgacggtatcataggaggaagccccggctaaatacgtgccagcagccgcggtaatacgtatggggcgagcgttgtccggaattattgggcgtaaagggtacgtaggcggttttttaagtcaggtgtgaaagcgtgaggcttaacctcattaagcacttgaaactggaagacttgagtgaaggagaggaaagtggaattcctagtgtagcggtgaaatgcgtagatattaggaggaataccggtggcgaaggcgactttctggacttttactgacgctcaggtacgaaagcgtggggagcaaacaggattagataccctggtagtccacgccgtaaacgatgaatgctaggtgttgggagtcaaatctcggtgccgaagttaacacattaagcattccgcctggggagtacggtggcaacactgaaactcaaaggaattgacggggacccgcacaagcagcggagcatgtggtttaattcgaagcaacgcgaagaaccttaccaaggcttgacatatagttgagttattgagaaattgataagtccctcgggacaactatacaggtggtgcatggttgtcgtcagctcgtgtcgtgagatgttgggttaagtcccgcaacgagcgcaacccttatcttcagttgccagcacgtagaggtgggaactctggagagactgccgatgacaaatcggaggaaggtggggatgacgtcaaatcatcatgccctttatgtcttgggctacacacgtgctacaatggttggtacaacgagaagcgagatagagatgttaagcgaaactctaaaaaccaatctcagttcggattgtaggctgcaactcgcctacatgaagtcggagttgctagtaatcgcgaatcagaatgtcgcggtgaatgcgttcccgggtcttgtacacaccgcccgtcacaccatgggagttggcaatacccgaagccgccgatctaaccgcaaggaggaaggcgtcgaaggtagggttaatgactggggtgaagtcgtaacaaggtagccgtatcggaaggtgcggctggatcacctcctttct

SEQ ID NO: 171 - DNA, 1481 bp, Micromonas micros
  misc_feature: Micromonas micros ACM 5086
gacgaacgctggcggcgtgcttaacacatgcaagtcgaacgtgatttttgtggaaattctttcgggaatggaaatgaaatgaaagtggcgaacgggtgagtaacacgtgagcaacctaccttacacagggggatagccgttggaaacgacgattaataccgcatgagaccacagaatcgcatgatataggggtcaaagatttatcggtgtaagaagggctcgcgtctgattagctagttggaagggtaaaggcctaccaaggcgacgatcagtagccggtctgagaggatgaacggccacattggaactgagacacggtccaaactcctacgggaggcagcagtggggaatattgcacaatggggggaaccctgatgcagcgacgccgcgtgagcgaagaaggttttcgaatcgtaaagctctgtcctatgagaagataatgacggtatcataggaggaagccccggctaaatacgtgccagcagccgcggtaatacgtatggggcgagcgttgtccggaattattgggcgtaaagggtacgtaggcggttttttaagtcaggtgtgaaagcgtgaggcttaacctcattaagcacttgaaactggaagacttgagtgaaggagaggaaagtggaattcctagtgtagcggtgaaatgcgtagatattaggaggaataccggtggcgaaggcgactttctggacttttactgacgctcaggtacgaaagcgtggggagcaaacaggattagataccctggtagtccacgccgtaaacgatgaatgctaggtgttgggagtcaaatctcggtgccgaagttaacacattaagcattccgcctggggagtacggtggcaacactgaaactcaaaggaattgacggggacccgcacaagcagcggagcatgtggtttaattcgaagcaacgcgaagaaccttaccaaggcttgacatatagttgagttattgagaaattgataagtccctcgggacaactatacaggtggtgcatggttgtcgtcagctcgtgtcgtgagatgttgggttaagtcccgcaacgagcgcaacccttatcttcagttgccagcacgtagaggtgggaactctggagagactgccgatgacaaatcggaggaaggtggggatgacgtcaaatcatcatgccctttatgtcttgggctacacacgtgctacaatggttggtacaacgagaagcgagatagagatgttaagcgaaactctaaaaaccaatctcagttcggattgtaggctgcaactcgcctacatgaagtcggagttgctagtaatcgcgaatcagaatgtcgcggtgaatgcgttcccgggtcttgtacacaccgcccgtcacaccatgggagttggcaatacccgaagccgccgatctaaccgcaaggaggaaggcgtcgaaggtagggttaatgactggggtgaagtcctaacaaggtagccgtatcggaaggtgc

SEQ ID NO: 172 - DNA, 1483 bp, Paraprevotella clara
  misc_feature: Paraprevotella clara DSM 19731
gatgaacgctagctacaggcttaacacatgcaagtcgaggggcagcatgrrcycagctttgctgggtttgatggcgaccggcgcacgggtgagtaacgcgtatccaacctgccctttactccgggatagtctcctgaaagggagtttaataccggatgtgtttgtctttccgcatgggagcgacaaataaagattgattggtaaaggatggggatgcgtcccattagctrgttggcggggtaacggcccaccaaggcracgatgggtaggggttctgagaggaaggtcccccacattggaactgagacacggtccaaactcctacgggaggcagcagtgaggaatattggtcaatgggcgagagcctgaaccagccaagtagcgtgaaggacgacggccctacgggttgtaaacttcttttataagggaataaagttcgccacgcgtggtgttttgtatgtaccttatgaataagcatcggctaattccgtgccagcagccgcggtaatacggaagatgcgagcgttatccggatttattgggtttaaagggagcgtaggcgggcttttaagtcagcggtcaaatgtcacggctcaaccgtggccagccgttgaaactgtaagccttgagtctgcacagggcacatggaattcgtggtgtagcggtgaaatgcttagatatcacgaagaactccgatcgcgaaggcattgtgccggggcagcactgacgctgaggctcgaaagtgcgggtatcaaacaggattagataccctggtagtccgcacggtaaacgatgaatgctcgctatgggcgatayawtgtccgtggccaagcgaaagcgttaagcattccacctggggagtacgccggcaacggtgaaactcaaaggaattgacgggggcccgcacaagcggaggaacatgtggtttaattcgatgatacgcgaggaaccttacccgggcttgaattgcaggtgcatgagtcagagatgattctttccttcgggactcctgtgaaggtgctgcatggttgtcgtcagctcgtgccgtgaggtgtcggcttaagtgccataacgagcgcaacccttctccccagttgccatcgggtaatgccgggccctctggggacactgccatcgtaagatgcgaggaaggtggggatgacgtcaaatcagcacggcccttacgtccggggctacacacgtgttacaatggggggtacagagggccgctgtccggtgacggttggccaatccctaaaacccctctcagttcggactggagtctgcaacccgactccacgaagctggattcgctagtaatcgcgcatcagccatggcgcggtgaatacgttcccgggccttgtacacaccgcccgtcaagccatgaaagccgggggtgcctgaagtccgtgaccgcgagggtcggcctagggtaaaactggtgattggggctaagtcgtaacaaggtagccgtaccggaagg

SEQ ID NO: 173 - DNA, 1480 bp, Paraprevotella clara
  misc_feature: Paraprevotella clara DSM 19731
tggctcaggatgaacgctagctacaggcttaacacatgcaagtcgaggggcagcatggacccagctttgctgggtttgatggcgaccggcgcacgggtgagtaacgcgtatccaacctgccctttactccgggatagtctcctgaaagggagtttaataccggatgtgtttgtctttccgcatgggagcgacaaataaagattgattggtaaaggatggggatgcgtcccattagctagttggcggggtaacggcccaccaaggcaacgatgggtaggggttctgagaggaaggtcccccacattggaactgagacacggtccaaactcctacgggaggcagcagtgaggaatattggtcaatgggcgagagcctgaaccagccaagtagcgtgaaggacgacggccctacgggttgtaaacttcttttataagggaataaagttcgccacgcgtggtgttttgtatgtaccttatgaataagcatcggctaattccgtgccagcagccgcggtaatacggaagatgcgagcgttatccggatttattgggtttaaagggagcgtaggcgggcttttaagtcagcggtcaaatgtcacggctcaaccgtggccagccgttgaaactgtaagccttgagtctgcacagggcacatggaattcgtggtgtagcggtgaaatgcttagatatcacgaagaactccgatcgcgaaggcattgtgccggggcagcactgacgctgaggctcgaaagtgcgggtatcaaacaggattagataccctggtagtccgcacggtaaacgatgaatgctcgctatgggcgatacattgtccgtggccaagcgaaagcgttaagcattccacctggggagtacgccggcaacggtgaaactcaaaggaattgacgggggcccgcacaagcggaggaacatgtggtttaattcgatgatacgcgaggaaccttacccgggcttgaattgcaggtgcatgagtcagagatgattctttccttcgggactcctgtgaaggtgctgcatggttgtcgtcagctcgtgccgtgaggtgtcggcttaagtgccataacgagcgcaacccttctccccagttgccatcgggtaatgccgggccctctggggacactgccatcgtaagatgcgaggaaggtggggatgacgtcaaatcagcacggcccttacgtccggggctacacacgtgttacaatggggggtacagagggccgctgtccggtgacggttggccaatccctaaaacccctctcagttcggactggagtctgcaacccgactccacgaagctggattcgctagtaatcgcgcatcagccatggcgcggtgaatacgttcccgggccttgtacacaccgcccgtcaagccatgaaagccgggggtgcctgaagtccgtgaccgcgagggtcggcctagggtaaaactggtgattggggctaagtcgtaacaaggtaacc

SEQ ID NO: 174 - DNA, 1366 bp, Peptostreptococcus stomatis
  misc_feature: Peptostreptococcus stomatis W2278
  misc_feature (1354)..(1354): n is a, c, g, or t
agcggcggacgggtgagtaacgcgtgggtaacctgccctatacacatggataacatactgaaaagtttactaatacatgataatatatatttgcggcatcgcagatatatcaaagtgttagcggtataggatggacccgcgtctgattagctagttggtgagataactgcccaccaaggcgacgatcagtagccgacctgagagggtgatcggccacattggaactgagacacggtccaaactcctacgggaggcagcagtggggaatattgcacaatgggcgaaagcctgatgcagcaacgccgcgtgaacgatgaaggtcttcggatcgtaaagttctgttgcaggggaagataatgacggtaccctgtgaggaagccccggctaactacgtgccagcagccgcggtaatacgtagggggctagcgttatccggatttactgggcgtaaagggtgcgtaggtggtccttcaagtcggtggttaaaggctacggctcaaccgtagtaagccgccgaaactggaggacttgagtgcaggagaggaaagtggaattcccagtgtagcggtgaaatgcgtagatattgggaggaacaccagtagcgaaggcggctttctggactgcaactgacactgaggcacgaaagcgtgggtagcaaacaggattagataccctggtagtccacgctgtaaacgatgagtactaggtgtcgggggttacccccctcggtgccgcagctaacgcattaagtactccgcctggggagtacgcacgcaagtgtgaaactcaaaggaattgacggggacccgcacaagtagcggagcatgtggtttaattcgaagcaacgcgaagaaccttacctaagcttgacatccctcggacaggtgtttaatcacacccttccttcgggactgaggtgacaggtggtgcatggttgtcgtcagctcgtgtcgtgagatgttgggttaagtcccgcaacgagcgcaacccttgtctttagttgccagcattcagttgggcactctagagagactgccagggataacctggaggaaggtggggatgacgtcaaatcatcatgccccttatgcttagggctacacacgtgctacaatgggtggtacagagggttgccaaaccgtgaggtggagccaatcccttaaagccattctcagttcggattgtaggctgaaactcgcctacatgaagctggagttactagtaatcgcagatcagaatgctgcggtgaatgcgttcccgggtcttgtacacaccgcccgtcacaccacgggagtcggaaacacccgaagccgattatccaaccgcaaggaggaagtcgtcgaaggtggcgtcnataactggggtg

SEQ ID NO: 175 - DNA, 1419 bp, Peptostreptococcus stomatis
  misc_feature: Peptostreptococcus stomatis W2278
agcggcggacgggtgagtaacgcgtgggtaacctgccctatacacatggataacatactgaaaagtttactaatacatgataatatatatttgcggcatcgcagatatatcaaagtgttagcggtataggatggacccgcgtctgattagctagttggtgagataactgcccaccaaggcgacgatcagtagccgacctgagagggtgatcggccacattggaactgagacacggtccaaactcctacgggaggcagcagtggggaatattgcacaatgggcgaaagcctgatgcagcaacgccgcgtgaacgatgaaggtcttcggatcgtaaagttctgttgcaggggaagataatgacggtaccctgtgaggaagccccggctaactacgtgccagcagccgcggtaatacgtagggggctagcgttatccggatttactgggcgtaaagggtgcgtaggtggtccttcaagtcggtggttaaaggctacggctcaaccgtagtaagccgccgaaactggaggacttgagtgcaggagaggaaagtggaattcccagtgtagcggtgaaatgcgtagatattgggaggaacaccagtagcgaaggcggctttctggactgcaactgacactgaggcacgaaagcgtgggtagcaaacaggattagataccctggtagtccacgctgtaaacgatgagtactaggtgtcgggggttacccccctcggtgccgcagctaacgcattaagtactccgcctggggagtacgcacgcaagtgtgaaactcaaaggaattgacggggacccgcacaagtagcggagcatgtggtttaattcgaagcaacgcgaagaaccttacctaagcttgacatccctcggacaggtgtttaatcacacccttccttcgggactgaggtgacaggtggtgcatggttgtcgtcagctcgtgtcgtgagatgttgggttaagtcccgcaacgagcgcaacccttgtctttagttgccagcattcagttgggcactctagagagactgccagggataacctggaggaaggtggggatgacgtcaaatcatcatgccccttatgcttagggctacacacgtgctacaatgggtggtacagagggttgccaaaccgtgaggtggagccaatcccttaaagccattctcagttcggattgtaggctgaaactcgcctacatgaagctggagttactagtaatcgcagatcagaatgctgcggtgaatgcgttcccgggtcttgtacacaccgcccgtcacaccacgggagtcggaaacacccgaagccgattatccaaccgcaaggaggaagtcgtcgaaggtggcgtcgataactggggtgaagtcgtaacaaggtagccgtatcggaaggtgcggctggatcacctcctttct

SEQ ID NO: 176 - DNA, 1419 bp, Peptostreptococcus stomatis; identical to SEQ ID NO: 175
  misc_feature: Peptostreptococcus stomatis W2278

SEQ ID NO: 177 - DNA, 1466 bp, Porphyromonas somerae
  misc_feature: Porphyromonas somerae WAL 6690
  misc_feature (185)..(185): n is a, c, g, or t
  misc_feature (999)..(999): n is a, c, g, or t
ctcaggatgaacgctagcgataggcttaacacatgcaagtcgaggggtaacatggagagaagcttgcttcactctgatgacgaccggcggatgggtgagtaacgcgtatgcaacttgcctcttagtgggggataacccggagaaatccggactaataccgcatattcttaatttctcgcatgagntattaaggaaagatttattgctaagagataggcatgcgttccattagttagttggtgaggtaacggctcaccaaggcatcgatggataggggagctgagaggctgatcccccacattgggactgagacacggcccaaactcctacgggaggcagcagtgaggaatattggtcaatggaggaaactctgaaccagccaagtcgcgtgaaggatgacggtcctatggattgtaaacttctttagtaggtgagtaaggataggtacgtgtacctattagaaagtagcctaagaataagtatcggctaactccgtgccagcagccgcggtaatacggaggatacgagcgttatccggatttattgggtttaaagggtgcgtaggtggctgattaagtcagtggtgaaaagctatggctcaaccatagtcttgccgttgatactggttagcttgagtgtagatgatgtaggcggaatgcgtagtgtagcggtgaaatgcatagatattacgcagaactccgattgcgaaggcagcttacaaaactactactgacactgaggcacgaaagcgtgggtatcaaacaggattagataccctggtagtccacgccgtaaactatgattactcagagtatgcgatatacagtatgctctcaagcgaaagcattaagtaatccacctggggagtacgtcggcaacgatgaaactcaaaggaattgacgggggcccgcacaagcggaggaacatgtggtttaattcgatgatacgcgaggaaccttacctgggattgaaatgtataggctggcaactggaaacagntgctgttcacttcggtgacttatatgtaggtgctgcatggttgtcgtcagctcgtgccgtgaggtgtcggcttaagtgccataacgagcgcaacccttatcgttagttgctaacaggttaagctgaggactctggcgagactgccgtcgtaaggcgagaggaaggtggggatgacgtcaaatcagcacggcccttatatccagggcgacacacgtgttacaatggtgaggacaaagggtagctacctggtgacaggatgccaatctccaaacctcatctcagttcggatcgaagtctgcaactcgacttcgtgaagctggattcgctagtaatcgcgcatcagccatggcgcggtgaatacgttcccgggccttgtacacaccgcccgtcaagccatggaagctgggggtacctgaagtgcgtgaccgcgaggagcgtccgagggtaagaccggtgactggggctaa

SEQ ID NO: 178 - DNA, 1486 bp, Porphyromonas somerae
  misc_feature: Porphyromonas somerae WAL 6690
tggctcaggatgaacgctagcgataggcttaacacatgcaagtcgaggggtaacatggagagaagcttgcttcactctgatgacgaccggcggatgggtgagtaacgcgtatgcaacttgcctcttagtgggggataacccggagaaatccggactaataccgcatattcttaatttctcgcatgagttattaaggaaagatttattgctaagagataggcatgcgttccattagttagttggtgaggtaacggctcaccaaggcatcgatggataggggagctgagaggctgatcccccacattgggactgagacacggcccaaactcctacgggaggcagcagtgaggaatattggtcaatggaggaaactctgaaccagccaagtcgcgtgaaggatgacggtcctatggattgtaaacttctttagtaggtgagtaaggataggtacgtgtacctattagaaagtagcctaagaataagtatcggctaactccgtgccagcagccgcggtaatacggaggatacgagcgttatccggatttattgggtttaaagggtgcgtaggtggctgattaagtcagtggtgaaaagctatggctcaaccatagtcttgccgttgatactggttagcttgagtgtagatgatgtaggcggaatgcgtagtgtagcggtgaaatgcatagatattacgcagaactccgattgcgaaggcagcttacaaaactactactgacactgaggcacgaaagcgtgggtatcaaacaggattagataccctggtagtccacgccgtaaactatgattactcagagtatgcgatatacagtatgctctcaagcgaaagcattaagtaatccacctggggagtacgtcggcaacgatgaaactcaaaggaattgacgggggcccgcacaagcggaggaacatgtggtttaattcgatgatacgcgaggaaccttacctgggattgaaatgtataggctggcaactggaaacagatgctgttcacttcggtgacttatatgtaggtgctgcatggttgtcgtcagctcgtgccgtgaggtgtcggcttaagtgccataacgagcgcaacccttatcgttagttgctaacaggttaagctgaggactctggcgagactgccgtcgtaaggcgagaggaaggtggggatgacgtcaaatcagcacggcccttatatccagggcgacacacgtgttacaatggtgaggacaaagggtagctacctggtgacaggatgccaatctccaaacctcatctcagttcggatcgaagtctgcaactcgacttcgtgaagctggattcgctagtaatcgcgcatcagccatggcgcggtgaatacgttcccgggccttgtacacaccgcccgtcaagccatggaagctgggggtacctgaagtgcgtgaccgcgaggagcgtccgagggtaagaccggtgactggggctaagtcgtaacaaggtaacc

SEQ ID NO: 179 - DNA, 1444 bp, Roseburia inulinivorans
  misc_feature: Roseburia inulinivorans A2-194
ttgatcctggctcaggatgaacgctggcggcgtgcttaacacatgcaagtcgaacgaagcacttttacagatttcttcggaatgaagttttagtgactgagtggcggacgggtgagtaacgcgtgggtaacctgcctcacacagggggataacagttggaaacggctgctaataccgcataagcgcacagtaccgcatggtacagtgtgaaaaactccggtggtgtgagatggacccgcgtctgattagctagttggcagggcaacggcctaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaaatgacggtacctgactaagaagcaccggctaaatacgtgccagcagccgcggtaatacgtatggtgcaagcgttatccggatttactgggtgtaaagggagcgcaggcggaaggctaagtctgatgtgaaagcccggggctcaaccccggtactgcattggaaactggtcatctagagtgtcggaggggtaagtggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgtcggaaagcacagcttttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggacagtaatgtgtcctttccttcgggacagaagtgacaggtggtgcatggttgtcgtcagctcgtgtcgtgagatgttgggttaagtcccgcaacgagcgcaacccttatccccagtagccagcggttcggacgggcactctgaggagactgccagggataacctggaggaaggtggggatgacgtcaaatcatcatgccccttatgacttgggctacacacgtgctacaatggcgtaaacaaagggaagcgagaccgtgaggtggagcaaatcccaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttggaaatgcccgaagtcagtgacccaaccgcaaggagggagctgc

SEQ ID NO: 180 - DNA, 1423 bp, Ruminococcus gnavus
  misc_feature: Ruminococcus gnavus VPI C7-9
  misc_feature (1148)..(1148): n is a, c, g, or t
  misc_feature (1188)..(1188): n is a, c, g, or t
  misc_feature (1257)..(1257): n is a, c, g, or t
cctggctcaggatgaacgctggcggcgtgcttaacacatgcaagtcgagcgaagcaccttgacggatttcttcggattgaagccttggtgactgagcggcggacgggtgagtaacgcgtgggtaacctgcctcatacagggggataacagttggaaacggctgctaataccgcataagcgcacagtaccgcatggtacggtgtgaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgtcgggtggaaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccctctgaccgctctttaatcggagctttccttcgggacagaggagacaggtggtgcatggttgtcgtcagctcgtgtcgtgagatgttgggttaagtcccgcaacgagcgcaacccctatctttagtagccagcattttggatgggcactctagagagactvccagggataacctggnggaaggtggggatgacgtcaaatcatcatgccccttatgnccagggctacacacgtgctacaatggcgtaaacaaagggaagcgagcccgcgagggggagcaaatcccnaaaataacgtctcagttcggattgtagtctgcaactcgactacatgaagctggaatcgctagtaatcgcgaatcagaatgtcgcggtgaatacgttcccgggtcttgtacacaccscccgtcacaccatgggagtmagtaacgcccgaagtcagtgacccaaccgc

SEQ ID NO: 181 - DNA, 1481 bp, Ruminococcus gnavus
  misc_feature: Ruminococcus gnavus VPI C7-9
  misc_feature (148)..(148): n is a, c, g, or t
  misc_feature (1071)..(1071): n is a, c, g, or t
  misc_feature (1341)..(1341): n is a, c, g, or t
tgaacgctggcggcgtgcttaacacatgcaagtcgagcgaagcaccttgacggatttcttcggattgaagccttggtgactgagcggcggacgggtgagtaacgcgtgggtaacctgcctcatacagggggataacagttggaaacgnctgctaataccgcataagcgcacagtaccgcatggtacggtgtgaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccctctgaccgctctttaatcggagctttccttcgggacagaggagacaggtggtgcatggttgtcgtcagctcgtgtcgtgagatgttgggttaagtcccgcaacgagcgcancccctatctttagtagccagcattttggatgggcactctagagagactgccagggataacctggaggaaggtggggatgacgtcaaatcatcatgccccttatgaccagggctacacacgtgctacaatggcgtaaacaaagggaagcgagcccgcgagggggagcaaatcccaaaaataacgtctcagttcggattgtagtctgcaactcgactacatgaagctggaatcgctagtaatcgcgaatcagaatgtcgcggtgaatacgtncccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcggaagg

SEQ ID NO: 182 - DNA, 1527 bp, Ruminococcus gnavus
  misc_feature: Ruminococcus gnavus VPI C7-9
gagagtttgatcctggctcaggatgaacgctggcggcgtgcttaacacatgcaagtcgagcgaagcaccttgacggatttcttcggattgaagccttggtgactgagcggcggacgggtgagtaacgcgtgggtaacctgcctcgtacagggggataacagttggaaacggctgctaataccgcataagcgcacagtaccgcatggtacggtgtgaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccctctgaccgctctttaatcggagctttccttcgggacagaggagacaggtggtgcatggttgtcgtcagctcgtgtcgtgagatgttgggttaagtcccgcaacgagcgcaacccctatctttagtagccagcattttggatgggcactctagagagactgccagggataacctggaggaaggtggggatgacgtcaaatcatcatgccccttatgaccagggctacacacgtgctacaatggcgtaaacaaagggaagcgagcccgcgagggggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcggaaggtgcggctggatcacctcctttct

SEQ ID NO: 183 - DNA, 1527 bp, Ruminococcus gnavus
  misc_feature: Ruminococcus gnavus VPI C7-9
gagagtttgatcctggctcaggatgaacgctggcggcgtgcttaacacatgcaagtcgagcgaagcaccttgacggatttcttcggattgaagccttggtgactgagcggcggacgggtgagtaacgcgtgggtaacctgcctcatacagggggataacagttggaaacggctgctaataccgcataagcgcacagtaccgcatggtacggtgtgaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccctctgaccgctctttaatcggagctttccttcgggacagaggagacaggtggtgcatggttgtcgtcagctcgtgtcgtgagatgttgggttaagtcccgcaacgagcgcaacccctatctttagtagccagcattttggatgggcactctagagagactgccagggataacctggaggaaggtggggatgacgtcaaatcatcatgccccttatgaccagggctacacacgtgctacaatggcgtaaacaaagggaagcgagcccgcgagggggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcggaaggtgcggctggatcacctcctttct

SEQ ID NO: 184 - DNA, 1527 bp, Ruminococcus gnavus; identical to SEQ ID NO: 182
  misc_feature: Ruminococcus gnavus VPI C7-9

SEQ ID NO: 185 - DNA, 1527 bp, Ruminococcus gnavus; identical to SEQ ID NO: 183
  misc_feature: Ruminococcus gnavus VPI C7-9

SEQ ID NO: 186 - DNA, 1487 bp, Shigella dysenteriae
  misc_feature: Shigella dysenteriae CECT 584
tggctcagattgaacgctggcggcaggcctaacacatgcaagtcgaacggtaacagaaagcagcttgctgtttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaaaactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacagaaccttgtagagatacgagggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgtcacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggta

SEQ ID NO: 187 - DNA, 1426 bp, Subdoligranulum variabile
  misc_feature: Subdoligranulum variabile BI-114
  misc_feature (1189)..(1189): n is a, c, g, or t
tgcaagtcgaacggagttatttcggttgaagttttcggatggatactggtttaacttagtggcgaacgggtgagtaacgcgtgagtaacctgccctggagtgggggacaacagttggaaacgactgctaataccgcataagcccacgatccggcatcggattgagggaaaaggatttattcgcttcaggatggactcgcgtccaattagctagttggtgaggtaacggcccaccaaggcgacgattggtagccggactgagaggttgaacggccacattgggactgagacacggcccagactcctacgggaggcagcagtgggggatattgcacaatgggggaaaccctgatgcagcgacgccgcgtggaggaagaaggttttcggattgtaaactcctgtcgttagggacgaatcttgacggtacctaacaagaaagcaccggctaactacgtgccagcagccgcggtaaaacgtagggtgcaagcgttgtccggaattactgggtgtaaagggagcgcaggcggaccggcaagttggaagtgaaatctatgggctcaacccataaattgctttcaaaactgctggccttgagtagtgcagaggtaggtggaattcccggtgtagcggtggaatgcgtagatatcgggaggaacaccagtggcgaaggcgacctactgggcaccaactgacgctgaggctcgaaagcatgggtagcaaacaggattagataccctggtagtccatgccgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccgatgcatagtgcagagatgcatgaagtccttcgggacatcgagacaggtggtgcatggttgtcgtcagctcgtgtcgtgagatgttgggttaagtcccgcaacgagcgcaacccttattgccagttactacgcaagaggactctggcgagactgccgttgacaaaacggaggaaggtggggatgacgtcaaatcatcatgccctttatgacctgggctacacacgtactacaatggcgtttaacaaagagangcaagaccgcgaggtggagcaaaactcaaaaacaacgtctcagttcagattgcaggctgcaactcgcctgcatgaagtcggaattgctagtaatcgcggatcagcatgccgcggtgaatacgttcccgggccttgtacacaccgcccgtcacaccatgagagccgggggggacccgaagtcggtaagtaagtctaaccgcaaggaggacgccgccgaaggtaaaactggtgattggg

SEQ ID NO: 188 - DNA, 1524 bp, Subdoligranulum variabile
  misc_feature: Subdoligranulum variabile BI-114
agagtttgatcctggctcaggacgaacgctggcggcgcgcctaacacatgcaagtcgaacggagttatttcggttgaagttttcggatggatactggtttaacttagtggcgaacgggtgagtaacgcgtgagtaacctgccctggagtgggggacaacagttggaaacgactgctaataccgcataagcccacggcccggcatcgggctgagggaaaaggatttattcgcttcaggatggactcgcgtccaattagctagttggtgaggtaacggcccaccaaggcgacgattggtagccggactgagaggttgaacggccacattgggactgagacacggcccagactcctacgggaggcagcagtgggggatattgcacaatgggggaaaccctgatgcagcgacgccgcgtggaggaagaaggttttcggattgtaaactcctgtcgttagggacgaatcttgacggtacctaacaagaaagcaccggctaactacgtgccagcagccgcggtaaaacgtagggtgcaagcgttgtccggaattactgggtgtaaagggagcgcaggcggaccggcaagttggaagtgaaatctatgggctcaacccataaattgctttcaaaactgctggccttgagtagtgcagaggtaggtggaattcccggtgtagcggtggaatgcgtagatatcgggaggaacaccagtggcgaaggcgacctactgggcaccaactgacgctgaggctcgaaagcatgggtagcaaacaggattagataccctggtagtccatgccgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccgatgcatagtgcagagatgcatgaagtccttcgggacatcgagacaggtggtgcatggttgtcgtcagctcgtgtcgtgagatgttgggttaagtcccgcaacgagcgcaacccttattgccagttactacgcaagaggactctggcgagactgccgttgacaaaacggaggaaggtggggatgacgtcaaatcatcatgccctttatgacctgggctacacacgtactacaatggcgtttaacaaagagaagcaagaccgcgaggtggagcaaaactcaaaaacaacgtctcagttcagattgcaggctgcaactcgcctgcatgaagtcggaattgctagtaatcgcggatcagcatgccgcggtgaatacgttcccgggccttgtacacaccgcccgtcacaccatgagagccggggggacccgaagtcggtagtctaaccgcaaggaggacgccgccgaaggtaaaactggtgattggggtgaagtcgtaacaaggtagccgtatcggaaggtgcggctggatcacctcctttct

SEQ ID NO: 189 - DNA, 1495 bp, Veillonella alcalescens
  misc_feature: Veillonella alcalescens 259
gctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatctaatctcggcatcgaggatagatgaaaggtggcctctacatgtaagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaaaggccttcttgcgaatagtgagaaggattgacggtaccggaatagaaagccacggctaactacgtgccagcagccgcggtaatacgtaggtggcaagcgttgtccggaattattgggcgtaaagcgcgcgcaggcggatmggtcagtctgtcttaaaagttcggggcttaaccccgtgatgggatggaaactgccaatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaaccagagatggttcctcttcttcggaagccagaaaacaggtggtgcacggttgtcgtcagctcgtgtcgtgagatgttgggttaagtcccgcaacgagcgcaacccctatcttatgttgccagcactttgggtggggactcatgagagactgccgcagacaatgcggaggaaggcggggatgacgtcaaatcatcatgccccttatgacctgggctacacacgtactacaatgggagttaatagacggaagcgagatcgcgagatggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtgaagtcgtaacaaggtag

SEQ ID NO: 190 - DNA, 1515 bp, Veillonella alcalescens
  misc_feature: Veillonella alcalescens 259
cgaacgctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatctaatctcggcatcgaggatagatgaaaggtggcctctacatgtaagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaaaggccttcttgcgaatagtgagaaggattgacggtaccggaatagaaagccacggctaactacgtgccagcagccgcggtaatacgtaggtggcaagcgttgtccggaattattgggcgtaaagcgcgcgcaggcggatcaggtcagtctgtcttaaaagttcggggcttaaccccgtgatgggatggaaactgccaatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaaccagagatggttcctcttcttcggaagccagaaaacaggtggtgcacggttgtcgtcagctcgtgtcgtgagatgttgggttaagtcccgcaacgagcgcaacccctatcttatgttgccagcactttgggtggggaactcatgagagactgccgcagacaatgcggaggaaggcggggatgacgtcaaatcatcatgccccttatgacctgggctacacacgtactacaatgggagttaatagacggaagcgagatcgcgagatggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtgaagtcgtaacaaggtagccgtatcggaagg

SEQ ID NO: 191 - DNA, 1571 bp, Veillonella alcalescens
  misc_feature: Veillonella alcalescens 259
ttggagagtttgatcctggctcaggacgaacgctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatctaatctcggcatcgaggatagatgaaaggtggcctctacatgtaagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaaaggccttcttgcgaatagtgagaaggattgacggtaccggaatagaaagccacggctaactacgtgccagcagccgcggtaatacgtaggtggcaagcgttgtccggaattattgggcgtaaagcgcgcgcaggcggatcaggtcagtctgtcttaaaagttcggggcttaaccccgtgatgggatggaaactgccaatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaaccagagatggttcctcttcttcggaagccagaaaacaggtggtgcacggttgtcgtcagctcgtgtcgtgagatgttgggttaagtcccgcaacgagcgcaacccctatcttatgttgccagcactttgggtggggaactcatgagagactgccgcagacaatgcggaggaaggcggggatgacgtcaaatcatcatgccccttatgacctgggctacacacgtactacaatgggagttaatagacggaagcgagatcgcgagatggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtgaagtcgtaacaaggtagccgtatcggaaggtgcggctggatcacctcctttctagggaga

SEQ ID NO: 192 - DNA, 1564 bp, Veillonella alcalescens
  misc_feature: Veillonella alcalescens 259
ttggagagtttgatcctggctcaggacgaacgctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatctaatctcggcatcgaggatagatgaaaggtggcctctacatgtaagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaaaggccttcttgcgaatagtgagaaggattgacggtaccggaatagaaagccacggctaactacgtgccagcagccgcggtaatacgtaggtggcaagcgttgtccggaattattgggcgtaaagcgcgcgcaggcggatcaggtcagtctgtcttaaaagttcggggcttaaccccgtgatgggatggaaactgccaatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaaccagagatggttcctcttcttcggaagccagaaaacaggtggtgcacggttgtcgtcagctcgtgtcgtgagatgttgggttaagtcccgcaacgagcgcaacccctatcttatgttgccagcactttgggtggggaactcatgagagactgccgcagacaatgcggaggaaggcggggatgacgtcaaatcatcatgccccttatgacctgggctacacacgtactacaatgggagttaatagacggaagcgagatcgcgagatggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtgaagtcgtaacaaggtagccgtatcggaaggtgcggctggatcacctcctttct